SEQUENCE LISTING (42 sequences)

SEQ ID NO: 1 - DNA, 604 bp, synthetic construct
  source 1..604: mol_type = other DNA
ggtcatttagaggaagtaaaagtcgtaacaaggtctccgttggtgaaccagcggagggatcattaccgagttttcaactccctaacccttctgtgaacctacctatcgttgcttcggcggactcgccccagcccggacgcggactggaccagcggcccgccggggacctcaaactcttgtattccagcatcttctgaatacgccgcaaggcaaaacaaatgaatcaaaactttcaacaacggatctcttggctctggcatcgatgaagaacgcagcgaaatgcgataagtaatgtgaattgcagaatccagtgaatcatcgaatctttgaacgcacattgcgcccgccagcattctggcgggcatgcctgttcgagcgtcatttcaaccctcgacctcccctgggggaggtcggcgttggggaccggcagcacaccgccggccctgaaatggagtggcggcccgtccgcggcgacctctgcgtagtaatacagctcgcaccggaaccccgacgcggccacgccgtaaaacacccaacttctgaacgttgacctcgaatcaggtaggactacccgctgaacttaagcatatcaataagcggagga

SEQ ID NO: 2 - DNA, 604 bp, synthetic construct; identical to SEQ ID NO: 1
  source 1..604: mol_type = other DNA

SEQ ID NO: 3 - DNA, 604 bp, synthetic construct; identical to SEQ ID NO: 1
  source 1..604: mol_type = other DNA

SEQ ID NO: 4 - DNA, 604 bp, synthetic construct; identical to SEQ ID NO: 1
  source 1..604: mol_type = other DNA

SEQ ID NO: 5 - DNA, 604 bp, synthetic construct
  source 1..604: mol_type = other DNA
ggtcatttagaggaagtaaaagtcgtaacaaggtctccgttggtgaaccagcggagggatcattaccgagttttcaactccctaacccttctgtgaacctacctatcgttgcttcggcggactcgccccagcccggacgcggactggaccagcggcccgccggggacctcaaactcttgtattccagcatcttctgaatacgccgcaaggcaaaacaaatgaatcaaaactttcaacaacggatctcttggctctggcatcgatgaagaacgcagcgaaacgcgataagtaatgtgaattgcagaatccagtgaatcatcgaatctttgaacgcacattgcgcccgccagcattctggcgggcatgcctgttcgagcgtcatttcaaccctcgacctccccaaggggaggtcggcgttggggaccggcagcacaccgccggccctgaaatggagtggcggcccgtccgcggcgacctctgcgcagtaatacagctcgcaccggaaccccgacgcggccacgccgtaaaacacccaacttctgaacgttgacctcgaatcaggtaggactacccgctgaacttaagcatatcaataagcggagga

SEQ ID NO: 6 - DNA, 604 bp, synthetic construct; identical to SEQ ID NO: 1
  source 1..604: mol_type = other DNA

SEQ ID NO: 7 - DNA, 604 bp, synthetic construct; identical to SEQ ID NO: 1
  source 1..604: mol_type = other DNA

SEQ ID NO: 8 - DNA, 604 bp, synthetic construct; identical to SEQ ID NO: 1
  source 1..604: mol_type = other DNA

SEQ ID NO: 9 - DNA, 1034 bp, synthetic construct
  source 1..1034: mol_type = other DNA
gggccacgcggagcgactttgtcaaagggacaatgacctggagcgaccgaagcgtgcgcgctgcgacaattcagtcatgcccgatatgctgcgcctgctcgcctcccgacaggatctgctggaggatcttcaggcgaggttcattgcgagcggcgcaagaatcaagccttggaagcagcaagatctctctgttcgtaaaagggcatcagtttcgcccaactcaacagacactggtgcggaatgcgtggagcgcggctcggccacttcggggagtgcaagcggtacagcggccttgcccacagaaaccaacgcgccaaagtcattggcattcacattgcagtgtccagccgcactttccatcgtctttgtactcgctctcactctagtcctccactgagagagtaatgctcacaggtcaaagagttgacggttaaactcacacagcaagagaaagcttgtataagtagatgtatcaaatgtatttatgaaataattaccaaagccggaaaaccatatttacaaagccggcgaatatagctgtgcacttcctggagccgaccacctcttagagtaggtctgattaaagcccgagtagcgacccaggctaggaggcaaatcgcccaccgcggtcccccagctactctcattcttccccagcgtgagaacgagttccttgccctcggtaaagaatgagtgatcaatccagttctttgtgtagggcttgccgttgagcgtcgcactctgaatatagatattcttgtacgcggcgtcaaagtggacgttgcgaatcttggccgtcttgccggtgacggggtgcttgacggtcacgctggggaaaaagggcggcgtgatgaggtagacgtcttgtcccgggttgggaaagagacccatcatgctaaaggcgacaaaggagcccatggcgccgctgtcgtcgttgccgggacgccgggagggtcgctgagagcgcgatgtaaaagtggctgcgcttgcggagagggccggacgcgcgtatgatatatctgtgtgaaaacaca

SEQ ID NO: 10 - DNA, 1266 bp, synthetic construct
  source 1..1266: mol_type = other DNA
gtgctcgattctatattcagagtgcgacgctcaacggcaagccctacacaaagaactggattgatcactcattctttaccgagggcaaggaactcgttctcacgctggggaagaatgagagtagctgggggaccgcggtgggcgatttgcctcctagcctgggtcgctactcgggctttaatcagacctactctaagaggtggtcggctccaggaagtgcacagctatattcgccggctttgtaaatatggttttccggctttggtaattatttcataaatacatttgatacatctacttatacaagctttctcttgctgtgtgagtttaaccgtcaactctttgacctgtgagcattactctctcagtggaggactagagtgagagcgagtacaaagacgatggaaagtgcggctggacactgcaatgtgaatgccaatgactttggcgcgttggtttctgtgggcaaggccgctgtaccgcttgcactccccgaagtggccgagccgcgctccacgcattccgcaccagtgtctgttgagttgggcgaaactgatgcccttttacgaacagagagatcttgctgcttccaaggcttgattcttgcgccgctcgcaatgaacctcgcctgaagatcctccagcagatcctgtcgggaggcgagcaggcgcagcatatcgggcatgactgaattgtcgcagcgcgcacgcttcggtcgctccaggtcattgtcccttttgacaaaggtcgctccgggcttaatcttgtggcagttgctctggctgcgaccggcacgcagctggcagatttcctcctccttgcagtctccctggcaacctttcttgatccacccccggctcttgcgagacacgtattcgtcaaacagcgtgcgttgctttcaaaagctaaagtgacattgtgccagaaagccggtgtgagctcggcctcggcgcgatgggcggagaaagtagtgagccataggactctttggcagagtagtatttcttccatgtcggcccggactttgatactcgggatcggtcatgtcggcatgtagtacgcatcgagatgcaaatgtgtcgggatcgacgtcgtataccgcgaagcggcatgcgacgtggcagatgctgcgatgtagactgcgtgcctgccatgcatccgcctcagtagtcgagatagctgactcgatgatcatgtgccggttggccaagacgtcgaatgtgtccgtacgcgactgtcagaagtatttttgggctggatactaat

SEQ ID NO: 11 - DNA, 5280 bp, synthetic construct
  source 1..5280: mol_type = other DNA
atggcgaaccttccctttccgcattcgagcgcgccgctcaaaactgttgaggaaatacagtttggtatgttatccccggaggaaatcaaaaacatgagcgtcgcgcacattctctatcctgaaacaatggaagagaacaagacaacaccacgcgacggcggtctgaacgatcctcttcttggttccatcgatcgtcagttcaagtgcaagacttgctctcagccgatgagcgagtgtccaggtcattttggccatatagagctggcgaaacctgtctatcaccctggtttcatcaagaaagtgaaaaaggttttggagattgtctgccacaactgcagcaaagtgttggccgatgaaagcgatcccgaattcgtcacagctattcatactcgcgatccgaaactccgattcaagcgcgtttgggccgtatgcaagaagaagcgcaaatgcgagaatgaggagcggcaagacaagaataaagacgaagagttcgctccaggtgtcaagaacgtcgttctcgaaggacatggcggatgtggcaatatgcagccgcaggtgagacaggccgcgctgcaactcaaagctgccttcgaggttacttcggaagagggtcccaagaggaaagagacggttaatatcagcgccgagatggcgcatggtatccttcgccgcatctctgagcgcgatctgcacaatattggtcttaactcagactatgctcgtcctgagtggatgatcatcactgtcctgcctgtaccccctcctcccgtgcgtcctagtatttccatggatggtactggtactggcacgagaaacgaggatgatctgacctacaagcttggtgacattatccgcgccaacggtaatgtcaagcaggccattcgcgaaggatcaccgcaacacatcgcgcgtgattttgaggagctgctgcagtatcatgttgccacctacatggataatgatattgctggtcagccgcgggccctccaaaagagcggtcgtcctgtcaaggcgattcgcgcccgtctcaagggcaaggagggtcgtctgcgaggcaacttgatgggtaagcgtgtcgacttttccgcgcgtaccgttatcactggtgacgccaacttgtctctgcacgaagttggtgttccgcgaagcattgctcgtactctcacctatcctgagactgtcacaccctataacattgccaagctgcatcaactcgttgagaacggacctaacgagcaccctggtgccaaatacgtcattcgctctgatggcaccagaatcgatctacgacaccacagacgcgcggctcaaatttctttggaatatggatggaaggttgagcgtcatctaatcgatggcgactacatcatcttcaaccgtcagccctccctgcacaaggaatccatgatgggtcatcgtgtcaaggtcatgccctactctaccttccgactcaacctttccgtcacttccccctataatgccgattttgatggtgatgaaatgaacttgcacgttccccagactgaggaaactcgggctgaaatcaaggagctgtgtctcgttcccaacaatattgtgtcgcctcagaaaaacggtcccctaatgggtattgttcaagactctctggccggtgtctacaagctctgtcgtcgtgacacatttatcgataaagagcttgttcaaaatctcatgctctgggtacccaactgggatggcgtcattccacagccggctattctgaagcctagacctcgctggaccggcaagcagattatcagcatggtcatccccccagagatcagtctgtacagcaaggaagataagctggacaatcctagacatgacgctggcttgcttattcagagcggtgagctcatgtacggtctgctcaagaagaagagtgttggtgctgccgcaggaggcatcattcatttgtgctacaacgaactcggacccgagggcgccatggctttcttgaatggtgtccaacaagttgtcacctactggctactgaacaccggtttcagtattggtattggcgacactgttcccgacaaggctaccatcaataagatccaggagcatatcgacgaacacaaagcagaggtcgctcgcttgactgcccaggctacagccaacgagctggaggctttacctggtatgaatgtccgcgccactttcgagaacaaagtctccatggctctcaacatggcccgtgaccaggctggtacttcaaccatgaagagtttgaaggattccaacaatgccgtcaccatggctgattctggttccaagggttcttccatcaacatctcgcaaatgacggccctcgtcggacagcagattgttgaaggaaagcgtatccctttcggcttcaagtaccgaacactgccgcatttcacaaaggacgattattcgcctgaagctcgtggtttcgtcgagaactcgtaccttcgtggcttgactccttcggagttcttcttccacgccatggctggtagagagggtcttattgatactgcggtcaagaccgctgagacgggttacattcaacgacgtctcgtcaaggcgctcgaagatctcagcgcgcgctacgacggaactgtacgtaactcattgggcgacgttattcaattcttgtacggtgaagatggtctcgatgctatgtgcattgaaaaacagagacttggcaccatcaagatgtctaatgctgccttcgaaagccaatatcgtttggatcttgccaacccccctgagtggttccgcaaggactatgagtacggcaatgagctggcaggcgatagggcgtccatggaactgctggacatggagtgggatgcccttgttgctgacagacgccttgtccgtgatatcaacaagagcaagatgggtgaagagatgatgcagcttccactcaatattggccgtatcatcgagagtgccaagcgtgtcttcaacattcgcgaaactgaccgcagcaatctgcggccgtccgatgtcatcacaactgttcaaaatttgctggccaacatgcagattgtgcgtggcacggaccccatctcagttgaggccgattacaatgctaccattctgttcaaggcactcctgcgctctcgtctcgctttcaaagagattgtctacgtgcatcgcttaaacaagcttgcctttaaccacgtcatcggcgagcttcagaatcgttgggatagagcttttgttagccctggtgagatggttggtgttctcgctgcccagtctattggtgagcctgctactcagatgacactcaacacttttcactttgctggtgtgtcttcgaagaacgttactctgggtgtgccgcgtctcaaggagattctgaacttggccaaaaatatcaagactcccagcatggccgtctacctggacaccaaactgggtacgcaggagcaggccaagaagctccgtagtttggtcgagtataccaacttgcgctccgtcacatcggtcaccgaaatctactacgatcccgaggtgcaggcaacgaacattgctgaggatgtggacatggtcgaatcttacttcatgattcccgatgatgcccaggataccattcaccggcagtcgcgctggctcctccgcattactcttgaccgccagaaactcctggacaaggaaatcaagattgacgatgtcgctgcgtgcatcaaggaggaatactcaaacgatctggccatcatcttctctgacaacaatgccgacgagcaagtcattcgtatccgtaccattcgtcaaagtgatgacaaggacgaggattctgacacgaagattgaagacgatgtcatgctcaagcggctagaagctcacttgcttgatactatgactttacgcggtgttcctggcatcgaaagggccttcttgaccaagggtacccgcctggttgaggatgaagacggctccgaactggctctcaaggataacccgaagtgcacacagtggtacttggataccagtggttcagcactccgcgaggtactcgctgtccccggtgtcgatcccaccagaacctacagcaacgacctgtaccagatcactgaggtgttcggtgtcgaggctgtacgatctgcgctggtgaaggaattgaccaacgtgttggcttttgacggttcgtacgtcaatcatcgtcatattgctttgctgtgcgacattatgacctatcgtggtgtcatttcggccgtcacacgtcacggtattaaccgggccgacactggtgctctgatgcgttgttccttcgaagagactgtcgaaatcctgctcgaggctgccgctaccggcgaactcgacgactgccgtggtatctccgagaacgtcatgcttggtcagcttgcgcccatgggcaccggcaactttgatgtctacttggatcctaagatgctggagactgtcatttccgacaattctcgcatgggcctcatgcctggcatgcccaccaaggaaggcgagggcgagggtgctgccacaccatacgatagtggctcgccgatgggcgactctggttacctcagcatggcttcgcctgctgctggtaacttttctcctattcagggtgctggctcagaaacacctaccgggttcaataccgagtatggcggtggcttcggagccaatggttcgatgagcccgtactcgcgcggcgctactagtcccttcagcacgtcgcccacgtcgccattcagcgcaggcatggggggctattcgccatcgtcgcctaacacgggttactctcctacgtcgccgatgcttgacggcggtgctcgttacgccacctctccgtcattcagtccgtcgtcgccgtctttctcgccgacgtcgccgatgctgcgacccaccagcccggccagccccaactacagcccaacgtctcctagttactcgccgacgtcgccgacatcaccgcgacactactcgcctacatctcctgctcagttcaactcgccgacgtctcccagctactccccggccagtcccaattacagtcctgcgtctcccaacttccagggagctggtcctacttcgccctcgtactcgcctgcgtcgccatcgtggtcaccaacctcacccgacgcttactcacccacgagtccaagcttcggacgcagccccggccagcagcagtcgcccacgagccctggttactctcccacctcgccgcagttctctcctcgcacgcctggtccctcgggctctggtggaggttctggaactggcaaccaatactcgcctagctcgccgaataacgagtga

SEQ ID NO: 12 - DNA, 5280 bp, synthetic construct
  source 1..5280: mol_type = other DNA
atggcgaaccttccctttccgcattcgagcgcgccgctcaaaactgttgaggaaatacagtttggtatgttatccccggaggaaatcaaaaacatgagcgtcgcgcacattctctatcctgaaacaatggaagagaacaagacaacaccacgcgacggcggtctgaacgatcctcttcttggttccatcgatcgtcagttcaagtgcaagacttgctctcagccgatgagcgagtgtccaggtcattttggccatatagagctggcgaaacctgtctatcaccctggtttcatcaagaaagtgaaaaaggttttggagattgtctgccacaactgcagcaaagtgttggccgatgaaagcgatcccgaattcgtcacagctattcatactcgcgatccgaaactccgattcaagcgcgtttgggccgtatgcaagaagaagcgcaaatgcgagaatgaggagcggcaagacaagaataaagacgaagagttcgctccaggtgtcaagaacgtcgttctcgaaggacatggcggatgtggcaatatgcagccgcaggtgagacaggccgcgctgcaactcaaagctgccttcgaggttacttcggaagagggtcccaagaggaaagagacggttaatatcagcgccgagatggcgcatggtatccttcgccgcatctctgagcgcgatctgcacaatattggtcttaactcagactatgctcgtcccgagtggatgatcatcactgtcctgcctgtaccccctcctcccgtgcgtcctagtatttccatggatggtactggtactggcacgagaaacgaggatgatctgacctacaagcttggtgacattatccgcgccaacggtaatgtcaagcaggccattcgcgaaggatcaccgcaacacatcgcgcgtgattttgaggagctgctgcagtatcatgttgccacctacatggataatgatattgctggtcagccgcgggccctccaaaagagcggtcgtcctgtcaaggcgattcgcgcccgtctcaagggcaaggagggtcgtctgcgaggcaacttgatgggtaagcgtgtcgacttttccgcgcgtaccgttatcactggtgacgccaacttgtctctgcacgaagttggtgttccgcgaagcattgctcgtactctcacctatcctgagactgtcacaccctataacattgccaagctgcatcaactcgttgagaacggacctaacgagcaccctggtgccaaatacgtcattcgctctgatggcaccagaatcgatctacgacaccacagacgcgcggctcaaatttctttggaatatggatggaaggttgagcgtcatctaatcgatggcgactacatcatcttcaaccgtcagccctccctgcacaaggaatccatgatgggtcatcgtgtcaaggtcatgccctactctaccttccgactcaacctttccgtcacttccccctataatgccgattttgatggtgatgaaatgaacttgcacgttccccagactgaggaaactcgggctgaaatcaaggagctgtgtctcgttcccaacaatattgtgtcgcctcagaaaaacggtcccctaatgggtattgttcaagactctctggccggtgtctacaagctctgtcgtcgtgacacatttatcgataaagagcttgttcaaaatctcatgctctgggtacccaactgggatggcgtcattccacagccggctattctgaagcctagacctcgctggaccggcaagcagattatcagcatggtcatccccccagagatcagtctgtacagcaaggaagataagctggacaatcctagacatgacgctggcttgcttattcagagcggtgagctcatgtacggtctgctcaagaagaagagtgttggtgctgccgcaggaggcatcattcatttgtgctacaacgaactcggacccgagggcgccatggctttcttgaatggtgtccaacaagttgtcacctactggctactgaacaccggtttcagtattggtattggcgacactgttcccgacaaggctaccatcaataagatccaggagcatatcgacgaacacaaagcagaggtcgctcgcttgactgcccaggctacagccaacgagctggaggctttacctggtatgaatgtccgcgccactttcgagaacaaagtctccatggctctcaacatggcccgtgaccaggctggtacttcaaccatgaagagtttgaaggattccaacaatgccgtcaccatggctgattctggttccaagggttcttccatcaacatctcgcaaatgacggccctcgtcggacagcagattgttgaaggaaagcgtatccctttcggcttcaagtaccgaacactgccgcatttcacaaaggacgattattcgcctgaagctcgtggtttcgtcgagaactcgtaccttcgtggcttgactccttcggagttcttcttccacgccatggctggtagagagggtcttattgatactgcggtcaagaccgctgagacgggttacattcaacgacgtcttgtcaaggcgctcgaagatctcagcgcgcgctacgacggaactgtacgtaactcattgggcgacgttattcaattcttgtacggtgaagatggtctcgatgctatgtgcattgaaaaacagagacttggcaccatcaagatgtctaatgctgccttcgaaagccaatatcgtttggatcttgccaacccccctgagtggttccgcaaggactatgagtacggcaatgagctggcaggcgatagggcgtccatggaactgctggacatggagtgggatgcccttgttgctgacagacgccttgtccgtgatatcaacaagagcaagatgggtgaagagatgatgcagcttccactcaatattggccgtatcatcgagagtgccaagcgtgtcttcaacattcgcgaaactgaccgcagcaatctgcggccgtccgatgtcatcacaactgttcaaaatttgctggccaacatgcagattgtgcgtggcacggaccccatctcagttgaggccgattacaatgctaccattctgttcaaggcactcctgcgctctcgtctcgctttcaaagagattgtctacgtgcatcgcttaaacaagcttgcctttaaccacgtcatcggcgagcttcagaatcgttgggatagagcttttgttagccctggtgagatggttggtgttctcgctgcccagtctattggtgagcctgctactcagatgacactcaacacttttcactttgctggtgtgtcttcgaagaacgttactctgggtgtgccgcgtctcaaggagattctgaacttggccaaaaatatcaagactcccagcatggccgtctacctggacaccaaactgggtacgcaggagcaggccaagaagctccgtagtttggtcgagtataccaacttgcgctccgtcacatcggtcaccgaaatctactacgatcccgaggtgcaggcaacgaacattgctgaggatgtggacatggtcgaatcttacttcatgattcccgatgatgcccaggataccattcaccggcagtcgcgctggctcctccgcattactcttgaccgccagaaactcctggacaaggaaatcaagattgacgatgtcgctgcgtgcatcaaggaggaatactcaaacgatctggccatcatcttctctgacaacaatgccgacgagcaagtcattcgtatccgtaccattcgtcaaagtgatgacaaggacgaggattctgacacgaagattgaagacgatgtcatgctcaagcggctagaagctcacttgcttgatactatgactttacgcggtgttcctggcatcgaaagggccttcttgaccaagggtacccgcctggttgaggatgaagacggctccgaactggctctcaaggataacccgaagtgcacacagtggtacttggataccagtggttcagcactccgcgaggtactcgctgtccccggtgtcgatcccaccagaacctacagcaacgacctgtaccagatcactgaggtgttcggtgtcgaggctgtacgatctgcgctggtgaaggaattgaccaacgtgttggcttttgacggttcgtacgtcaatcatcgtcatattgctttgctgtgcgacattatgacctatcgtggtgtcatttcggccgtcacacgtcacggtattaaccgggccgacactggtgctctgatgcgttgttccttcgaagagactgtcgaaatcctgctcgaggctgccgctaccggcgaactcgacgactgccgtggtatctccgagaacgtcatgcttggtcagcttgcgcccatgggcaccggcaactttgatgtctacttggatcctaagatgctggagactgtcatttccgacaattctcgcatgggcctcatgcctggcatgcccaccaaggaaggcgagggcgagggtgctgccacaccatacgatagtggctcgccgatgggcgactctggttacctcagcatggcttcgcctgctgctggtaacttttctcctattcagggtgctggctcagaaacacctaccgggttcaataccgagtatggcggtggcttcggagccaatggttcgatgagcccgtactcgcgcggcgctactagtcccttcagcacgtcgcccacgtcgccattcagcgcaggcatggggggctattcgccatcgtcgcctaacacgggttactctcctacgtcgccgatgcttgacggcggtgctcgttacgccacctctccgtcattcagtccgtcgtcgccgtctttctcgccgacgtcgccgatgctgcgacccaccagcccggccagccccaactacagcccaacgtctcctagttactcgccgacgtcgccgacatcaccgcgacactactcgcctacatctcctgctcagttcaactcgccgacgtctcccagctactccccggccagtcccaattacagtcctgcgtctcccaacttccagggagctggtcctacttcgccctcgtactcgcctgcgtcgccatcgtggtcaccaacctcacccgacgcttactcacccacgagtccaagcttcggacgcagccccggccagcagcagtcgcccacgagccctggttactctcccacctcgccgcagttctctcctcgcacgcctggtccctcgggctctggtggaggttctggaactggcaaccaatactcgcctagctcgccgaataacgagtga

SEQ ID NO: 13 - DNA, 5280 bp, synthetic construct; identical to SEQ ID NO: 12
  source 1..5280: mol_type = other DNA

SEQ ID NO: 14 - DNA, 5280 bp, synthetic construct; identical to SEQ ID NO: 12
  source 1..5280: mol_type = other DNA

SEQ ID NO: 15 - DNA, 5280 bp, synthetic construct
  source 1..5280: mol_type = other DNA
atggcgaaccttccctttccgcattcgagcgcgccgctcaaaactgttgaggaaatacagtttggtatgttatccccggaggaaatcaaaaacatgagcgtcgcgcacattctctatcctgaaacaatggaagagaacaagacaacaccacgcgacggcggtctgaacgatcctcttcttggttccatcgatcgtcagttcaagtgcaagacttgctctcagccgatgagcgagtgtccaggtcattttggccatatagagctggcgaaacctgtctatcaccctggtttcatcaagaaagtgaaaaaggttttggagattgtctgccacaactgcagcaaagtgttggccgatgaaagcgatcccgaattcgtcacagctattcatactcgcgatccgaaactccgattcaagcgcgtttgggccgtatgcaagaagaagcgcaaatgtgagaatgaggagcggcaagacaagaataaagacgaagagttcgctccaggtgtcaagaacgtcgttctcgaaggacatggcggatgtggcaatatgcagccgcaggtgagacaggccgcgctgcaactcaaagctgccttcgaggttacttcggaagagggtcccaagaggaaagagacggttaatatcagcgccgagatggcgcatggtatccttcgccgcatctctgagcgcgatctgcacaatattggtcttaactcagactatgctcgtcccgagtggatgatcatcactgtcctgcctgtaccccctcctcccgtgcgtcctagtatttccatggatggtactggtactggcacgagaaacgaggatgatctgacctacaagcttggtgacattatccgcgccaacggtaatgtcaagcaggccattcgcgaaggatcaccgcaacacatcgcgcgtgattttgaggagctgctgcagtatcatgttgccacctacatggataatgatattgctggtcagccgcgggccctccaaaagagcggtcgtcctgtcaaggcgattcgcgcccgtctcaagggcaaggagggtcgtctgcgaggcaacttgatgggtaagcgtgtcgacttttccgcgcgtaccgttatcactggtgacgccaacttgtctctgcacgaagttggtgttccgcgaagcattgctcgtactctcacctatcctgagactgtcacaccctataacattgccaagctgcatcaactcgttgagaacggacctaacgagcaccctggtgccaaatacgtcattcgctctgatggcaccagaatcgatctacgacaccacagacgcgcggctcaaatttctttggaatatggatggaaggttgagcgtcatctaatcgatggcgactacatcatcttcaaccgtcagccctccctgcacaaggaatccatgatgggtcatcgtgtcaaggtcatgccctactctaccttccgactcaacctttccgtcacttccccctataatgccgattttgatggtgatgaaatgaacttgcacgttccccagactgaggaaactcgggctgaaatcaaggagctgtgtctcgttcccaacaatattgtgtcgcctcagaaaaacggtcccctaatgggtattgttcaagactctctggccggtgtctacaagctctgtcgtcgtgacacatttatcgataaagagcttgttcaaaatctcatgctctgggtacccaactgggatggcgtcattccacagccggctattctgaagcctagacctcgctggaccggcaagcagattatcagcatggtcatccccccagagatcagtctgtacagcaaggaagataagctggacaatcctagacatgacgctggcttgcttattcagagcggtgagctcatgtacggtctgctcaagaagaagagtgttggtgctgccgcaggaggcatcattcatttgtgctacaacgaactcggacccgagggcgccatggctttcttgaatggtgtccaacaagttgtcacctactggctactgaacaccggtttcagtattggtattggcgacactgttcccgacaaggctaccatcaataagatccaggagcatatcgacgaacacaaagcagaggtcgctcgcttgactgcccaggctacagccaacgagctggaggctttacctggtatgaatgtccgcgccactttcgagaacaaagtctccatggctctcaacatggcccgtgaccaggctggtacttcaaccatgaagagtttgaaggattccaacaatgccgtcaccatggctgattctggttccaagggttcttccatcaacatctcgcaaatgacggccctcgtcggacagcagattgttgaaggaaagcgtatccctttcggcttcaagtaccgaacactgccgcatttcacaaaggacgattattcgcctgaagctcgtggtttcgtcgagaactcgtaccttcgtggcttgactccttcggagttcttcttccacgccatggctggtagagagggtcttattgatactgcggtcaagaccgctgagacgggttacattcaacgacgtctcgtcaaggcgctcgaagatctcagcgcgcgctacgacggaactgtacgtaactcattgggcgacgttattcaattcttgtacggtgaagatggtctcgatgctatgtgcattgaaaaacagagacttggcaccatcaagatgtctaatgctgccttcgaaagccaatatcgtttggatcttgccaacccccctgagtggttccgcaaggactatgagtacggcaatgagctggcaggcgatagggcgtccatggaactgctggacatggagtgggatgcccttgttgctgacagacgccttgtccgtgatatcaacaagagcaagatgggtgaagagatgatgcagcttccactcaatattggccgtatcatcgagagtgccaagcgtgtcttcaacattcgcgaaactgaccgcagcaatctgcggccgtccgatgtcatcacaactgttcaaaatttgctggccaacatgcagattgtgcgtggcacggaccccatctcagttgaggccgattacaatgctaccattctgttcaaggcactcctgcgctctcgtctcgctttcaaagagattgtctacgtgcatcgcttaaacaagcttgcctttaaccacgtcatcggcgagcttcagaatcgttgggatagagcttttgttagccctggtgagatggttggtgttctcgctgcccagtctattggtgagcctgctactcagatgacactcaacacttttcactttgctggtgtgtcttcgaagaacgttactctgggtgtgccgcgtctcaaggagattctgaacttggccaaaaatatcaagactcccagcatggccgtctacctggacaccaaactgggtacgcaggagcaggccaagaagctccgtagtttggtcgagtataccaacttgcgctccgtcacatcggtcaccgaaatctactacgatcccgaggtgcaggcaacgaacattgctgaggatgtggacatggtcgaatcttacttcatgattcccgatgatgcccaggataccattcaccggcagtcgcgctggctcctccgcattactcttgaccgccagaaactcctggacaaggaaatcaagattgacgatgtcgctgcgtgcatcaaggatgaatactcaaacgatctggccatcatcttctctgacaacaatgccgacgagcaagtcattcgtatccgtaccattcgtcaaagtgatgacaaggacgaggattctgacacgaagattgaagacgatgtcatgctcaagcggctagaagctcacttgcttgatactatgactttacgcggtgttcctggcatcgaaagggccttcttgaccaagggtacccgcctggttgaggatgaagacggctccgaactggctctcaaggataacccgaagtgcacacagtggtacttggataccagtggttcagcactccgcgaggtactcgctgtccccggtgtcgatcccaccagaacctacagcaacgacctgtaccagatcactgaggtgttcggtgtcgaggctgtacgatctgcgctggtgaaggaattgaccaacgtgttggcttttgacggttcgtacgtcaatcatcgtcatattgctttgctgtgcgacattatgacctatcgtggtgtcatttcggccgtcacacgtcacggtattaaccgggccgacactggtgctctgatgcgttgttccttcgaagagactgtcgaaatcctgctcgaggctgccgctaccggcgaactcgacgactgccgtggtatctccgagaacgtcatgcttggtcagcttgcgcccatgggcaccggcaactttgatgtctacttggatcctaagatgctggagactgtcatttccgacaattctcgcatgggcctcatgcctggcatgcccaccaaggaaggcgagggcgagggtgctgccacaccatacgatagtggctcgccgatgggcgactctggttacctcagcatggcttcgcctgctgctggtaacttttctcctattcagggtgctggctcagaaacacctaccgggttcaataccgagtatggcggtggcttcggagccaatggttcgatgagcccgtactcgcgcggcgctactagtcccttcagcacgtcgcccacgtcgccatttagcgcaggcatggggggctattcgccatcgtcgcctaacacgggttactctcctacgtcgccgatgcttgacggcggtgctcgttacgccacctctccgtcattcagtccgtcgtcgccgtctttctcgccgacgtcgccgatgctgcgacccaccagcccggccagccccaactacagcccaacgtctcctagttactcgccgacgtcgccgacatcaccgcgacactactcgcctacatctcctgctcagttcaactcgccgacgtctcccagctactccccggccagtcccaattacagtcctgcgtctcccaacttccagggagctggtcctacttcgccctcgtactcgcctgcgtcgccatcgtggtcaccaacctcacccgacgcttactcacccacgagtccaagcttcggacgcagccccggccagcagcagtcgcccacgagccctggttactctcccacctcgccgcagttctctcctcgcacgcctggtccctcgggctctggtggaggttctggaactggcaaccaatactcgcctagctcgccgaataacgagtga

SEQ ID NO: 16 - DNA, 5280 bp, synthetic construct; identical to SEQ ID NO: 12
  source 1..5280: mol_type = other DNA

SEQ ID NO: 17 - DNA, 5280 bp, synthetic construct; identical to SEQ ID NO: 12
  source 1..5280: mol_type = other DNA

SEQ ID NO: 18 - DNA, 5280 bp, synthetic construct; identical to SEQ ID NO: 12
  source 1..5280: mol_type = other DNA

SEQ ID NO: 19 - DNA, 3714 bp, synthetic construct
  source 1..3714: mol_type = other DNA
atggcagactacgaggatgaatacgactacgagaattacgaggaagaagattcgggcatcactcccgaagattgttggaccgtcatctcctctttcttcgaatccaagggcctcgtctctcagcaaactgcctcctttgatgaatttactcagtcaactatccaagaccttgtcagcgagtactcaagaatatctctcgaccaacccaacccgccctccaacgacgaccggaagataagcgtccgccgatacgagatcgagttcggcagcatcatggtatcgaggccttcgatcagtgagaccgatgggaccgtgacttcgcttctaccatacgagtgccgcgatcgtaacctgacctatgcagcgccggtttacattaagattaccaaaaaagtgcaagctgctgtggaaacggaaatacctttgcacgagctagatgatgctcaacagacagagtatgccaaaactggtgagctgcctacacggttggcctggagatcggaggatgcgccagagcccgaaaattccaacaagcctgatgcctggaaggacatggtcttcgtcgggaaaatgccggtcatggtcaaatctaaagtctgccatttgagccgcgagcatgacgagaatttatttcttgtcaatgagtgcccatacgaccagggcggttactttgttatcaatggaagtgaaaaggtgcttatcgcccaagagcgttccgccgccaatattgttcaggtcttcaagaaggctcagcccagcccttatacatacactgctgaaatcagaagcgctctggagaaaggctcgcgtctcatctccagcatgatgctcaagctctacgggaagggtgagtcagctcgtggcggcttcgggcaaacgattcatacaactcttccgtttgttaaagcagaccttcctgtcgccattgtatttagagcacttggcgttgtctccgacgaggatattctcaaccacatttgttacgaccgaaaagactctcagatgctggaaatgcttcggccttgtatcgaggaagctttctgcgtccaagaccgtgaagtcgccctcgacttcattggtaagcgtggtaatcgggatcaggctggtctcggacgcgataaacgtattcgtgtggcacgcgacattcttcagaaagaaactttgcctcacatctcccaggaggctggtagcgagacgcgcaaagccttctttatcggttatatggtccacaaactactccagtgcgctctggggcgtcgggagcctgacgacagagaccacttcggcaaaaagcgtcttgatttagcaggtccactacttgcgaaactcttccgcggtatcatgcgccgaatgcactcagagctcgccaattacctgcgacgctgcgtcgagggtaaccgccacttcaatctagctgtcggtgttaagcctggcacgctttcgaacggcctcaagtactctcttgctactggtaattggggtgaccagaaaaaagctatgagctcaactgctggtgtgtcccaggttttgaaccgatatacttttgcctcaacactatcccaccttcgacgtaccaatacacccattggcagagatggcaaactggcaaaacctcgtcagcttcataatactcattggggtcttgtctgccctgctgagacacctgaaggccaggcttgcggtcttgtcaagaatctgtctctcatgtgctacgtcagcgtcggctccccagccgagccgctcattgacttcatgatcaacaggggtatggaggtcattgaggagtacgaaccactcagatacccacacgctaccaagatttttgtcaacgggacctgggttggagttcatcaggaccccaagcaccttgctgaccaggtattcgacacccgccgcaagtcctacctgcagtatgaggtgtctcttgtcagagaaatccgtgaccaggaattcaagatcttctctgatgctggccgagtcatgcggcctgtttttactgtgcaaagtaaaaatgacccggagactggccttgaaaagggacagctcggtctcaccaaggatttagtcaacagactggcgcaagagcaagccgacccgccagatgatccagaaatgaagacgggttgggagggcttgatcaaagctggcgctgtcgagtatctagacgctgaagaagaggagacgtcaatgatttgcatgaccccggaggatctggagctttatagacttcagaaagcgggtgtagctgtcgacgacgaccatggcgatgatctgaacaagcgcctgaagactaaaaccaacccaactacacacatgtacactcactgtgaaattcacccgaaccacaatcaatcgcctcgtaacacctaccaatccgcaatgggtaaacaagccatgggcttcttcctgacaaattattctcgtcgtatggatactatggcaaatatcctgtactacccacaaaagcctttggcaacgactagatccatggagttcttgaagttccgagaattgcctgcaggtcaaaacgccatcgtcgcgattgcttgttattccggttataatcaggaggactctgtgattatgaatcaaagcagtatcgatcgcggactcttcagaagtttgttctttcgttcgtactctgatcaagagaaaaaggtcggtttgaactacacagagatattcgaaaagccgttccaccaaagcaccctgcgcatgaaacacggtacttacgacaagctcgatgaggacggtattgtcgctcctggcgttcgtgtgtctggagaagacattatcattggcaaaactgcgccaattgacccagagacgcaagacttgggcgcgcgtacaactgcgcatcagcgccgtgatatctctacgcctctgcgtagtaccgaaaatggtattgttgatcaagtcattgtgactgtcaacgccgacaacgtcaaatacgtcaaggtcagggttcgtacgaccaagataccccagatcggtgataagtttgcttcacgacacggccaaaaaggtaccattggtgtcacataccgacaggaagatatgccattcacgagagagggggtcaccccagatatcattatcaacccgcacgccattccctctcgaatgacaattgctcatctgatcgaatgtcttttgagcaaggtttcgactttagaaggtatggaaggcgatgctactccatttaccgacgtcactgtcgattctgtctccgacttgcttcgcaagcacggctatcagtcccgcgggtttgagatcatgtacaacggtcacactggtaagaaacttcgtgctcaggttttcttcggaccgacctattaccagcgtctgcgtcacatggtggacgacaaaattcacgcccgtgcccgtggccctgtgcagatcatgactagacaacctgtggagggtcgtgcaagggatggggggttgcgtttcggcgaaatggaacgtgattgcatgattgcgcatggtgcagcagcattcttgaaggagcgcctctttgaagtgtctgatgcgttccgagtgcacatctgcgagatttgcggcttgatgacgccgattgcaccttgcaagaacaagaccaaaattgcccagattcacattccgtacgcagccaagctattgttccaagaacttcaatccatgaacattgcagcacgtatgtttacggacagatctggtgcctcgatcaggtag

SEQ ID NO: 20 - DNA, 3627 bp, synthetic construct
  source 1..3627: mol_type = other DNA
atggcagactacgaggatgaatacgactacgagaattacgaggaagaagattcgggcatcactcccgaagattgttggaccgtcatctcctctttcttcgaatccaagggcctcgtctctcagcaaactgcctcctttgatgaatttactcagtcaactatccaagaccttgtcagcgagtactcaagaatatctctcgaccaacccaacccgccctccaacgacgaccggaagataagcgtccgccgatacgagatcgagttcggcagcatcatggtatcgaggccttcgatcagtgagaccgatgggaccgtgacttcgcttctaccatacgagtgccgcgatcgtaacctgacctatgcagcgccggtttacattaagattaccaaaaaagtgcaagctgctgtggaaacggaaatacctttgcacgagctagatgatgctcaacagacagagtatgccaaaactggtgagctgcctacacggttggcctggagatcggaggatgcgccagagcccgaaaattccaacaagcctgatgcctggaaggacatggtcttcgtcgggaaaatgccggtcatggtcaaatctaaagtctgccatttgagccgcgagcatgacgagaatttatttcttgtcaatgagtgcccatacgaccagggcggttactttgttatcaatggaagtgaaaaggtgcttatcgcccaagagcgttccgccgccaatattgttcaggtcttcaagaaggctcagcccagcccttatacatacactgctgaaatcagaagcgctctggagaaaggctcgcgtctcatctccagcatgatgctcaagctctacgggaagggtgagtcagctcgtggcggcttcgggcaaacgattcatacaactcttccgtttgttaaagcagaccttcctgtcgccattgtatttagagcacttggcgttgtctccgacgaggatattctcaaccacatttgttacgaccgaaaagactctcagatgctggaaatgcttcggccttgtatcgaggaagctttctgcgtccaagaccgtgaagtcgccctcgacttcattggtaagcgtggtaatcgggatcaggctggtctcggacgcgataaacgtattcgtgtggcacgcgacattcttcagaaagaaactttgcctcacatctcccaggaggctggtagcgagacgcgcaaagccttctttatcggttatatggtccacaaactactccagtgcgctctggggcgtcgggagcctgacgacagagaccacttcggcaaaaagcgtcttgatttagcaggtccactacttgcgaaactcttccgcggtatcatgcgccgaatgcactcagagctcgccaattacctgcgacgctgcgtcgagggtaaccgccacttcaatctagctgtcggtgttaagcctggcacgctttcgaacggcctcaagtactctcttgctactggtaattggggtgaccagaaaaaagctatgagctcaactgctggtgtgtcccaggttttgaaccgatatacttttgcctcaacactatcccaccttcgacgtaccaatacacccattggcagagatggcaaactggcaaaacctcgtcagcttcataatactcattggggtcttgtctgccctgctgagacacctgaaggccaggcttgcggtcttgtcaagaatctgtctctcatgtgctacgtcagcgtcggctccccagccgagccgctcattgacttcatgatcaacaggggtatggaggtcattgaggagtacgaaccactcagatacccacacgctaccaagatttttgtcaacgggacctgggttggagttcatcaggaccccaagcaccttgctgaccaggtattcgacacccgccgcaagtcctacctgcagtatgaggtgtctcttgtcagagaaatccgtgaccaggaattcaagatcttctctgatgctggccgagtcatgcggcctgtttttactgtgcaaagtaaaaatgacccggagactggccttgaaaagggacagctcggtctcaccaaggatttagtcaacagactggcgcaagagcaagccgacccgccagatgatccagaaatgaagacgggttgggagggcttgatcaaagctggcgctgtcgagtatctagacgctgaagaagaggagacgtcaatgatttgcatgaccccggaggatctggagctttatagacttcagaaagcgggtgtagctgtcgacgacgaccatggcgatgatctgaacaagcgcctgaagactaaaaccaacccaactacacacatgtacactcactgtgaaattcacccgagtatgatcttgggtatctgcgcaagcattattcctttcccagaccacaatcaatcgcctcgtaacacctaccaatccgcaatgggtaaacaagccatgggcttcttcctgacaaattattctcgtcgtatggatactatggcaaatatcctgtactacccacaaaagcctttggcaacgactagatccatggagttcttgaagttccgagaattgcctgcaggtcaaaacgccatcgtcgcgattgcttgttattccggttataatcaggaggactctgtgattatgaatcaaagcagtatcgatcgcggactcttcagaagtttgttctttcgttcgtactctgatcaagagaaaaaggtcggtttgaactacacagagatattcgaaaagccgttccaccaaagcaccctgcgcatgaaacacggtacttacgacaagctcgatgaggacggtattgtcgctcctggcgttcgtgtgtctggagaagacattatcattggcaaaactgcgccaattgacccagagacgcaagacttgggcgcgcgtacaactgcgcatcagcgccgtgatatctctacgcctctgcgtagtaccgaaaatggtattgttgatcaagtcattgtgactgtcaacgccgacaacgtcaaatacgtcaaggtcagggttcgtacgaccaagataccccagatcggtgataagtttgcttcacgacacggccaaaaaggtaccattggtgtcacataccgacaggaagatatgccattcacgagagagggggtcaccccagatatcattatcaacccgcacgccattccctctcgaatgacaattgctcatctgatcgaatgtcttttgagcaaggtttcgactttagaaggtatggaaggcgatgctactccatttaccgacgtcactgtcgattctgtctccgacttgcttcgcaagcacggctatcagtcccgcgggtttgagatcatgtacaacggtcacactggtaagaaacttcgtgctcaggttttcttcggaccgacctattaccagcgtctgcgtcacatggtggacgacaaaattcacgcccgtgcccgtggccctgtgcagatcatgactagacaacctgtggagggtcgtgcaagggatggggggttgcgtttcggcgaaatggaacgtgattgcatgattgcgcatggtgcagcagcattcttgaaggagcgcctctttgaagtgtctgatgcgttccgagtgcacatctgcgagatttgcggcttgatgacgccgattgcgtaa

SEQ ID NO: 21 - DNA, 3627 bp, synthetic construct; identical to SEQ ID NO: 20
  source 1..3627: mol_type = other DNA

SEQ ID NO: 22 - DNA, 3627 bp, synthetic construct; identical to SEQ ID NO: 20
  source 1..3627: mol_type = other DNA

SEQ ID NO: 23 - DNA, 3786 bp, synthetic construct
  source 1..3786: mol_type = other DNA
atggcagactacgaggatgaatacgactacgagaattacgaggaagaagattcgggcatcactcccgaagattgttggaccgtcatctcctctttcttcgaatccaagggcctcgtctctcagcaaactgcctcctttgatgaatttactcagtcaactatccaagaccttgtcagcgagtactcaaaaatatctctcgaccaacccaacccgccctccaacgacgaccggaagataagcgtccgccgatacgagatcgagttcggcagcatcatggtatcgaggccttcgatcagtgagaccgatgggaccgtgacttcgcttctaccatacgagtgccgcgatcgtaacctgacctatgcagcgccggtttacattaagattaccaaaaaagtgcaagctgctgtggaaacggaaatacctttgcacgagctagatgatgctcaacagacagagtatgccaaaactggtgagctgcctacacggttggcctggagatcggaggatgcgccagagcccgaaaattccaacaagcctgatgcctggaaggacatggtcttcgtcgggaaaatgccggtcatggtcaaatctaaagtctgccatttgagccgcgagcatgacgagaatttatttcttgtcaatgagtgcccatacgaccagggcggttactttgttatcaatggaagtgaaaaggtgcttatcgcccaagagcgttccgccgccaatattgttcaggtcttcaagaaggctcagcccagcccttatacatacactgctgaaatcagaagcgctctggagaaaggctcgcgtctcatctccagcatgatgctcaagctctacgggaagggtgagtcagctcgtggcggcttcgggcaaacgattcatacaactcttccgtttgttaaagcagaccttcctgtcgccattgtatttagagcacttggcgttgtctccgacgaggatattctcaaccacatttgttacgaccgaaaagactctcagatgctggaaatgcttcggccttgtatcgaggaagctttctgcgtccaagaccgtgaagtcgccctcgacttcattggtaagcgtggtaatcgggatcaggctggtctcggacgcgataaacgtattcgtgtggcacgcgacattcttcagaaagaaactttgcctcacatctcccaggaggctggtagcgagacgcgcaaagccttctttatcggttatatggtccacaaactactccagtgcgctctggggcgtcgggagcctgacgacagagaccacttcggcaaaaagcgtcttgatttagcaggtccactacttgcgaaactcttccgcggtatcatgcgccgaatgcactcagagctcgccaattacctgcgacgctgcgtcgagggtaaccgccacttcaatctagctgtcggtgttaagcctggcacgctttcgaacggcctcaagtactctcttgctactggtaattggggtgaccagaaaaaagctatgagctcaactgctggtgtgtcccaggttttgaaccgatatacttttgcctcaacactatcccaccttcgacgtaccaatacacccattggcagagatggcaaactggcaaaacctcgtcagcttcataatactcattggggtcttgtctgccctgctgagacacctgaaggccaggcttgcggtcttgtcaagaatctgtctctcatgtgctacgtcagcgtcggctccccagccgagccgctcattgacttcatgatcaacaggggtatggaggtcattgaggagtacgaaccactcagatacccacacgctaccaagatttttgtcaacgggacctgggttggagttcaccaggaccccaagcaccttgctgaccaggtattcgacacccgccgcaagtcctacctgcagtatgaggtgtctcttgtcagagaaatccgtgaccaggaattcaagatcttctctgatgctggccgagtcatgcggcctgtttttactgtgcaaagtaaaaatgacccggagactggccttgaaaagggacagctcggtctcaccaaggatttagtcaacagactggcgcaagagcaagccgacccgccagatgatccagaaatgaagacgggttgggagggcttgatcaaagctggcgctgtcgagtatctagacgctgaagaagaggagacgtcaatgatttgcatgaccccggaggatctggagctttatagacttcagaaagcgggtgtagctgtcgacgacgaccatggcgatgatctgaacaagcgcctgaagactaaaaccaacccaactacacacatgtacactcactgtgaaattcacccgagtatgatcttgggtatctgcgcaagcattattcctttcccagaccacaatcaatcgcctcgtaacacctaccaatccgcaatgggtaaacaagccatgggcttcttcctgacaaattattctcgtcgtatggatactatggcaaatatcctgtactacccacaaaagcctttggcaacgactagatccatggagttcttgaagttccgagaattgcctgcaggtcaaaacgccatcgtcgcgattgcttgttattccggttataatcaggaggactctgtgattatgaatcaaagcagtatcgatcgcggactcttcagaagtttgttctttcgttcgtactctgatcaagagaaaaaggtcggtttgaactacacagagatattcgaaaagccgttccaccaaagcaccctgcgcatgaaacacggtacttacgacaagctcgatgaggacggtattgtcgctcctggcgttcgtgtgtctggagaagacattatcattggcaaaactgcgccaattgacccagagacgcaagacttgggcgcgcgtacaactgcgcatcagcgccgtgatatctctacgcctctgcgtagtaccgaaaatggtattgttgatcaagtcattgtgactgtcaacgccgacaacgtcaaatacgtcaaggtcagggttcgtacgaccaagataccccagattggtgataagtttgcttcacgacacggccaaaaaggtaccattggtgtcacataccgacaggaagatatgccattcacgagagagggggtcaccccagatatcattatcaacccgcacgccattccctctcgaatgacaattgctcatctgatcgaatgtcttttgagcaaggtttcgactttagaaggtatggaaggcgatgctactccatttaccgacgtcactgtcgattctgtctccgacttgcttcgcaagcacggctatcagtcccgcgggtttgagatcatgtacaacggtcacactggtaagaaacttcgtgctcaggtcttcttcggaccgacctattaccagcgtctgcgtcacatggtggacgacaaaattcacgcccgtgcccgtggccctgtgcagatcatgactagacaacctgtggagggtcgtgcaagggatggggggttgcgtttcggcgaaatggaacgtgattgcatgattgcgcatggtgcagcagcattcttgaaggagcgcctctttgaagtgtctgatgcgttccgagtgcacatctgcgagatttgcggcttgatgacgccgattgcaaatctttcaaagcaatcttttgaatgcagaccttgcaagaacaagaccaaaattgcccagattcacattccgtacgcagccaagctattgttccaagaacttcaatccatgaacattgcagcacgtatgtttacggacagatctggtgcctcgatcaggtag

SEQ ID NO: 24 - DNA, 3627 bp, synthetic construct; identical to SEQ ID NO: 20
  source 1..3627: mol_type = other DNA

SEQ ID NO: 25 - DNA, 3627 bp, synthetic construct; identical to SEQ ID NO: 20
  source 1..3627: mol_type = other DNA

SEQ ID NO: 26 - DNA, 3786 bp, synthetic construct
  source 1..3786: mol_type = other DNA
atggcagactacgaggatgaatacgactacgagaattacgaggaagaagattcgggcatcactcccgaagattgttggaccgtcatctcctctttcttcgaatccaagggcctcgtctctcagcaaactgcctcctttgatgaatttactcagtcaactatccaagaccttgtcagcgagtactcaagaatatctctcgaccaacccaacccgccctccaacgacgaccggaagataagcgtccgccgatacgagatcgagttcggcagcatcatggtatcgaggccttcgatcagtgagaccgatgggaccgtgacttcgcttctaccatacgagtgccgcgatcgtaacctgacctatgcagcgccggtttacattaagattaccaaaaaagtgcaagctgctgtggaaacggaaatacctttgcacgagctagatgatgctcaacagacagagtatgccaaaactggtgagctgcctacacggttggcctggagatcggaggatgcgccagagcccgaaaattccaacaagcctgatgcctggaaggacatggtcttcgtcgggaaaatgccggtcatggtcaaatctaaagtctgccatttgagccgcgagcatgacgagaatttatttcttgtcaatgagtgcccatacgaccagggcggttactttgttatcaatggaagtgaaaaggtgcttatcgcccaagagcgttccgccgccaatattgttcaggtcttcaagaaggctcagcccagcccttatacatacactgctgaaatcagaagcgctctggagaaaggctcgcgtctcatctccagcatgatgctcaagctctacgggaagggtgagtcagctcgtggcggcttcgggcaaacgattcatacaactcttccgtttgttaaagcagaccttcctgtcgccattgtatttagagcacttggcgttgtctccgacgaggatattctcaaccacatttgttacgaccgaaaagactctcagatgctggaaatgcttcggccttgtatcgaggaagctttctgcgtccaagaccgtgaagtcgccctcgacttcattggtaagcgtggtaatcgggatcaggctggtctcggacgcgataaacgtattcgtgtggcacgcgacattcttcagaaagaaactttgcctcacatctcccaggaggctggtagcgagacgcgcaaagccttctttatcggttatatggtccacaaactactccagtgcgctctggggcgtcgggagcctgacgacagagaccacttcggcaaaaagcgtcttgatttagcaggtccactacttgcgaaactcttccgcggtatcatgcgccgaatgcactcagagctcgccaattacctgcgacgctgcgtcgagggtaaccgccacttcaatctagctgtcggtgttaagcctggcacgctttcgaacggcctcaagtactctcttgctactggtaattggggtgaccagaaaaaagctatgagctcaactgctggtgtgtcccaggttttgaaccgatatacttttgcctcaacactatcccaccttcgacgtaccaatacacccattggcagagatggcaaactggcaaaacctcgtcagcttcataatactcattggggtcttgtctgccctgctgagacacctgaaggccaggcttgcggtcttgtcaagaatctgtctctcatgtgctacgtcagcgtcggctccccagccgagccgctcattgacttcatgatcaacaggggtatggaggtcattgaggagtacgaaccactcagatacccacacgctaccaagatttttgtcaacgggacctgggttggagttcatcaggaccccaagcaccttgctgaccaggtattcgacacccgccgcaagtcctacctgcagtatgaggtgtctcttgtcagagaaatccgtgaccaggaattcaagatcttctctgatgctggccgagtcatgcggcctgtttttactgtgcaaagtaaaaatgacccggagactggccttgaaaagggacagctcggtctcaccaaggatttagtcaacagactggcgcaagagcaagccgacccgccagatgatccagaaatgaagacgggttgggagggcttgatcaaagctggcgctgtcgagtatctagacgctgaagaagaggagacgtcaatgatttgcatgaccccggaggatctggagctttatagacttcagaaagcgggtgtagctgtcgacgacgaccatggcgatgatctgaacaagcgcctgaagactaaaaccaacccaactacacacatgtacactcactgtgaaattcacccgagtatgatcttgggtatctgcgcaagcattattcctttcccagaccacaatcaatcgcctcgtaacacctaccaatccgcaatgggtaaacaagccatgggcttcttcctgacaaattattctcgtcgtatggatactatggcaaatatcctgtactacccacaaaagcctttggcaacgactagatccatggagttcttgaagttccgagaattgcctgcaggtcaaaacgccatcgtcgcgattgcttgttattccggttataatcaggaggactctgtgattatgaatcaaagcagtatcgatcgcggactcttcagaagtttgttctttcgttcgtactctgatcaagagaaaaaggtcggtttgaactacacagagatattcgaaaagccgttccaccaaagcaccctgcgcatgaaacacggtacttacgacaagctcgatgaggacggtattgtcgctcctggcgttcgtgtgtctggagaagacattatcattggcaaaactgcgccaattgacccagagacgcaagacttgggcgcgcgtacaactgcgcatcagcgccgtgatatctctacgcctctgcgtagtaccgaaaatggtattgttgatcaagtcattgtgactgtcaacgccgacaacgtcaaatacgtcaaggtcagggttcgtacgaccaagataccccagatcggtgataagtttgcttcacgacacggccaaaaaggtaccattggtgtcacataccgacaggaagatatgccattcacgagagagggggtcaccccagatatcattatcaacccgcacgccattccctctcgaatgacaattgctcatctgatcgaatgtcttttgagcaaggtttcgactttagaaggtatggaaggcgatgctactccatttaccgacgtcactgtcgattctgtctccgacttgcttcgcaagcacggctatcagtcccgcgggtttgagatcatgtacaacggtcacactggtaagaaacttcgtgctcaggttttcttcggaccgacctattaccagcgtctgcgtcacatggtggacgacaaaattcacgcccgtgcccgtggccctgtgcagatcatgactagacaacctgtggagggtcgtgcaagggatggggggttgcgtttcggcgaaatggaacgtgattgcatgattgcgcatggtgcagcagcattcttgaaggagcgcctctttgaagtgtctgatgcgttccgagtgcacatctgcgagatttgcggcttgatgacgccgattgcaaatctttcaaagcaatcttttgaatgcagaccttgcaagaacaagaccaaaattgcccagattcacattccgtacgcagccaagctattgttccaagaacttcaatccatgaacattgcagcacgtatgtttacggacagatctggtgcctcgatcaggtag

SEQ ID NO: 27 - DNA, 1383 bp, synthetic construct
  source 1..1383: mol_type = other DNA
atgggcaaggacgacaagactcacatcaacgtcgtcgttatcggccacgtcgattccggcaagtctaccaccactggtcacttgatctaccagtgcggtggtattgacaagcgtaccattgagaagttcgagaaggaagccgctgaactcggcaagggttccttcaagtatgcctgggttcttgacaagctcaaggccgagcgtgagcgtggtatcaccattgatatcgctctctggaagttcgagactcccaagtaccacgtcaccgtcattgatgctcccggtcaccgtgatttcatcaagaacatgattactggtacttcccaggccgattgtgctattctcatcatcgccgccggtactggtgagttcgaggctggtatctccaaggatggccagacccgtgagcacgctctgctcgctttcaccctcggtgtcaagcagctcattgtcgccatcaacaagatggacaccaccaagtggtccgaggcccgttaccaggaaatcatcaaggagacttccagcttcatcaagaaggttggctacaaccccaaggctgttgctttcgtccccatctccggtttcaacggcgacaacatgcttgagccctccagcaactgcccctggtacaagggctgggagaaggagaccaaggctggcaagtctactggcaagacccttctcgaggccatcgacgctattgagccccccaagcgtcctaccgacaagcctctccgtcttcccctccaggatgtttacaagatcggtggtatcggaacggtgcccgtcggtcgtgttgagactggtatcatcaagcccggcatggtcgtcacctttgctcccgccaacgtcaccactgaagtcaagtccgtcgagatgcaccacgagcagcttactgagggtgttcccggtgacaacgtcggcttcaacgtgaagaacgtttccgtcaaggaaatccgtcgtggtaacgtcgctggtgactccaagaacgacccccccaatggcgctgcttccttcaacgcccaggtcattgtcatcaaccaccctggccagatcggtgccggctacgctcccgttcttgactgccacactgcccacattgcttgcaagttctccgagctcctcgagaagatcgaccgccgtaccggtaagtcggttgagaacaaccccaagttcatcaagtctggtgactccgccatcgtcaagatggttccctccaagcccatgtgcgttgaggctttcaccgactacccccctctgggccgtttcgccgtccgtgacatgcgtcagaccgtcgctgtcggtgtcatcaagtccgttgagaaggctgccgctggttcctccaaggtcaccaagtccgctgccaaggccaccaagaaataa

SEQ ID NO: 28 - DNA, 1383 bp, synthetic construct; identical to SEQ ID NO: 27
  source 1..1383: mol_type = other DNA

SEQ ID NO: 29 - DNA, 1383 bp, synthetic construct; identical to SEQ ID NO: 27
  source 1..1383: mol_type = other DNA

SEQ ID NO: 30 - DNA, 1383 bp, synthetic construct; identical to SEQ ID NO: 27
  source 1..1383: mol_type = other DNA

SEQ ID NO: 31 - DNA, 1383 bp, synthetic construct; identical to SEQ ID NO: 27
  source 1..1383: mol_type = other DNA

SEQ ID NO: 32 - DNA, 1383 bp, synthetic construct; identical to SEQ ID NO: 27
  source 1..1383: mol_type = other DNA

SEQ ID NO: 33 - DNA, 1383 bp, synthetic construct; identical to SEQ ID NO: 27
  source 1..1383: mol_type = other DNA

SEQ ID NO: 34 - DNA, 1383 bp, synthetic construct; identical to SEQ ID NO: 27
  source 1..1383: mol_type = other DNA

SEQ ID NO: 35 - DNA, 1350 bp, synthetic construct
  source 1..1350: mol_type = other DNA
atgcgtgagattgttcacctccagaccggccagtgcggtaaccaaatcggtgctgctttctggcagaccatctctggcgagcacggcctcgactccagcggtgtttacaatggcacttctgagcttcagctcgagcgcatgaatgtctacttcaacgaggcctccggcaacaagtatgtccctcgcgccgtcctcgtcgatcttgagcccggtaccatggatgctgtccgtgccggacccttcggtcagctcttccgtcccgacaacttcgttttcggtcagtccggtgccggcaacaactgggccaagggtcattacactgagggtgccgagctcgttgaccaggtcctcgacgttgttcgtcgtgaggccgaaggctgcgactgcctccagggtttccagatcacccactctcttggtggtggtactggtgctggtatgggtactctgctcatctccaagatccgcgaagagtttcccgaccgcatgatggccaccttttccgtggttccctctcccggcaactccgacaccgttgtcgagccctacaacgctactctctccgttcaccagctcgttgagaactccgacgagaccttctgtatcgacaaccaggctctgtacgatatctgcatgcgtaccctgaagctatccaatccttcgtacggtgacctgaaccacctcgtttccgtcgtcatgtccggcatcaccacctgcctgcgtttccctggtcagcttaactctgatcttcgcaagctcgccgtcaacatggttcctttccctcgtcttcactttttcatggtcggctttgctcccctgacgagccgtggtgcccactccttccgcgccgtctctgttcccgagctcactcagcagatgttcgaccctaagaacatgatggctgcttctgacttccgcaacggtcgctacctgacctgctctgccattttccgtggtaaggttgccatgaaggaggttgaggaccagatgcgtaatgtgcagaccaagaactccagctacttcgtcgagtggatccccaacaacatccagaacgctctctgcgccgttcccccccgcggacttaagatgtcgtctaccttcattggtaactcgacctctatccaggatctcttcaagcgtgtcggtgagcagttctccgccatgttccgtcgcaaggctttccttcattggtacactggtgaaggtatggacgagatggagttcactgaggctgagtccaacatgaacgatcttatctccgaataccagcagtaccaggacgctggcattgatgatgaggaagaggaatacgaggaggagctccctgtcgagggcgaggagcctttgtaa

SEQ ID NO: 36 - DNA, 1350 bp, synthetic construct; identical to SEQ ID NO: 35
  source 1..1350: mol_type = other DNA

SEQ ID NO: 37 - DNA, 1350 bp, synthetic construct; identical to SEQ ID NO: 35
  source 1..1350: mol_type = other DNA

SEQ ID NO: 38 - DNA, 1350 bp, synthetic construct; identical to SEQ ID NO: 35
  source 1..1350: mol_type = other DNA

SEQ ID NO: 39 - DNA, 1350 bp, synthetic construct
  source 1..1350: mol_type = other DNA
atgcgtgagattgttcacctccagaccggccagtgcggtaaccaaatcggtgctgctttctggcagaccatctctggcgagcacggcctcgactccagcggtgtttacaatggcacttctgagcttcagctcgagcgcatgaatgtctacttcaacgaggcctccggcaacaagtatgttcctcgcgccgtcctcgtcgatcttgagcccggtaccatggatgctgtccgtgccggtcccttcggtcagctcttccgtcccgacaacttcgttttcggtcagtccggtgccggcaacaactgggccaagggtcattacactgagggtgccgagctcgttgaccaggtcctcgacgttgttcgtcgtgaggccgaaggctgcgactgcctccagggtttccagatcacccactctcttggtggtggtactggtgctggtatgggtactctgctcatctccaagatccgcgaagagtttcccgaccgcatgatggccaccttttccgtggttccctctcccggcaactccgacaccgttgtcgagccctacaacgctactctctccgttcaccagctcgttgagaactccgacgagaccttctgtatcgacaaccaggctctgtacgatatctgcatgcgtaccctgaagctatctaatccttcgtacggtgacctgaaccacctcgtttccgtcgtcatgtccggcatcaccacctgcctgcgtttccctggtcagcttaactctgatcttcgcaagctcgccgtcaacatggttcctttccctcgtcttcactttttcatggtcggctttgctcccctgacgagccgtggtgcacactccttccgcgctgtctctgttcctgagctcactcagcagatgttcgaccctaagaacatgatggctgcttctgacttccgtaacggtcgctacctgacctgctctgccattttccgtggcaaggttgccatgaaggaggttgaggaccagatgcgtaatgtgcagaccaagaactccagctacttcgtcgagtggatccccaacaacatccagaacgctctctgcgccgttcccccccgcggacttaagatgtcgtctaccttcattggtaactcgacctctatccaggatctcttcaagcgtgtcggtgaacagttctccgccatgttccgtcgcaaggctttccttcattggtacactggcgaaggtatggacgagatggagttcactgaggctgagtccaacatgaacgatcttatctccgaataccagcagtaccaggacgctggcattgatgatgaggaagaggaatacgaggaggagctccctgtcgagggcgaggagcctttgtaa

SEQ ID NO: 40 - DNA, 1350 bp, synthetic construct; identical to SEQ ID NO: 35
  source 1..1350: mol_type = other DNA

SEQ ID NO: 41 - DNA, 1350 bp, synthetic construct; identical to SEQ ID NO: 35
  source 1..1350: mol_type = other DNA

SEQ ID NO: 42 - DNA, 1350 bp, synthetic construct; identical to SEQ ID NO: 35
  source 1..1350: mol_type = other DNA

(19) United States
(12) Patent Application Publication
(10) Pub. No.: US 2026/0271941 A1
(43) Pub. Date: Sep. 17, 2026

(54) COMPOSITIONS COMPRISING BEAUVERIA BASSIANA AND METHODS OF USE THEREOF

(71) Applicant: Loam Bio Pty Ltd, Orange (AU)

(72) Inventors: Tegan NOCK, Orange (AU); Gyongyver KOROSI, Orange (AU); Brooke BRUNING, Orange (AU); Suresh SUBASHCHANDRABOSE, Orange (AU); Neeraj PURUSHOTHAM, Orange (AU); Jamila NASIM, Orange (AU); James PRESNAIL, St. Paul, MN (US); Ray Riley, St. Paul, MN (US); Venkatachalam LAKSHMANAN, St. Paul, MN (US); Karen PACO, St. Paul, MN (US); Tristan YANG, St. Paul, MN (US)

(21) Appl. No.: 19/168,316

(22) PCT Filed: Mar. 25, 2024
(86) PCT No.: PCT/US2024/021389

§ 371 Date: Sep. 23, 2025

Related U.S. Application Data
(60) Provisional application No. 63/491,907, filed on Mar. 23, 2023.

(51) Int. Cl. A01N 63/30; A01P 21/00

(52) U.S. Cl. CPC A01N 63/30; A01P 21/00

(57) ABSTRACT

The disclosure provides a method of increasing soil organic carbon (SOC), comprising: heterologously disposing one or more fungal strains to treat a plant element, wherein the one or more fungal strains comprises a nuclear ribosomal internal transcribed spacer (ITS) sequence that is at least 97% identical to the nucleotide sequence of any one of SEQ ID NOs: 1-8; and the one or more fungal strains are heterologously disposed in an effective amount to increase organic carbon in soil supporting a plant derived from the treated plant element relative to soil supporting a reference plant derived from a reference plant element.

### CROSS REFERENCE TO RELATED APPLICATIONS

This application claims the benefit of U.S. Provisional Patent Application No. 63/491,907, filed on Mar. 23, 2023, the contents of which are incorporated herein by reference in their entirety.

### TECHNICAL FIELD

The present disclosure relates to methods and compositions for increasing carbon content in the soil, mitigating atmospheric carbon dioxide, and increasing plant yield with Beauveria bassiana.

### BACKGROUND

Carbon dioxide and methane absorb and retain heat in the atmosphere; therefore, both gasses play a pivotal role in the greenhouse effect. As methane is much more short-lived than carbon dioxide, carbon dioxide is often considered more important than that methane to the greenhouse effect.

The life cycle of carbon includes the removal of carbon dioxide from the atmosphere by plants through photosynthesis. During photosynthesis, the carbon dioxide gets absorbed through the stroma of leaves, and the carbon dioxide is further converted into sugars. Such sugars become nutrients for plants and microbes present in the soil. Finally, carbon enters back into the atmosphere in the form of carbon dioxide by respiration and combustion. Hence, a balanced amount of carbon dioxide release and absorption is an essential step for balancing the ecosystem.

Human activities such as the combustion of fuels, overpopulation, forest degradation, soil erosion, etc., have increased atmospheric carbon dioxide. Therefore, approaches for sequestering carbon dioxide from the atmosphere present an essential component of a strategy for reducing or controlling atmospheric carbon dioxide. However, for this to be successful, there must also be a reduction in the release of carbon dioxide from the soil back into the atmosphere.

Decay of plants, animals, and microbes into the soil can lead to the build-up of soil organic carbon (SOC), an essential nutrient which promotes physical stability of the structure of the soil, soil aeration, water drainage and retention, thus reducing soil erosion and nutrient leaching. However, intensive cultivation has also led to a decline in SOC, eventually making the land unsuitable for commercial crop production. As such, the benefits associated with SOC can be seen as two-fold: the sequestration of atmospheric carbon, provided the soil, and the overall improvement of the soil quality retain the carbon.

It would be advantageous to develop compositions, treatments, and methods for increasing soil carbon in a manner that will produce more stable carbon in the soil by sequestering atmospheric carbon and provide benefits to commercial crop plants.

### SUMMARY

In some aspects, present disclosure provides a method of increasing soil organic carbon (SOC), comprising: heterologously disposing one or more fungal strains to treat a plant element, wherein the one or more fungal strains comprises a nuclear ribosomal internal transcribed spacer (ITS) sequence that is at least 97% identical to the nucleotide sequence of any one of SEQ ID NOs: 1-8; and the one or more fungal strains are heterologously disposed in an effective amount to increase organic carbon in soil supporting a plant derived from the treated plant element relative to soil supporting a reference plant derived from a reference plant element.

In other aspects, the disclosure provides a method of increasing soil organic carbon (SOC), comprising: mechanically inoculating a soil with a synthetic combination of one or more heterologously disposed fungal strains, wherein the one or more fungal strains comprises a nuclear ribosomal internal transcribed spacer (ITS) sequence that is at least 97% identical to the nucleotide sequence of any one of SEQ ID NOs: 1-8; and the one or more fungal strains are in an effective amount to increase organic carbon in the inoculated soil relative to a reference soil.

In one aspect, the one or more fungal strains further comprises: (i) a B Locus Nuclear Intergenic Region (Bloc) sequence that is at least 97% identical to the nucleotide sequence of SEQ ID NO: 9 or 10; (ii) an RPB1 sequence that is at least 97% identical to the nucleotide sequence of any one of SEQ ID NOs: 11-18; (iii) an RPB2 sequence that is at least 97% identical to the nucleotide sequence of any one of SEQ ID NOs: 19-26; (iv) a TEF1 sequence that is at least 97% identical to the nucleotide sequence of any one of SEQ ID NOs: 27-34; (v) a TUB2 sequence that is at least 97% identical to the nucleotide sequence of any one of SEQ ID NOs: 35-42; or (vi) any combination (i) to (v).

In another aspect, the one or more fungal strains are selected from the group consisting of: Beauveria bassiana AU-16727 (NMI Accession No. V23/003855), B. bassiana US-52 (ATCC Accession No. PTA-127541), B. bassiana US-675 (ATCC Accession No. PTA-127540), B. bassiana US-699 (ATCC Accession No. PTA-127538), B. bassiana US-707 (ATCC Accession No. PTA-127542), B. bassiana US-803 (ATCC Accession No. PTA-127539), B. bassiana US-670 (ATCC Accession No. PTA-127741), B. bassiana US-935 (ATCC Accession No. PTA-127743), and a mutant thereof having all identifying characteristics of the respective strain.

In some aspects, the method further comprises an initial step of identifying the soil as having a soil organic carbon (SOC) below a threshold level. In one aspect, the threshold level is an SOC (% wt/wt) below 5%, 4%, 3%, 2%, 1%, 0.9%, 0.8%, 0.7%, 0.6%, 0.5%, 0.4%, 0.3%, 0.2%, or 0.1%.

In some aspects, the soil and/or plant element are non-native to the one or more fungal strains. In one aspect, the non-native plant element is from a plant selected from the group consisting of wheat, rice, corn (maize), canola, rye, oats, barley, sorghum, millet, flax, hemp, jute, cotton, sugar cane, soybeans, alfalfa, clover, Desmanthus, peanuts, lentils, lupins, peas, and chickpea. In another aspect, the non-native plant element is from a plant selected from the group consisting of lucerne, arrow leaf clover, balansa clover, chicory, plantain, phalaris, cocksfoot, fescue, prairie grass, Warrego summer grass, Italian rye grass, perennial rye grass, biserrula, serradella, gland clover, bladder clover, switchgrass, radish, medic, buckwheat, cow pea, lablab, sunn hemp, sunflower, tillage radish, and subterranean clover.

In some aspects, the organic carbon in the soil is increased in the stable forms of aggregate carbon fraction (AggC), aggregate occluded particulate organic carbon (POC), and/or mineral-associated organic carbon (MAOC).

In certain aspects, the disclosure provides a method for sequestering atmospheric carbon for storage as soil organic carbon (SOC), comprising: heterologously disposing one or more fungal strains to treat a plant element, wherein the one or more fungal strains comprises a nuclear ribosomal internal transcribed spacer (ITS) sequence that is at least 97% identical to the nucleotide sequence of any one of SEQ ID NOs: 1-8; and the one or more fungal strains are in an effective amount to increase sequestered atmospheric carbon in soil supporting a plant derived from the treated plant element relative to soil supporting a reference plant derived from a reference plant element.

In one aspect, the disclosure provides a method for sequestering atmospheric carbon for storage as soil organic carbon (SOC), comprising: mechanically inoculating a soil with a synthetic combination of one or more heterologously disposed fungal strains, wherein the one or more fungal strains comprises a nuclear ribosomal internal transcribed spacer (ITS) sequence that is at least 97% identical to the nucleotide sequence of any one of SEQ ID NOs: 1-8; and the one or more fungal strains are in an effective amount to increase sequestered atmospheric carbon in the inoculated soil relative to a reference soil.

In certain aspects, the one or more fungal strains increase organic carbon in the soil by expressing enzymes involved in the melanin biosynthetic pathway, carbohydrate active enzymes, lignin degrading enzymes, or a combination thereof. In other aspects, the one or more fungal strains demonstrate decreased virulence compared to known insect pathogens of the species Beauveria bassiana.

In some aspects, the disclosure provides a method of enhancing plant growth, comprising: heterologously disposing one or more fungal strains to treat a plant element, wherein the one or more fungal strains comprises a nuclear ribosomal internal transcribed spacer (ITS) sequence that is at least 97% identical to the nucleotide sequence of any one of SEQ ID NOs: 1-8; and the one or more fungal strains are in an effective amount to enhance the growth of a plant derived from the treated plant element relative to a reference plant derived from a reference plant element.

In certain aspects, the plant exhibits at least one of increased root number, increased root length, increased root mass, increased root volume, increased leaf area, increased leaf number, increased pod number, increased plant height, increased shoot mass, increased chlorophyll content, increased nodulation, and increased yield, as compared to the reference plant.

In other aspects, the disclosure provides a synthetic combination comprising a purified population of one or more fungal strains heterologously disposed to a plant element, wherein the one or more fungal strains are heterologous to the plant element and comprise a nuclear ribosomal internal transcribed spacer (ITS) sequence that is at least 97% identical to the nucleotide sequence of any one of SEQ ID NOs: 1-8; and an agriculturally acceptable carrier; wherein the one or more fungal strains are present in an effective amount to increase soil organic carbon (SOC) in soil supporting a plant derived from the plant element in the synthetic combination relative to soil supporting a reference plant derived from a reference plant element.

In some aspects, the one or more fungal strains in the synthetic combination further comprises: (i) a B Locus Nuclear Intergenic Region (Bloc) sequence that is at least 97% identical to the nucleotide sequence of SEQ ID NO: 9 or 10; (ii) an RPB1 sequence that is at least 97% identical to the nucleotide sequence of any one of SEQ ID NOs: 11-18; (iii) an RPB2 sequence that is at least 97% identical to the nucleotide sequence of any one of SEQ ID NOs: 19-26; (iv) a TEF1 sequence that is at least 97% identical to the nucleotide sequence of any one of SEQ ID NOs: 27-34; (v) a TUB2 sequence that is at least 97% identical to the nucleotide sequence of any one of SEQ ID NOs: 35-42; or (vi) any combination (i) to (v).

In one aspect, (a) the combination is formulated as a solid, liquid or gel; (b) the combination is formulated as a powder, pellet or granules; or (c) the combination is formulated as an emulsion, colloid, suspension or solution. In another aspect, the one or more fungal strains are present in the combination at a concentration of at least 10³ colony forming units (CFU) per milliliter or gram.

In some aspects, the plant element is a whole plant, seedling, meristematic tissue, ground tissue, vascular tissue, dermal tissue, seed, leaf, root, shoot, stem, flower, fruit, stolon, bulb, tuber, corm, keikis, shoot, or bud.

In one aspect, the disclosure provides a plant grown from the disclosed synthetic combination, wherein soil supporting the plant exhibits increased soil organic carbon (SOC) relative to soil supporting a reference plant. In another aspect, the disclosure provides a bag or container comprising the disclosed synthetic combination. In another aspect, disclosure provides a kit comprising the disclosed synthetic combination.

In other aspects, the disclosure provides a cell or a biologically pure culture of one or more fungal strains selected from the group consisting of Beauveria bassiana AU-16727 (NMI Accession No. V23/003855), B. bassiana US-52 (ATCC Accession No. PTA-127541), B. bassiana US-675 (ATCC Accession No. PTA-127540), B. bassiana US-699 (ATCC Accession No. PTA-127538), B. bassiana US-707 (ATCC Accession No. PTA-127542), and B. bassiana US-803 (ATCC Accession No. PTA-127539), B. bassiana US-670 (ATCC Accession No. PTA-127741), B. bassiana US-935 (ATCC Accession No. PTA-127743), and a mutant thereof having all identifying characteristics of the respective strain.

In one aspect, the disclosure provides an agricultural composition comprising the disclosed cell or a biologically pure culture and, optionally, an agriculturally acceptable carrier. In some aspects, the agricultural composition is heterologously disposed on at least a portion of an outer surface of a plant, plant part or plant seed.

In another aspect, the disclosure provides a bioorganic soil conditioner comprising the disclosed cell or a biologically pure culture and, optionally, an agriculturally acceptable carrier. In one aspect, the agriculturally acceptable carrier comprises one or more of talc, an oil, kaolin clay, a dispersant, a surfactant, and a nutrient.

In one aspect, the one or more fungal strains increase organic carbon in the soil by expressing enzymes involved in the melanin biosynthetic pathway, carbohydrate active enzymes, lignin degrading enzymes, or a combination thereof. In another aspect, the one or more fungal strains demonstrate decreased virulence compared to known insect pathogens of the species Beauveria bassiana.

In other aspects, the plant exhibits at least one of increased root number, increased root length, increased root mass, increased root volume, increased leaf area, increased leaf number, increased pod number, increased plant height, increased shoot mass, increased chlorophyll content, increased nodulation, and increased yield, as compared to an untreated control plant or a reference plant.

The identifying characteristics of a strain include morphology; biochemical properties (e.g., metabolic pathways, enzyme production, and substrate utilization patterns); genetic makeup (e.g., genetic markers or sequences specific to the strain); physiological traits (e.g., growth requirements such as temperature, pH, and oxygen levels); growth rate; resistance to environmental stresses such as antibiotics and UV radiation; metabolic profile or fingerprint; plant host specificity; colonization patterns (e.g., colonization of plant tissues, including root colonization, systemic spread within the plant, or presence in aerial parts such as leaves and stems); mutualistic relationships (i.e., mutualistic interactions between the strain and its host plant, such as nutrient exchange, enhanced stress tolerance, or disease resistance); secondary metabolite production; molecular markers; environmental adaptations (e.g., adaptation to soil environments, including tolerance to soil pH, moisture levels, and nutrient availability); ecological role (e.g., impact on soil microbial communities, nutrient cycling, and overall soil health); and transmission dynamics (i.e., transmission of the strain between plants and within soil environments, including dispersal mechanisms and factors influencing establishment in new host plants).

### BRIEF DESCRIPTION OF THE DRAWINGS

FIG. 1 depicts a graph tracking the stability of a powder spore preparation of Beauveria bassiana US-52 (ATCC Accession No. PTA-127541) stored at 4° C., 24° C., or 30° C. for between 70 days and 113 days.

FIG. 2 depicts a Orthovenn3 diagram indicating expansions (indicated with “+”) and contractions (indicated with “−”) in the gene families for B. bassiana US-52 (ATCC Accession No. PTA-127541), B. bassiana US-675 (ATCC Accession No. PTA-127540), B. bassiana US-699 (ATCC Accession No. PTA-127538), B. bassiana US-707 (ATCC Accession No. PTA-127542), B. bassiana US-803 (ATCC Accession No. PTA-127539), B. bassiana AU-16727 (NMI Accession No. V23/003855), B. bassiana US-935 (ATCC Accession No. PTA-127743), B. bassiana US-670 (ATCC Accession No. PTA-127741), and B. bassiana GCA 001682635 (also known as “GCA”).

FIGS. 3A-3E depict phylogenetic trees generated using the genomic loci of ITS, RPB1, RPB2, TEF1, and TUB2, respectively, as DNA barcode markers for the taxonomic identification of B. bassiana US-52 (ATCC Accession No. PTA-127541), B. bassiana US-675 (ATCC Accession No. PTA-127540), B. bassiana US-699 (ATCC Accession No. PTA-127538), B. bassiana US-707 (ATCC Accession No. PTA-127542), B. bassiana US-803 (ATCC Accession No. PTA-127539), B. bassiana AU-16727 (NMI Accession No. V23/003855), B. bassiana US-935 (ATCC Accession No. PTA-127743), and B. bassiana US-670 (ATCC Accession No. PTA-127741).

### DETAILED DESCRIPTION

The present disclosure relates to methods and related technologies for increasing soil soil organic carbon (SOC) and/or increasing yield of a crop plant. The method comprises inoculating the soil and/or the plant with an effective amount of one or more compatible, non-pathogenic strains of fungal species.

It will be appreciated that the strains of fungi will be fungal strains that are crop-compatible with the crop plant to which they are to be applied, but the crop need not necessarily be a native host of the fungi. A fungal strain that is crop-compatible with a crop plant is a strain that is non-pathogenic to that crop plant. Methods for assessing whether a strain of fungus is non-pathogenic to a particular crop plant are known in the art.

An increase in soil organic carbon is an increase in the amount of organic carbon in soil associated with the crop plant inoculated with the one or more fungal species relative to the amount of organic carbon in uninoculated soil. In this context, the soil associated with the crop plant is soil surrounding the roots of the crop plant and from which the crop plant derives nutrients. An increase in plant yield is an increase in fruit, grain or vegetative tissue production of the plant relative to that of a plant that has not been treated with the one or more fungal species described herein. For example, an increase in yield of a soybean plant is an increase in the number and/or weight of seed pods produced by a soybean plant relative to that of an untreated soybean plant.

The inventors have found that growing a crop plant that has been inoculated with certain crop-compatible fungal strains results in an increase in soil organic carbon and/or an increase in yield of crop plants.

The inventors have found that various crop plants inoculated with fungal species, and in particular, endophytic fungal species, exhibit increased yield relative to uninoculated plants. The inventors have further found that the soil in which these plants are grown has increased organic carbon content relative to soil in which uninoculated plants are grown.

The term “endophytic” relates to a microbe that generally lives within a plant for at least part of its lifecycle, often due to the microbe being able to grow inward into plant tissues in finger-like projections from a superficial site of origin. These fungi can infiltrate plant living tissues for at least a portion of the fungal life cycle often without causing any apparent diseases or harm to the plant that is a native host, in that they are generally not pathogenic to their native hosts. It would be understood that the one or more fungal genera, species or strains of the methods described herein can exist during some portion of the fungal life cycle within the roots of a plant host as an endophyte and in other parts of its life cycle within the soil and will typically alternate or cycle between an endophytic root phase and a free-living soil phase.

Though some endophytic fungi are known for enriching the organic carbon in the soil, each fungal species will generally behave differently when associated with different, and/or non-native plant hosts and/or soil environments and will stabilize the organic carbon with varying efficiency.

In some embodiments, the one or more fungal species has a nuclear ribosomal internal transcribed spacer (ITS) sequence that is at least 90% identical, typically at least 91%, least 92%, at least 93%, at least 94%, at least 95%, at least 96%, at least 97%, at least 98%, or at least 99% identical, more typically 100% identical, with the nucleotide sequence of SEQ ID No: 1, 2, 3, 4, 5, or 6. In other embodiments, the one or more fungal species has a B Locus Nuclear Intergenic Region (Bloc) sequence that is at least 90% identical, typically at least 91%, least 92%, at least 93%, at least 94%, at least 95%, at least 96%, at least 97%, at least 98%, or at least 99% identical, more typically 100% identical, with the nucleotide sequence of SEQ ID No: 7 or 8.

The terms “identical” or “% identical,” in the context of two or more nucleic acids refers to two or more sequences that are the same or have a specified percentage of nucleotides that are the same (i.e., 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/, or the like).

Algorithms for determining % identity are known in the art. Examples of algorithms suitable for determining percent sequence identity are the BLAST and BLAST 2.0 algorithms, described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information.

A “soil conditioner” denotes a mixture of substances or a blend that can be added directly to the soil to improve soil characteristics or to an agricultural or fertilizer composition, which, in turn, is added to the soil. The soil conditioner can be applied to any type of soil, including black cotton soil, saline soil, medium to high saline soil, yellow soil, sandy soil, loamy soil, alluvial soil (delta soil), lava soil, topsoil, and subsoil that can be used in crop/plant production.

As used herein, the term “effective amount” means a sufficient quantity of a substance (e.g., fungus) to promote an increase in soil carbon in a treated soil and/or a plant trait (e.g., yield) in a treated plant compared to an untreated soil or untreated plant. This term is not to be construed to limit the disclosure to a specific quantity, e.g., the number of fungal cells. Rather, the present disclosure encompasses any amount of one or more fungal species sufficient to achieve the stated purpose. The amount of one or more fungal species should not be so large as to cause adverse effects on the plant. Generally, the amount of one or more fungal species may vary with how the fungi are applied (e.g., to the soil, to the seed or to the seedling) and can be determined by a person skilled in the art.

Throughout the specification and claims, unless the context requires otherwise, the term “substantially” or “about” will be understood to not be limited to the value for the range qualified by the terms. For example, the term “about” may include a range that is +5%, +2.5% or +1% of the value to which the term is applied.

The terms “US-52” and “NRRL 22864” are synonymous and refer to a strain of Beauveria bassiana. Certain microbial strains used in this work were provided by the USDA-ARS Culture Collection (NRRL).

A “plant element” is intended to generically reference either a whole plant or a plant component, including but not limited to plant tissues and regions thereof, plant parts and regions thereof, and to plant cell types. A plant element is preferably one of the following: whole plant, seedling, meristematic tissue, ground tissue, vascular tissue, dermal tissue, seed, leaf, root, shoot, stem, flower, fruit, stolon, bulb, tuber, corm, keikis, shoot, bud.

A treatment (e.g., a fungal strain) is “heterologously disposed” when mechanically or manually applied, artificially inoculated or disposed onto or into a plant element, seedling, plant or onto or into a plant growth medium or onto or into a treatment formulation so that the treatment exists on or in the plant element, seedling, plant, plant growth medium, or formulation in a manner not found in nature prior to the application of the treatment, e.g., said combination which is not found in nature in that plant variety, at that stage in plant development, in that plant tissue, in that abundance, or in that growth environment for example, drought, flood, cold, nutrient deficiency, etc.). In some embodiments, such a manner is contemplated to be selected from the group consisting of: the presence of the fungal strain; presence of the fungal strain in a different number of cells, concentration, or amount; the presence of the fungal strain in a different plant element, tissue, cell type, or other physical location in or on the plant; the presence of the fungal strain at different time period, e.g. developmental phase of the plant or plant element, time of day, time of season, and combinations thereof. In some embodiments, “heterologously disposed” means that the fungal strain is applied to a different tissue or cell type of the plant element than that in which the fungal strain is naturally found. In some embodiments, “heterologously disposed” means that the fungal strain is applied to a developmental stage of the plant element, seedling, or plant in which said fungal strain is not naturally associated but may be associated at other stages. For example, if a fungal strain is normally found at the flowering stage of a plant and no other stage, a fungal strain applied at the seedling stage may be considered to be heterologously disposed. In some embodiments, a fungal strain is heterologously disposed when the fungal strain is normally found in the root tissue of a plant element but not in the leaf tissue, and the fungal strain is applied to the leaf. In another non-limiting example, if a fungal strain is naturally found in the mesophyll layer of leaf tissue but is being applied to the epithelial layer, the fungal strain would be considered to be heterologously disposed. In some embodiments, “heterologously disposed” means that the native plant element, seedling, or plant does not contain detectable levels of the microbe in that same plant element, seedling, or plant. In some embodiments, “heterologously disposed” means that the fungal strain being applied is at a greater concentration, number, or amount on the plant element, seedling, or plant, than that which is naturally found in said plant element, seedling, or plant. For example, a fungal strain is heterologously disposed when present at a concentration that is at least 1.5 times greater, between 1.5 and 2 times greater, 2 times greater, between 2 and 3 times greater, 3 times greater, between 3 and 5 times greater, 5 times greater, between 5 and 7 times greater, 7 times greater, between 7 and 10 times greater, 10 times greater, or even greater than 10 times higher number, amount, or concentration than the concentration that was present prior to the disposition of said fungal strain. In another non-limiting example, a fungal strain that is naturally found in a leaf tissue of a cupressaceous tree would be considered heterologous to leaf tissue of a maize, wheat, cotton, soybean plant. In another example, a fungal strain that is naturally found in leaf tissue of a maize, spring wheat, cotton, soybean plant is considered heterologous to a leaf tissue of another maize, spring wheat, cotton, soybean plant that naturally lacks said fungal strain.

A “reference plant”, “reference plant element”, “reference agricultural plant” or “reference seed” is a similarly situated plant or seed of the same species, strain, or cultivar to which a treatment, formulation, composition or fungal endophyte preparation as described herein is not administered or contacted. A reference plant, therefore, is identical to the treated plant except for the presence of the active ingredient (e.g. fungal endophyte) to be tested and can serve as a control for detecting the effects of the treatment (e.g. active ingredient) conferred to the plant. A plurality of reference plants may be referred to as a “reference population”.

A “reference environment” refers to the environment, treatment or condition of the plant in which a measurement is made. For example, carbon sequestration or soil organic carbon accumulation with a plant heterologously disposed to a fungal endophyte can be measured in a reference environment of drought stress and compared with the carbon sequestration or soil organic carbon accumulation in a reference agricultural plant under the same conditions of drought stress. Alternatively, the carbon sequestration or soil organic carbon accumulation with a plant heterologously disposed to a fungal endophyte and reference agricultural plant can be measured under identical conditions of no stress.

A “reference soil” is a similarly situated soil with respect to composition (e.g., organic content or mineral composition), texture, pH, structure, moisture content, biological activity, compaction, nutrient levels, geological origin, etc. to which a treatment, formulation, composition or fungal endophyte preparation as described herein is not administered, applied, inoculated, or contacted. A reference soil, therefore, is similar or identical to the treated soil except for the presence of the active ingredient (e.g. fungal endophyte) to be tested and can serve as a control for detecting the effects of the treatment (e.g. active ingredient) conferred to the soil.

A “synthetic composition” (also known as a “synthetic combination”) comprises one or more fungal endophytes combined by human endeavor with a heterologously disposed plant element or a heterologously disposed treatment formulation, said combination which is not found in nature. In some embodiments, a synthetic composition comprises both one or more plant elements and one or more formulation components combined by human endeavor with an isolated, purified fungal endophyte composition. In some embodiments, said purified fungal endophyte composition is mechanically or manually applied, artificially inoculated or disposed on a plant element in a manner that is not found on or in the plant element before application of the purified fungal endophyte composition, e.g., said combination or association which is not found in nature.

A “biologically pure culture” refers to a carefully cultivated population of microorganisms containing only a single species or strain, devoid of any contamination by other microorganisms. This purity ensures consistency and reliability in the production of various commercial products such as biological inoculants or conditioners applied to plant elements or soil. The purity of the culture also allows for precise control over the microbial characteristics and performance, ensuring high-quality and standardized outcomes in industrial processes and product formulations.

In some embodiments, a synthetic composition is applied mechanically or manually or artificially inoculated to a plant element in a seed treatment, root wash, seedling soak, foliar application, soil inocula, in-furrow application, sidedress application, soil pre-treatment, wound inoculation, drip tape irrigation, vector-mediation via a pollinator, injection, osmopriming, hydroponics, aquaponics, aeroponics, and combinations thereof. Application to the plant may be achieved, for example, as a powder for surface deposition onto plant leaves or seeds, as a spray to the whole plant or selected plant element, as part of a drip to the soil or the roots, or as a coating onto the plant element prior to or after planting. Such examples are meant to be illustrative and not limiting to the scope of the invention.

An “effective amount” of one or more fungal endophytes is the amount capable of improving a trait of agronomic importance or tolerance by at least 0.1%, at least 0.5%, at least 1%, at least 2%, at least 3%, between 3% and 5%, at least 5%, between 5% and 10%, at least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, as compared to a reference plant element not further comprising said fungal endophyte. In some embodiments, an effective amount of treatment comprising an endophyte is at least 10 CFU per unit of plant element, at least 10² CFU per unit of plant element, between 10² and 10³ CFU per unit of plant element, at least about 10³ CFU per unit of plant element, between 10³ and 10⁴ CFU per unit of plant element, at least about 10⁴ CFU per unit of plant element, between 10⁴ and 10⁵ CFU per unit of plant element, at least about 10⁵ CFU, between 10⁵ and 10⁶ CFU per unit of plant element, at least about 10⁶ CFU per unit of plant element, between 10⁶ and 10⁷ CFU per unit of plant element, at least about 10⁷ CFU per unit of plant element, between 10⁷ and 10⁸ CFU per unit of plant element, or even greater than 10⁸ CFU per unit of plant element. A unit of a plant element may be an individual plant element, e.g. an individual seed, or a unit of area surface area of a plant element, e.g. a square centimeter of leaf tissue, or unit of surface area of a plant element, e.g. a cubic centimeter of root.

### Deposited Fungal Strains

Biological deposits of each of the fungal strains listed in Table 1 were made on the dates shown at the American Type Culture Collection (ATCC®), located at 10801 University Blvd., Manassas, VA 20110, USA, or the National Measurement Institute (NMI), 1/153 Bertie Street, Port Melbourne, Victoria 3207, Australia, under the provisions of the Budapest Treaty and assigned by each International Depositary Authority (IDA) the accession numbers indicated. Upon issuance of a patent, all restrictions upon the deposits will be irrevocably removed. The deposits are intended to meet the requirements of 37 CFR §§ 1.801-1.809. The deposits will be maintained in the IDAs for a period of 30 years, or 5 years after the last request, or for the effective, enforceable life of the patent, whichever is longer, and will be replaced, if necessary, during that period; and the requirements of 37 CFR §§ 1.801-1.809 are met.

TABLE 1

| Strain Number | Species | IDA | Date of Deposit | Accession No. |
|---|---|---|---|---|
| AU-16727 | Beauveria | NMI | 24 Feb. 2023 | V23/003855 |
| | bassiana | | | |
| US-52 | Beauveria | ATCC | 2 Mar. 2023 | PTA-127541 |
| | bassiana | | | |
| US-675 | Beauveria | ATCC | 2 Mar. 2023 | PTA-127540 |
| | bassiana | | | |
| US-699 | Beauveria | ATCC | 2 Mar. 2023 | PTA-127538 |
| | bassiana | | | |
| US-707 | Beauveria | ATCC | 2 Mar. 2023 | PTA-127542 |
| | bassiana | | | |
| US-803 | Beauveria | ATCC | 2 Mar. 2023 | PTA-127539 |
| | bassiana | | | |
| US-670 | Beauveria | ATCC | 14 Mar. 2024 | PTA-127741 |
| | bassiana | | | |
| US-935 | Beauveria | ATCC | 14 Mar. 2024 | PTA-127743 |
| | bassiana | | | |

It would be understood that fungal strains from the same species as those described herein would have similar desirable attributes and are encompassed by the treatments and methods of the present invention.

### Internal Transcribed Spacer (ITS) and B Locus Nuclear Intergenic Region (Bloc) Sequences of Fungal Strains

In one aspect, the fungal strain comprises a nuclear ribosomal internal transcribed spacer (ITS) sequence that is at least 80% identical, at least 85% identical, at least 90% identical, at least 91% identical, at least 92% identical, at least 93% identical, at least 94% identical, at least 95% identical, at least 96% identical, at least 97% identical, at least 98% identical, or at least 99% identical to the nucleotide sequence of any one of SEQ ID NOs: 1-8 (see Table 2).

It has been suggested that that B Locus Nuclear Intergenic Region (Bloc) sequences can be used for phylogenetic analysis of Beauveria bassiana (Rehner). In one aspect, the fungal strain comprises a Bloc sequence that is at least 80% identical, at least 85% identical, at least 90% identical, at least 91% identical, at least 92% identical, at least 93% identical, at least 94% identical, at least 95% identical, at least 96% identical, at least 97% identical, at least 98% identical, or at least 99% identical to the nucleotide sequence of SEQ ID NO: 9 or 10 (see Table 3).

In one aspect, the fungal strain comprises an RPB1 sequence that is at least 80% identical, at least 85% identical, at least 90% identical, at least 91% identical, at least 92% identical, at least 93% identical, at least 94% identical, at least 95% identical, at least 96% identical, at least 97% identical, at least 98% identical, or at least 99% identical to the nucleotide sequence of any one of SEQ ID NOs: 11-18 (see Table 4).

In one aspect, the fungal strain comprises an RPB2 sequence that is at least 80% identical, at least 85% identical, at least 90% identical, at least 91% identical, at least 92% identical, at least 93% identical, at least 94% identical, at least 95% identical, at least 96% identical, at least 97% identical, at least 98% identical, or at least 99% identical to the nucleotide sequence of any one of SEQ ID NOs: 19-26 (see Table 5).

In one aspect, the fungal strain comprises a TEF1 sequence that is at least 80% identical, at least 85% identical, at least 90% identical, at least 91% identical, at least 92% identical, at least 93% identical, at least 94% identical, at least 95% identical, at least 96% identical, at least 97% identical, at least 98% identical, or at least 99% identical to the nucleotide sequence of any one of SEQ ID NOs: 27-34 (see Table 6).

In one aspect, the fungal strain comprises a TUB2 sequence that is at least 80% identical, at least 85% identical, at least 90% identical, at least 91% identical, at least 92% identical, at least 93% identical, at least 94% identical, at least 95% identical, at least 96% identical, at least 97% identical, at least 98% identical, or at least 99% identical to the nucleotide sequence of any one of SEQ ID NOs: 35-42 (see Table 7).

TABLE 2

| SEQ | Strain | |
|---|---|---|
| ID No. | Number | ITS Sequence |
| 1 | AU-16727 | GGTCATTTAGAGGAAGTAAAAGTCGTAACAAGGTCTCCGTTGGTGAACCAGCGGAGGGATCATTACCGA |
| | | GTTTTCAACTCCCTAACCCTTCTGTGAACCTACCTATCGTTGCTTCGGCGGACTCGCCCCAGCCCGGACGC |
| | | GGACTGGACCAGCGGCCCGCCGGGGACCTCAAACTCTTGTATTCCAGCATCTTCTGAATACGCCGCAAGG |
| | | CAAAACAAATGAATCAAAACTTTCAACAACGGATCTCTTGGCTCTGGCATCGATGAAGAACGCAGCGAA |
| | | ATGCGATAAGTAATGTGAATTGCAGAATCCAGTGAATCATCGAATCTTTGAACGCACATTGCGCCCGCCA |
| | | GCATTCTGGCGGGCATGCCTGTTCGAGCGTCATTTCAACCCTCGACCTCCCCTGGGGGAGGTCGGCGTTG |
| | | GGGACCGGCAGCACACCGCCGGCCCTGAAATGGAGTGGCGGCCCGTCCGCGGCGACCTCTGCGTAGTAA |
| | | TACAGCTCGCACCGGAACCCCGACGCGGCCACGCCGTAAAACACCCAACTTCTGAACGTTGACCTCGAA |
| | | TCAGGTAGGACTACCCGCTGAACTTAAGCATATCAATAAGCGGAGGA |
| 2 | US-52 | GGTCATTTAGAGGAAGTAAAAGTCGTAACAAGGTCTCCGTTGGTGAACCAGCGGAGGGATCATTACCGA |
| | | GTTTTCAACTCCCTAACCCTTCTGTGAACCTACCTATCGTTGCTTCGGCGGACTCGCCCCAGCCCGGACGC |
| | | GGACTGGACCAGCGGCCCGCCGGGGACCTCAAACTCTTGTATTCCAGCATCTTCTGAATACGCCGCAAGG |
| | | CAAAACAAATGAATCAAAACTTTCAACAACGGATCTCTTGGCTCTGGCATCGATGAAGAACGCAGCGAA |
| | | ATGCGATAAGTAATGTGAATTGCAGAATCCAGTGAATCATCGAATCTTTGAACGCACATTGCGCCCGCCA |
| | | GCATTCTGGCGGGCATGCCTGTTCGAGCGTCATTTCAACCCTCGACCTCCCCTGGGGGAGGTCGGCGTTG |
| | | GGGACCGGCAGCACACCGCCGGCCCTGAAATGGAGTGGCGGCCCGTCCGCGGCGACCTCTGCGTAGTAA |
| | | TACAGCTCGCACCGGAACCCCGACGCGGCCACGCCGTAAAACACCCAACTTCTGAACGTTGACCTCGAA |
| | | TCAGGTAGGACTACCCGCTGAACTTAAGCATATCAATAAGCGGAGGA |
| 3 | US-675 | GGTCATTTAGAGGAAGTAAAAGTCGTAACAAGGTCTCCGTTGGTGAACCAGCGGAGGGATCATTACCGA |
| | | GTTTTCAACTCCCTAACCCTTCTGTGAACCTACCTATCGTTGCTTCGGCGGACTCGCCCCAGCCCGGACGC |
| | | GGACTGGACCAGCGGCCCGCCGGGGACCTCAAACTCTTGTATTCCAGCATCTTCTGAATACGCCGCAAGG |
| | | CAAAACAAATGAATCAAAACTTTCAACAACGGATCTCTTGGCTCTGGCATCGATGAAGAACGCAGCGAA |
| | | ATGCGATAAGTAATGTGAATTGCAGAATCCAGTGAATCATCGAATCTTTGAACGCACATTGCGCCCGCCA |
| | | GCATTCTGGCGGGCATGCCTGTTCGAGCGTCATTTCAACCCTCGACCTCCCCTGGGGGAGGTCGGCGTTG |
| | | GGGACCGGCAGCACACCGCCGGCCCTGAAATGGAGTGGCGGCCCGTCCGCGGCGACCTCTGCGTAGTAA |
| | | TACAGCTCGCACCGGAACCCCGACGCGGCCACGCCGTAAAACACCCAACTTCTGAACGTTGACCTCGAA |
| | | TCAGGTAGGACTACCCGCTGAACTTAAGCATATCAATAAGCGGAGGA |
| 4 | US-699 | GGTCATTTAGAGGAAGTAAAAGTCGTAACAAGGTCTCCGTTGGTGAACCAGCGGAGGGATCATTACCGA |
| | | GTTTTCAACTCCCTAACCCTTCTGTGAACCTACCTATCGTTGCTTCGGCGGACTCGCCCCAGCCCGGACGC |
| | | GGACTGGACCAGCGGCCCGCCGGGGACCTCAAACTCTTGTATTCCAGCATCTTCTGAATACGCCGCAAGG |
| | | CAAAACAAATGAATCAAAACTTTCAACAACGGATCTCTTGGCTCTGGCATCGATGAAGAACGCAGCGAA |
| | | ATGCGATAAGTAATGTGAATTGCAGAATCCAGTGAATCATCGAATCTTTGAACGCACATTGCGCCCGCCA |
| | | GCATTCTGGCGGGCATGCCTGTTCGAGCGTCATTTCAACCCTCGACCTCCCCTGGGGGAGGTCGGCGTTG |
| | | GGGACCGGCAGCACACCGCCGGCCCTGAAATGGAGTGGCGGCCCGTCCGCGGCGACCTCTGCGTAGTAA |
| | | TACAGCTCGCACCGGAACCCCGACGCGGCCACGCCGTAAAACACCCAACTTCTGAACGTTGACCTCGAA |
| | | TCAGGTAGGACTACCCGCTGAACTTAAGCATATCAATAAGCGGAGGA |
| 5 | US-707 | GGTCATTTAGAGGAAGTAAAAGTCGTAACAAGGTCTCCGTTGGTGAACCAGCGGAGGGATCATTACCGA |
| | | GTTTTCAACTCCCTAACCCTTCTGTGAACCTACCTATCGTTGCTTCGGCGGACTCGCCCCAGCCCGGACGC |
| | | GGACTGGACCAGCGGCCCGCCGGGGACCTCAAACTCTTGTATTCCAGCATCTTCTGAATACGCCGCAAGG |
| | | CAAAACAAATGAATCAAAACTTTCAACAACGGATCTCTTGGCTCTGGCATCGATGAAGAACGCAGCGAA |
| | | ACGCGATAAGTAATGTGAATTGCAGAATCCAGTGAATCATCGAATCTTTGAACGCACATTGCGCCCGCCA |
| | | GCATTCTGGCGGGCATGCCTGTTCGAGCGTCATTTCAACCCTCGACCTCCCCAAGGGGAGGTCGGCGTTG |
| | | GGGACCGGCAGCACACCGCCGGCCCTGAAATGGAGTGGCGGCCCGTCCGCGGCGACCTCTGCGCAGTAA |
| | | TACAGCTCGCACCGGAACCCCGACGCGGCCACGCCGTAAAACACCCAACTTCTGAACGTTGACCTCGAA |
| | | TCAGGTAGGACTACCCGCTGAACTTAAGCATATCAATAAGCGGAGGA |
| 6 | US-803 | GGTCATTTAGAGGAAGTAAAAGTCGTAACAAGGTCTCCGTTGGTGAACCAGCGGAGGGATCATTACCGA |
| | | GTTTTCAACTCCCTAACCCTTCTGTGAACCTACCTATCGTTGCTTCGGCGGACTCGCCCCAGCCCGGACGC |
| | | GGACTGGACCAGCGGCCCGCCGGGGACCTCAAACTCTTGTATTCCAGCATCTTCTGAATACGCCGCAAGG |
| | | CAAAACAAATGAATCAAAACTTTCAACAACGGATCTCTTGGCTCTGGCATCGATGAAGAACGCAGCGAA |
| | | ATGCGATAAGTAATGTGAATTGCAGAATCCAGTGAATCATCGAATCTTTGAACGCACATTGCGCCCGCCA |
| | | GCATTCTGGCGGGCATGCCTGTTCGAGCGTCATTTCAACCCTCGACCTCCCCTGGGGGAGGTCGGCGTTG |
| | | GGGACCGGCAGCACACCGCCGGCCCTGAAATGGAGTGGCGGCCCGTCCGCGGCGACCTCTGCGTAGTAA |
| | | TACAGCTCGCACCGGAACCCCGACGCGGCCACGCCGTAAAACACCCAACTTCTGAACGTTGACCTCGAA |
| | | TCAGGTAGGACTACCCGCTGAACTTAAGCATATCAATAAGCGGAGGA |
| 7 | US-670 | GGTCATTTAGAGGAAGTAAAAGTCGTAACAAGGTCTCCGTTGGTGAACCAGCGGAGGGATCATTACCGA |
| | | GTTTTCAACTCCCTAACCCTTCTGTGAACCTACCTATCGTTGCTTCGGCGGACTCGCCCCAGCCCGGACGC |
| | | GGACTGGACCAGCGGCCCGCCGGGGACCTCAAACTCTTGTATTCCAGCATCTTCTGAATACGCCGCAAGG |
| | | CAAAACAAATGAATCAAAACTTTCAACAACGGATCTCTTGGCTCTGGCATCGATGAAGAACGCAGCGAA |
| | | ATGCGATAAGTAATGTGAATTGCAGAATCCAGTGAATCATCGAATCTTTGAACGCACATTGCGCCCGCCA |
| | | GCATTCTGGCGGGCATGCCTGTTCGAGCGTCATTTCAACCCTCGACCTCCCCTGGGGGAGGTCGGCGTTG |
| | | GGGACCGGCAGCACACCGCCGGCCCTGAAATGGAGTGGCGGCCCGTCCGCGGCGACCTCTGCGTAGTAA |
| | | TACAGCTCGCACCGGAACCCCGACGCGGCCACGCCGTAAAACACCCAACTTCTGAACGTTGACCTCGAA |
| | | TCAGGTAGGACTACCCGCTGAACTTAAGCATATCAATAAGCGGAGGA |
| 8 | US-935 | GGTCATTTAGAGGAAGTAAAAGTCGTAACAAGGTCTCCGTTGGTGAACCAGCGGAGGGATCATTACCGA |
| | | GTTTTCAACTCCCTAACCCTTCTGTGAACCTACCTATCGTTGCTTCGGCGGACTCGCCCCAGCCCGGACGC |
| | | GGACTGGACCAGCGGCCCGCCGGGGACCTCAAACTCTTGTATTCCAGCATCTTCTGAATACGCCGCAAGG |
| | | CAAAACAAATGAATCAAAACTTTCAACAACGGATCTCTTGGCTCTGGCATCGATGAAGAACGCAGCGAA |
| | | ATGCGATAAGTAATGTGAATTGCAGAATCCAGTGAATCATCGAATCTTTGAACGCACATTGCGCCCGCCA |
| | | GCATTCTGGCGGGCATGCCTGTTCGAGCGTCATTTCAACCCTCGACCTCCCCTGGGGGAGGTCGGCGTTG |
| | | GGGACCGGCAGCACACCGCCGGCCCTGAAATGGAGTGGCGGCCCGTCCGCGGCGACCTCTGCGTAGTAA |
| | | TACAGCTCGCACCGGAACCCCGACGCGGCCACGCCGTAAAACACCCAACTTCTGAACGTTGACCTCGAA |
| | | TCAGGTAGGACTACCCGCTGAACTTAAGCATATCAATAAGCGGAGGA |

TABLE 3

| SEQ | Strain | |
|---|---|---|
| ID No. | Number | Bloc Sequence |
| 9 | AU-16727 | GGGCCACGCGGAGCGACTTTGTCAAAGGGACAATGACCTGGAGCGACCGAAGCGTGCGCGCTGCGACA |
| | | ATTCAGTCATGCCCGATATGCTGCGCCTGCTCGCCTCCCGACAGGATCTGCTGGAGGATCTTCAGGCGAG |
| | | GTTCATTGCGAGCGGCGCAAGAATCAAGCCTTGGAAGCAGCAAGATCTCTCTGTTCGTAAAAGGGCATC |
| | | AGTTTCGCCCAACTCAACAGACACTGGTGCGGAATGCGTGGAGCGCGGCTCGGCCACTTCGGGGAGTGC |
| | | AAGCGGTACAGCGGCCTTGCCCACAGAAACCAACGCGCCAAAGTCATTGGCATTCACATTGCAGTGTCC |
| | | AGCCGCACTTTCCATCGTCTTTGTACTCGCTCTCACTCTAGTCCTCCACTGAGAGAGTAATGCTCACAGGT |
| | | CAAAGAGTTGACGGTTAAACTCACACAGCAAGAGAAAGCTTGTATAAGTAGATGTATCAAATGTATTTA |
| | | TGAAATAATTACCAAAGCCGGAAAACCATATTTACAAAGCCGGCGAATATAGCTGTGCACTTCCTGGAG |
| | | CCGACCACCTCTTAGAGTAGGTCTGATTAAAGCCCGAGTAGCGACCCAGGCTAGGAGGCAAATCGCCCA |
| | | CCGCGGTCCCCCAGCTACTCTCATTCTTCCCCAGCGTGAGAACGAGTTCCTTGCCCTCGGTAAAGAATGA |
| | | GTGATCAATCCAGTTCTTTGTGTAGGGCTTGCCGTTGAGCGTCGCACTCTGAATATAGATATTCTTGTACG |
| | | CGGCGTCAAAGTGGACGTTGCGAATCTTGGCCGTCTTGCCGGTGACGGGGTGCTTGACGGTCACGCTGGG |
| | | GAAAAAGGGCGGCGTGATGAGGTAGACGTCTTGTCCCGGGTTGGGAAAGAGACCCATCATGCTAAAGGC |
| | | GACAAAGGAGCCCATGGCGCCGCTGTCGTCGTTGCCGGGACGCCGGGAGGGTCGCTGAGAGCGCGATGT |
| | | AAAAGTGGCTGCGCTTGCGGAGAGGGCCGGACGCGCGTATGATATATCTGTGTGAAAACACA |
| 10 | AU-16727 | GTGCTCGATTCTATATTCAGAGTGCGACGCTCAACGGCAAGCCCTACACAAAGAACTGGATTGATCACTC |
| | | ATTCTTTACCGAGGGCAAGGAACTCGTTCTCACGCTGGGGAAGAATGAGAGTAGCTGGGGGACCGCGGT |
| | | GGGCGATTTGCCTCCTAGCCTGGGTCGCTACTCGGGCTTTAATCAGACCTACTCTAAGAGGTGGTCGGCT |
| | | CCAGGAAGTGCACAGCTATATTCGCCGGCTTTGTAAATATGGTTTTCCGGCTTTGGTAATTATTTCATAAA |
| | | TACATTTGATACATCTACTTATACAAGCTTTCTCTTGCTGTGTGAGTTTAACCGTCAACTCTTTGACCTGT |
| | | GAGCATTACTCTCTCAGTGGAGGACTAGAGTGAGAGCGAGTACAAAGACGATGGAAAGTGCGGCTGGAC |
| | | ACTGCAATGTGAATGCCAATGACTTTGGCGCGTTGGTTTCTGTGGGCAAGGCCGCTGTACCGCTTGCACT |
| | | CCCCGAAGTGGCCGAGCCGCGCTCCACGCATTCCGCACCAGTGTCTGTTGAGTTGGGCGAAACTGATGCC |
| | | CTTTTACGAACAGAGAGATCTTGCTGCTTCCAAGGCTTGATTCTTGCGCCGCTCGCAATGAACCTCGCCT |
| | | GAAGATCCTCCAGCAGATCCTGTCGGGAGGCGAGCAGGCGCAGCATATCGGGCATGACTGAATTGTCGC |
| | | AGCGCGCACGCTTCGGTCGCTCCAGGTCATTGTCCCTTTTGACAAAGGTCGCTCCGGGCTTAATCTTGTG |
| | | GCAGTTGCTCTGGCTGCGACCGGCACGCAGCTGGCAGATTTCCTCCTCCTTGCAGTCTCCCTGGCAACCTT |
| | | TCTTGATCCACCCCCGGCTCTTGCGAGACACGTATTCGTCAAACAGCGTGCGTTGCTTTCAAAAGCTAAA |
| | | GTGACATTGTGCCAGAAAGCCGGTGTGAGCTCGGCCTCGGCGCGATGGGCGGAGAAAGTAGTGAGCCAT |
| | | AGGACTCTTTGGCAGAGTAGTATTTCTTCCATGTCGGCCCGGACTTTGATACTCGGGATCGGTCATGTCG |
| | | GCATGTAGTACGCATCGAGATGCAAATGTGTCGGGATCGACGTCGTATACCGCGAAGCGGCATGCGACG |
| | | TGGCAGATGCTGCGATGTAGACTGCGTGCCTGCCATGCATCCGCCTCAGTAGTCGAGATAGCTGACTCGA |
| | | TGATCATGTGCCGGTTGGCCAAGACGTCGAATGTGTCCGTACGCGACTGTCAGAAGTATTTTTGGGCTGG |
| | | ATACTAAT |

TABLE 4

| SEQ | Strain | |
|---|---|---|
| ID No. | Number | RPB1 Sequence |
| 11 | AU-16727 | ATGGCGAACCTTCCCTTTCCGCATTCGAGCGCGCCGCTCAAAACTGTTGAGGAAATACAGTTTGGTATGT |
| | | TATCCCCGGAGGAAATCAAAAACATGAGCGTCGCGCACATTCTCTATCCTGAAACAATGGAAGAGAACA |
| | | AGACAACACCACGCGACGGCGGTCTGAACGATCCTCTTCTTGGTTCCATCGATCGTCAGTTCAAGTGCAA |
| | | GACTTGCTCTCAGCCGATGAGCGAGTGTCCAGGTCATTTTGGCCATATAGAGCTGGCGAAACCTGTCTAT |
| | | CACCCTGGTTTCATCAAGAAAGTGAAAAAGGTTTTGGAGATTGTCTGCCACAACTGCAGCAAAGTGTTGG |
| | | CCGATGAAAGCGATCCCGAATTCGTCACAGCTATTCATACTCGCGATCCGAAACTCCGATTCAAGCGCGT |
| | | TTGGGCCGTATGCAAGAAGAAGCGCAAATGCGAGAATGAGGAGCGGCAAGACAAGAATAAAGACGAAG |
| | | AGTTCGCTCCAGGTGTCAAGAACGTCGTTCTCGAAGGACATGGCGGATGTGGCAATATGCAGCCGCAGG |
| | | TGAGACAGGCCGCGCTGCAACTCAAAGCTGCCTTCGAGGTTACTTCGGAAGAGGGTCCCAAGAGGAAAG |
| | | AGACGGTTAATATCAGCGCCGAGATGGCGCATGGTATCCTTCGCCGCATCTCTGAGCGCGATCTGCACAA |
| | | TATTGGTCTTAACTCAGACTATGCTCGTCCTGAGTGGATGATCATCACTGTCCTGCCTGTACCCCCTCCTC |
| | | CCGTGCGTCCTAGTATTTCCATGGATGGTACTGGTACTGGCACGAGAAACGAGGATGATCTGACCTACAA |
| | | GCTTGGTGACATTATCCGCGCCAACGGTAATGTCAAGCAGGCCATTCGCGAAGGATCACCGCAACACAT |
| | | CGCGCGTGATTTTGAGGAGCTGCTGCAGTATCATGTTGCCACCTACATGGATAATGATATTGCTGGTCAG |
| | | CCGCGGGCCCTCCAAAAGAGCGGTCGTCCTGTCAAGGCGATTCGCGCCCGTCTCAAGGGCAAGGAGGGT |
| | | CGTCTGCGAGGCAACTTGATGGGTAAGCGTGTCGACTTTTCCGCGCGTACCGTTATCACTGGTGACGCCA |
| | | ACTTGTCTCTGCACGAAGTTGGTGTTCCGCGAAGCATTGCTCGTACTCTCACCTATCCTGAGACTGTCACA |
| | | CCCTATAACATTGCCAAGCTGCATCAACTCGTTGAGAACGGACCTAACGAGCACCCTGGTGCCAAATAC |
| | | GTCATTCGCTCTGATGGCACCAGAATCGATCTACGACACCACAGACGCGCGGCTCAAATTTCTTTGGAAT |
| | | ATGGATGGAAGGTTGAGCGTCATCTAATCGATGGCGACTACATCATCTTCAACCGTCAGCCCTCCCTGCA |
| | | CAAGGAATCCATGATGGGTCATCGTGTCAAGGTCATGCCCTACTCTACCTTCCGACTCAACCTTTCCGTC |
| | | ACTTCCCCCTATAATGCCGATTTTGATGGTGATGAAATGAACTTGCACGTTCCCCAGACTGAGGAAACTC |
| | | GGGCTGAAATCAAGGAGCTGTGTCTCGTTCCCAACAATATTGTGTCGCCTCAGAAAAACGGTCCCCTAAT |
| | | GGGTATTGTTCAAGACTCTCTGGCCGGTGTCTACAAGCTCTGTCGTCGTGACACATTTATCGATAAAGAG |
| | | CTTGTTCAAAATCTCATGCTCTGGGTACCCAACTGGGATGGCGTCATTCCACAGCCGGCTATTCTGAAGC |
| | | CTAGACCTCGCTGGACCGGCAAGCAGATTATCAGCATGGTCATCCCCCCAGAGATCAGTCTGTACAGCA |
| | | AGGAAGATAAGCTGGACAATCCTAGACATGACGCTGGCTTGCTTATTCAGAGCGGTGAGCTCATGTACG |
| | | GTCTGCTCAAGAAGAAGAGTGTTGGTGCTGCCGCAGGAGGCATCATTCATTTGTGCTACAACGAACTCGG |
| | | ACCCGAGGGCGCCATGGCTTTCTTGAATGGTGTCCAACAAGTIGTCACCTACTGGCTACTGAACACCGGT |
| | | TTCAGTATTGGTATTGGCGACACTGTTCCCGACAAGGCTACCATCAATAAGATCCAGGAGCATATCGACG |
| | | AACACAAAGCAGAGGTCGCTCGCTTGACTGCCCAGGCTACAGCCAACGAGCTGGAGGCTTTACCTGGTA |
| | | TGAATGTCCGCGCCACTTTCGAGAACAAAGTCTCCATGGCTCTCAACATGGCCCGTGACCAGGCTGGTAC |
| | | TTCAACCATGAAGAGTTTGAAGGATTCCAACAATGCCGTCACCATGGCTGATTCTGGTTCCAAGGGTTCT |
| | | TCCATCAACATCTCGCAAATGACGGCCCTCGTCGGACAGCAGATTGTTGAAGGAAAGCGTATCCCTTTCG |
| | | GCTTCAAGTACCGAACACTGCCGCATTTCACAAAGGACGATTATTCGCCTGAAGCTCGTGGTTTCGTCGA |
| | | GAACTCGTACCTTCGTGGCTTGACTCCTTCGGAGTTCTTCTTCCACGCCATGGCTGGTAGAGAGGGTCTTA |
| | | TTGATACTGCGGTCAAGACCGCTGAGACGGGTTACATTCAACGACGTCTCGTCAAGGCGCTCGAAGATCT |
| | | CAGCGCGCGCTACGACGGAACTGTACGTAACTCATTGGGCGACGTTATTCAATTCTTGTACGGTGAAGAT |
| | | GGTCTCGATGCTATGTGCATTGAAAAACAGAGACTTGGCACCATCAAGATGTCTAATGCTGCCTTCGAAA |
| | | GCCAATATCGTTTGGATCTTGCCAACCCCCCTGAGTGGTTCCGCAAGGACTATGAGTACGGCAATGAGCT |
| | | GGCAGGCGATAGGGCGTCCATGGAACTGCTGGACATGGAGTGGGATGCCCTTGTTGCTGACAGACGCCT |
| | | TGTCCGTGATATCAACAAGAGCAAGATGGGTGAAGAGATGATGCAGCTTCCACTCAATATTGGCCGTAT |
| | | CATCGAGAGTGCCAAGCGTGTCTTCAACATTCGCGAAACTGACCGCAGCAATCTGCGGCCGTCCGATGTC |
| | | ATCACAACTGTTCAAAATTTGCTGGCCAACATGCAGATTGTGCGTGGCACGGACCCCATCTCAGTTGAGG |
| | | CCGATTACAATGCTACCATTCTGTTCAAGGCACTCCTGCGCTCTCGTCTCGCTTTCAAAGAGATTGTCTAC |
| | | GTGCATCGCTTAAACAAGCTTGCCTTTAACCACGTCATCGGCGAGCTTCAGAATCGTTGGGATAGAGCTT |
| | | TTGTTAGCCCTGGTGAGATGGTTGGTGTTCTCGCTGCCCAGTCTATTGGTGAGCCTGCTACTCAGATGACA |
| | | CTCAACACTTTTCACTTTGCTGGTGTGTCTTCGAAGAACGTTACTCTGGGTGTGCCGCGTCTCAAGGAGAT |
| | | TCTGAACTTGGCCAAAAATATCAAGACTCCCAGCATGGCCGTCTACCTGGACACCAAACTGGGTACGCA |
| | | GGAGCAGGCCAAGAAGCTCCGTAGTTTGGTCGAGTATACCAACTTGCGCTCCGTCACATCGGTCACCGA |
| | | AATCTACTACGATCCCGAGGTGCAGGCAACGAACATTGCTGAGGATGTGGACATGGTCGAATCTTACTTC |
| | | ATGATTCCCGATGATGCCCAGGATACCATTCACCGGCAGTCGCGCTGGCTCCTCCGCATTACTCTTGACC |
| | | GCCAGAAACTCCTGGACAAGGAAATCAAGATTGACGATGTCGCTGCGTGCATCAAGGAGGAATACTCAA |
| | | ACGATCTGGCCATCATCTTCTCTGACAACAATGCCGACGAGCAAGTCATTCGTATCCGTACCATTCGTCA |
| | | AAGTGATGACAAGGACGAGGATTCTGACACGAAGATTGAAGACGATGTCATGCTCAAGCGGCTAGAAGC |
| | | TCACTTGCTTGATACTATGACTTTACGCGGTGTTCCTGGCATCGAAAGGGCCTTCTTGACCAAGGGTACC |
| | | CGCCTGGTTGAGGATGAAGACGGCTCCGAACTGGCTCTCAAGGATAACCCGAAGTGCACACAGTGGTAC |
| | | TTGGATACCAGTGGTTCAGCACTCCGCGAGGTACTCGCTGTCCCCGGTGTCGATCCCACCAGAACCTACA |
| | | GCAACGACCTGTACCAGATCACTGAGGTGTTCGGTGTCGAGGCTGTACGATCTGCGCTGGTGAAGGAATT |
| | | GACCAACGTGTTGGCTTTTGACGGTTCGTACGTCAATCATCGTCATATTGCTTTGCTGTGCGACATTATGA |
| | | CCTATCGTGGTGTCATTTCGGCCGTCACACGTCACGGTATTAACCGGGCCGACACTGGTGCTCTGATGCG |
| | | TTGTTCCTTCGAAGAGACTGTCGAAATCCTGCTCGAGGCTGCCGCTACCGGCGAACTCGACGACTGCCGT |
| | | GGTATCTCCGAGAACGTCATGCTTGGTCAGCTTGCGCCCATGGGCACCGGCAACTTTGATGTCTACTTGG |
| | | ATCCTAAGATGCTGGAGACTGTCATTTCCGACAATTCTCGCATGGGCCTCATGCCTGGCATGCCCACCAA |
| | | GGAAGGCGAGGGCGAGGGTGCTGCCACACCATACGATAGTGGCTCGCCGATGGGCGACTCTGGTTACCT |
| | | CAGCATGGCTTCGCCTGCTGCTGGTAACTTTTCTCCTATTCAGGGTGCTGGCTCAGAAACACCTACCGGG |
| | | TTCAATACCGAGTATGGCGGTGGCTTCGGAGCCAATGGTTCGATGAGCCCGTACTCGCGCGGCGCTACTA |
| | | GTCCCTTCAGCACGTCGCCCACGTCGCCATTCAGCGCAGGCATGGGGGGCTATTCGCCATCGTCGCCTAA |
| | | CACGGGTTACTCTCCTACGTCGCCGATGCTTGACGGCGGTGCTCGTTACGCCACCTCTCCGTCATTCAGTC |
| | | CGTCGTCGCCGTCTTTCTCGCCGACGTCGCCGATGCTGCGACCCACCAGCCCGGCCAGCCCCAACTACAG |
| | | CCCAACGTCTCCTAGTTACTCGCCGACGTCGCCGACATCACCGCGACACTACTCGCCTACATCTCCTGCTC |
| | | AGTTCAACTCGCCGACGTCTCCCAGCTACTCCCCGGCCAGTCCCAATTACAGTCCTGCGTCTCCCAACTTC |
| | | CAGGGAGCTGGTCCTACTTCGCCCTCGTACTCGCCTGCGTCGCCATCGTGGTCACCAACCTCACCCGACG |
| | | CTTACTCACCCACGAGTCCAAGCTTCGGACGCAGCCCCGGCCAGCAGCAGTCGCCCACGAGCCCTGGTTA |
| | | CTCTCCCACCTCGCCGCAGTTCTCTCCTCGCACGCCTGGTCCCTCGGGCTCTGGTGGAGGTTCTGGAACTG |
| | | GCAACCAATACTCGCCTAGCTCGCCGAATAACGAGTGA |
| 12 | US-52 | ATGGCGAACCTTCCCTTTCCGCATTCGAGCGCGCCGCTCAAAACTGTTGAGGAAATACAGTTTGGTATGT |
| | | TATCCCCGGAGGAAATCAAAAACATGAGCGTCGCGCACATTCTCTATCCTGAAACAATGGAAGAGAACA |
| | | AGACAACACCACGCGACGGCGGTCTGAACGATCCTCTTCTTGGTTCCATCGATCGTCAGTTCAAGTGCAA |
| | | GACTTGCTCTCAGCCGATGAGCGAGTGTCCAGGTCATTTTGGCCATATAGAGCTGGCGAAACCTGTCTAT |
| | | CACCCTGGTTTCATCAAGAAAGTGAAAAAGGTTTTGGAGATTGTCTGCCACAACTGCAGCAAAGTGTTGG |
| | | CCGATGAAAGCGATCCCGAATTCGTCACAGCTATTCATACTCGCGATCCGAAACTCCGATTCAAGCGCGT |
| | | TTGGGCCGTATGCAAGAAGAAGCGCAAATGCGAGAATGAGGAGCGGCAAGACAAGAATAAAGACGAAG |
| | | AGTTCGCTCCAGGTGTCAAGAACGTCGTTCTCGAAGGACATGGCGGATGTGGCAATATGCAGCCGCAGG |
| | | TGAGACAGGCCGCGCTGCAACTCAAAGCTGCCTTCGAGGTTACTTCGGAAGAGGGTCCCAAGAGGAAAG |
| | | AGACGGTTAATATCAGCGCCGAGATGGCGCATGGTATCCTTCGCCGCATCTCTGAGCGCGATCTGCACAA |
| | | TATTGGTCTTAACTCAGACTATGCTCGTCCCGAGTGGATGATCATCACTGTCCTGCCTGTACCCCCTCCTC |
| | | CCGTGCGTCCTAGTATTTCCATGGATGGTACTGGTACTGGCACGAGAAACGAGGATGATCTGACCTACAA |
| | | GCTTGGTGACATTATCCGCGCCAACGGTAATGTCAAGCAGGCCATTCGCGAAGGATCACCGCAACACAT |
| | | CGCGCGTGATTTTGAGGAGCTGCTGCAGTATCATGTTGCCACCTACATGGATAATGATATTGCTGGTCAG |
| | | CCGCGGGCCCTCCAAAAGAGCGGTCGTCCTGTCAAGGCGATTCGCGCCCGTCTCAAGGGCAAGGAGGGT |
| | | CGTCTGCGAGGCAACTTGATGGGTAAGCGTGTCGACTTTTCCGCGCGTACCGTTATCACTGGTGACGCCA |
| | | ACTTGTCTCTGCACGAAGTTGGTGTTCCGCGAAGCATTGCTCGTACTCTCACCTATCCTGAGACTGTCACA |
| | | CCCTATAACATTGCCAAGCTGCATCAACTCGTTGAGAACGGACCTAACGAGCACCCTGGTGCCAAATAC |
| | | GTCATTCGCTCTGATGGCACCAGAATCGATCTACGACACCACAGACGCGCGGCTCAAATTTCTTTGGAAT |
| | | ATGGATGGAAGGTTGAGCGTCATCTAATCGATGGCGACTACATCATCTTCAACCGTCAGCCCTCCCTGCA |
| | | CAAGGAATCCATGATGGGTCATCGTGTCAAGGTCATGCCCTACTCTACCTTCCGACTCAACCTTTCCGTC |
| | | ACTTCCCCCTATAATGCCGATTTTGATGGTGATGAAATGAACTTGCACGTTCCCCAGACTGAGGAAACTC |
| | | GGGCTGAAATCAAGGAGCTGTGTCTCGTTCCCAACAATATTGTGTCGCCTCAGAAAAACGGTCCCCTAAT |
| | | GGGTATTGTTCAAGACTCTCTGGCCGGTGTCTACAAGCTCTGTCGTCGTGACACATTTATCGATAAAGAG |
| | | CTTGTTCAAAATCTCATGCTCTGGGTACCCAACTGGGATGGCGTCATTCCACAGCCGGCTATTCTGAAGC |
| | | CTAGACCTCGCTGGACCGGCAAGCAGATTATCAGCATGGTCATCCCCCCAGAGATCAGTCTGTACAGCA |
| | | AGGAAGATAAGCTGGACAATCCTAGACATGACGCTGGCTTGCTTATTCAGAGCGGTGAGCTCATGTACG |
| | | GTCTGCTCAAGAAGAAGAGTGTTGGTGCTGCCGCAGGAGGCATCATTCATTTGTGCTACAACGAACTCGG |
| | | ACCCGAGGGCGCCATGGCTTTCTTGAATGGTGTCCAACAAGTTGTCACCTACTGGCTACTGAACACCGGT |
| | | TTCAGTATTGGTATTGGCGACACTGTTCCCGACAAGGCTACCATCAATAAGATCCAGGAGCATATCGACG |
| | | AACACAAAGCAGAGGTCGCTCGCTTGACTGCCCAGGCTACAGCCAACGAGCTGGAGGCTTTACCTGGTA |
| | | TGAATGTCCGCGCCACTTTCGAGAACAAAGTCTCCATGGCTCTCAACATGGCCCGTGACCAGGCTGGTAC |
| | | TTCAACCATGAAGAGTTTGAAGGATTCCAACAATGCCGTCACCATGGCTGATTCTGGTTCCAAGGGTTCT |
| | | TCCATCAACATCTCGCAAATGACGGCCCTCGTCGGACAGCAGATTGTTGAAGGAAAGCGTATCCCTTTCG |
| | | GCTTCAAGTACCGAACACTGCCGCATTTCACAAAGGACGATTATTCGCCTGAAGCTCGTGGTTTCGTCGA |
| | | GAACTCGTACCTTCGTGGCTTGACTCCTTCGGAGTTCTTCTTCCACGCCATGGCTGGTAGAGAGGGTCTTA |
| | | TTGATACTGCGGTCAAGACCGCTGAGACGGGTTACATTCAACGACGTCTTGTCAAGGCGCTCGAAGATCT |
| | | CAGCGCGCGCTACGACGGAACTGTACGTAACTCATTGGGCGACGTTATTCAATTCTTGTACGGTGAAGAT |
| | | GGTCTCGATGCTATGTGCATTGAAAAACAGAGACTTGGCACCATCAAGATGTCTAATGCTGCCTTCGAAA |
| | | GCCAATATCGTTTGGATCTTGCCAACCCCCCTGAGTGGTTCCGCAAGGACTATGAGTACGGCAATGAGCT |
| | | GGCAGGCGATAGGGCGTCCATGGAACTGCTGGACATGGAGTGGGATGCCCTTGTTGCTGACAGACGCCT |
| | | TGTCCGTGATATCAACAAGAGCAAGATGGGTGAAGAGATGATGCAGCTTCCACTCAATATTGGCCGTAT |
| | | CATCGAGAGTGCCAAGCGTGTCTTCAACATTCGCGAAACTGACCGCAGCAATCTGCGGCCGTCCGATGTC |
| | | ATCACAACTGTTCAAAATTTGCTGGCCAACATGCAGATTGTGCGTGGCACGGACCCCATCTCAGTTGAGG |
| | | CCGATTACAATGCTACCATTCTGTTCAAGGCACTCCTGCGCTCTCGTCTCGCTTTCAAAGAGATTGTCTAC |
| | | GTGCATCGCTTAAACAAGCTTGCCTTTAACCACGTCATCGGCGAGCTTCAGAATCGTTGGGATAGAGCTT |
| | | TTGTTAGCCCTGGTGAGATGGTTGGTGTTCTCGCTGCCCAGTCTATTGGTGAGCCTGCTACTCAGATGACA |
| | | CTCAACACTTTTCACTTTGCTGGTGTGTCTTCGAAGAACGTTACTCTGGGTGTGCCGCGTCTCAAGGAGAT |
| | | TCTGAACTTGGCCAAAAATATCAAGACTCCCAGCATGGCCGTCTACCTGGACACCAAACTGGGTACGCA |
| | | GGAGCAGGCCAAGAAGCTCCGTAGTTTGGTCGAGTATACCAACTTGCGCTCCGTCACATCGGTCACCGA |
| | | AATCTACTACGATCCCGAGGTGCAGGCAACGAACATTGCTGAGGATGTGGACATGGTCGAATCTTACTTC |
| | | ATGATTCCCGATGATGCCCAGGATACCATTCACCGGCAGTCGCGCTGGCTCCTCCGCATTACTCTTGACC |
| | | GCCAGAAACTCCTGGACAAGGAAATCAAGATTGACGATGTCGCTGCGTGCATCAAGGAGGAATACTCAA |
| | | ACGATCTGGCCATCATCTTCTCTGACAACAATGCCGACGAGCAAGTCATTCGTATCCGTACCATTCGTCA |
| | | AAGTGATGACAAGGACGAGGATTCTGACACGAAGATTGAAGACGATGTCATGCTCAAGCGGCTAGAAGC |
| | | TCACTTGCTTGATACTATGACTTTACGCGGTGTTCCTGGCATCGAAAGGGCCTTCTTGACCAAGGGTACC |
| | | CGCCTGGTTGAGGATGAAGACGGCTCCGAACTGGCTCTCAAGGATAACCCGAAGTGCACACAGTGGTAC |
| | | TTGGATACCAGTGGTTCAGCACTCCGCGAGGTACTCGCTGTCCCCGGTGTCGATCCCACCAGAACCTACA |
| | | GCAACGACCTGTACCAGATCACTGAGGTGTTCGGTGTCGAGGCTGTACGATCTGCGCTGGTGAAGGAATT |
| | | GACCAACGTGTTGGCTTTTGACGGTTCGTACGTCAATCATCGTCATATTGCTTTGCTGTGCGACATTATGA |
| | | CCTATCGTGGTGTCATTTCGGCCGTCACACGTCACGGTATTAACCGGGCCGACACTGGTGCTCTGATGCG |
| | | TTGTTCCTTCGAAGAGACTGTCGAAATCCTGCTCGAGGCTGCCGCTACCGGCGAACTCGACGACTGCCGT |
| | | GGTATCTCCGAGAACGTCATGCTTGGTCAGCTTGCGCCCATGGGCACCGGCAACTTTGATGTCTACTTGG |
| | | ATCCTAAGATGCTGGAGACTGTCATTTCCGACAATTCTCGCATGGGCCTCATGCCTGGCATGCCCACCAA |
| | | GGAAGGCGAGGGCGAGGGTGCTGCCACACCATACGATAGTGGCTCGCCGATGGGCGACTCTGGTTACCT |
| | | CAGCATGGCTTCGCCTGCTGCTGGTAACTTTTCTCCTATTCAGGGTGCTGGCTCAGAAACACCTACCGGG |
| | | TTCAATACCGAGTATGGCGGTGGCTTCGGAGCCAATGGTTCGATGAGCCCGTACTCGCGCGGCGCTACTA |
| | | GTCCCTTCAGCACGTCGCCCACGTCGCCATTCAGCGCAGGCATGGGGGGCTATTCGCCATCGTCGCCTAA |
| | | CACGGGTTACTCTCCTACGTCGCCGATGCTTGACGGCGGTGCTCGTTACGCCACCTCTCCGTCATTCAGTC |
| | | CGTCGTCGCCGTCTTTCTCGCCGACGTCGCCGATGCTGCGACCCACCAGCCCGGCCAGCCCCAACTACAG |
| | | CCCAACGTCTCCTAGTTACTCGCCGACGTCGCCGACATCACCGCGACACTACTCGCCTACATCTCCTGCTC |
| | | AGTTCAACTCGCCGACGTCTCCCAGCTACTCCCCGGCCAGTCCCAATTACAGTCCTGCGTCTCCCAACTTC |
| | | CAGGGAGCTGGTCCTACTTCGCCCTCGTACTCGCCTGCGTCGCCATCGTGGTCACCAACCTCACCCGACG |
| | | CTTACTCACCCACGAGTCCAAGCTTCGGACGCAGCCCCGGCCAGCAGCAGTCGCCCACGAGCCCTGGTTA |
| | | CTCTCCCACCTCGCCGCAGTTCTCTCCTCGCACGCCTGGTCCCTCGGGCTCTGGTGGAGGTTCTGGAACTG |
| | | GCAACCAATACTCGCCTAGCTCGCCGAATAACGAGTGA |
| 13 | US-675 | ATGGCGAACCTTCCCTTTCCGCATTCGAGCGCGCCGCTCAAAACTGTTGAGGAAATACAGTTTGGTATGT |
| | | TATCCCCGGAGGAAATCAAAAACATGAGCGTCGCGCACATTCTCTATCCTGAAACAATGGAAGAGAACA |
| | | AGACAACACCACGCGACGGCGGTCTGAACGATCCTCTTCTTGGTTCCATCGATCGTCAGTTCAAGTGCAA |
| | | GACTTGCTCTCAGCCGATGAGCGAGTGTCCAGGTCATTTTGGCCATATAGAGCTGGCGAAACCTGTCTAT |
| | | CACCCTGGTTTCATCAAGAAAGTGAAAAAGGTTTTGGAGATTGTCTGCCACAACTGCAGCAAAGTGTTGG |
| | | CCGATGAAAGCGATCCCGAATTCGTCACAGCTATTCATACTCGCGATCCGAAACTCCGATTCAAGCGCGT |
| | | TTGGGCCGTATGCAAGAAGAAGCGCAAATGCGAGAATGAGGAGCGGCAAGACAAGAATAAAGACGAAG |
| | | AGTTCGCTCCAGGTGTCAAGAACGTCGTTCTCGAAGGACATGGCGGATGTGGCAATATGCAGCCGCAGG |
| | | TGAGACAGGCCGCGCTGCAACTCAAAGCTGCCTTCGAGGTTACTTCGGAAGAGGGTCCCAAGAGGAAAG |
| | | AGACGGTTAATATCAGCGCCGAGATGGCGCATGGTATCCTTCGCCGCATCTCTGAGCGCGATCTGCACAA |
| | | TATTGGTCTTAACTCAGACTATGCTCGTCCCGAGTGGATGATCATCACTGTCCTGCCTGTACCCCCTCCTC |
| | | CCGTGCGTCCTAGTATTTCCATGGATGGTACTGGTACTGGCACGAGAAACGAGGATGATCTGACCTACAA |
| | | GCTTGGTGACATTATCCGCGCCAACGGTAATGTCAAGCAGGCCATTCGCGAAGGATCACCGCAACACAT |
| | | CGCGCGTGATTTTGAGGAGCTGCTGCAGTATCATGTTGCCACCTACATGGATAATGATATTGCTGGTCAG |
| | | CCGCGGGCCCTCCAAAAGAGCGGTCGTCCTGTCAAGGCGATTCGCGCCCGTCTCAAGGGCAAGGAGGGT |
| | | CGTCTGCGAGGCAACTTGATGGGTAAGCGTGTCGACTTTTCCGCGCGTACCGTTATCACTGGTGACGCCA |
| | | ACTTGTCTCTGCACGAAGTTGGTGTTCCGCGAAGCATTGCTCGTACTCTCACCTATCCTGAGACTGTCACA |
| | | CCCTATAACATTGCCAAGCTGCATCAACTCGTTGAGAACGGACCTAACGAGCACCCTGGTGCCAAATAC |
| | | GTCATTCGCTCTGATGGCACCAGAATCGATCTACGACACCACAGACGCGCGGCTCAAATTTCTTTGGAAT |
| | | ATGGATGGAAGGTTGAGCGTCATCTAATCGATGGCGACTACATCATCTTCAACCGTCAGCCCTCCCTGCA |
| | | CAAGGAATCCATGATGGGTCATCGTGTCAAGGTCATGCCCTACTCTACCTTCCGACTCAACCTTTCCGTC |
| | | ACTTCCCCCTATAATGCCGATTTTGATGGTGATGAAATGAACTTGCACGTTCCCCAGACTGAGGAAACTC |
| | | GGGCTGAAATCAAGGAGCTGTGTCTCGTTCCCAACAATATTGTGTCGCCTCAGAAAAACGGTCCCCTAAT |
| | | GGGTATTGTTCAAGACTCTCTGGCCGGTGTCTACAAGCTCTGTCGTCGTGACACATTTATCGATAAAGAG |
| | | CTTGTTCAAAATCTCATGCTCTGGGTACCCAACTGGGATGGCGTCATTCCACAGCCGGCTATTCTGAAGC |
| | | CTAGACCTCGCTGGACCGGCAAGCAGATTATCAGCATGGTCATCCCCCCAGAGATCAGTCTGTACAGCA |
| | | AGGAAGATAAGCTGGACAATCCTAGACATGACGCTGGCTTGCTTATTCAGAGCGGTGAGCTCATGTACG |
| | | GTCTGCTCAAGAAGAAGAGTGTTGGTGCTGCCGCAGGAGGCATCATTCATTTGTGCTACAACGAACTCGG |
| | | ACCCGAGGGCGCCATGGCTTTCTTGAATGGTGTCCAACAAGTTGTCACCTACTGGCTACTGAACACCGGT |
| | | TTCAGTATTGGTATTGGCGACACTGTTCCCGACAAGGCTACCATCAATAAGATCCAGGAGCATATCGACG |
| | | AACACAAAGCAGAGGTCGCTCGCTTGACTGCCCAGGCTACAGCCAACGAGCTGGAGGCTTTACCTGGTA |
| | | TGAATGTCCGCGCCACTTTCGAGAACAAAGTCTCCATGGCTCTCAACATGGCCCGTGACCAGGCTGGTAC |
| | | TTCAACCATGAAGAGTTTGAAGGATTCCAACAATGCCGTCACCATGGCTGATTCTGGTTCCAAGGGTTCT |
| | | TCCATCAACATCTCGCAAATGACGGCCCTCGTCGGACAGCAGATTGTTGAAGGAAAGCGTATCCCTTTCG |
| | | GCTTCAAGTACCGAACACTGCCGCATTTCACAAAGGACGATTATTCGCCTGAAGCTCGTGGTTTCGTCGA |
| | | GAACTCGTACCTTCGTGGCTTGACTCCTTCGGAGTTCTTCTTCCACGCCATGGCTGGTAGAGAGGGTCTTA |
| | | TTGATACTGCGGTCAAGACCGCTGAGACGGGTTACATTCAACGACGTCTTGTCAAGGCGCTCGAAGATCT |
| | | CAGCGCGCGCTACGACGGAACTGTACGTAACTCATTGGGCGACGTTATTCAATTCTTGTACGGTGAAGAT |
| | | GGTCTCGATGCTATGTGCATTGAAAAACAGAGACTTGGCACCATCAAGATGTCTAATGCTGCCTTCGAAA |
| | | GCCAATATCGTTTGGATCTTGCCAACCCCCCTGAGTGGTTCCGCAAGGACTATGAGTACGGCAATGAGCT |
| | | GGCAGGCGATAGGGCGTCCATGGAACTGCTGGACATGGAGTGGGATGCCCTTGTTGCTGACAGACGCCT |
| | | TGTCCGTGATATCAACAAGAGCAAGATGGGTGAAGAGATGATGCAGCTTCCACTCAATATTGGCCGTAT |
| | | CATCGAGAGTGCCAAGCGTGTCTTCAACATTCGCGAAACTGACCGCAGCAATCTGCGGCCGTCCGATGTC |
| | | ATCACAACTGTTCAAAATTTGCTGGCCAACATGCAGATTGTGCGTGGCACGGACCCCATCTCAGTTGAGG |
| | | CCGATTACAATGCTACCATTCTGTTCAAGGCACTCCTGCGCTCTCGTCTCGCTTTCAAAGAGATTGTCTAC |
| | | GTGCATCGCTTAAACAAGCTTGCCTTTAACCACGTCATCGGCGAGCTTCAGAATCGTTGGGATAGAGCTT |
| | | TTGTTAGCCCTGGTGAGATGGTTGGTGTTCTCGCTGCCCAGTCTATTGGTGAGCCTGCTACTCAGATGACA |
| | | CTCAACACTTTTCACTTTGCTGGTGTGTCTTCGAAGAACGTTACTCTGGGTGTGCCGCGTCTCAAGGAGAT |
| | | TCTGAACTTGGCCAAAAATATCAAGACTCCCAGCATGGCCGTCTACCTGGACACCAAACTGGGTACGCA |
| | | GGAGCAGGCCAAGAAGCTCCGTAGTTTGGTCGAGTATACCAACTTGCGCTCCGTCACATCGGTCACCGA |
| | | AATCTACTACGATCCCGAGGTGCAGGCAACGAACATTGCTGAGGATGTGGACATGGTCGAATCTTACTTC |
| | | ATGATTCCCGATGATGCCCAGGATACCATTCACCGGCAGTCGCGCTGGCTCCTCCGCATTACTCTTGACC |
| | | GCCAGAAACTCCTGGACAAGGAAATCAAGATTGACGATGTCGCTGCGTGCATCAAGGAGGAATACTCAA |
| | | ACGATCTGGCCATCATCTTCTCTGACAACAATGCCGACGAGCAAGTCATTCGTATCCGTACCATTCGTCA |
| | | AAGTGATGACAAGGACGAGGATTCTGACACGAAGATTGAAGACGATGTCATGCTCAAGCGGCTAGAAGC |
| | | TCACTTGCTTGATACTATGACTTTACGCGGTGTTCCTGGCATCGAAAGGGCCTTCTTGACCAAGGGTACC |
| | | CGCCTGGTTGAGGATGAAGACGGCTCCGAACTGGCTCTCAAGGATAACCCGAAGTGCACACAGTGGTAC |
| | | TTGGATACCAGTGGTTCAGCACTCCGCGAGGTACTCGCTGTCCCCGGTGTCGATCCCACCAGAACCTACA |
| | | GCAACGACCTGTACCAGATCACTGAGGTGTTCGGTGTCGAGGCTGTACGATCTGCGCTGGTGAAGGAATT |
| | | GACCAACGTGTTGGCTTTTGACGGTTCGTACGTCAATCATCGTCATATTGCTTTGCTGTGCGACATTATGA |
| | | CCTATCGTGGTGTCATTTCGGCCGTCACACGTCACGGTATTAACCGGGCCGACACTGGTGCTCTGATGCG |
| | | TTGTTCCTTCGAAGAGACTGTCGAAATCCTGCTCGAGGCTGCCGCTACCGGCGAACTCGACGACTGCCGT |
| | | GGTATCTCCGAGAACGTCATGCTTGGTCAGCTTGCGCCCATGGGCACCGGCAACTTTGATGTCTACTTGG |
| | | ATCCTAAGATGCTGGAGACTGTCATTTCCGACAATTCTCGCATGGGCCTCATGCCTGGCATGCCCACCAA |
| | | GGAAGGCGAGGGCGAGGGTGCTGCCACACCATACGATAGTGGCTCGCCGATGGGCGACTCTGGTTACCT |
| | | CAGCATGGCTTCGCCTGCTGCTGGTAACTTTTCTCCTATTCAGGGTGCTGGCTCAGAAACACCTACCGGG |
| | | TTCAATACCGAGTATGGCGGTGGCTTCGGAGCCAATGGTTCGATGAGCCCGTACTCGCGCGGCGCTACTA |
| | | GTCCCTTCAGCACGTCGCCCACGTCGCCATTCAGCGCAGGCATGGGGGGCTATTCGCCATCGTCGCCTAA |
| | | CACGGGTTACTCTCCTACGTCGCCGATGCTTGACGGCGGTGCTCGTTACGCCACCTCTCCGTCATTCAGTC |
| | | CGTCGTCGCCGTCTTTCTCGCCGACGTCGCCGATGCTGCGACCCACCAGCCCGGCCAGCCCCAACTACAG |
| | | CCCAACGTCTCCTAGTTACTCGCCGACGTCGCCGACATCACCGCGACACTACTCGCCTACATCTCCTGCTC |
| | | AGTTCAACTCGCCGACGTCTCCCAGCTACTCCCCGGCCAGTCCCAATTACAGTCCTGCGTCTCCCAACTTC |
| | | CAGGGAGCTGGTCCTACTTCGCCCTCGTACTCGCCTGCGTCGCCATCGTGGTCACCAACCTCACCCGACG |
| | | CTTACTCACCCACGAGTCCAAGCTTCGGACGCAGCCCCGGCCAGCAGCAGTCGCCCACGAGCCCTGGTTA |
| | | CTCTCCCACCTCGCCGCAGTTCTCTCCTCGCACGCCTGGTCCCTCGGGCTCTGGTGGAGGTTCTGGAACTG |
| | | GCAACCAATACTCGCCTAGCTCGCCGAATAACGAGTGA |
| 14 | US-699 | ATGGCGAACCTTCCCTTTCCGCATTCGAGCGCGCCGCTCAAAACTGTTGAGGAAATACAGTTTGGTATGT |
| | | TATCCCCGGAGGAAATCAAAAACATGAGCGTCGCGCACATTCTCTATCCTGAAACAATGGAAGAGAACA |
| | | AGACAACACCACGCGACGGCGGTCTGAACGATCCTCTTCTTGGTTCCATCGATCGTCAGTTCAAGTGCAA |
| | | GACTTGCTCTCAGCCGATGAGCGAGTGTCCAGGTCATTTTGGCCATATAGAGCTGGCGAAACCTGTCTAT |
| | | CACCCTGGTTTCATCAAGAAAGTGAAAAAGGTTTTGGAGATTGTCTGCCACAACTGCAGCAAAGTGTTGG |
| | | CCGATGAAAGCGATCCCGAATTCGTCACAGCTATTCATACTCGCGATCCGAAACTCCGATTCAAGCGCGT |
| | | TTGGGCCGTATGCAAGAAGAAGCGCAAATGCGAGAATGAGGAGCGGCAAGACAAGAATAAAGACGAAG |
| | | AGTTCGCTCCAGGTGTCAAGAACGTCGTTCTCGAAGGACATGGCGGATGTGGCAATATGCAGCCGCAGG |
| | | TGAGACAGGCCGCGCTGCAACTCAAAGCTGCCTTCGAGGTTACTTCGGAAGAGGGTCCCAAGAGGAAAG |
| | | AGACGGTTAATATCAGCGCCGAGATGGCGCATGGTATCCTTCGCCGCATCTCTGAGCGCGATCTGCACAA |
| | | TATTGGTCTTAACTCAGACTATGCTCGTCCCGAGTGGATGATCATCACTGTCCTGCCTGTACCCCCTCCTC |
| | | CCGTGCGTCCTAGTATTTCCATGGATGGTACTGGTACTGGCACGAGAAACGAGGATGATCTGACCTACAA |
| | | GCTTGGTGACATTATCCGCGCCAACGGTAATGTCAAGCAGGCCATTCGCGAAGGATCACCGCAACACAT |
| | | CGCGCGTGATTTTGAGGAGCTGCTGCAGTATCATGTTGCCACCTACATGGATAATGATATTGCTGGTCAG |
| | | CCGCGGGCCCTCCAAAAGAGCGGTCGTCCTGTCAAGGCGATTCGCGCCCGTCTCAAGGGCAAGGAGGGT |
| | | CGTCTGCGAGGCAACTTGATGGGTAAGCGTGTCGACTTTTCCGCGCGTACCGTTATCACTGGTGACGCCA |
| | | ACTTGTCTCTGCACGAAGTTGGTGTTCCGCGAAGCATTGCTCGTACTCTCACCTATCCTGAGACTGTCACA |
| | | CCCTATAACATTGCCAAGCTGCATCAACTCGTTGAGAACGGACCTAACGAGCACCCTGGTGCCAAATAC |
| | | GTCATTCGCTCTGATGGCACCAGAATCGATCTACGACACCACAGACGCGCGGCTCAAATTTCTTTGGAAT |
| | | ATGGATGGAAGGTTGAGCGTCATCTAATCGATGGCGACTACATCATCTTCAACCGTCAGCCCTCCCTGCA |
| | | CAAGGAATCCATGATGGGTCATCGTGTCAAGGTCATGCCCTACTCTACCTTCCGACTCAACCTTTCCGTC |
| | | ACTTCCCCCTATAATGCCGATTTTGATGGTGATGAAATGAACTTGCACGTTCCCCAGACTGAGGAAACTC |
| | | GGGCTGAAATCAAGGAGCTGTGTCTCGTTCCCAACAATATTGTGTCGCCTCAGAAAAACGGTCCCCTAAT |
| | | GGGTATTGTTCAAGACTCTCTGGCCGGTGTCTACAAGCTCTGTCGTCGTGACACATTTATCGATAAAGAG |
| | | CTTGTTCAAAATCTCATGCTCTGGGTACCCAACTGGGATGGCGTCATTCCACAGCCGGCTATTCTGAAGC |
| | | CTAGACCTCGCTGGACCGGCAAGCAGATTATCAGCATGGTCATCCCCCCAGAGATCAGTCTGTACAGCA |
| | | AGGAAGATAAGCTGGACAATCCTAGACATGACGCTGGCTTGCTTATTCAGAGCGGTGAGCTCATGTACG |
| | | GTCTGCTCAAGAAGAAGAGTGTTGGTGCTGCCGCAGGAGGCATCATTCATTTGTGCTACAACGAACTCGG |
| | | ACCCGAGGGCGCCATGGCTTTCTTGAATGGTGTCCAACAAGTIGTCACCTACTGGCTACTGAACACCGGT |
| | | TTCAGTATTGGTATTGGCGACACTGTTCCCGACAAGGCTACCATCAATAAGATCCAGGAGCATATCGACG |
| | | AACACAAAGCAGAGGTCGCTCGCTTGACTGCCCAGGCTACAGCCAACGAGCTGGAGGCTTTACCTGGTA |
| | | TGAATGTCCGCGCCACTTTCGAGAACAAAGTCTCCATGGCTCTCAACATGGCCCGTGACCAGGCTGGTAC |
| | | TTCAACCATGAAGAGTTTGAAGGATTCCAACAATGCCGTCACCATGGCTGATTCTGGTTCCAAGGGTTCT |
| | | TCCATCAACATCTCGCAAATGACGGCCCTCGTCGGACAGCAGATTGTTGAAGGAAAGCGTATCCCTTTCG |
| | | GCTTCAAGTACCGAACACTGCCGCATTTCACAAAGGACGATTATTCGCCTGAAGCTCGTGGTTTCGTCGA |
| | | GAACTCGTACCTTCGTGGCTTGACTCCTTCGGAGTTCTTCTTCCACGCCATGGCTGGTAGAGAGGGTCTTA |
| | | TTGATACTGCGGTCAAGACCGCTGAGACGGGTTACATTCAACGACGTCTTGTCAAGGCGCTCGAAGATCT |
| | | CAGCGCGCGCTACGACGGAACTGTACGTAACTCATTGGGCGACGTTATTCAATTCTTGTACGGTGAAGAT |
| | | GGTCTCGATGCTATGTGCATTGAAAAACAGAGACTTGGCACCATCAAGATGTCTAATGCTGCCTTCGAAA |
| | | GCCAATATCGTTTGGATCTTGCCAACCCCCCTGAGTGGTTCCGCAAGGACTATGAGTACGGCAATGAGCT |
| | | GGCAGGCGATAGGGCGTCCATGGAACTGCTGGACATGGAGTGGGATGCCCTTGTTGCTGACAGACGCCT |
| | | TGTCCGTGATATCAACAAGAGCAAGATGGGTGAAGAGATGATGCAGCTTCCACTCAATATTGGCCGTAT |
| | | CATCGAGAGTGCCAAGCGTGTCTTCAACATTCGCGAAACTGACCGCAGCAATCTGCGGCCGTCCGATGTC |
| | | ATCACAACTGTTCAAAATTTGCTGGCCAACATGCAGATTGTGCGTGGCACGGACCCCATCTCAGTTGAGG |
| | | CCGATTACAATGCTACCATTCTGTTCAAGGCACTCCTGCGCTCTCGTCTCGCTTTCAAAGAGATTGTCTAC |
| | | GTGCATCGCTTAAACAAGCTTGCCTTTAACCACGTCATCGGCGAGCTTCAGAATCGTTGGGATAGAGCTT |
| | | TTGTTAGCCCTGGTGAGATGGTTGGTGTTCTCGCTGCCCAGTCTATTGGTGAGCCTGCTACTCAGATGACA |
| | | CTCAACACTTTTCACTTTGCTGGTGTGTCTTCGAAGAACGTTACTCTGGGTGTGCCGCGTCTCAAGGAGAT |
| | | TCTGAACTTGGCCAAAAATATCAAGACTCCCAGCATGGCCGTCTACCTGGACACCAAACTGGGTACGCA |
| | | GGAGCAGGCCAAGAAGCTCCGTAGTTTGGTCGAGTATACCAACTTGCGCTCCGTCACATCGGTCACCGA |
| | | AATCTACTACGATCCCGAGGTGCAGGCAACGAACATTGCTGAGGATGTGGACATGGTCGAATCTTACTTC |
| | | ATGATTCCCGATGATGCCCAGGATACCATTCACCGGCAGTCGCGCTGGCTCCTCCGCATTACTCTTGACC |
| | | GCCAGAAACTCCTGGACAAGGAAATCAAGATTGACGATGTCGCTGCGTGCATCAAGGAGGAATACTCAA |
| | | ACGATCTGGCCATCATCTTCTCTGACAACAATGCCGACGAGCAAGTCATTCGTATCCGTACCATTCGTCA |
| | | AAGTGATGACAAGGACGAGGATTCTGACACGAAGATTGAAGACGATGTCATGCTCAAGCGGCTAGAAGC |
| | | TCACTTGCTTGATACTATGACTTTACGCGGTGTTCCTGGCATCGAAAGGGCCTTCTTGACCAAGGGTACC |
| | | CGCCTGGTTGAGGATGAAGACGGCTCCGAACTGGCTCTCAAGGATAACCCGAAGTGCACACAGTGGTAC |
| | | TTGGATACCAGTGGTTCAGCACTCCGCGAGGTACTCGCTGTCCCCGGTGTCGATCCCACCAGAACCTACA |
| | | GCAACGACCTGTACCAGATCACTGAGGTGTTCGGTGTCGAGGCTGTACGATCTGCGCTGGTGAAGGAATT |
| | | GACCAACGTGTTGGCTTTTGACGGTTCGTACGTCAATCATCGTCATATTGCTTTGCTGTGCGACATTATGA |
| | | CCTATCGTGGTGTCATTTCGGCCGTCACACGTCACGGTATTAACCGGGCCGACACTGGTGCTCTGATGCG |
| | | TTGTTCCTTCGAAGAGACTGTCGAAATCCTGCTCGAGGCTGCCGCTACCGGCGAACTCGACGACTGCCGT |
| | | GGTATCTCCGAGAACGTCATGCTTGGTCAGCTTGCGCCCATGGGCACCGGCAACTTTGATGTCTACTTGG |
| | | ATCCTAAGATGCTGGAGACTGTCATTTCCGACAATTCTCGCATGGGCCTCATGCCTGGCATGCCCACCAA |
| | | GGAAGGCGAGGGCGAGGGTGCTGCCACACCATACGATAGTGGCTCGCCGATGGGCGACTCTGGTTACCT |
| | | CAGCATGGCTTCGCCTGCTGCTGGTAACTTTTCTCCTATTCAGGGTGCTGGCTCAGAAACACCTACCGGG |
| | | TTCAATACCGAGTATGGCGGTGGCTTCGGAGCCAATGGTTCGATGAGCCCGTACTCGCGCGGCGCTACTA |
| | | GTCCCTTCAGCACGTCGCCCACGTCGCCATTCAGCGCAGGCATGGGGGGCTATTCGCCATCGTCGCCTAA |
| | | CACGGGTTACTCTCCTACGTCGCCGATGCTTGACGGCGGTGCTCGTTACGCCACCTCTCCGTCATTCAGTC |
| | | CGTCGTCGCCGTCTTTCTCGCCGACGTCGCCGATGCTGCGACCCACCAGCCCGGCCAGCCCCAACTACAG |
| | | CCCAACGTCTCCTAGTTACTCGCCGACGTCGCCGACATCACCGCGACACTACTCGCCTACATCTCCTGCTC |
| | | AGTTCAACTCGCCGACGTCTCCCAGCTACTCCCCGGCCAGTCCCAATTACAGTCCTGCGTCTCCCAACTTC |
| | | CAGGGAGCTGGTCCTACTTCGCCCTCGTACTCGCCTGCGTCGCCATCGTGGTCACCAACCTCACCCGACG |
| | | CTTACTCACCCACGAGTCCAAGCTTCGGACGCAGCCCCGGCCAGCAGCAGTCGCCCACGAGCCCTGGTTA |
| | | CTCTCCCACCTCGCCGCAGTTCTCTCCTCGCACGCCTGGTCCCTCGGGCTCTGGTGGAGGTTCTGGAACTG |
| | | GCAACCAATACTCGCCTAGCTCGCCGAATAACGAGTGA |
| 15 | US-707 | ATGGCGAACCTTCCCTTTCCGCATTCGAGCGCGCCGCTCAAAACTGTTGAGGAAATACAGTTTGGTATGT |
| | | TATCCCCGGAGGAAATCAAAAACATGAGCGTCGCGCACATTCTCTATCCTGAAACAATGGAAGAGAACA |
| | | AGACAACACCACGCGACGGCGGTCTGAACGATCCTCTTCTTGGTTCCATCGATCGTCAGTTCAAGTGCAA |
| | | GACTTGCTCTCAGCCGATGAGCGAGTGTCCAGGTCATTTTGGCCATATAGAGCTGGCGAAACCTGTCTAT |
| | | CACCCTGGTTTCATCAAGAAAGTGAAAAAGGTTTTGGAGATTGTCTGCCACAACTGCAGCAAAGTGTTGG |
| | | CCGATGAAAGCGATCCCGAATTCGTCACAGCTATTCATACTCGCGATCCGAAACTCCGATTCAAGCGCGT |
| | | TTGGGCCGTATGCAAGAAGAAGCGCAAATGTGAGAATGAGGAGCGGCAAGACAAGAATAAAGACGAAG |
| | | AGTTCGCTCCAGGTGTCAAGAACGTCGTTCTCGAAGGACATGGCGGATGTGGCAATATGCAGCCGCAGG |
| | | TGAGACAGGCCGCGCTGCAACTCAAAGCTGCCTTCGAGGTTACTTCGGAAGAGGGTCCCAAGAGGAAAG |
| | | AGACGGTTAATATCAGCGCCGAGATGGCGCATGGTATCCTTCGCCGCATCTCTGAGCGCGATCTGCACAA |
| | | TATTGGTCTTAACTCAGACTATGCTCGTCCCGAGTGGATGATCATCACTGTCCTGCCTGTACCCCCTCCTC |
| | | CCGTGCGTCCTAGTATTTCCATGGATGGTACTGGTACTGGCACGAGAAACGAGGATGATCTGACCTACAA |
| | | GCTTGGTGACATTATCCGCGCCAACGGTAATGTCAAGCAGGCCATTCGCGAAGGATCACCGCAACACAT |
| | | CGCGCGTGATTTTGAGGAGCTGCTGCAGTATCATGTTGCCACCTACATGGATAATGATATTGCTGGTCAG |
| | | CCGCGGGCCCTCCAAAAGAGCGGTCGTCCTGTCAAGGCGATTCGCGCCCGTCTCAAGGGCAAGGAGGGT |
| | | CGTCTGCGAGGCAACTTGATGGGTAAGCGTGTCGACTTTTCCGCGCGTACCGTTATCACTGGTGACGCCA |
| | | ACTTGTCTCTGCACGAAGTTGGTGTTCCGCGAAGCATTGCTCGTACTCTCACCTATCCTGAGACTGTCACA |
| | | CCCTATAACATTGCCAAGCTGCATCAACTCGTTGAGAACGGACCTAACGAGCACCCTGGTGCCAAATAC |
| | | GTCATTCGCTCTGATGGCACCAGAATCGATCTACGACACCACAGACGCGCGGCTCAAATTTCTTTGGAAT |
| | | ATGGATGGAAGGTTGAGCGTCATCTAATCGATGGCGACTACATCATCTTCAACCGTCAGCCCTCCCTGCA |
| | | CAAGGAATCCATGATGGGTCATCGTGTCAAGGTCATGCCCTACTCTACCTTCCGACTCAACCTTTCCGTC |
| | | ACTTCCCCCTATAATGCCGATTTTGATGGTGATGAAATGAACTTGCACGTTCCCCAGACTGAGGAAACTC |
| | | GGGCTGAAATCAAGGAGCTGTGTCTCGTTCCCAACAATATTGTGTCGCCTCAGAAAAACGGTCCCCTAAT |
| | | GGGTATTGTTCAAGACTCTCTGGCCGGTGTCTACAAGCTCTGTCGTCGTGACACATTTATCGATAAAGAG |
| | | CTTGTTCAAAATCTCATGCTCTGGGTACCCAACTGGGATGGCGTCATTCCACAGCCGGCTATTCTGAAGC |
| | | CTAGACCTCGCTGGACCGGCAAGCAGATTATCAGCATGGTCATCCCCCCAGAGATCAGTCTGTACAGCA |
| | | AGGAAGATAAGCTGGACAATCCTAGACATGACGCTGGCTTGCTTATTCAGAGCGGTGAGCTCATGTACG |
| | | GTCTGCTCAAGAAGAAGAGTGTTGGTGCTGCCGCAGGAGGCATCATTCATTTGTGCTACAACGAACTCGG |
| | | ACCCGAGGGCGCCATGGCTTTCTTGAATGGTGTCCAACAAGTTGTCACCTACTGGCTACTGAACACCGGT |
| | | TTCAGTATTGGTATTGGCGACACTGTTCCCGACAAGGCTACCATCAATAAGATCCAGGAGCATATCGACG |
| | | AACACAAAGCAGAGGTCGCTCGCTTGACTGCCCAGGCTACAGCCAACGAGCTGGAGGCTTTACCTGGTA |
| | | TGAATGTCCGCGCCACTTTCGAGAACAAAGTCTCCATGGCTCTCAACATGGCCCGTGACCAGGCTGGTAC |
| | | TTCAACCATGAAGAGTTTGAAGGATTCCAACAATGCCGTCACCATGGCTGATTCTGGTTCCAAGGGTTCT |
| | | TCCATCAACATCTCGCAAATGACGGCCCTCGTCGGACAGCAGATTGTTGAAGGAAAGCGTATCCCTTTCG |
| | | GCTTCAAGTACCGAACACTGCCGCATTTCACAAAGGACGATTATTCGCCTGAAGCTCGTGGTTTCGTCGA |
| | | GAACTCGTACCTTCGTGGCTTGACTCCTTCGGAGTTCTTCTTCCACGCCATGGCTGGTAGAGAGGGTCTTA |
| | | TTGATACTGCGGTCAAGACCGCTGAGACGGGTTACATTCAACGACGTCTCGTCAAGGCGCTCGAAGATCT |
| | | CAGCGCGCGCTACGACGGAACTGTACGTAACTCATTGGGCGACGTTATTCAATTCTTGTACGGTGAAGAT |
| | | GGTCTCGATGCTATGTGCATTGAAAAACAGAGACTTGGCACCATCAAGATGTCTAATGCTGCCTTCGAAA |
| | | GCCAATATCGTTTGGATCTTGCCAACCCCCCTGAGTGGTTCCGCAAGGACTATGAGTACGGCAATGAGCT |
| | | GGCAGGCGATAGGGCGTCCATGGAACTGCTGGACATGGAGTGGGATGCCCTTGTTGCTGACAGACGCCT |
| | | TGTCCGTGATATCAACAAGAGCAAGATGGGTGAAGAGATGATGCAGCTTCCACTCAATATTGGCCGTAT |
| | | CATCGAGAGTGCCAAGCGTGTCTTCAACATTCGCGAAACTGACCGCAGCAATCTGCGGCCGTCCGATGTC |
| | | ATCACAACTGTTCAAAATTTGCTGGCCAACATGCAGATTGTGCGTGGCACGGACCCCATCTCAGTTGAGG |
| | | CCGATTACAATGCTACCATTCTGTTCAAGGCACTCCTGCGCTCTCGTCTCGCTTTCAAAGAGATTGTCTAC |
| | | GTGCATCGCTTAAACAAGCTTGCCTTTAACCACGTCATCGGCGAGCTTCAGAATCGTTGGGATAGAGCTT |
| | | TTGTTAGCCCTGGTGAGATGGTTGGTGTTCTCGCTGCCCAGTCTATTGGTGAGCCTGCTACTCAGATGACA |
| | | CTCAACACTTTTCACTTTGCTGGTGTGTCTTCGAAGAACGTTACTCTGGGTGTGCCGCGTCTCAAGGAGAT |
| | | TCTGAACTTGGCCAAAAATATCAAGACTCCCAGCATGGCCGTCTACCTGGACACCAAACTGGGTACGCA |
| | | GGAGCAGGCCAAGAAGCTCCGTAGTTTGGTCGAGTATACCAACTTGCGCTCCGTCACATCGGTCACCGA |
| | | AATCTACTACGATCCCGAGGTGCAGGCAACGAACATTGCTGAGGATGTGGACATGGTCGAATCTTACTTC |
| | | ATGATTCCCGATGATGCCCAGGATACCATTCACCGGCAGTCGCGCTGGCTCCTCCGCATTACTCTTGACC |
| | | GCCAGAAACTCCTGGACAAGGAAATCAAGATTGACGATGTCGCTGCGTGCATCAAGGATGAATACTCAA |
| | | ACGATCTGGCCATCATCTTCTCTGACAACAATGCCGACGAGCAAGTCATTCGTATCCGTACCATTCGTCA |
| | | AAGTGATGACAAGGACGAGGATTCTGACACGAAGATTGAAGACGATGTCATGCTCAAGCGGCTAGAAGC |
| | | TCACTTGCTTGATACTATGACTTTACGCGGTGTTCCTGGCATCGAAAGGGCCTTCTTGACCAAGGGTACC |
| | | CGCCTGGTTGAGGATGAAGACGGCTCCGAACTGGCTCTCAAGGATAACCCGAAGTGCACACAGTGGTAC |
| | | TTGGATACCAGTGGTTCAGCACTCCGCGAGGTACTCGCTGTCCCCGGTGTCGATCCCACCAGAACCTACA |
| | | GCAACGACCTGTACCAGATCACTGAGGTGTTCGGTGTCGAGGCTGTACGATCTGCGCTGGTGAAGGAATT |
| | | GACCAACGTGTTGGCTTTTGACGGTTCGTACGTCAATCATCGTCATATTGCTTTGCTGTGCGACATTATGA |
| | | CCTATCGTGGTGTCATTTCGGCCGTCACACGTCACGGTATTAACCGGGCCGACACTGGTGCTCTGATGCG |
| | | TTGTTCCTTCGAAGAGACTGTCGAAATCCTGCTCGAGGCTGCCGCTACCGGCGAACTCGACGACTGCCGT |
| | | GGTATCTCCGAGAACGTCATGCTTGGTCAGCTTGCGCCCATGGGCACCGGCAACTTTGATGTCTACTTGG |
| | | ATCCTAAGATGCTGGAGACTGTCATTTCCGACAATTCTCGCATGGGCCTCATGCCTGGCATGCCCACCAA |
| | | GGAAGGCGAGGGCGAGGGTGCTGCCACACCATACGATAGTGGCTCGCCGATGGGCGACTCTGGTTACCT |
| | | CAGCATGGCTTCGCCTGCTGCTGGTAACTTTTCTCCTATTCAGGGTGCTGGCTCAGAAACACCTACCGGG |
| | | TTCAATACCGAGTATGGCGGTGGCTTCGGAGCCAATGGTTCGATGAGCCCGTACTCGCGCGGCGCTACTA |
| | | GTCCCTTCAGCACGTCGCCCACGTCGCCATTTAGCGCAGGCATGGGGGGCTATTCGCCATCGTCGCCTAA |
| | | CACGGGTTACTCTCCTACGTCGCCGATGCTTGACGGCGGTGCTCGTTACGCCACCTCTCCGTCATTCAGTC |
| | | CGTCGTCGCCGTCTTTCTCGCCGACGTCGCCGATGCTGCGACCCACCAGCCCGGCCAGCCCCAACTACAG |
| | | CCCAACGTCTCCTAGTTACTCGCCGACGTCGCCGACATCACCGCGACACTACTCGCCTACATCTCCTGCTC |
| | | AGTTCAACTCGCCGACGTCTCCCAGCTACTCCCCGGCCAGTCCCAATTACAGTCCTGCGTCTCCCAACTTC |
| | | CAGGGAGCTGGTCCTACTTCGCCCTCGTACTCGCCTGCGTCGCCATCGTGGTCACCAACCTCACCCGACG |
| | | CTTACTCACCCACGAGTCCAAGCTTCGGACGCAGCCCCGGCCAGCAGCAGTCGCCCACGAGCCCTGGTTA |
| | | CTCTCCCACCTCGCCGCAGTTCTCTCCTCGCACGCCTGGTCCCTCGGGCTCTGGTGGAGGTTCTGGAACTG |
| | | GCAACCAATACTCGCCTAGCTCGCCGAATAACGAGTGA |
| 16 | US-803 | ATGGCGAACCTTCCCTTTCCGCATTCGAGCGCGCCGCTCAAAACTGTTGAGGAAATACAGTTTGGTATGT |
| | | TATCCCCGGAGGAAATCAAAAACATGAGCGTCGCGCACATTCTCTATCCTGAAACAATGGAAGAGAACA |
| | | AGACAACACCACGCGACGGCGGTCTGAACGATCCTCTTCTTGGTTCCATCGATCGTCAGTTCAAGTGCAA |
| | | GACTTGCTCTCAGCCGATGAGCGAGTGTCCAGGTCATTTTGGCCATATAGAGCTGGCGAAACCTGTCTAT |
| | | CACCCTGGTTTCATCAAGAAAGTGAAAAAGGTTTTGGAGATTGTCTGCCACAACTGCAGCAAAGTGTTGG |
| | | CCGATGAAAGCGATCCCGAATTCGTCACAGCTATTCATACTCGCGATCCGAAACTCCGATTCAAGCGCGT |
| | | TTGGGCCGTATGCAAGAAGAAGCGCAAATGCGAGAATGAGGAGCGGCAAGACAAGAATAAAGACGAAG |
| | | AGTTCGCTCCAGGTGTCAAGAACGTCGTTCTCGAAGGACATGGCGGATGTGGCAATATGCAGCCGCAGG |
| | | TGAGACAGGCCGCGCTGCAACTCAAAGCTGCCTTCGAGGTTACTTCGGAAGAGGGTCCCAAGAGGAAAG |
| | | AGACGGTTAATATCAGCGCCGAGATGGCGCATGGTATCCTTCGCCGCATCTCTGAGCGCGATCTGCACAA |
| | | TATTGGTCTTAACTCAGACTATGCTCGTCCCGAGTGGATGATCATCACTGTCCTGCCTGTACCCCCTCCTC |
| | | CCGTGCGTCCTAGTATTTCCATGGATGGTACTGGTACTGGCACGAGAAACGAGGATGATCTGACCTACAA |
| | | GCTTGGTGACATTATCCGCGCCAACGGTAATGTCAAGCAGGCCATTCGCGAAGGATCACCGCAACACAT |
| | | CGCGCGTGATTTTGAGGAGCTGCTGCAGTATCATGTTGCCACCTACATGGATAATGATATTGCTGGTCAG |
| | | CCGCGGGCCCTCCAAAAGAGCGGTCGTCCTGTCAAGGCGATTCGCGCCCGTCTCAAGGGCAAGGAGGGT |
| | | CGTCTGCGAGGCAACTTGATGGGTAAGCGTGTCGACTTTTCCGCGCGTACCGTTATCACTGGTGACGCCA |
| | | ACTTGTCTCTGCACGAAGTTGGTGTTCCGCGAAGCATTGCTCGTACTCTCACCTATCCTGAGACTGTCACA |
| | | CCCTATAACATTGCCAAGCTGCATCAACTCGTTGAGAACGGACCTAACGAGCACCCTGGTGCCAAATAC |
| | | GTCATTCGCTCTGATGGCACCAGAATCGATCTACGACACCACAGACGCGCGGCTCAAATTTCTTTGGAAT |
| | | ATGGATGGAAGGTTGAGCGTCATCTAATCGATGGCGACTACATCATCTTCAACCGTCAGCCCTCCCTGCA |
| | | CAAGGAATCCATGATGGGTCATCGTGTCAAGGTCATGCCCTACTCTACCTTCCGACTCAACCTTTCCGTC |
| | | ACTTCCCCCTATAATGCCGATTTTGATGGTGATGAAATGAACTTGCACGTTCCCCAGACTGAGGAAACTC |
| | | GGGCTGAAATCAAGGAGCTGTGTCTCGTTCCCAACAATATTGTGTCGCCTCAGAAAAACGGTCCCCTAAT |
| | | GGGTATTGTTCAAGACTCTCTGGCCGGTGTCTACAAGCTCTGTCGTCGTGACACATTTATCGATAAAGAG |
| | | CTTGTTCAAAATCTCATGCTCTGGGTACCCAACTGGGATGGCGTCATTCCACAGCCGGCTATTCTGAAGC |
| | | CTAGACCTCGCTGGACCGGCAAGCAGATTATCAGCATGGTCATCCCCCCAGAGATCAGTCTGTACAGCA |
| | | AGGAAGATAAGCTGGACAATCCTAGACATGACGCTGGCTTGCTTATTCAGAGCGGTGAGCTCATGTACG |
| | | GTCTGCTCAAGAAGAAGAGTGTTGGTGCTGCCGCAGGAGGCATCATTCATTTGTGCTACAACGAACTCGG |
| | | ACCCGAGGGCGCCATGGCTTTCTTGAATGGTGTCCAACAAGTTGTCACCTACTGGCTACTGAACACCGGT |
| | | TTCAGTATTGGTATTGGCGACACTGTTCCCGACAAGGCTACCATCAATAAGATCCAGGAGCATATCGACG |
| | | AACACAAAGCAGAGGTCGCTCGCTTGACTGCCCAGGCTACAGCCAACGAGCTGGAGGCTTTACCTGGTA |
| | | TGAATGTCCGCGCCACTTTCGAGAACAAAGTCTCCATGGCTCTCAACATGGCCCGTGACCAGGCTGGTAC |
| | | TTCAACCATGAAGAGTTTGAAGGATTCCAACAATGCCGTCACCATGGCTGATTCTGGTTCCAAGGGTTCT |
| | | TCCATCAACATCTCGCAAATGACGGCCCTCGTCGGACAGCAGATTGTTGAAGGAAAGCGTATCCCTTTCG |
| | | GCTTCAAGTACCGAACACTGCCGCATTTCACAAAGGACGATTATTCGCCTGAAGCTCGTGGTTTCGTCGA |
| | | GAACTCGTACCTTCGTGGCTTGACTCCTTCGGAGTTCTTCTTCCACGCCATGGCTGGTAGAGAGGGTCTTA |
| | | TTGATACTGCGGTCAAGACCGCTGAGACGGGTTACATTCAACGACGTCTTGTCAAGGCGCTCGAAGATCT |
| | | CAGCGCGCGCTACGACGGAACTGTACGTAACTCATTGGGCGACGTTATTCAATTCTTGTACGGTGAAGAT |
| | | GGTCTCGATGCTATGTGCATTGAAAAACAGAGACTTGGCACCATCAAGATGTCTAATGCTGCCTTCGAAA |
| | | GCCAATATCGTTTGGATCTTGCCAACCCCCCTGAGTGGTTCCGCAAGGACTATGAGTACGGCAATGAGCT |
| | | GGCAGGCGATAGGGCGTCCATGGAACTGCTGGACATGGAGTGGGATGCCCTTGTTGCTGACAGACGCCT |
| | | TGTCCGTGATATCAACAAGAGCAAGATGGGTGAAGAGATGATGCAGCTTCCACTCAATATTGGCCGTAT |
| | | CATCGAGAGTGCCAAGCGTGTCTTCAACATTCGCGAAACTGACCGCAGCAATCTGCGGCCGTCCGATGTC |
| | | ATCACAACTGTTCAAAATTTGCTGGCCAACATGCAGATTGTGCGTGGCACGGACCCCATCTCAGTTGAGG |
| | | CCGATTACAATGCTACCATTCTGTTCAAGGCACTCCTGCGCTCTCGTCTCGCTTTCAAAGAGATTGTCTAC |
| | | GTGCATCGCTTAAACAAGCTTGCCTTTAACCACGTCATCGGCGAGCTTCAGAATCGTTGGGATAGAGCTT |
| | | TTGTTAGCCCTGGTGAGATGGTTGGTGTTCTCGCTGCCCAGTCTATTGGTGAGCCTGCTACTCAGATGACA |
| | | CTCAACACTTTTCACTTTGCTGGTGTGTCTTCGAAGAACGTTACTCTGGGTGTGCCGCGTCTCAAGGAGAT |
| | | TCTGAACTTGGCCAAAAATATCAAGACTCCCAGCATGGCCGTCTACCTGGACACCAAACTGGGTACGCA |
| | | GGAGCAGGCCAAGAAGCTCCGTAGTTTGGTCGAGTATACCAACTTGCGCTCCGTCACATCGGTCACCGA |
| | | AATCTACTACGATCCCGAGGTGCAGGCAACGAACATTGCTGAGGATGTGGACATGGTCGAATCTTACTTC |
| | | ATGATTCCCGATGATGCCCAGGATACCATTCACCGGCAGTCGCGCTGGCTCCTCCGCATTACTCTTGACC |
| | | GCCAGAAACTCCTGGACAAGGAAATCAAGATTGACGATGTCGCTGCGTGCATCAAGGAGGAATACTCAA |
| | | ACGATCTGGCCATCATCTTCTCTGACAACAATGCCGACGAGCAAGTCATTCGTATCCGTACCATTCGTCA |
| | | AAGTGATGACAAGGACGAGGATTCTGACACGAAGATTGAAGACGATGTCATGCTCAAGCGGCTAGAAGC |
| | | TCACTTGCTTGATACTATGACTTTACGCGGTGTTCCTGGCATCGAAAGGGCCTTCTTGACCAAGGGTACC |
| | | CGCCTGGTTGAGGATGAAGACGGCTCCGAACTGGCTCTCAAGGATAACCCGAAGTGCACACAGTGGTAC |
| | | TTGGATACCAGTGGTTCAGCACTCCGCGAGGTACTCGCTGTCCCCGGTGTCGATCCCACCAGAACCTACA |
| | | GCAACGACCTGTACCAGATCACTGAGGTGTTCGGTGTCGAGGCTGTACGATCTGCGCTGGTGAAGGAATT |
| | | GACCAACGTGTTGGCTTTTGACGGTTCGTACGTCAATCATCGTCATATTGCTTTGCTGTGCGACATTATGA |
| | | CCTATCGTGGTGTCATTTCGGCCGTCACACGTCACGGTATTAACCGGGCCGACACTGGTGCTCTGATGCG |
| | | TTGTTCCTTCGAAGAGACTGTCGAAATCCTGCTCGAGGCTGCCGCTACCGGCGAACTCGACGACTGCCGT |
| | | GGTATCTCCGAGAACGTCATGCTTGGTCAGCTTGCGCCCATGGGCACCGGCAACTTTGATGTCTACTTGG |
| | | ATCCTAAGATGCTGGAGACTGTCATTTCCGACAATTCTCGCATGGGCCTCATGCCTGGCATGCCCACCAA |
| | | GGAAGGCGAGGGCGAGGGTGCTGCCACACCATACGATAGTGGCTCGCCGATGGGCGACTCTGGTTACCT |
| | | CAGCATGGCTTCGCCTGCTGCTGGTAACTTTTCTCCTATTCAGGGTGCTGGCTCAGAAACACCTACCGGG |
| | | TTCAATACCGAGTATGGCGGTGGCTTCGGAGCCAATGGTTCGATGAGCCCGTACTCGCGCGGCGCTACTA |
| | | GTCCCTTCAGCACGTCGCCCACGTCGCCATTCAGCGCAGGCATGGGGGGCTATTCGCCATCGTCGCCTAA |
| | | CACGGGTTACTCTCCTACGTCGCCGATGCTTGACGGCGGTGCTCGTTACGCCACCTCTCCGTCATTCAGTC |
| | | CGTCGTCGCCGTCTTTCTCGCCGACGTCGCCGATGCTGCGACCCACCAGCCCGGCCAGCCCCAACTACAG |
| | | CCCAACGTCTCCTAGTTACTCGCCGACGTCGCCGACATCACCGCGACACTACTCGCCTACATCTCCTGCTC |
| | | AGTTCAACTCGCCGACGTCTCCCAGCTACTCCCCGGCCAGTCCCAATTACAGTCCTGCGTCTCCCAACTTC |
| | | CAGGGAGCTGGTCCTACTTCGCCCTCGTACTCGCCTGCGTCGCCATCGTGGTCACCAACCTCACCCGACG |
| | | CTTACTCACCCACGAGTCCAAGCTTCGGACGCAGCCCCGGCCAGCAGCAGTCGCCCACGAGCCCTGGTTA |
| | | CTCTCCCACCTCGCCGCAGTTCTCTCCTCGCACGCCTGGTCCCTCGGGCTCTGGTGGAGGTTCTGGAACTG |
| | | GCAACCAATACTCGCCTAGCTCGCCGAATAACGAGTGA |
| 17 | US-670 | ATGGCGAACCTTCCCTTTCCGCATTCGAGCGCGCCGCTCAAAACTGTTGAGGAAATACAGTTTGGTATGT |
| | | TATCCCCGGAGGAAATCAAAAACATGAGCGTCGCGCACATTCTCTATCCTGAAACAATGGAAGAGAACA |
| | | AGACAACACCACGCGACGGCGGTCTGAACGATCCTCTTCTTGGTTCCATCGATCGTCAGTTCAAGTGCAA |
| | | GACTTGCTCTCAGCCGATGAGCGAGTGTCCAGGTCATTTTGGCCATATAGAGCTGGCGAAACCTGTCTAT |
| | | CACCCTGGTTTCATCAAGAAAGTGAAAAAGGTTTTGGAGATTGTCTGCCACAACTGCAGCAAAGTGTTGG |
| | | CCGATGAAAGCGATCCCGAATTCGTCACAGCTATTCATACTCGCGATCCGAAACTCCGATTCAAGCGCGT |
| | | TTGGGCCGTATGCAAGAAGAAGCGCAAATGCGAGAATGAGGAGCGGCAAGACAAGAATAAAGACGAAG |
| | | AGTTCGCTCCAGGTGTCAAGAACGTCGTTCTCGAAGGACATGGCGGATGTGGCAATATGCAGCCGCAGG |
| | | TGAGACAGGCCGCGCTGCAACTCAAAGCTGCCTTCGAGGTTACTTCGGAAGAGGGTCCCAAGAGGAAAG |
| | | AGACGGTTAATATCAGCGCCGAGATGGCGCATGGTATCCTTCGCCGCATCTCTGAGCGCGATCTGCACAA |
| | | TATTGGTCTTAACTCAGACTATGCTCGTCCCGAGTGGATGATCATCACTGTCCTGCCTGTACCCCCTCCTC |
| | | CCGTGCGTCCTAGTATTTCCATGGATGGTACTGGTACTGGCACGAGAAACGAGGATGATCTGACCTACAA |
| | | GCTTGGTGACATTATCCGCGCCAACGGTAATGTCAAGCAGGCCATTCGCGAAGGATCACCGCAACACAT |
| | | CGCGCGTGATTTTGAGGAGCTGCTGCAGTATCATGTTGCCACCTACATGGATAATGATATTGCTGGTCAG |
| | | CCGCGGGCCCTCCAAAAGAGCGGTCGTCCTGTCAAGGCGATTCGCGCCCGTCTCAAGGGCAAGGAGGGT |
| | | CGTCTGCGAGGCAACTTGATGGGTAAGCGTGTCGACTTTTCCGCGCGTACCGTTATCACTGGTGACGCCA |
| | | ACTTGTCTCTGCACGAAGTTGGTGTTCCGCGAAGCATTGCTCGTACTCTCACCTATCCTGAGACTGTCACA |
| | | CCCTATAACATTGCCAAGCTGCATCAACTCGTTGAGAACGGACCTAACGAGCACCCTGGTGCCAAATAC |
| | | GTCATTCGCTCTGATGGCACCAGAATCGATCTACGACACCACAGACGCGCGGCTCAAATTTCTTTGGAAT |
| | | ATGGATGGAAGGTTGAGCGTCATCTAATCGATGGCGACTACATCATCTTCAACCGTCAGCCCTCCCTGCA |
| | | CAAGGAATCCATGATGGGTCATCGTGTCAAGGTCATGCCCTACTCTACCTTCCGACTCAACCTTTCCGTC |
| | | ACTTCCCCCTATAATGCCGATTTTGATGGTGATGAAATGAACTTGCACGTTCCCCAGACTGAGGAAACTC |
| | | GGGCTGAAATCAAGGAGCTGTGTCTCGTTCCCAACAATATTGTGTCGCCTCAGAAAAACGGTCCCCTAAT |
| | | GGGTATTGTTCAAGACTCTCTGGCCGGTGTCTACAAGCTCTGTCGTCGTGACACATTTATCGATAAAGAG |
| | | CTTGTTCAAAATCTCATGCTCTGGGTACCCAACTGGGATGGCGTCATTCCACAGCCGGCTATTCTGAAGC |
| | | CTAGACCTCGCTGGACCGGCAAGCAGATTATCAGCATGGTCATCCCCCCAGAGATCAGTCTGTACAGCA |
| | | AGGAAGATAAGCTGGACAATCCTAGACATGACGCTGGCTTGCTTATTCAGAGCGGTGAGCTCATGTACG |
| | | GTCTGCTCAAGAAGAAGAGTGTTGGTGCTGCCGCAGGAGGCATCATTCATTTGTGCTACAACGAACTCGG |
| | | ACCCGAGGGCGCCATGGCTTTCTTGAATGGTGTCCAACAAGTIGTCACCTACTGGCTACTGAACACCGGT |
| | | TTCAGTATTGGTATTGGCGACACTGTTCCCGACAAGGCTACCATCAATAAGATCCAGGAGCATATCGACG |
| | | AACACAAAGCAGAGGTCGCTCGCTTGACTGCCCAGGCTACAGCCAACGAGCTGGAGGCTTTACCTGGTA |
| | | TGAATGTCCGCGCCACTTTCGAGAACAAAGTCTCCATGGCTCTCAACATGGCCCGTGACCAGGCTGGTAC |
| | | TTCAACCATGAAGAGTTTGAAGGATTCCAACAATGCCGTCACCATGGCTGATTCTGGTTCCAAGGGTTCT |
| | | TCCATCAACATCTCGCAAATGACGGCCCTCGTCGGACAGCAGATTGTTGAAGGAAAGCGTATCCCTTTCG |
| | | GCTTCAAGTACCGAACACTGCCGCATTTCACAAAGGACGATTATTCGCCTGAAGCTCGTGGTTTCGTCGA |
| | | GAACTCGTACCTTCGTGGCTTGACTCCTTCGGAGTTCTTCTTCCACGCCATGGCTGGTAGAGAGGGTCTTA |
| | | TTGATACTGCGGTCAAGACCGCTGAGACGGGTTACATTCAACGACGTCTTGTCAAGGCGCTCGAAGATCT |
| | | CAGCGCGCGCTACGACGGAACTGTACGTAACTCATTGGGCGACGTTATTCAATTCTTGTACGGTGAAGAT |
| | | GGTCTCGATGCTATGTGCATTGAAAAACAGAGACTTGGCACCATCAAGATGTCTAATGCTGCCTTCGAAA |
| | | GCCAATATCGTTTGGATCTTGCCAACCCCCCTGAGTGGTTCCGCAAGGACTATGAGTACGGCAATGAGCT |
| | | GGCAGGCGATAGGGCGTCCATGGAACTGCTGGACATGGAGTGGGATGCCCTTGTTGCTGACAGACGCCT |
| | | TGTCCGTGATATCAACAAGAGCAAGATGGGTGAAGAGATGATGCAGCTTCCACTCAATATTGGCCGTAT |
| | | CATCGAGAGTGCCAAGCGTGTCTTCAACATTCGCGAAACTGACCGCAGCAATCTGCGGCCGTCCGATGTC |
| | | ATCACAACTGTTCAAAATTTGCTGGCCAACATGCAGATTGTGCGTGGCACGGACCCCATCTCAGTTGAGG |
| | | CCGATTACAATGCTACCATTCTGTTCAAGGCACTCCTGCGCTCTCGTCTCGCTTTCAAAGAGATTGTCTAC |
| | | GTGCATCGCTTAAACAAGCTTGCCTTTAACCACGTCATCGGCGAGCTTCAGAATCGTTGGGATAGAGCTT |
| | | TTGTTAGCCCTGGTGAGATGGTTGGTGTTCTCGCTGCCCAGTCTATTGGTGAGCCTGCTACTCAGATGACA |
| | | CTCAACACTTTTCACTTTGCTGGTGTGTCTTCGAAGAACGTTACTCTGGGTGTGCCGCGTCTCAAGGAGAT |
| | | TCTGAACTTGGCCAAAAATATCAAGACTCCCAGCATGGCCGTCTACCTGGACACCAAACTGGGTACGCA |
| | | GGAGCAGGCCAAGAAGCTCCGTAGTTTGGTCGAGTATACCAACTTGCGCTCCGTCACATCGGTCACCGA |
| | | AATCTACTACGATCCCGAGGTGCAGGCAACGAACATTGCTGAGGATGTGGACATGGTCGAATCTTACTTC |
| | | ATGATTCCCGATGATGCCCAGGATACCATTCACCGGCAGTCGCGCTGGCTCCTCCGCATTACTCTTGACC |
| | | GCCAGAAACTCCTGGACAAGGAAATCAAGATTGACGATGTCGCTGCGTGCATCAAGGAGGAATACTCAA |
| | | ACGATCTGGCCATCATCTTCTCTGACAACAATGCCGACGAGCAAGTCATTCGTATCCGTACCATTCGTCA |
| | | AAGTGATGACAAGGACGAGGATTCTGACACGAAGATTGAAGACGATGTCATGCTCAAGCGGCTAGAAGC |
| | | TCACTTGCTTGATACTATGACTTTACGCGGTGTTCCTGGCATCGAAAGGGCCTTCTTGACCAAGGGTACC |
| | | CGCCTGGTTGAGGATGAAGACGGCTCCGAACTGGCTCTCAAGGATAACCCGAAGTGCACACAGTGGTAC |
| | | TTGGATACCAGTGGTTCAGCACTCCGCGAGGTACTCGCTGTCCCCGGTGTCGATCCCACCAGAACCTACA |
| | | GCAACGACCTGTACCAGATCACTGAGGTGTTCGGTGTCGAGGCTGTACGATCTGCGCTGGTGAAGGAATT |
| | | GACCAACGTGTTGGCTTTTGACGGTTCGTACGTCAATCATCGTCATATTGCTTTGCTGTGCGACATTATGA |
| | | CCTATCGTGGTGTCATTTCGGCCGTCACACGTCACGGTATTAACCGGGCCGACACTGGTGCTCTGATGCG |
| | | TTGTTCCTTCGAAGAGACTGTCGAAATCCTGCTCGAGGCTGCCGCTACCGGCGAACTCGACGACTGCCGT |
| | | GGTATCTCCGAGAACGTCATGCTTGGTCAGCTTGCGCCCATGGGCACCGGCAACTTTGATGTCTACTTGG |
| | | ATCCTAAGATGCTGGAGACTGTCATTTCCGACAATTCTCGCATGGGCCTCATGCCTGGCATGCCCACCAA |
| | | GGAAGGCGAGGGCGAGGGTGCTGCCACACCATACGATAGTGGCTCGCCGATGGGCGACTCTGGTTACCT |
| | | CAGCATGGCTTCGCCTGCTGCTGGTAACTTTTCTCCTATTCAGGGTGCTGGCTCAGAAACACCTACCGGG |
| | | TTCAATACCGAGTATGGCGGTGGCTTCGGAGCCAATGGTTCGATGAGCCCGTACTCGCGCGGCGCTACTA |
| | | GTCCCTTCAGCACGTCGCCCACGTCGCCATTCAGCGCAGGCATGGGGGGCTATTCGCCATCGTCGCCTAA |
| | | CACGGGTTACTCTCCTACGTCGCCGATGCTTGACGGCGGTGCTCGTTACGCCACCTCTCCGTCATTCAGTC |
| | | CGTCGTCGCCGTCTTTCTCGCCGACGTCGCCGATGCTGCGACCCACCAGCCCGGCCAGCCCCAACTACAG |
| | | CCCAACGTCTCCTAGTTACTCGCCGACGTCGCCGACATCACCGCGACACTACTCGCCTACATCTCCTGCTC |
| | | AGTTCAACTCGCCGACGTCTCCCAGCTACTCCCCGGCCAGTCCCAATTACAGTCCTGCGTCTCCCAACTTC |
| | | CAGGGAGCTGGTCCTACTTCGCCCTCGTACTCGCCTGCGTCGCCATCGTGGTCACCAACCTCACCCGACG |
| | | CTTACTCACCCACGAGTCCAAGCTTCGGACGCAGCCCCGGCCAGCAGCAGTCGCCCACGAGCCCTGGTTA |
| | | CTCTCCCACCTCGCCGCAGTTCTCTCCTCGCACGCCTGGTCCCTCGGGCTCTGGTGGAGGTTCTGGAACTG |
| | | GCAACCAATACTCGCCTAGCTCGCCGAATAACGAGTGA |
| 18 | US-935 | ATGGCGAACCTTCCCTTTCCGCATTCGAGCGCGCCGCTCAAAACTGTTGAGGAAATACAGTTTGGTATGT |
| | | TATCCCCGGAGGAAATCAAAAACATGAGCGTCGCGCACATTCTCTATCCTGAAACAATGGAAGAGAACA |
| | | AGACAACACCACGCGACGGCGGTCTGAACGATCCTCTTCTTGGTTCCATCGATCGTCAGTTCAAGTGCAA |
| | | GACTTGCTCTCAGCCGATGAGCGAGTGTCCAGGTCATTTTGGCCATATAGAGCTGGCGAAACCTGTCTAT |
| | | CACCCTGGTTTCATCAAGAAAGTGAAAAAGGTTTTGGAGATTGTCTGCCACAACTGCAGCAAAGTGTTGG |
| | | CCGATGAAAGCGATCCCGAATTCGTCACAGCTATTCATACTCGCGATCCGAAACTCCGATTCAAGCGCGT |
| | | TTGGGCCGTATGCAAGAAGAAGCGCAAATGCGAGAATGAGGAGCGGCAAGACAAGAATAAAGACGAAG |
| | | AGTTCGCTCCAGGTGTCAAGAACGTCGTTCTCGAAGGACATGGCGGATGTGGCAATATGCAGCCGCAGG |
| | | TGAGACAGGCCGCGCTGCAACTCAAAGCTGCCTTCGAGGTTACTTCGGAAGAGGGTCCCAAGAGGAAAG |
| | | AGACGGTTAATATCAGCGCCGAGATGGCGCATGGTATCCTTCGCCGCATCTCTGAGCGCGATCTGCACAA |
| | | TATTGGTCTTAACTCAGACTATGCTCGTCCCGAGTGGATGATCATCACTGTCCTGCCTGTACCCCCTCCTC |
| | | CCGTGCGTCCTAGTATTTCCATGGATGGTACTGGTACTGGCACGAGAAACGAGGATGATCTGACCTACAA |
| | | GCTTGGTGACATTATCCGCGCCAACGGTAATGTCAAGCAGGCCATTCGCGAAGGATCACCGCAACACAT |
| | | CGCGCGTGATTTTGAGGAGCTGCTGCAGTATCATGTTGCCACCTACATGGATAATGATATTGCTGGTCAG |
| | | CCGCGGGCCCTCCAAAAGAGCGGTCGTCCTGTCAAGGCGATTCGCGCCCGTCTCAAGGGCAAGGAGGGT |
| | | CGTCTGCGAGGCAACTTGATGGGTAAGCGTGTCGACTTTTCCGCGCGTACCGTTATCACTGGTGACGCCA |
| | | ACTTGTCTCTGCACGAAGTTGGTGTTCCGCGAAGCATTGCTCGTACTCTCACCTATCCTGAGACTGTCACA |
| | | CCCTATAACATTGCCAAGCTGCATCAACTCGTTGAGAACGGACCTAACGAGCACCCTGGTGCCAAATAC |
| | | GTCATTCGCTCTGATGGCACCAGAATCGATCTACGACACCACAGACGCGCGGCTCAAATTTCTTTGGAAT |
| | | ATGGATGGAAGGTTGAGCGTCATCTAATCGATGGCGACTACATCATCTTCAACCGTCAGCCCTCCCTGCA |
| | | CAAGGAATCCATGATGGGTCATCGTGTCAAGGTCATGCCCTACTCTACCTTCCGACTCAACCTTTCCGTC |
| | | ACTTCCCCCTATAATGCCGATTTTGATGGTGATGAAATGAACTTGCACGTTCCCCAGACTGAGGAAACTC |
| | | GGGCTGAAATCAAGGAGCTGTGTCTCGTTCCCAACAATATTGTGTCGCCTCAGAAAAACGGTCCCCTAAT |
| | | GGGTATTGTTCAAGACTCTCTGGCCGGTGTCTACAAGCTCTGTCGTCGTGACACATTTATCGATAAAGAG |
| | | CTTGTTCAAAATCTCATGCTCTGGGTACCCAACTGGGATGGCGTCATTCCACAGCCGGCTATTCTGAAGC |
| | | CTAGACCTCGCTGGACCGGCAAGCAGATTATCAGCATGGTCATCCCCCCAGAGATCAGTCTGTACAGCA |
| | | AGGAAGATAAGCTGGACAATCCTAGACATGACGCTGGCTTGCTTATTCAGAGCGGTGAGCTCATGTACG |
| | | GTCTGCTCAAGAAGAAGAGTGTTGGTGCTGCCGCAGGAGGCATCATTCATTTGTGCTACAACGAACTCGG |
| | | ACCCGAGGGCGCCATGGCTTTCTTGAATGGTGTCCAACAAGTIGTCACCTACTGGCTACTGAACACCGGT |
| | | TTCAGTATTGGTATTGGCGACACTGTTCCCGACAAGGCTACCATCAATAAGATCCAGGAGCATATCGACG |
| | | AACACAAAGCAGAGGTCGCTCGCTTGACTGCCCAGGCTACAGCCAACGAGCTGGAGGCTTTACCTGGTA |
| | | TGAATGTCCGCGCCACTTTCGAGAACAAAGTCTCCATGGCTCTCAACATGGCCCGTGACCAGGCTGGTAC |
| | | TTCAACCATGAAGAGTTTGAAGGATTCCAACAATGCCGTCACCATGGCTGATTCTGGTTCCAAGGGTTCT |
| | | TCCATCAACATCTCGCAAATGACGGCCCTCGTCGGACAGCAGATTGTTGAAGGAAAGCGTATCCCTTTCG |
| | | GCTTCAAGTACCGAACACTGCCGCATTTCACAAAGGACGATTATTCGCCTGAAGCTCGTGGTTTCGTCGA |
| | | GAACTCGTACCTTCGTGGCTTGACTCCTTCGGAGTTCTTCTTCCACGCCATGGCTGGTAGAGAGGGTCTTA |
| | | TTGATACTGCGGTCAAGACCGCTGAGACGGGTTACATTCAACGACGTCTTGTCAAGGCGCTCGAAGATCT |
| | | CAGCGCGCGCTACGACGGAACTGTACGTAACTCATTGGGCGACGTTATTCAATTCTTGTACGGTGAAGAT |
| | | GGTCTCGATGCTATGTGCATTGAAAAACAGAGACTTGGCACCATCAAGATGTCTAATGCTGCCTTCGAAA |
| | | GCCAATATCGTTTGGATCTTGCCAACCCCCCTGAGTGGTTCCGCAAGGACTATGAGTACGGCAATGAGCT |
| | | GGCAGGCGATAGGGCGTCCATGGAACTGCTGGACATGGAGTGGGATGCCCTTGTTGCTGACAGACGCCT |
| | | TGTCCGTGATATCAACAAGAGCAAGATGGGTGAAGAGATGATGCAGCTTCCACTCAATATTGGCCGTAT |
| | | CATCGAGAGTGCCAAGCGTGTCTTCAACATTCGCGAAACTGACCGCAGCAATCTGCGGCCGTCCGATGTC |
| | | ATCACAACTGTTCAAAATTTGCTGGCCAACATGCAGATTGTGCGTGGCACGGACCCCATCTCAGTTGAGG |
| | | CCGATTACAATGCTACCATTCTGTTCAAGGCACTCCTGCGCTCTCGTCTCGCTTTCAAAGAGATTGTCTAC |
| | | GTGCATCGCTTAAACAAGCTTGCCTTTAACCACGTCATCGGCGAGCTTCAGAATCGTTGGGATAGAGCTT |
| | | TTGTTAGCCCTGGTGAGATGGTTGGTGTTCTCGCTGCCCAGTCTATTGGTGAGCCTGCTACTCAGATGACA |
| | | CTCAACACTTTTCACTTTGCTGGTGTGTCTTCGAAGAACGTTACTCTGGGTGTGCCGCGTCTCAAGGAGAT |
| | | TCTGAACTTGGCCAAAAATATCAAGACTCCCAGCATGGCCGTCTACCTGGACACCAAACTGGGTACGCA |
| | | GGAGCAGGCCAAGAAGCTCCGTAGTTTGGTCGAGTATACCAACTTGCGCTCCGTCACATCGGTCACCGA |
| | | AATCTACTACGATCCCGAGGTGCAGGCAACGAACATTGCTGAGGATGTGGACATGGTCGAATCTTACTTC |
| | | ATGATTCCCGATGATGCCCAGGATACCATTCACCGGCAGTCGCGCTGGCTCCTCCGCATTACTCTTGACC |
| | | GCCAGAAACTCCTGGACAAGGAAATCAAGATTGACGATGTCGCTGCGTGCATCAAGGAGGAATACTCAA |
| | | ACGATCTGGCCATCATCTTCTCTGACAACAATGCCGACGAGCAAGTCATTCGTATCCGTACCATTCGTCA |
| | | AAGTGATGACAAGGACGAGGATTCTGACACGAAGATTGAAGACGATGTCATGCTCAAGCGGCTAGAAGC |
| | | TCACTTGCTTGATACTATGACTTTACGCGGTGTTCCTGGCATCGAAAGGGCCTTCTTGACCAAGGGTACC |
| | | CGCCTGGTTGAGGATGAAGACGGCTCCGAACTGGCTCTCAAGGATAACCCGAAGTGCACACAGTGGTAC |
| | | TTGGATACCAGTGGTTCAGCACTCCGCGAGGTACTCGCTGTCCCCGGTGTCGATCCCACCAGAACCTACA |
| | | GCAACGACCTGTACCAGATCACTGAGGTGTTCGGTGTCGAGGCTGTACGATCTGCGCTGGTGAAGGAATT |
| | | GACCAACGTGTTGGCTTTTGACGGTTCGTACGTCAATCATCGTCATATTGCTTTGCTGTGCGACATTATGA |
| | | CCTATCGTGGTGTCATTTCGGCCGTCACACGTCACGGTATTAACCGGGCCGACACTGGTGCTCTGATGCG |
| | | TTGTTCCTTCGAAGAGACTGTCGAAATCCTGCTCGAGGCTGCCGCTACCGGCGAACTCGACGACTGCCGT |
| | | GGTATCTCCGAGAACGTCATGCTTGGTCAGCTTGCGCCCATGGGCACCGGCAACTTTGATGTCTACTTGG |
| | | ATCCTAAGATGCTGGAGACTGTCATTTCCGACAATTCTCGCATGGGCCTCATGCCTGGCATGCCCACCAA |
| | | GGAAGGCGAGGGCGAGGGTGCTGCCACACCATACGATAGTGGCTCGCCGATGGGCGACTCTGGTTACCT |
| | | CAGCATGGCTTCGCCTGCTGCTGGTAACTTTTCTCCTATTCAGGGTGCTGGCTCAGAAACACCTACCGGG |
| | | TTCAATACCGAGTATGGCGGTGGCTTCGGAGCCAATGGTTCGATGAGCCCGTACTCGCGCGGCGCTACTA |
| | | GTCCCTTCAGCACGTCGCCCACGTCGCCATTCAGCGCAGGCATGGGGGGCTATTCGCCATCGTCGCCTAA |
| | | CACGGGTTACTCTCCTACGTCGCCGATGCTTGACGGCGGTGCTCGTTACGCCACCTCTCCGTCATTCAGTC |
| | | CGTCGTCGCCGTCTTTCTCGCCGACGTCGCCGATGCTGCGACCCACCAGCCCGGCCAGCCCCAACTACAG |
| | | CCCAACGTCTCCTAGTTACTCGCCGACGTCGCCGACATCACCGCGACACTACTCGCCTACATCTCCTGCTC |
| | | AGTTCAACTCGCCGACGTCTCCCAGCTACTCCCCGGCCAGTCCCAATTACAGTCCTGCGTCTCCCAACTTC |
| | | CAGGGAGCTGGTCCTACTTCGCCCTCGTACTCGCCTGCGTCGCCATCGTGGTCACCAACCTCACCCGACG |
| | | CTTACTCACCCACGAGTCCAAGCTTCGGACGCAGCCCCGGCCAGCAGCAGTCGCCCACGAGCCCTGGTTA |
| | | CTCTCCCACCTCGCCGCAGTTCTCTCCTCGCACGCCTGGTCCCTCGGGCTCTGGTGGAGGTTCTGGAACTG |
| | | GCAACCAATACTCGCCTAGCTCGCCGAATAACGAGTGA |

TABLE 5

| SEQ | Strain | |
|---|---|---|
| ID No. | Number | RPB2 Sequence |
| 19 | AU-16727 | ATGGCAGACTACGAGGATGAATACGACTACGAGAATTACGAGGAAGAAGATTCGGGCATCACTCCCGAA |
| | | GATTGTTGGACCGTCATCTCCTCTTTCTTCGAATCCAAGGGCCTCGTCTCTCAGCAAACTGCCTCCTTTGA |
| | | TGAATTTACTCAGTCAACTATCCAAGACCTTGTCAGCGAGTACTCAAGAATATCTCTCGACCAACCCAAC |
| | | CCGCCCTCCAACGACGACCGGAAGATAAGCGTCCGCCGATACGAGATCGAGTTCGGCAGCATCATGGTA |
| | | TCGAGGCCTTCGATCAGTGAGACCGATGGGACCGTGACTTCGCTTCTACCATACGAGTGCCGCGATCGTA |
| | | ACCTGACCTATGCAGCGCCGGTTTACATTAAGATTACCAAAAAAGTGCAAGCTGCTGTGGAAACGGAAA |
| | | TACCTTTGCACGAGCTAGATGATGCTCAACAGACAGAGTATGCCAAAACTGGTGAGCTGCCTACACGGTT |
| | | GGCCTGGAGATCGGAGGATGCGCCAGAGCCCGAAAATTCCAACAAGCCTGATGCCTGGAAGGACATGGT |
| | | CTTCGTCGGGAAAATGCCGGTCATGGTCAAATCTAAAGTCTGCCATTTGAGCCGCGAGCATGACGAGAA |
| | | TTTATTTCTTGTCAATGAGTGCCCATACGACCAGGGCGGTTACTTTGTTATCAATGGAAGTGAAAAGGTG |
| | | CTTATCGCCCAAGAGCGTTCCGCCGCCAATATTGTTCAGGTCTTCAAGAAGGCTCAGCCCAGCCCTTATA |
| | | CATACACTGCTGAAATCAGAAGCGCTCTGGAGAAAGGCTCGCGTCTCATCTCCAGCATGATGCTCAAGCT |
| | | CTACGGGAAGGGTGAGTCAGCTCGTGGCGGCTTCGGGCAAACGATTCATACAACTCTTCCGTTTGTTAAA |
| | | GCAGACCTTCCTGTCGCCATTGTATTTAGAGCACTTGGCGTTGTCTCCGACGAGGATATTCTCAACCACAT |
| | | TTGTTACGACCGAAAAGACTCTCAGATGCTGGAAATGCTTCGGCCTTGTATCGAGGAAGCTTTCTGCGTC |
| | | CAAGACCGTGAAGTCGCCCTCGACTTCATTGGTAAGCGTGGTAATCGGGATCAGGCTGGTCTCGGACGC |
| | | GATAAACGTATTCGTGTGGCACGCGACATTCTTCAGAAAGAAACTTTGCCTCACATCTCCCAGGAGGCTG |
| | | GTAGCGAGACGCGCAAAGCCTTCTTTATCGGTTATATGGTCCACAAACTACTCCAGTGCGCTCTGGGGCG |
| | | TCGGGAGCCTGACGACAGAGACCACTTCGGCAAAAAGCGTCTTGATTTAGCAGGTCCACTACTTGCGAA |
| | | ACTCTTCCGCGGTATCATGCGCCGAATGCACTCAGAGCTCGCCAATTACCTGCGACGCTGCGTCGAGGGT |
| | | AACCGCCACTTCAATCTAGCTGTCGGTGTTAAGCCTGGCACGCTTTCGAACGGCCTCAAGTACTCTCTTG |
| | | CTACTGGTAATTGGGGTGACCAGAAAAAAGCTATGAGCTCAACTGCTGGTGTGTCCCAGGTTTTGAACCG |
| | | ATATACTTTTGCCTCAACACTATCCCACCTTCGACGTACCAATACACCCATTGGCAGAGATGGCAAACTG |
| | | GCAAAACCTCGTCAGCTTCATAATACTCATTGGGGTCTTGTCTGCCCTGCTGAGACACCTGAAGGCCAGG |
| | | CTTGCGGTCTTGTCAAGAATCTGTCTCTCATGTGCTACGTCAGCGTCGGCTCCCCAGCCGAGCCGCTCATT |
| | | GACTTCATGATCAACAGGGGTATGGAGGTCATTGAGGAGTACGAACCACTCAGATACCCACACGCTACC |
| | | AAGATTTTTGTCAACGGGACCTGGGTTGGAGTTCATCAGGACCCCAAGCACCTTGCTGACCAGGTATTCG |
| | | ACACCCGCCGCAAGTCCTACCTGCAGTATGAGGTGTCTCTTGTCAGAGAAATCCGTGACCAGGAATTCAA |
| | | GATCTTCTCTGATGCTGGCCGAGTCATGCGGCCTGTTTTTACTGTGCAAAGTAAAAATGACCCGGAGACT |
| | | GGCCTTGAAAAGGGACAGCTCGGTCTCACCAAGGATTTAGTCAACAGACTGGCGCAAGAGCAAGCCGAC |
| | | CCGCCAGATGATCCAGAAATGAAGACGGGTTGGGAGGGCTTGATCAAAGCTGGCGCTGTCGAGTATCTA |
| | | GACGCTGAAGAAGAGGAGACGTCAATGATTTGCATGACCCCGGAGGATCTGGAGCTTTATAGACTTCAG |
| | | AAAGCGGGTGTAGCTGTCGACGACGACCATGGCGATGATCTGAACAAGCGCCTGAAGACTAAAACCAAC |
| | | CCAACTACACACATGTACACTCACTGTGAAATTCACCCGAACCACAATCAATCGCCTCGTAACACCTACC |
| | | AATCCGCAATGGGTAAACAAGCCATGGGCTTCTTCCTGACAAATTATTCTCGTCGTATGGATACTATGGC |
| | | AAATATCCTGTACTACCCACAAAAGCCTTTGGCAACGACTAGATCCATGGAGTTCTTGAAGTTCCGAGAA |
| | | TTGCCTGCAGGTCAAAACGCCATCGTCGCGATTGCTTGTTATTCCGGTTATAATCAGGAGGACTCTGTGA |
| | | TTATGAATCAAAGCAGTATCGATCGCGGACTCTTCAGAAGTTTGTTCTTTCGTTCGTACTCTGATCAAGAG |
| | | AAAAAGGTCGGTTTGAACTACACAGAGATATTCGAAAAGCCGTTCCACCAAAGCACCCTGCGCATGAAA |
| | | CACGGTACTTACGACAAGCTCGATGAGGACGGTATTGTCGCTCCTGGCGTTCGTGTGTCTGGAGAAGACA |
| | | TTATCATTGGCAAAACTGCGCCAATTGACCCAGAGACGCAAGACTTGGGCGCGCGTACAACTGCGCATC |
| | | AGCGCCGTGATATCTCTACGCCTCTGCGTAGTACCGAAAATGGTATTGTTGATCAAGTCATTGTGACTGT |
| | | CAACGCCGACAACGTCAAATACGTCAAGGTCAGGGTTCGTACGACCAAGATACCCCAGATCGGTGATAA |
| | | GTTTGCTTCACGACACGGCCAAAAAGGTACCATTGGTGTCACATACCGACAGGAAGATATGCCATTCAC |
| | | GAGAGAGGGGGTCACCCCAGATATCATTATCAACCCGCACGCCATTCCCTCTCGAATGACAATTGCTCAT |
| | | CTGATCGAATGTCTTTTGAGCAAGGTTTCGACTTTAGAAGGTATGGAAGGCGATGCTACTCCATTTACCG |
| | | ACGTCACTGTCGATTCTGTCTCCGACTTGCTTCGCAAGCACGGCTATCAGTCCCGCGGGTTTGAGATCAT |
| | | GTACAACGGTCACACTGGTAAGAAACTTCGTGCTCAGGTTTTCTTCGGACCGACCTATTACCAGCGTCTG |
| | | CGTCACATGGTGGACGACAAAATTCACGCCCGTGCCCGTGGCCCTGTGCAGATCATGACTAGACAACCT |
| | | GTGGAGGGTCGTGCAAGGGATGGGGGGTTGCGTTTCGGCGAAATGGAACGTGATTGCATGATTGCGCAT |
| | | GGTGCAGCAGCATTCTTGAAGGAGCGCCTCTTTGAAGTGTCTGATGCGTTCCGAGTGCACATCTGCGAGA |
| | | TTTGCGGCTTGATGACGCCGATTGCACCTTGCAAGAACAAGACCAAAATTGCCCAGATTCACATTCCGTA |
| | | CGCAGCCAAGCTATTGTTCCAAGAACTTCAATCCATGAACATTGCAGCACGTATGTTTACGGACAGATCT |
| | | GGTGCCTCGATCAGGTAG |
| 20 | US-52 | ATGGCAGACTACGAGGATGAATACGACTACGAGAATTACGAGGAAGAAGATTCGGGCATCACTCCCGAA |
| | | GATTGTTGGACCGTCATCTCCTCTTTCTTCGAATCCAAGGGCCTCGTCTCTCAGCAAACTGCCTCCTTTGA |
| | | TGAATTTACTCAGTCAACTATCCAAGACCTTGTCAGCGAGTACTCAAGAATATCTCTCGACCAACCCAAC |
| | | CCGCCCTCCAACGACGACCGGAAGATAAGCGTCCGCCGATACGAGATCGAGTTCGGCAGCATCATGGTA |
| | | TCGAGGCCTTCGATCAGTGAGACCGATGGGACCGTGACTTCGCTTCTACCATACGAGTGCCGCGATCGTA |
| | | ACCTGACCTATGCAGCGCCGGTTTACATTAAGATTACCAAAAAAGTGCAAGCTGCTGTGGAAACGGAAA |
| | | TACCTTTGCACGAGCTAGATGATGCTCAACAGACAGAGTATGCCAAAACTGGTGAGCTGCCTACACGGTT |
| | | GGCCTGGAGATCGGAGGATGCGCCAGAGCCCGAAAATTCCAACAAGCCTGATGCCTGGAAGGACATGGT |
| | | CTTCGTCGGGAAAATGCCGGTCATGGTCAAATCTAAAGTCTGCCATTTGAGCCGCGAGCATGACGAGAA |
| | | TTTATTTCTTGTCAATGAGTGCCCATACGACCAGGGCGGTTACTTTGTTATCAATGGAAGTGAAAAGGTG |
| | | CTTATCGCCCAAGAGCGTTCCGCCGCCAATATTGTTCAGGTCTTCAAGAAGGCTCAGCCCAGCCCTTATA |
| | | CATACACTGCTGAAATCAGAAGCGCTCTGGAGAAAGGCTCGCGTCTCATCTCCAGCATGATGCTCAAGCT |
| | | CTACGGGAAGGGTGAGTCAGCTCGTGGCGGCTTCGGGCAAACGATTCATACAACTCTTCCGTTTGTTAAA |
| | | GCAGACCTTCCTGTCGCCATTGTATTTAGAGCACTTGGCGTTGTCTCCGACGAGGATATTCTCAACCACAT |
| | | TTGTTACGACCGAAAAGACTCTCAGATGCTGGAAATGCTTCGGCCTTGTATCGAGGAAGCTTTCTGCGTC |
| | | CAAGACCGTGAAGTCGCCCTCGACTTCATTGGTAAGCGTGGTAATCGGGATCAGGCTGGTCTCGGACGC |
| | | GATAAACGTATTCGTGTGGCACGCGACATTCTTCAGAAAGAAACTTTGCCTCACATCTCCCAGGAGGCTG |
| | | GTAGCGAGACGCGCAAAGCCTTCTTTATCGGTTATATGGTCCACAAACTACTCCAGTGCGCTCTGGGGCG |
| | | TCGGGAGCCTGACGACAGAGACCACTTCGGCAAAAAGCGTCTTGATTTAGCAGGTCCACTACTTGCGAA |
| | | ACTCTTCCGCGGTATCATGCGCCGAATGCACTCAGAGCTCGCCAATTACCTGCGACGCTGCGTCGAGGGT |
| | | AACCGCCACTTCAATCTAGCTGTCGGTGTTAAGCCTGGCACGCTTTCGAACGGCCTCAAGTACTCTCTTG |
| | | CTACTGGTAATTGGGGTGACCAGAAAAAAGCTATGAGCTCAACTGCTGGTGTGTCCCAGGTTTTGAACCG |
| | | ATATACTTTTGCCTCAACACTATCCCACCTTCGACGTACCAATACACCCATTGGCAGAGATGGCAAACTG |
| | | GCAAAACCTCGTCAGCTTCATAATACTCATTGGGGTCTTGTCTGCCCTGCTGAGACACCTGAAGGCCAGG |
| | | CTTGCGGTCTTGTCAAGAATCTGTCTCTCATGTGCTACGTCAGCGTCGGCTCCCCAGCCGAGCCGCTCATT |
| | | GACTTCATGATCAACAGGGGTATGGAGGTCATTGAGGAGTACGAACCACTCAGATACCCACACGCTACC |
| | | AAGATTTTTGTCAACGGGACCTGGGTTGGAGTTCATCAGGACCCCAAGCACCTTGCTGACCAGGTATTCG |
| | | ACACCCGCCGCAAGTCCTACCTGCAGTATGAGGTGTCTCTTGTCAGAGAAATCCGTGACCAGGAATTCAA |
| | | GATCTTCTCTGATGCTGGCCGAGTCATGCGGCCTGTTTTTACTGTGCAAAGTAAAAATGACCCGGAGACT |
| | | GGCCTTGAAAAGGGACAGCTCGGTCTCACCAAGGATTTAGTCAACAGACTGGCGCAAGAGCAAGCCGAC |
| | | CCGCCAGATGATCCAGAAATGAAGACGGGTTGGGAGGGCTTGATCAAAGCTGGCGCTGTCGAGTATCTA |
| | | GACGCTGAAGAAGAGGAGACGTCAATGATTTGCATGACCCCGGAGGATCTGGAGCTTTATAGACTTCAG |
| | | AAAGCGGGTGTAGCTGTCGACGACGACCATGGCGATGATCTGAACAAGCGCCTGAAGACTAAAACCAAC |
| | | CCAACTACACACATGTACACTCACTGTGAAATTCACCCGAGTATGATCTTGGGTATCTGCGCAAGCATTA |
| | | TTCCTTTCCCAGACCACAATCAATCGCCTCGTAACACCTACCAATCCGCAATGGGTAAACAAGCCATGGG |
| | | CTTCTTCCTGACAAATTATTCTCGTCGTATGGATACTATGGCAAATATCCTGTACTACCCACAAAAGCCTT |
| | | TGGCAACGACTAGATCCATGGAGTTCTTGAAGTTCCGAGAATTGCCTGCAGGTCAAAACGCCATCGTCGC |
| | | GATTGCTTGTTATTCCGGTTATAATCAGGAGGACTCTGTGATTATGAATCAAAGCAGTATCGATCGCGGA |
| | | CTCTTCAGAAGTTTGTTCTTTCGTTCGTACTCTGATCAAGAGAAAAAGGTCGGTTTGAACTACACAGAGA |
| | | TATTCGAAAAGCCGTTCCACCAAAGCACCCTGCGCATGAAACACGGTACTTACGACAAGCTCGATGAGG |
| | | ACGGTATTGTCGCTCCTGGCGTTCGTGTGTCTGGAGAAGACATTATCATTGGCAAAACTGCGCCAATTGA |
| | | CCCAGAGACGCAAGACTTGGGCGCGCGTACAACTGCGCATCAGCGCCGTGATATCTCTACGCCTCTGCGT |
| | | AGTACCGAAAATGGTATTGTTGATCAAGTCATTGTGACTGTCAACGCCGACAACGTCAAATACGTCAAG |
| | | GTCAGGGTTCGTACGACCAAGATACCCCAGATCGGTGATAAGTTTGCTTCACGACACGGCCAAAAAGGT |
| | | ACCATTGGTGTCACATACCGACAGGAAGATATGCCATTCACGAGAGAGGGGGTCACCCCAGATATCATT |
| | | ATCAACCCGCACGCCATTCCCTCTCGAATGACAATTGCTCATCTGATCGAATGTCTTTTGAGCAAGGTTTC |
| | | GACTTTAGAAGGTATGGAAGGCGATGCTACTCCATTTACCGACGTCACTGTCGATTCTGTCTCCGACTTG |
| | | CTTCGCAAGCACGGCTATCAGTCCCGCGGGTTTGAGATCATGTACAACGGTCACACTGGTAAGAAACTTC |
| | | GTGCTCAGGTTTTCTTCGGACCGACCTATTACCAGCGTCTGCGTCACATGGTGGACGACAAAATTCACGC |
| | | CCGTGCCCGTGGCCCTGTGCAGATCATGACTAGACAACCTGTGGAGGGTCGTGCAAGGGATGGGGGGTT |
| | | GCGTTTCGGCGAAATGGAACGTGATTGCATGATTGCGCATGGTGCAGCAGCATTCTTGAAGGAGCGCCTC |
| | | TTTGAAGTGTCTGATGCGTTCCGAGTGCACATCTGCGAGATTTGCGGCTTGATGACGCCGATTGCGTAA |
| 21 | US-675 | ATGGCAGACTACGAGGATGAATACGACTACGAGAATTACGAGGAAGAAGATTCGGGCATCACTCCCGAA |
| | | GATTGTTGGACCGTCATCTCCTCTTTCTTCGAATCCAAGGGCCTCGTCTCTCAGCAAACTGCCTCCTTTGA |
| | | TGAATTTACTCAGTCAACTATCCAAGACCTTGTCAGCGAGTACTCAAGAATATCTCTCGACCAACCCAAC |
| | | CCGCCCTCCAACGACGACCGGAAGATAAGCGTCCGCCGATACGAGATCGAGTTCGGCAGCATCATGGTA |
| | | TCGAGGCCTTCGATCAGTGAGACCGATGGGACCGTGACTTCGCTTCTACCATACGAGTGCCGCGATCGTA |
| | | ACCTGACCTATGCAGCGCCGGTTTACATTAAGATTACCAAAAAAGTGCAAGCTGCTGTGGAAACGGAAA |
| | | TACCTTTGCACGAGCTAGATGATGCTCAACAGACAGAGTATGCCAAAACTGGTGAGCTGCCTACACGGTT |
| | | GGCCTGGAGATCGGAGGATGCGCCAGAGCCCGAAAATTCCAACAAGCCTGATGCCTGGAAGGACATGGT |
| | | CTTCGTCGGGAAAATGCCGGTCATGGTCAAATCTAAAGTCTGCCATTTGAGCCGCGAGCATGACGAGAA |
| | | TTTATTTCTTGTCAATGAGTGCCCATACGACCAGGGCGGTTACTTTGTTATCAATGGAAGTGAAAAGGTG |
| | | CTTATCGCCCAAGAGCGTTCCGCCGCCAATATTGTTCAGGTCTTCAAGAAGGCTCAGCCCAGCCCTTATA |
| | | CATACACTGCTGAAATCAGAAGCGCTCTGGAGAAAGGCTCGCGTCTCATCTCCAGCATGATGCTCAAGCT |
| | | CTACGGGAAGGGTGAGTCAGCTCGTGGCGGCTTCGGGCAAACGATTCATACAACTCTTCCGTTTGTTAAA |
| | | GCAGACCTTCCTGTCGCCATTGTATTTAGAGCACTTGGCGTTGTCTCCGACGAGGATATTCTCAACCACAT |
| | | TTGTTACGACCGAAAAGACTCTCAGATGCTGGAAATGCTTCGGCCTTGTATCGAGGAAGCTTTCTGCGTC |
| | | CAAGACCGTGAAGTCGCCCTCGACTTCATTGGTAAGCGTGGTAATCGGGATCAGGCTGGTCTCGGACGC |
| | | GATAAACGTATTCGTGTGGCACGCGACATTCTTCAGAAAGAAACTTTGCCTCACATCTCCCAGGAGGCTG |
| | | GTAGCGAGACGCGCAAAGCCTTCTTTATCGGTTATATGGTCCACAAACTACTCCAGTGCGCTCTGGGGCG |
| | | TCGGGAGCCTGACGACAGAGACCACTTCGGCAAAAAGCGTCTTGATTTAGCAGGTCCACTACTTGCGAA |
| | | ACTCTTCCGCGGTATCATGCGCCGAATGCACTCAGAGCTCGCCAATTACCTGCGACGCTGCGTCGAGGGT |
| | | AACCGCCACTTCAATCTAGCTGTCGGTGTTAAGCCTGGCACGCTTTCGAACGGCCTCAAGTACTCTCTTG |
| | | CTACTGGTAATTGGGGTGACCAGAAAAAAGCTATGAGCTCAACTGCTGGTGTGTCCCAGGTTTTGAACCG |
| | | ATATACTTTTGCCTCAACACTATCCCACCTTCGACGTACCAATACACCCATTGGCAGAGATGGCAAACTG |
| | | GCAAAACCTCGTCAGCTTCATAATACTCATTGGGGTCTTGTCTGCCCTGCTGAGACACCTGAAGGCCAGG |
| | | CTTGCGGTCTTGTCAAGAATCTGTCTCTCATGTGCTACGTCAGCGTCGGCTCCCCAGCCGAGCCGCTCATT |
| | | GACTTCATGATCAACAGGGGTATGGAGGTCATTGAGGAGTACGAACCACTCAGATACCCACACGCTACC |
| | | AAGATTTTTGTCAACGGGACCTGGGTTGGAGTTCATCAGGACCCCAAGCACCTTGCTGACCAGGTATTCG |
| | | ACACCCGCCGCAAGTCCTACCTGCAGTATGAGGTGTCTCTTGTCAGAGAAATCCGTGACCAGGAATTCAA |
| | | GATCTTCTCTGATGCTGGCCGAGTCATGCGGCCTGTTTTTACTGTGCAAAGTAAAAATGACCCGGAGACT |
| | | GGCCTTGAAAAGGGACAGCTCGGTCTCACCAAGGATTTAGTCAACAGACTGGCGCAAGAGCAAGCCGAC |
| | | CCGCCAGATGATCCAGAAATGAAGACGGGTTGGGAGGGCTTGATCAAAGCTGGCGCTGTCGAGTATCTA |
| | | GACGCTGAAGAAGAGGAGACGTCAATGATTTGCATGACCCCGGAGGATCTGGAGCTTTATAGACTTCAG |
| | | AAAGCGGGTGTAGCTGTCGACGACGACCATGGCGATGATCTGAACAAGCGCCTGAAGACTAAAACCAAC |
| | | CCAACTACACACATGTACACTCACTGTGAAATTCACCCGAGTATGATCTTGGGTATCTGCGCAAGCATTA |
| | | TTCCTTTCCCAGACCACAATCAATCGCCTCGTAACACCTACCAATCCGCAATGGGTAAACAAGCCATGGG |
| | | CTTCTTCCTGACAAATTATTCTCGTCGTATGGATACTATGGCAAATATCCTGTACTACCCACAAAAGCCTT |
| | | TGGCAACGACTAGATCCATGGAGTTCTTGAAGTTCCGAGAATTGCCTGCAGGTCAAAACGCCATCGTCGC |
| | | GATTGCTTGTTATTCCGGTTATAATCAGGAGGACTCTGTGATTATGAATCAAAGCAGTATCGATCGCGGA |
| | | CTCTTCAGAAGTTTGTTCTTTCGTTCGTACTCTGATCAAGAGAAAAAGGTCGGTTTGAACTACACAGAGA |
| | | TATTCGAAAAGCCGTTCCACCAAAGCACCCTGCGCATGAAACACGGTACTTACGACAAGCTCGATGAGG |
| | | ACGGTATTGTCGCTCCTGGCGTTCGTGTGTCTGGAGAAGACATTATCATTGGCAAAACTGCGCCAATTGA |
| | | CCCAGAGACGCAAGACTTGGGCGCGCGTACAACTGCGCATCAGCGCCGTGATATCTCTACGCCTCTGCGT |
| | | AGTACCGAAAATGGTATTGTTGATCAAGTCATTGTGACTGTCAACGCCGACAACGTCAAATACGTCAAG |
| | | GTCAGGGTTCGTACGACCAAGATACCCCAGATCGGTGATAAGTTTGCTTCACGACACGGCCAAAAAGGT |
| | | ACCATTGGTGTCACATACCGACAGGAAGATATGCCATTCACGAGAGAGGGGGTCACCCCAGATATCATT |
| | | ATCAACCCGCACGCCATTCCCTCTCGAATGACAATTGCTCATCTGATCGAATGTCTTTTGAGCAAGGTTTC |
| | | GACTTTAGAAGGTATGGAAGGCGATGCTACTCCATTTACCGACGTCACTGTCGATTCTGTCTCCGACTTG |
| | | CTTCGCAAGCACGGCTATCAGTCCCGCGGGTTTGAGATCATGTACAACGGTCACACTGGTAAGAAACTTC |
| | | GTGCTCAGGTTTTCTTCGGACCGACCTATTACCAGCGTCTGCGTCACATGGTGGACGACAAAATTCACGC |
| | | CCGTGCCCGTGGCCCTGTGCAGATCATGACTAGACAACCTGTGGAGGGTCGTGCAAGGGATGGGGGGTT |
| | | GCGTTTCGGCGAAATGGAACGTGATTGCATGATTGCGCATGGTGCAGCAGCATTCTTGAAGGAGCGCCTC |
| | | TTTGAAGTGTCTGATGCGTTCCGAGTGCACATCTGCGAGATTTGCGGCTTGATGACGCCGATTGCGTAA |
| 22 | US-699 | ATGGCAGACTACGAGGATGAATACGACTACGAGAATTACGAGGAAGAAGATTCGGGCATCACTCCCGAA |
| | | GATTGTTGGACCGTCATCTCCTCTTTCTTCGAATCCAAGGGCCTCGTCTCTCAGCAAACTGCCTCCTTTGA |
| | | TGAATTTACTCAGTCAACTATCCAAGACCTTGTCAGCGAGTACTCAAGAATATCTCTCGACCAACCCAAC |
| | | CCGCCCTCCAACGACGACCGGAAGATAAGCGTCCGCCGATACGAGATCGAGTTCGGCAGCATCATGGTA |
| | | TCGAGGCCTTCGATCAGTGAGACCGATGGGACCGTGACTTCGCTTCTACCATACGAGTGCCGCGATCGTA |
| | | ACCTGACCTATGCAGCGCCGGTTTACATTAAGATTACCAAAAAAGTGCAAGCTGCTGTGGAAACGGAAA |
| | | TACCTTTGCACGAGCTAGATGATGCTCAACAGACAGAGTATGCCAAAACTGGTGAGCTGCCTACACGGTT |
| | | GGCCTGGAGATCGGAGGATGCGCCAGAGCCCGAAAATTCCAACAAGCCTGATGCCTGGAAGGACATGGT |
| | | CTTCGTCGGGAAAATGCCGGTCATGGTCAAATCTAAAGTCTGCCATTTGAGCCGCGAGCATGACGAGAA |
| | | TTTATTTCTTGTCAATGAGTGCCCATACGACCAGGGCGGTTACTTTGTTATCAATGGAAGTGAAAAGGTG |
| | | CTTATCGCCCAAGAGCGTTCCGCCGCCAATATTGTTCAGGTCTTCAAGAAGGCTCAGCCCAGCCCTTATA |
| | | CATACACTGCTGAAATCAGAAGCGCTCTGGAGAAAGGCTCGCGTCTCATCTCCAGCATGATGCTCAAGCT |
| | | CTACGGGAAGGGTGAGTCAGCTCGTGGCGGCTTCGGGCAAACGATTCATACAACTCTTCCGTTTGTTAAA |
| | | GCAGACCTTCCTGTCGCCATTGTATTTAGAGCACTTGGCGTTGTCTCCGACGAGGATATTCTCAACCACAT |
| | | TTGTTACGACCGAAAAGACTCTCAGATGCTGGAAATGCTTCGGCCTTGTATCGAGGAAGCTTTCTGCGTC |
| | | CAAGACCGTGAAGTCGCCCTCGACTTCATTGGTAAGCGTGGTAATCGGGATCAGGCTGGTCTCGGACGC |
| | | GATAAACGTATTCGTGTGGCACGCGACATTCTTCAGAAAGAAACTTTGCCTCACATCTCCCAGGAGGCTG |
| | | GTAGCGAGACGCGCAAAGCCTTCTTTATCGGTTATATGGTCCACAAACTACTCCAGTGCGCTCTGGGGCG |
| | | TCGGGAGCCTGACGACAGAGACCACTTCGGCAAAAAGCGTCTTGATTTAGCAGGTCCACTACTTGCGAA |
| | | ACTCTTCCGCGGTATCATGCGCCGAATGCACTCAGAGCTCGCCAATTACCTGCGACGCTGCGTCGAGGGT |
| | | AACCGCCACTTCAATCTAGCTGTCGGTGTTAAGCCTGGCACGCTTTCGAACGGCCTCAAGTACTCTCTTG |
| | | CTACTGGTAATTGGGGTGACCAGAAAAAAGCTATGAGCTCAACTGCTGGTGTGTCCCAGGTTTTGAACCG |
| | | ATATACTTTTGCCTCAACACTATCCCACCTTCGACGTACCAATACACCCATTGGCAGAGATGGCAAACTG |
| | | GCAAAACCTCGTCAGCTTCATAATACTCATTGGGGTCTTGTCTGCCCTGCTGAGACACCTGAAGGCCAGG |
| | | CTTGCGGTCTTGTCAAGAATCTGTCTCTCATGTGCTACGTCAGCGTCGGCTCCCCAGCCGAGCCGCTCATT |
| | | GACTTCATGATCAACAGGGGTATGGAGGTCATTGAGGAGTACGAACCACTCAGATACCCACACGCTACC |
| | | AAGATTTTTGTCAACGGGACCTGGGTTGGAGTTCATCAGGACCCCAAGCACCTTGCTGACCAGGTATTCG |
| | | ACACCCGCCGCAAGTCCTACCTGCAGTATGAGGTGTCTCTTGTCAGAGAAATCCGTGACCAGGAATTCAA |
| | | GATCTTCTCTGATGCTGGCCGAGTCATGCGGCCTGTTTTTACTGTGCAAAGTAAAAATGACCCGGAGACT |
| | | GGCCTTGAAAAGGGACAGCTCGGTCTCACCAAGGATTTAGTCAACAGACTGGCGCAAGAGCAAGCCGAC |
| | | CCGCCAGATGATCCAGAAATGAAGACGGGTTGGGAGGGCTTGATCAAAGCTGGCGCTGTCGAGTATCTA |
| | | GACGCTGAAGAAGAGGAGACGTCAATGATTTGCATGACCCCGGAGGATCTGGAGCTTTATAGACTTCAG |
| | | AAAGCGGGTGTAGCTGTCGACGACGACCATGGCGATGATCTGAACAAGCGCCTGAAGACTAAAACCAAC |
| | | CCAACTACACACATGTACACTCACTGTGAAATTCACCCGAGTATGATCTTGGGTATCTGCGCAAGCATTA |
| | | TTCCTTTCCCAGACCACAATCAATCGCCTCGTAACACCTACCAATCCGCAATGGGTAAACAAGCCATGGG |
| | | CTTCTTCCTGACAAATTATTCTCGTCGTATGGATACTATGGCAAATATCCTGTACTACCCACAAAAGCCTT |
| | | TGGCAACGACTAGATCCATGGAGTTCTTGAAGTTCCGAGAATTGCCTGCAGGTCAAAACGCCATCGTCGC |
| | | GATTGCTTGTTATTCCGGTTATAATCAGGAGGACTCTGTGATTATGAATCAAAGCAGTATCGATCGCGGA |
| | | CTCTTCAGAAGTTTGTTCTTTCGTTCGTACTCTGATCAAGAGAAAAAGGTCGGTTTGAACTACACAGAGA |
| | | TATTCGAAAAGCCGTTCCACCAAAGCACCCTGCGCATGAAACACGGTACTTACGACAAGCTCGATGAGG |
| | | ACGGTATTGTCGCTCCTGGCGTTCGTGTGTCTGGAGAAGACATTATCATTGGCAAAACTGCGCCAATTGA |
| | | CCCAGAGACGCAAGACTTGGGCGCGCGTACAACTGCGCATCAGCGCCGTGATATCTCTACGCCTCTGCGT |
| | | AGTACCGAAAATGGTATTGTTGATCAAGTCATTGTGACTGTCAACGCCGACAACGTCAAATACGTCAAG |
| | | GTCAGGGTTCGTACGACCAAGATACCCCAGATCGGTGATAAGTTTGCTTCACGACACGGCCAAAAAGGT |
| | | ACCATTGGTGTCACATACCGACAGGAAGATATGCCATTCACGAGAGAGGGGGTCACCCCAGATATCATT |
| | | ATCAACCCGCACGCCATTCCCTCTCGAATGACAATTGCTCATCTGATCGAATGTCTTTTGAGCAAGGTTTC |
| | | GACTTTAGAAGGTATGGAAGGCGATGCTACTCCATTTACCGACGTCACTGTCGATTCTGTCTCCGACTTG |
| | | CTTCGCAAGCACGGCTATCAGTCCCGCGGGTTTGAGATCATGTACAACGGTCACACTGGTAAGAAACTTC |
| | | GTGCTCAGGTTTTCTTCGGACCGACCTATTACCAGCGTCTGCGTCACATGGTGGACGACAAAATTCACGC |
| | | CCGTGCCCGTGGCCCTGTGCAGATCATGACTAGACAACCTGTGGAGGGTCGTGCAAGGGATGGGGGGTT |
| | | GCGTTTCGGCGAAATGGAACGTGATTGCATGATTGCGCATGGTGCAGCAGCATTCTTGAAGGAGCGCCTC |
| | | TTTGAAGTGTCTGATGCGTTCCGAGTGCACATCTGCGAGATTTGCGGCTTGATGACGCCGATTGCGTAA |
| 23 | US-707 | ATGGCAGACTACGAGGATGAATACGACTACGAGAATTACGAGGAAGAAGATTCGGGCATCACTCCCGAA |
| | | GATTGTTGGACCGTCATCTCCTCTTTCTTCGAATCCAAGGGCCTCGTCTCTCAGCAAACTGCCTCCTTTGA |
| | | TGAATTTACTCAGTCAACTATCCAAGACCTTGTCAGCGAGTACTCAAAAATATCTCTCGACCAACCCAAC |
| | | CCGCCCTCCAACGACGACCGGAAGATAAGCGTCCGCCGATACGAGATCGAGTTCGGCAGCATCATGGTA |
| | | TCGAGGCCTTCGATCAGTGAGACCGATGGGACCGTGACTTCGCTTCTACCATACGAGTGCCGCGATCGTA |
| | | ACCTGACCTATGCAGCGCCGGTTTACATTAAGATTACCAAAAAAGTGCAAGCTGCTGTGGAAACGGAAA |
| | | TACCTTTGCACGAGCTAGATGATGCTCAACAGACAGAGTATGCCAAAACTGGTGAGCTGCCTACACGGTT |
| | | GGCCTGGAGATCGGAGGATGCGCCAGAGCCCGAAAATTCCAACAAGCCTGATGCCTGGAAGGACATGGT |
| | | CTTCGTCGGGAAAATGCCGGTCATGGTCAAATCTAAAGTCTGCCATTTGAGCCGCGAGCATGACGAGAA |
| | | TTTATTTCTTGTCAATGAGTGCCCATACGACCAGGGCGGTTACTTTGTTATCAATGGAAGTGAAAAGGTG |
| | | CTTATCGCCCAAGAGCGTTCCGCCGCCAATATTGTTCAGGTCTTCAAGAAGGCTCAGCCCAGCCCTTATA |
| | | CATACACTGCTGAAATCAGAAGCGCTCTGGAGAAAGGCTCGCGTCTCATCTCCAGCATGATGCTCAAGCT |
| | | CTACGGGAAGGGTGAGTCAGCTCGTGGCGGCTTCGGGCAAACGATTCATACAACTCTTCCGTTTGTTAAA |
| | | GCAGACCTTCCTGTCGCCATTGTATTTAGAGCACTTGGCGTTGTCTCCGACGAGGATATTCTCAACCACAT |
| | | TTGTTACGACCGAAAAGACTCTCAGATGCTGGAAATGCTTCGGCCTTGTATCGAGGAAGCTTTCTGCGTC |
| | | CAAGACCGTGAAGTCGCCCTCGACTTCATTGGTAAGCGTGGTAATCGGGATCAGGCTGGTCTCGGACGC |
| | | GATAAACGTATTCGTGTGGCACGCGACATTCTTCAGAAAGAAACTTTGCCTCACATCTCCCAGGAGGCTG |
| | | GTAGCGAGACGCGCAAAGCCTTCTTTATCGGTTATATGGTCCACAAACTACTCCAGTGCGCTCTGGGGCG |
| | | TCGGGAGCCTGACGACAGAGACCACTTCGGCAAAAAGCGTCTTGATTTAGCAGGTCCACTACTTGCGAA |
| | | ACTCTTCCGCGGTATCATGCGCCGAATGCACTCAGAGCTCGCCAATTACCTGCGACGCTGCGTCGAGGGT |
| | | AACCGCCACTTCAATCTAGCTGTCGGTGTTAAGCCTGGCACGCTTTCGAACGGCCTCAAGTACTCTCTTG |
| | | CTACTGGTAATTGGGGTGACCAGAAAAAAGCTATGAGCTCAACTGCTGGTGTGTCCCAGGTTTTGAACCG |
| | | ATATACTTTTGCCTCAACACTATCCCACCTTCGACGTACCAATACACCCATTGGCAGAGATGGCAAACTG |
| | | GCAAAACCTCGTCAGCTTCATAATACTCATTGGGGTCTTGTCTGCCCTGCTGAGACACCTGAAGGCCAGG |
| | | CTTGCGGTCTTGTCAAGAATCTGTCTCTCATGTGCTACGTCAGCGTCGGCTCCCCAGCCGAGCCGCTCATT |
| | | GACTTCATGATCAACAGGGGTATGGAGGTCATTGAGGAGTACGAACCACTCAGATACCCACACGCTACC |
| | | AAGATTTTTGTCAACGGGACCTGGGTTGGAGTTCACCAGGACCCCAAGCACCTTGCTGACCAGGTATTCG |
| | | ACACCCGCCGCAAGTCCTACCTGCAGTATGAGGTGTCTCTTGTCAGAGAAATCCGTGACCAGGAATTCAA |
| | | GATCTTCTCTGATGCTGGCCGAGTCATGCGGCCTGTTTTTACTGTGCAAAGTAAAAATGACCCGGAGACT |
| | | GGCCTTGAAAAGGGACAGCTCGGTCTCACCAAGGATTTAGTCAACAGACTGGCGCAAGAGCAAGCCGAC |
| | | CCGCCAGATGATCCAGAAATGAAGACGGGTTGGGAGGGCTTGATCAAAGCTGGCGCTGTCGAGTATCTA |
| | | GACGCTGAAGAAGAGGAGACGTCAATGATTTGCATGACCCCGGAGGATCTGGAGCTTTATAGACTTCAG |
| | | AAAGCGGGTGTAGCTGTCGACGACGACCATGGCGATGATCTGAACAAGCGCCTGAAGACTAAAACCAAC |
| | | CCAACTACACACATGTACACTCACTGTGAAATTCACCCGAGTATGATCTTGGGTATCTGCGCAAGCATTA |
| | | TTCCTTTCCCAGACCACAATCAATCGCCTCGTAACACCTACCAATCCGCAATGGGTAAACAAGCCATGGG |
| | | CTTCTTCCTGACAAATTATTCTCGTCGTATGGATACTATGGCAAATATCCTGTACTACCCACAAAAGCCTT |
| | | TGGCAACGACTAGATCCATGGAGTTCTTGAAGTTCCGAGAATTGCCTGCAGGTCAAAACGCCATCGTCGC |
| | | GATTGCTTGTTATTCCGGTTATAATCAGGAGGACTCTGTGATTATGAATCAAAGCAGTATCGATCGCGGA |
| | | CTCTTCAGAAGTTTGTTCTTTCGTTCGTACTCTGATCAAGAGAAAAAGGTCGGTTTGAACTACACAGAGA |
| | | TATTCGAAAAGCCGTTCCACCAAAGCACCCTGCGCATGAAACACGGTACTTACGACAAGCTCGATGAGG |
| | | ACGGTATTGTCGCTCCTGGCGTTCGTGTGTCTGGAGAAGACATTATCATTGGCAAAACTGCGCCAATTGA |
| | | CCCAGAGACGCAAGACTTGGGCGCGCGTACAACTGCGCATCAGCGCCGTGATATCTCTACGCCTCTGCGT |
| | | AGTACCGAAAATGGTATTGTTGATCAAGTCATTGTGACTGTCAACGCCGACAACGTCAAATACGTCAAG |
| | | GTCAGGGTTCGTACGACCAAGATACCCCAGATTGGTGATAAGTTTGCTTCACGACACGGCCAAAAAGGT |
| | | ACCATTGGTGTCACATACCGACAGGAAGATATGCCATTCACGAGAGAGGGGGTCACCCCAGATATCATT |
| | | ATCAACCCGCACGCCATTCCCTCTCGAATGACAATTGCTCATCTGATCGAATGTCTTTTGAGCAAGGTTTC |
| | | GACTTTAGAAGGTATGGAAGGCGATGCTACTCCATTTACCGACGTCACTGTCGATTCTGTCTCCGACTTG |
| | | CTTCGCAAGCACGGCTATCAGTCCCGCGGGTTTGAGATCATGTACAACGGTCACACTGGTAAGAAACTTC |
| | | GTGCTCAGGTCTTCTTCGGACCGACCTATTACCAGCGTCTGCGTCACATGGTGGACGACAAAATTCACGC |
| | | CCGTGCCCGTGGCCCTGTGCAGATCATGACTAGACAACCTGTGGAGGGTCGTGCAAGGGATGGGGGGTT |
| | | GCGTTTCGGCGAAATGGAACGTGATTGCATGATTGCGCATGGTGCAGCAGCATTCTTGAAGGAGCGCCTC |
| | | TTTGAAGTGTCTGATGCGTTCCGAGTGCACATCTGCGAGATTTGCGGCTTGATGACGCCGATTGCAAATC |
| | | TTTCAAAGCAATCTTTTGAATGCAGACCTTGCAAGAACAAGACCAAAATTGCCCAGATTCACATTCCGTA |
| | | CGCAGCCAAGCTATTGTTCCAAGAACTTCAATCCATGAACATTGCAGCACGTATGTTTACGGACAGATCT |
| | | GGTGCCTCGATCAGGTAG |
| 24 | US-803 | ATGGCAGACTACGAGGATGAATACGACTACGAGAATTACGAGGAAGAAGATTCGGGCATCACTCCCGAA |
| | | GATTGTTGGACCGTCATCTCCTCTTTCTTCGAATCCAAGGGCCTCGTCTCTCAGCAAACTGCCTCCTTTGA |
| | | TGAATTTACTCAGTCAACTATCCAAGACCTTGTCAGCGAGTACTCAAGAATATCTCTCGACCAACCCAAC |
| | | CCGCCCTCCAACGACGACCGGAAGATAAGCGTCCGCCGATACGAGATCGAGTTCGGCAGCATCATGGTA |
| | | TCGAGGCCTTCGATCAGTGAGACCGATGGGACCGTGACTTCGCTTCTACCATACGAGTGCCGCGATCGTA |
| | | ACCTGACCTATGCAGCGCCGGTTTACATTAAGATTACCAAAAAAGTGCAAGCTGCTGTGGAAACGGAAA |
| | | TACCTTTGCACGAGCTAGATGATGCTCAACAGACAGAGTATGCCAAAACTGGTGAGCTGCCTACACGGTT |
| | | GGCCTGGAGATCGGAGGATGCGCCAGAGCCCGAAAATTCCAACAAGCCTGATGCCTGGAAGGACATGGT |
| | | CTTCGTCGGGAAAATGCCGGTCATGGTCAAATCTAAAGTCTGCCATTTGAGCCGCGAGCATGACGAGAA |
| | | TTTATTTCTTGTCAATGAGTGCCCATACGACCAGGGCGGTTACTTTGTTATCAATGGAAGTGAAAAGGTG |
| | | CTTATCGCCCAAGAGCGTTCCGCCGCCAATATTGTTCAGGTCTTCAAGAAGGCTCAGCCCAGCCCTTATA |
| | | CATACACTGCTGAAATCAGAAGCGCTCTGGAGAAAGGCTCGCGTCTCATCTCCAGCATGATGCTCAAGCT |
| | | CTACGGGAAGGGTGAGTCAGCTCGTGGCGGCTTCGGGCAAACGATTCATACAACTCTTCCGTTTGTTAAA |
| | | GCAGACCTTCCTGTCGCCATTGTATTTAGAGCACTTGGCGTTGTCTCCGACGAGGATATTCTCAACCACAT |
| | | TTGTTACGACCGAAAAGACTCTCAGATGCTGGAAATGCTTCGGCCTTGTATCGAGGAAGCTTTCTGCGTC |
| | | CAAGACCGTGAAGTCGCCCTCGACTTCATTGGTAAGCGTGGTAATCGGGATCAGGCTGGTCTCGGACGC |
| | | GATAAACGTATTCGTGTGGCACGCGACATTCTTCAGAAAGAAACTTTGCCTCACATCTCCCAGGAGGCTG |
| | | GTAGCGAGACGCGCAAAGCCTTCTTTATCGGTTATATGGTCCACAAACTACTCCAGTGCGCTCTGGGGCG |
| | | TCGGGAGCCTGACGACAGAGACCACTTCGGCAAAAAGCGTCTTGATTTAGCAGGTCCACTACTTGCGAA |
| | | ACTCTTCCGCGGTATCATGCGCCGAATGCACTCAGAGCTCGCCAATTACCTGCGACGCTGCGTCGAGGGT |
| | | AACCGCCACTTCAATCTAGCTGTCGGTGTTAAGCCTGGCACGCTTTCGAACGGCCTCAAGTACTCTCTTG |
| | | CTACTGGTAATTGGGGTGACCAGAAAAAAGCTATGAGCTCAACTGCTGGTGTGTCCCAGGTTTTGAACCG |
| | | ATATACTTTTGCCTCAACACTATCCCACCTTCGACGTACCAATACACCCATTGGCAGAGATGGCAAACTG |
| | | GCAAAACCTCGTCAGCTTCATAATACTCATTGGGGTCTTGTCTGCCCTGCTGAGACACCTGAAGGCCAGG |
| | | CTTGCGGTCTTGTCAAGAATCTGTCTCTCATGTGCTACGTCAGCGTCGGCTCCCCAGCCGAGCCGCTCATT |
| | | GACTTCATGATCAACAGGGGTATGGAGGTCATTGAGGAGTACGAACCACTCAGATACCCACACGCTACC |
| | | AAGATTTTTGTCAACGGGACCTGGGTTGGAGTTCATCAGGACCCCAAGCACCTTGCTGACCAGGTATTCG |
| | | ACACCCGCCGCAAGTCCTACCTGCAGTATGAGGTGTCTCTTGTCAGAGAAATCCGTGACCAGGAATTCAA |
| | | GATCTTCTCTGATGCTGGCCGAGTCATGCGGCCTGTTTTTACTGTGCAAAGTAAAAATGACCCGGAGACT |
| | | GGCCTTGAAAAGGGACAGCTCGGTCTCACCAAGGATTTAGTCAACAGACTGGCGCAAGAGCAAGCCGAC |
| | | CCGCCAGATGATCCAGAAATGAAGACGGGTTGGGAGGGCTTGATCAAAGCTGGCGCTGTCGAGTATCTA |
| | | GACGCTGAAGAAGAGGAGACGTCAATGATTTGCATGACCCCGGAGGATCTGGAGCTTTATAGACTTCAG |
| | | AAAGCGGGTGTAGCTGTCGACGACGACCATGGCGATGATCTGAACAAGCGCCTGAAGACTAAAACCAAC |
| | | CCAACTACACACATGTACACTCACTGTGAAATTCACCCGAGTATGATCTTGGGTATCTGCGCAAGCATTA |
| | | TTCCTTTCCCAGACCACAATCAATCGCCTCGTAACACCTACCAATCCGCAATGGGTAAACAAGCCATGGG |
| | | CTTCTTCCTGACAAATTATTCTCGTCGTATGGATACTATGGCAAATATCCTGTACTACCCACAAAAGCCTT |
| | | TGGCAACGACTAGATCCATGGAGTTCTTGAAGTTCCGAGAATTGCCTGCAGGTCAAAACGCCATCGTCGC |
| | | GATTGCTTGTTATTCCGGTTATAATCAGGAGGACTCTGTGATTATGAATCAAAGCAGTATCGATCGCGGA |
| | | CTCTTCAGAAGTTTGTTCTTTCGTTCGTACTCTGATCAAGAGAAAAAGGTCGGTTTGAACTACACAGAGA |
| | | TATTCGAAAAGCCGTTCCACCAAAGCACCCTGCGCATGAAACACGGTACTTACGACAAGCTCGATGAGG |
| | | ACGGTATTGTCGCTCCTGGCGTTCGTGTGTCTGGAGAAGACATTATCATTGGCAAAACTGCGCCAATTGA |
| | | CCCAGAGACGCAAGACTTGGGCGCGCGTACAACTGCGCATCAGCGCCGTGATATCTCTACGCCTCTGCGT |
| | | AGTACCGAAAATGGTATTGTTGATCAAGTCATTGTGACTGTCAACGCCGACAACGTCAAATACGTCAAG |
| | | GTCAGGGTTCGTACGACCAAGATACCCCAGATCGGTGATAAGTTTGCTTCACGACACGGCCAAAAAGGT |
| | | ACCATTGGTGTCACATACCGACAGGAAGATATGCCATTCACGAGAGAGGGGGTCACCCCAGATATCATT |
| | | ATCAACCCGCACGCCATTCCCTCTCGAATGACAATTGCTCATCTGATCGAATGTCTTTTGAGCAAGGTTTC |
| | | GACTTTAGAAGGTATGGAAGGCGATGCTACTCCATTTACCGACGTCACTGTCGATTCTGTCTCCGACTTG |
| | | CTTCGCAAGCACGGCTATCAGTCCCGCGGGTTTGAGATCATGTACAACGGTCACACTGGTAAGAAACTTC |
| | | GTGCTCAGGTTTTCTTCGGACCGACCTATTACCAGCGTCTGCGTCACATGGTGGACGACAAAATTCACGC |
| | | CCGTGCCCGTGGCCCTGTGCAGATCATGACTAGACAACCTGTGGAGGGTCGTGCAAGGGATGGGGGGTT |
| | | GCGTTTCGGCGAAATGGAACGTGATTGCATGATTGCGCATGGTGCAGCAGCATTCTTGAAGGAGCGCCTC |
| | | TTTGAAGTGTCTGATGCGTTCCGAGTGCACATCTGCGAGATTTGCGGCTTGATGACGCCGATTGCGTAA |
| 25 | US-670 | ATGGCAGACTACGAGGATGAATACGACTACGAGAATTACGAGGAAGAAGATTCGGGCATCACTCCCGAA |
| | | GATTGTTGGACCGTCATCTCCTCTTTCTTCGAATCCAAGGGCCTCGTCTCTCAGCAAACTGCCTCCTTTGA |
| | | TGAATTTACTCAGTCAACTATCCAAGACCTTGTCAGCGAGTACTCAAGAATATCTCTCGACCAACCCAAC |
| | | CCGCCCTCCAACGACGACCGGAAGATAAGCGTCCGCCGATACGAGATCGAGTTCGGCAGCATCATGGTA |
| | | TCGAGGCCTTCGATCAGTGAGACCGATGGGACCGTGACTTCGCTTCTACCATACGAGTGCCGCGATCGTA |
| | | ACCTGACCTATGCAGCGCCGGTTTACATTAAGATTACCAAAAAAGTGCAAGCTGCTGTGGAAACGGAAA |
| | | TACCTTTGCACGAGCTAGATGATGCTCAACAGACAGAGTATGCCAAAACTGGTGAGCTGCCTACACGGTT |
| | | GGCCTGGAGATCGGAGGATGCGCCAGAGCCCGAAAATTCCAACAAGCCTGATGCCTGGAAGGACATGGT |
| | | CTTCGTCGGGAAAATGCCGGTCATGGTCAAATCTAAAGTCTGCCATTTGAGCCGCGAGCATGACGAGAA |
| | | TTTATTTCTTGTCAATGAGTGCCCATACGACCAGGGCGGTTACTTTGTTATCAATGGAAGTGAAAAGGTG |
| | | CTTATCGCCCAAGAGCGTTCCGCCGCCAATATTGTTCAGGTCTTCAAGAAGGCTCAGCCCAGCCCTTATA |
| | | CATACACTGCTGAAATCAGAAGCGCTCTGGAGAAAGGCTCGCGTCTCATCTCCAGCATGATGCTCAAGCT |
| | | CTACGGGAAGGGTGAGTCAGCTCGTGGCGGCTTCGGGCAAACGATTCATACAACTCTTCCGTTTGTTAAA |
| | | GCAGACCTTCCTGTCGCCATTGTATTTAGAGCACTTGGCGTTGTCTCCGACGAGGATATTCTCAACCACAT |
| | | TTGTTACGACCGAAAAGACTCTCAGATGCTGGAAATGCTTCGGCCTTGTATCGAGGAAGCTTTCTGCGTC |
| | | CAAGACCGTGAAGTCGCCCTCGACTTCATTGGTAAGCGTGGTAATCGGGATCAGGCTGGTCTCGGACGC |
| | | GATAAACGTATTCGTGTGGCACGCGACATTCTTCAGAAAGAAACTTTGCCTCACATCTCCCAGGAGGCTG |
| | | GTAGCGAGACGCGCAAAGCCTTCTTTATCGGTTATATGGTCCACAAACTACTCCAGTGCGCTCTGGGGCG |
| | | TCGGGAGCCTGACGACAGAGACCACTTCGGCAAAAAGCGTCTTGATTTAGCAGGTCCACTACTTGCGAA |
| | | ACTCTTCCGCGGTATCATGCGCCGAATGCACTCAGAGCTCGCCAATTACCTGCGACGCTGCGTCGAGGGT |
| | | AACCGCCACTTCAATCTAGCTGTCGGTGTTAAGCCTGGCACGCTTTCGAACGGCCTCAAGTACTCTCTTG |
| | | CTACTGGTAATTGGGGTGACCAGAAAAAAGCTATGAGCTCAACTGCTGGTGTGTCCCAGGTTTTGAACCG |
| | | ATATACTTTTGCCTCAACACTATCCCACCTTCGACGTACCAATACACCCATTGGCAGAGATGGCAAACTG |
| | | GCAAAACCTCGTCAGCTTCATAATACTCATTGGGGTCTTGTCTGCCCTGCTGAGACACCTGAAGGCCAGG |
| | | CTTGCGGTCTTGTCAAGAATCTGTCTCTCATGTGCTACGTCAGCGTCGGCTCCCCAGCCGAGCCGCTCATT |
| | | GACTTCATGATCAACAGGGGTATGGAGGTCATTGAGGAGTACGAACCACTCAGATACCCACACGCTACC |
| | | AAGATTTTTGTCAACGGGACCTGGGTTGGAGTTCATCAGGACCCCAAGCACCTTGCTGACCAGGTATTCG |
| | | ACACCCGCCGCAAGTCCTACCTGCAGTATGAGGTGTCTCTTGTCAGAGAAATCCGTGACCAGGAATTCAA |
| | | GATCTTCTCTGATGCTGGCCGAGTCATGCGGCCTGTTTTTACTGTGCAAAGTAAAAATGACCCGGAGACT |
| | | GGCCTTGAAAAGGGACAGCTCGGTCTCACCAAGGATTTAGTCAACAGACTGGCGCAAGAGCAAGCCGAC |
| | | CCGCCAGATGATCCAGAAATGAAGACGGGTTGGGAGGGCTTGATCAAAGCTGGCGCTGTCGAGTATCTA |
| | | GACGCTGAAGAAGAGGAGACGTCAATGATTTGCATGACCCCGGAGGATCTGGAGCTTTATAGACTTCAG |
| | | AAAGCGGGTGTAGCTGTCGACGACGACCATGGCGATGATCTGAACAAGCGCCTGAAGACTAAAACCAAC |
| | | CCAACTACACACATGTACACTCACTGTGAAATTCACCCGAGTATGATCTTGGGTATCTGCGCAAGCATTA |
| | | TTCCTTTCCCAGACCACAATCAATCGCCTCGTAACACCTACCAATCCGCAATGGGTAAACAAGCCATGGG |
| | | CTTCTTCCTGACAAATTATTCTCGTCGTATGGATACTATGGCAAATATCCTGTACTACCCACAAAAGCCTT |
| | | TGGCAACGACTAGATCCATGGAGTTCTTGAAGTTCCGAGAATTGCCTGCAGGTCAAAACGCCATCGTCGC |
| | | GATTGCTTGTTATTCCGGTTATAATCAGGAGGACTCTGTGATTATGAATCAAAGCAGTATCGATCGCGGA |
| | | CTCTTCAGAAGTTTGTTCTTTCGTTCGTACTCTGATCAAGAGAAAAAGGTCGGTTTGAACTACACAGAGA |
| | | TATTCGAAAAGCCGTTCCACCAAAGCACCCTGCGCATGAAACACGGTACTTACGACAAGCTCGATGAGG |
| | | ACGGTATTGTCGCTCCTGGCGTTCGTGTGTCTGGAGAAGACATTATCATTGGCAAAACTGCGCCAATTGA |
| | | CCCAGAGACGCAAGACTTGGGCGCGCGTACAACTGCGCATCAGCGCCGTGATATCTCTACGCCTCTGCGT |
| | | AGTACCGAAAATGGTATTGTTGATCAAGTCATTGTGACTGTCAACGCCGACAACGTCAAATACGTCAAG |
| | | GTCAGGGTTCGTACGACCAAGATACCCCAGATCGGTGATAAGTTTGCTTCACGACACGGCCAAAAAGGT |
| | | ACCATTGGTGTCACATACCGACAGGAAGATATGCCATTCACGAGAGAGGGGGTCACCCCAGATATCATT |
| | | ATCAACCCGCACGCCATTCCCTCTCGAATGACAATTGCTCATCTGATCGAATGTCTTTTGAGCAAGGTTTC |
| | | GACTTTAGAAGGTATGGAAGGCGATGCTACTCCATTTACCGACGTCACTGTCGATTCTGTCTCCGACTTG |
| | | CTTCGCAAGCACGGCTATCAGTCCCGCGGGTTTGAGATCATGTACAACGGTCACACTGGTAAGAAACTTC |
| | | GTGCTCAGGTTTTCTTCGGACCGACCTATTACCAGCGTCTGCGTCACATGGTGGACGACAAAATTCACGC |
| | | CCGTGCCCGTGGCCCTGTGCAGATCATGACTAGACAACCTGTGGAGGGTCGTGCAAGGGATGGGGGGTT |
| | | GCGTTTCGGCGAAATGGAACGTGATTGCATGATTGCGCATGGTGCAGCAGCATTCTTGAAGGAGCGCCTC |
| | | TTTGAAGTGTCTGATGCGTTCCGAGTGCACATCTGCGAGATTTGCGGCTTGATGACGCCGATTGCGTAA |
| 26 | US-935 | ATGGCAGACTACGAGGATGAATACGACTACGAGAATTACGAGGAAGAAGATTCGGGCATCACTCCCGAA |
| | | GATTGTTGGACCGTCATCTCCTCTTTCTTCGAATCCAAGGGCCTCGTCTCTCAGCAAACTGCCTCCTTTGA |
| | | TGAATTTACTCAGTCAACTATCCAAGACCTTGTCAGCGAGTACTCAAGAATATCTCTCGACCAACCCAAC |
| | | CCGCCCTCCAACGACGACCGGAAGATAAGCGTCCGCCGATACGAGATCGAGTTCGGCAGCATCATGGTA |
| | | TCGAGGCCTTCGATCAGTGAGACCGATGGGACCGTGACTTCGCTTCTACCATACGAGTGCCGCGATCGTA |
| | | ACCTGACCTATGCAGCGCCGGTTTACATTAAGATTACCAAAAAAGTGCAAGCTGCTGTGGAAACGGAAA |
| | | TACCTTTGCACGAGCTAGATGATGCTCAACAGACAGAGTATGCCAAAACTGGTGAGCTGCCTACACGGTT |
| | | GGCCTGGAGATCGGAGGATGCGCCAGAGCCCGAAAATTCCAACAAGCCTGATGCCTGGAAGGACATGGT |
| | | CTTCGTCGGGAAAATGCCGGTCATGGTCAAATCTAAAGTCTGCCATTTGAGCCGCGAGCATGACGAGAA |
| | | TTTATTTCTTGTCAATGAGTGCCCATACGACCAGGGCGGTTACTTTGTTATCAATGGAAGTGAAAAGGTG |
| | | CTTATCGCCCAAGAGCGTTCCGCCGCCAATATTGTTCAGGTCTTCAAGAAGGCTCAGCCCAGCCCTTATA |
| | | CATACACTGCTGAAATCAGAAGCGCTCTGGAGAAAGGCTCGCGTCTCATCTCCAGCATGATGCTCAAGCT |
| | | CTACGGGAAGGGTGAGTCAGCTCGTGGCGGCTTCGGGCAAACGATTCATACAACTCTTCCGTTTGTTAAA |
| | | GCAGACCTTCCTGTCGCCATTGTATTTAGAGCACTTGGCGTTGTCTCCGACGAGGATATTCTCAACCACAT |
| | | TTGTTACGACCGAAAAGACTCTCAGATGCTGGAAATGCTTCGGCCTTGTATCGAGGAAGCTTTCTGCGTC |
| | | CAAGACCGTGAAGTCGCCCTCGACTTCATTGGTAAGCGTGGTAATCGGGATCAGGCTGGTCTCGGACGC |
| | | GATAAACGTATTCGTGTGGCACGCGACATTCTTCAGAAAGAAACTTTGCCTCACATCTCCCAGGAGGCTG |
| | | GTAGCGAGACGCGCAAAGCCTTCTTTATCGGTTATATGGTCCACAAACTACTCCAGTGCGCTCTGGGGCG |
| | | TCGGGAGCCTGACGACAGAGACCACTTCGGCAAAAAGCGTCTTGATTTAGCAGGTCCACTACTTGCGAA |
| | | ACTCTTCCGCGGTATCATGCGCCGAATGCACTCAGAGCTCGCCAATTACCTGCGACGCTGCGTCGAGGGT |
| | | AACCGCCACTTCAATCTAGCTGTCGGTGTTAAGCCTGGCACGCTTTCGAACGGCCTCAAGTACTCTCTTG |
| | | CTACTGGTAATTGGGGTGACCAGAAAAAAGCTATGAGCTCAACTGCTGGTGTGTCCCAGGTTTTGAACCG |
| | | ATATACTTTTGCCTCAACACTATCCCACCTTCGACGTACCAATACACCCATTGGCAGAGATGGCAAACTG |
| | | GCAAAACCTCGTCAGCTTCATAATACTCATTGGGGTCTTGTCTGCCCTGCTGAGACACCTGAAGGCCAGG |
| | | CTTGCGGTCTTGTCAAGAATCTGTCTCTCATGTGCTACGTCAGCGTCGGCTCCCCAGCCGAGCCGCTCATT |
| | | GACTTCATGATCAACAGGGGTATGGAGGTCATTGAGGAGTACGAACCACTCAGATACCCACACGCTACC |
| | | AAGATTTTTGTCAACGGGACCTGGGTTGGAGTTCATCAGGACCCCAAGCACCTTGCTGACCAGGTATTCG |
| | | ACACCCGCCGCAAGTCCTACCTGCAGTATGAGGTGTCTCTTGTCAGAGAAATCCGTGACCAGGAATTCAA |
| | | GATCTTCTCTGATGCTGGCCGAGTCATGCGGCCTGTTTTTACTGTGCAAAGTAAAAATGACCCGGAGACT |
| | | GGCCTTGAAAAGGGACAGCTCGGTCTCACCAAGGATTTAGTCAACAGACTGGCGCAAGAGCAAGCCGAC |
| | | CCGCCAGATGATCCAGAAATGAAGACGGGTTGGGAGGGCTTGATCAAAGCTGGCGCTGTCGAGTATCTA |
| | | GACGCTGAAGAAGAGGAGACGTCAATGATTTGCATGACCCCGGAGGATCTGGAGCTTTATAGACTTCAG |
| | | AAAGCGGGTGTAGCTGTCGACGACGACCATGGCGATGATCTGAACAAGCGCCTGAAGACTAAAACCAAC |
| | | CCAACTACACACATGTACACTCACTGTGAAATTCACCCGAGTATGATCTTGGGTATCTGCGCAAGCATTA |
| | | TTCCTTTCCCAGACCACAATCAATCGCCTCGTAACACCTACCAATCCGCAATGGGTAAACAAGCCATGGG |
| | | CTTCTTCCTGACAAATTATTCTCGTCGTATGGATACTATGGCAAATATCCTGTACTACCCACAAAAGCCTT |
| | | TGGCAACGACTAGATCCATGGAGTTCTTGAAGTTCCGAGAATTGCCTGCAGGTCAAAACGCCATCGTCGC |
| | | GATTGCTTGTTATTCCGGTTATAATCAGGAGGACTCTGTGATTATGAATCAAAGCAGTATCGATCGCGGA |
| | | CTCTTCAGAAGTTTGTTCTTTCGTTCGTACTCTGATCAAGAGAAAAAGGTCGGTTTGAACTACACAGAGA |
| | | TATTCGAAAAGCCGTTCCACCAAAGCACCCTGCGCATGAAACACGGTACTTACGACAAGCTCGATGAGG |
| | | ACGGTATTGTCGCTCCTGGCGTTCGTGTGTCTGGAGAAGACATTATCATTGGCAAAACTGCGCCAATTGA |
| | | CCCAGAGACGCAAGACTTGGGCGCGCGTACAACTGCGCATCAGCGCCGTGATATCTCTACGCCTCTGCGT |
| | | AGTACCGAAAATGGTATTGTTGATCAAGTCATTGTGACTGTCAACGCCGACAACGTCAAATACGTCAAG |
| | | GTCAGGGTTCGTACGACCAAGATACCCCAGATCGGTGATAAGTTTGCTTCACGACACGGCCAAAAAGGT |
| | | ACCATTGGTGTCACATACCGACAGGAAGATATGCCATTCACGAGAGAGGGGGTCACCCCAGATATCATT |
| | | ATCAACCCGCACGCCATTCCCTCTCGAATGACAATTGCTCATCTGATCGAATGTCTTTTGAGCAAGGTTTC |
| | | GACTTTAGAAGGTATGGAAGGCGATGCTACTCCATTTACCGACGTCACTGTCGATTCTGTCTCCGACTTG |
| | | CTTCGCAAGCACGGCTATCAGTCCCGCGGGTTTGAGATCATGTACAACGGTCACACTGGTAAGAAACTTC |
| | | GTGCTCAGGTTTTCTTCGGACCGACCTATTACCAGCGTCTGCGTCACATGGTGGACGACAAAATTCACGC |
| | | CCGTGCCCGTGGCCCTGTGCAGATCATGACTAGACAACCTGTGGAGGGTCGTGCAAGGGATGGGGGGTT |
| | | GCGTTTCGGCGAAATGGAACGTGATTGCATGATTGCGCATGGTGCAGCAGCATTCTTGAAGGAGCGCCTC |
| | | TTTGAAGTGTCTGATGCGTTCCGAGTGCACATCTGCGAGATTTGCGGCTTGATGACGCCGATTGCAAATC |
| | | TTTCAAAGCAATCTTTTGAATGCAGACCTTGCAAGAACAAGACCAAAATTGCCCAGATTCACATTCCGTA |
| | | CGCAGCCAAGCTATTGTTCCAAGAACTTCAATCCATGAACATTGCAGCACGTATGTTTACGGACAGATCT |
| | | GGTGCCTCGATCAGGTAG |

TABLE 6

| SEQ | Strain | |
|---|---|---|
| ID No. | Number | TEF1 Sequence |
| 27 | AU-16727 | ATGGGCAAGGACGACAAGACTCACATCAACGTCGTCGTTATCGGCCACGTCGATTCCGGCAAGTCTACC |
| | | ACCACTGGTCACTTGATCTACCAGTGCGGTGGTATTGACAAGCGTACCATTGAGAAGTTCGAGAAGGAA |
| | | GCCGCTGAACTCGGCAAGGGTTCCTTCAAGTATGCCTGGGTTCTTGACAAGCTCAAGGCCGAGCGTGAGC |
| | | GTGGTATCACCATTGATATCGCTCTCTGGAAGTTCGAGACTCCCAAGTACCACGTCACCGTCATTGATGC |
| | | TCCCGGTCACCGTGATTTCATCAAGAACATGATTACTGGTACTTCCCAGGCCGATTGTGCTATTCTCATCA |
| | | TCGCCGCCGGTACTGGTGAGTTCGAGGCTGGTATCTCCAAGGATGGCCAGACCCGTGAGCACGCTCTGCT |
| | | CGCTTTCACCCTCGGTGTCAAGCAGCTCATTGTCGCCATCAACAAGATGGACACCACCAAGTGGTCCGAG |
| | | GCCCGTTACCAGGAAATCATCAAGGAGACTTCCAGCTTCATCAAGAAGGTTGGCTACAACCCCAAGGCT |
| | | GTTGCTTTCGTCCCCATCTCCGGTTTCAACGGCGACAACATGCTTGAGCCCTCCAGCAACTGCCCCTGGTA |
| | | CAAGGGCTGGGAGAAGGAGACCAAGGCTGGCAAGTCTACTGGCAAGACCCTTCTCGAGGCCATCGACGC |
| | | TATTGAGCCCCCCAAGCGTCCTACCGACAAGCCTCTCCGTCTTCCCCTCCAGGATGTTTACAAGATCGGT |
| | | GGTATCGGAACGGTGCCCGTCGGTCGTGTTGAGACTGGTATCATCAAGCCCGGCATGGTCGTCACCTTTG |
| | | CTCCCGCCAACGTCACCACTGAAGTCAAGTCCGTCGAGATGCACCACGAGCAGCTTACTGAGGGTGTTCC |
| | | CGGTGACAACGTCGGCTTCAACGTGAAGAACGTTTCCGTCAAGGAAATCCGTCGTGGTAACGTCGCTGGT |
| | | GACTCCAAGAACGACCCCCCCAATGGCGCTGCTTCCTTCAACGCCCAGGTCATTGTCATCAACCACCCTG |
| | | GCCAGATCGGTGCCGGCTACGCTCCCGTTCTTGACTGCCACACTGCCCACATTGCTTGCAAGTTCTCCGA |
| | | GCTCCTCGAGAAGATCGACCGCCGTACCGGTAAGTCGGTTGAGAACAACCCCAAGTTCATCAAGTCTGG |
| | | TGACTCCGCCATCGTCAAGATGGTTCCCTCCAAGCCCATGTGCGTTGAGGCTTTCACCGACTACCCCCCTC |
| | | TGGGCCGTTTCGCCGTCCGTGACATGCGTCAGACCGTCGCTGTCGGTGTCATCAAGTCCGTTGAGAAGGC |
| | | TGCCGCTGGTTCCTCCAAGGTCACCAAGTCCGCTGCCAAGGCCACCAAGAAATAA |
| 28 | US-52 | ATGGGCAAGGACGACAAGACTCACATCAACGTCGTCGTTATCGGCCACGTCGATTCCGGCAAGTCTACC |
| | | ACCACTGGTCACTTGATCTACCAGTGCGGTGGTATTGACAAGCGTACCATTGAGAAGTTCGAGAAGGAA |
| | | GCCGCTGAACTCGGCAAGGGTTCCTTCAAGTATGCCTGGGTTCTTGACAAGCTCAAGGCCGAGCGTGAGC |
| | | GTGGTATCACCATTGATATCGCTCTCTGGAAGTTCGAGACTCCCAAGTACCACGTCACCGTCATTGATGC |
| | | TCCCGGTCACCGTGATTTCATCAAGAACATGATTACTGGTACTTCCCAGGCCGATTGTGCTATTCTCATCA |
| | | TCGCCGCCGGTACTGGTGAGTTCGAGGCTGGTATCTCCAAGGATGGCCAGACCCGTGAGCACGCTCTGCT |
| | | CGCTTTCACCCTCGGTGTCAAGCAGCTCATTGTCGCCATCAACAAGATGGACACCACCAAGTGGTCCGAG |
| | | GCCCGTTACCAGGAAATCATCAAGGAGACTTCCAGCTTCATCAAGAAGGTTGGCTACAACCCCAAGGCT |
| | | GTTGCTTTCGTCCCCATCTCCGGTTTCAACGGCGACAACATGCTTGAGCCCTCCAGCAACTGCCCCTGGTA |
| | | CAAGGGCTGGGAGAAGGAGACCAAGGCTGGCAAGTCTACTGGCAAGACCCTTCTCGAGGCCATCGACGC |
| | | TATTGAGCCCCCCAAGCGTCCTACCGACAAGCCTCTCCGTCTTCCCCTCCAGGATGTTTACAAGATCGGT |
| | | GGTATCGGAACGGTGCCCGTCGGTCGTGTTGAGACTGGTATCATCAAGCCCGGCATGGTCGTCACCTTTG |
| | | CTCCCGCCAACGTCACCACTGAAGTCAAGTCCGTCGAGATGCACCACGAGCAGCTTACTGAGGGTGTTCC |
| | | CGGTGACAACGTCGGCTTCAACGTGAAGAACGTTTCCGTCAAGGAAATCCGTCGTGGTAACGTCGCTGGT |
| | | GACTCCAAGAACGACCCCCCCAATGGCGCTGCTTCCTTCAACGCCCAGGTCATTGTCATCAACCACCCTG |
| | | GCCAGATCGGTGCCGGCTACGCTCCCGTTCTTGACTGCCACACTGCCCACATTGCTTGCAAGTTCTCCGA |
| | | GCTCCTCGAGAAGATCGACCGCCGTACCGGTAAGTCGGTTGAGAACAACCCCAAGTTCATCAAGTCTGG |
| | | TGACTCCGCCATCGTCAAGATGGTTCCCTCCAAGCCCATGTGCGTTGAGGCTTTCACCGACTACCCCCCTC |
| | | TGGGCCGTTTCGCCGTCCGTGACATGCGTCAGACCGTCGCTGTCGGTGTCATCAAGTCCGTTGAGAAGGC |
| | | TGCCGCTGGTTCCTCCAAGGTCACCAAGTCCGCTGCCAAGGCCACCAAGAAATAA |
| 29 | US-675 | ATGGGCAAGGACGACAAGACTCACATCAACGTCGTCGTTATCGGCCACGTCGATTCCGGCAAGTCTACC |
| | | ACCACTGGTCACTTGATCTACCAGTGCGGTGGTATTGACAAGCGTACCATTGAGAAGTTCGAGAAGGAA |
| | | GCCGCTGAACTCGGCAAGGGTTCCTTCAAGTATGCCTGGGTTCTTGACAAGCTCAAGGCCGAGCGTGAGC |
| | | GTGGTATCACCATTGATATCGCTCTCTGGAAGTTCGAGACTCCCAAGTACCACGTCACCGTCATTGATGC |
| | | TCCCGGTCACCGTGATTTCATCAAGAACATGATTACTGGTACTTCCCAGGCCGATTGTGCTATTCTCATCA |
| | | TCGCCGCCGGTACTGGTGAGTTCGAGGCTGGTATCTCCAAGGATGGCCAGACCCGTGAGCACGCTCTGCT |
| | | CGCTTTCACCCTCGGTGTCAAGCAGCTCATTGTCGCCATCAACAAGATGGACACCACCAAGTGGTCCGAG |
| | | GCCCGTTACCAGGAAATCATCAAGGAGACTTCCAGCTTCATCAAGAAGGTTGGCTACAACCCCAAGGCT |
| | | GTTGCTTTCGTCCCCATCTCCGGTTTCAACGGCGACAACATGCTTGAGCCCTCCAGCAACTGCCCCTGGTA |
| | | CAAGGGCTGGGAGAAGGAGACCAAGGCTGGCAAGTCTACTGGCAAGACCCTTCTCGAGGCCATCGACGC |
| | | TATTGAGCCCCCCAAGCGTCCTACCGACAAGCCTCTCCGTCTTCCCCTCCAGGATGTTTACAAGATCGGT |
| | | GGTATCGGAACGGTGCCCGTCGGTCGTGTTGAGACTGGTATCATCAAGCCCGGCATGGTCGTCACCTTTG |
| | | CTCCCGCCAACGTCACCACTGAAGTCAAGTCCGTCGAGATGCACCACGAGCAGCTTACTGAGGGTGTTCC |
| | | CGGTGACAACGTCGGCTTCAACGTGAAGAACGTTTCCGTCAAGGAAATCCGTCGTGGTAACGTCGCTGGT |
| | | GACTCCAAGAACGACCCCCCCAATGGCGCTGCTTCCTTCAACGCCCAGGTCATTGTCATCAACCACCCTG |
| | | GCCAGATCGGTGCCGGCTACGCTCCCGTTCTTGACTGCCACACTGCCCACATTGCTTGCAAGTTCTCCGA |
| | | GCTCCTCGAGAAGATCGACCGCCGTACCGGTAAGTCGGTTGAGAACAACCCCAAGTTCATCAAGTCTGG |
| | | TGACTCCGCCATCGTCAAGATGGTTCCCTCCAAGCCCATGTGCGTTGAGGCTTTCACCGACTACCCCCCTC |
| | | TGGGCCGTTTCGCCGTCCGTGACATGCGTCAGACCGTCGCTGTCGGTGTCATCAAGTCCGTTGAGAAGGC |
| | | TGCCGCTGGTTCCTCCAAGGTCACCAAGTCCGCTGCCAAGGCCACCAAGAAATAA |
| 30 | US-699 | ATGGGCAAGGACGACAAGACTCACATCAACGTCGTCGTTATCGGCCACGTCGATTCCGGCAAGTCTACC |
| | | ACCACTGGTCACTTGATCTACCAGTGCGGTGGTATTGACAAGCGTACCATTGAGAAGTTCGAGAAGGAA |
| | | GCCGCTGAACTCGGCAAGGGTTCCTTCAAGTATGCCTGGGTTCTTGACAAGCTCAAGGCCGAGCGTGAGC |
| | | GTGGTATCACCATTGATATCGCTCTCTGGAAGTTCGAGACTCCCAAGTACCACGTCACCGTCATTGATGC |
| | | TCCCGGTCACCGTGATTTCATCAAGAACATGATTACTGGTACTTCCCAGGCCGATTGTGCTATTCTCATCA |
| | | TCGCCGCCGGTACTGGTGAGTTCGAGGCTGGTATCTCCAAGGATGGCCAGACCCGTGAGCACGCTCTGCT |
| | | CGCTTTCACCCTCGGTGTCAAGCAGCTCATTGTCGCCATCAACAAGATGGACACCACCAAGTGGTCCGAG |
| | | GCCCGTTACCAGGAAATCATCAAGGAGACTTCCAGCTTCATCAAGAAGGTTGGCTACAACCCCAAGGCT |
| | | GTTGCTTTCGTCCCCATCTCCGGTTTCAACGGCGACAACATGCTTGAGCCCTCCAGCAACTGCCCCTGGTA |
| | | CAAGGGCTGGGAGAAGGAGACCAAGGCTGGCAAGTCTACTGGCAAGACCCTTCTCGAGGCCATCGACGC |
| | | TATTGAGCCCCCCAAGCGTCCTACCGACAAGCCTCTCCGTCTTCCCCTCCAGGATGTTTACAAGATCGGT |
| | | GGTATCGGAACGGTGCCCGTCGGTCGTGTTGAGACTGGTATCATCAAGCCCGGCATGGTCGTCACCTTTG |
| | | CTCCCGCCAACGTCACCACTGAAGTCAAGTCCGTCGAGATGCACCACGAGCAGCTTACTGAGGGTGTTCC |
| | | CGGTGACAACGTCGGCTTCAACGTGAAGAACGTTTCCGTCAAGGAAATCCGTCGTGGTAACGTCGCTGGT |
| | | GACTCCAAGAACGACCCCCCCAATGGCGCTGCTTCCTTCAACGCCCAGGTCATTGTCATCAACCACCCTG |
| | | GCCAGATCGGTGCCGGCTACGCTCCCGTTCTTGACTGCCACACTGCCCACATTGCTTGCAAGTTCTCCGA |
| | | GCTCCTCGAGAAGATCGACCGCCGTACCGGTAAGTCGGTTGAGAACAACCCCAAGTTCATCAAGTCTGG |
| | | TGACTCCGCCATCGTCAAGATGGTTCCCTCCAAGCCCATGTGCGTTGAGGCTTTCACCGACTACCCCCCTC |
| | | TGGGCCGTTTCGCCGTCCGTGACATGCGTCAGACCGTCGCTGTCGGTGTCATCAAGTCCGTTGAGAAGGC |
| | | TGCCGCTGGTTCCTCCAAGGTCACCAAGTCCGCTGCCAAGGCCACCAAGAAATAA |
| 31 | US-707 | ATGGGCAAGGACGACAAGACTCACATCAACGTCGTCGTTATCGGCCACGTCGATTCCGGCAAGTCTACC |
| | | ACCACTGGTCACTTGATCTACCAGTGCGGTGGTATTGACAAGCGTACCATTGAGAAGTTCGAGAAGGAA |
| | | GCCGCTGAACTCGGCAAGGGTTCCTTCAAGTATGCCTGGGTTCTTGACAAGCTCAAGGCCGAGCGTGAGC |
| | | GTGGTATCACCATTGATATCGCTCTCTGGAAGTTCGAGACTCCCAAGTACCACGTCACCGTCATTGATGC |
| | | TCCCGGTCACCGTGATTTCATCAAGAACATGATTACTGGTACTTCCCAGGCCGATTGTGCTATTCTCATCA |
| | | TCGCCGCCGGTACTGGTGAGTTCGAGGCTGGTATCTCCAAGGATGGCCAGACCCGTGAGCACGCTCTGCT |
| | | CGCTTTCACCCTCGGTGTCAAGCAGCTCATTGTCGCCATCAACAAGATGGACACCACCAAGTGGTCCGAG |
| | | GCCCGTTACCAGGAAATCATCAAGGAGACTTCCAGCTTCATCAAGAAGGTTGGCTACAACCCCAAGGCT |
| | | GTTGCTTTCGTCCCCATCTCCGGTTTCAACGGCGACAACATGCTTGAGCCCTCCAGCAACTGCCCCTGGTA |
| | | CAAGGGCTGGGAGAAGGAGACCAAGGCTGGCAAGTCTACTGGCAAGACCCTTCTCGAGGCCATCGACGC |
| | | TATTGAGCCCCCCAAGCGTCCTACCGACAAGCCTCTCCGTCTTCCCCTCCAGGATGTTTACAAGATCGGT |
| | | GGTATCGGAACGGTGCCCGTCGGTCGTGTTGAGACTGGTATCATCAAGCCCGGCATGGTCGTCACCTTTG |
| | | CTCCCGCCAACGTCACCACTGAAGTCAAGTCCGTCGAGATGCACCACGAGCAGCTTACTGAGGGTGTTCC |
| | | CGGTGACAACGTCGGCTTCAACGTGAAGAACGTTTCCGTCAAGGAAATCCGTCGTGGTAACGTCGCTGGT |
| | | GACTCCAAGAACGACCCCCCCAATGGCGCTGCTTCCTTCAACGCCCAGGTCATTGTCATCAACCACCCTG |
| | | GCCAGATCGGTGCCGGCTACGCTCCCGTTCTTGACTGCCACACTGCCCACATTGCTTGCAAGTTCTCCGA |
| | | GCTCCTCGAGAAGATCGACCGCCGTACCGGTAAGTCGGTTGAGAACAACCCCAAGTTCATCAAGTCTGG |
| | | TGACTCCGCCATCGTCAAGATGGTTCCCTCCAAGCCCATGTGCGTTGAGGCTTTCACCGACTACCCCCCTC |
| | | TGGGCCGTTTCGCCGTCCGTGACATGCGTCAGACCGTCGCTGTCGGTGTCATCAAGTCCGTTGAGAAGGC |
| | | TGCCGCTGGTTCCTCCAAGGTCACCAAGTCCGCTGCCAAGGCCACCAAGAAATAA |
| 32 | US-803 | ATGGGCAAGGACGACAAGACTCACATCAACGTCGTCGTTATCGGCCACGTCGATTCCGGCAAGTCTACC |
| | | ACCACTGGTCACTTGATCTACCAGTGCGGTGGTATTGACAAGCGTACCATTGAGAAGTTCGAGAAGGAA |
| | | GCCGCTGAACTCGGCAAGGGTTCCTTCAAGTATGCCTGGGTTCTTGACAAGCTCAAGGCCGAGCGTGAGC |
| | | GTGGTATCACCATTGATATCGCTCTCTGGAAGTTCGAGACTCCCAAGTACCACGTCACCGTCATTGATGC |
| | | TCCCGGTCACCGTGATTTCATCAAGAACATGATTACTGGTACTTCCCAGGCCGATTGTGCTATTCTCATCA |
| | | TCGCCGCCGGTACTGGTGAGTTCGAGGCTGGTATCTCCAAGGATGGCCAGACCCGTGAGCACGCTCTGCT |
| | | CGCTTTCACCCTCGGTGTCAAGCAGCTCATTGTCGCCATCAACAAGATGGACACCACCAAGTGGTCCGAG |
| | | GCCCGTTACCAGGAAATCATCAAGGAGACTTCCAGCTTCATCAAGAAGGTTGGCTACAACCCCAAGGCT |
| | | GTTGCTTTCGTCCCCATCTCCGGTTTCAACGGCGACAACATGCTTGAGCCCTCCAGCAACTGCCCCTGGTA |
| | | CAAGGGCTGGGAGAAGGAGACCAAGGCTGGCAAGTCTACTGGCAAGACCCTTCTCGAGGCCATCGACGC |
| | | TATTGAGCCCCCCAAGCGTCCTACCGACAAGCCTCTCCGTCTTCCCCTCCAGGATGTTTACAAGATCGGT |
| | | GGTATCGGAACGGTGCCCGTCGGTCGTGTTGAGACTGGTATCATCAAGCCCGGCATGGTCGTCACCTTTG |
| | | CTCCCGCCAACGTCACCACTGAAGTCAAGTCCGTCGAGATGCACCACGAGCAGCTTACTGAGGGTGTTCC |
| | | CGGTGACAACGTCGGCTTCAACGTGAAGAACGTTTCCGTCAAGGAAATCCGTCGTGGTAACGTCGCTGGT |
| | | GACTCCAAGAACGACCCCCCCAATGGCGCTGCTTCCTTCAACGCCCAGGTCATTGTCATCAACCACCCTG |
| | | GCCAGATCGGTGCCGGCTACGCTCCCGTTCTTGACTGCCACACTGCCCACATTGCTTGCAAGTTCTCCGA |
| | | GCTCCTCGAGAAGATCGACCGCCGTACCGGTAAGTCGGTTGAGAACAACCCCAAGTTCATCAAGTCTGG |
| | | TGACTCCGCCATCGTCAAGATGGTTCCCTCCAAGCCCATGTGCGTTGAGGCTTTCACCGACTACCCCCCTC |
| | | TGGGCCGTTTCGCCGTCCGTGACATGCGTCAGACCGTCGCTGTCGGTGTCATCAAGTCCGTTGAGAAGGC |
| | | TGCCGCTGGTTCCTCCAAGGTCACCAAGTCCGCTGCCAAGGCCACCAAGAAATAA |
| 33 | US-670 | ATGGGCAAGGACGACAAGACTCACATCAACGTCGTCGTTATCGGCCACGTCGATTCCGGCAAGTCTACC |
| | | ACCACTGGTCACTTGATCTACCAGTGCGGTGGTATTGACAAGCGTACCATTGAGAAGTTCGAGAAGGAA |
| | | GCCGCTGAACTCGGCAAGGGTTCCTTCAAGTATGCCTGGGTTCTTGACAAGCTCAAGGCCGAGCGTGAGC |
| | | GTGGTATCACCATTGATATCGCTCTCTGGAAGTTCGAGACTCCCAAGTACCACGTCACCGTCATTGATGC |
| | | TCCCGGTCACCGTGATTTCATCAAGAACATGATTACTGGTACTTCCCAGGCCGATTGTGCTATTCTCATCA |
| | | TCGCCGCCGGTACTGGTGAGTTCGAGGCTGGTATCTCCAAGGATGGCCAGACCCGTGAGCACGCTCTGCT |
| | | CGCTTTCACCCTCGGTGTCAAGCAGCTCATTGTCGCCATCAACAAGATGGACACCACCAAGTGGTCCGAG |
| | | GCCCGTTACCAGGAAATCATCAAGGAGACTTCCAGCTTCATCAAGAAGGTTGGCTACAACCCCAAGGCT |
| | | GTTGCTTTCGTCCCCATCTCCGGTTTCAACGGCGACAACATGCTTGAGCCCTCCAGCAACTGCCCCTGGTA |
| | | CAAGGGCTGGGAGAAGGAGACCAAGGCTGGCAAGTCTACTGGCAAGACCCTTCTCGAGGCCATCGACGC |
| | | TATTGAGCCCCCCAAGCGTCCTACCGACAAGCCTCTCCGTCTTCCCCTCCAGGATGTTTACAAGATCGGT |
| | | GGTATCGGAACGGTGCCCGTCGGTCGTGTTGAGACTGGTATCATCAAGCCCGGCATGGTCGTCACCTTTG |
| | | CTCCCGCCAACGTCACCACTGAAGTCAAGTCCGTCGAGATGCACCACGAGCAGCTTACTGAGGGTGTTCC |
| | | CGGTGACAACGTCGGCTTCAACGTGAAGAACGTTTCCGTCAAGGAAATCCGTCGTGGTAACGTCGCTGGT |
| | | GACTCCAAGAACGACCCCCCCAATGGCGCTGCTTCCTTCAACGCCCAGGTCATTGTCATCAACCACCCTG |
| | | GCCAGATCGGTGCCGGCTACGCTCCCGTTCTTGACTGCCACACTGCCCACATTGCTTGCAAGTTCTCCGA |
| | | GCTCCTCGAGAAGATCGACCGCCGTACCGGTAAGTCGGTTGAGAACAACCCCAAGTTCATCAAGTCTGG |
| | | TGACTCCGCCATCGTCAAGATGGTTCCCTCCAAGCCCATGTGCGTTGAGGCTTTCACCGACTACCCCCCTC |
| | | TGGGCCGTTTCGCCGTCCGTGACATGCGTCAGACCGTCGCTGTCGGTGTCATCAAGTCCGTTGAGAAGGC |
| | | TGCCGCTGGTTCCTCCAAGGTCACCAAGTCCGCTGCCAAGGCCACCAAGAAATAA |
| 34 | US-935 | ATGGGCAAGGACGACAAGACTCACATCAACGTCGTCGTTATCGGCCACGTCGATTCCGGCAAGTCTACC |
| | | ACCACTGGTCACTTGATCTACCAGTGCGGTGGTATTGACAAGCGTACCATTGAGAAGTTCGAGAAGGAA |
| | | GCCGCTGAACTCGGCAAGGGTTCCTTCAAGTATGCCTGGGTTCTTGACAAGCTCAAGGCCGAGCGTGAGC |
| | | GTGGTATCACCATTGATATCGCTCTCTGGAAGTTCGAGACTCCCAAGTACCACGTCACCGTCATTGATGC |
| | | TCCCGGTCACCGTGATTTCATCAAGAACATGATTACTGGTACTTCCCAGGCCGATTGTGCTATTCTCATCA |
| | | TCGCCGCCGGTACTGGTGAGTTCGAGGCTGGTATCTCCAAGGATGGCCAGACCCGTGAGCACGCTCTGCT |
| | | CGCTTTCACCCTCGGTGTCAAGCAGCTCATTGTCGCCATCAACAAGATGGACACCACCAAGTGGTCCGAG |
| | | GCCCGTTACCAGGAAATCATCAAGGAGACTTCCAGCTTCATCAAGAAGGTTGGCTACAACCCCAAGGCT |
| | | GTTGCTTTCGTCCCCATCTCCGGTTTCAACGGCGACAACATGCTTGAGCCCTCCAGCAACTGCCCCTGGTA |
| | | CAAGGGCTGGGAGAAGGAGACCAAGGCTGGCAAGTCTACTGGCAAGACCCTTCTCGAGGCCATCGACGC |
| | | TATTGAGCCCCCCAAGCGTCCTACCGACAAGCCTCTCCGTCTTCCCCTCCAGGATGTTTACAAGATCGGT |
| | | GGTATCGGAACGGTGCCCGTCGGTCGTGTTGAGACTGGTATCATCAAGCCCGGCATGGTCGTCACCTTTG |
| | | CTCCCGCCAACGTCACCACTGAAGTCAAGTCCGTCGAGATGCACCACGAGCAGCTTACTGAGGGTGTTCC |
| | | CGGTGACAACGTCGGCTTCAACGTGAAGAACGTTTCCGTCAAGGAAATCCGTCGTGGTAACGTCGCTGGT |
| | | GACTCCAAGAACGACCCCCCCAATGGCGCTGCTTCCTTCAACGCCCAGGTCATTGTCATCAACCACCCTG |
| | | GCCAGATCGGTGCCGGCTACGCTCCCGTTCTTGACTGCCACACTGCCCACATTGCTTGCAAGTTCTCCGA |
| | | GCTCCTCGAGAAGATCGACCGCCGTACCGGTAAGTCGGTTGAGAACAACCCCAAGTTCATCAAGTCTGG |
| | | TGACTCCGCCATCGTCAAGATGGTTCCCTCCAAGCCCATGTGCGTTGAGGCTTTCACCGACTACCCCCCTC |
| | | TGGGCCGTTTCGCCGTCCGTGACATGCGTCAGACCGTCGCTGTCGGTGTCATCAAGTCCGTTGAGAAGGC |
| | | TGCCGCTGGTTCCTCCAAGGTCACCAAGTCCGCTGCCAAGGCCACCAAGAAATAA |

TABLE 7

| SEQ | Strain | |
|---|---|---|
| ID No. | Number | TUB2 Sequence |
| 35 | AU-16727 | ATGCGTGAGATTGTTCACCTCCAGACCGGCCAGTGCGGTAACCAAATCGGTGCTGCTTTCTGGCAGACCA |
| | | TCTCTGGCGAGCACGGCCTCGACTCCAGCGGTGTTTACAATGGCACTTCTGAGCTTCAGCTCGAGCGCAT |
| | | GAATGTCTACTTCAACGAGGCCTCCGGCAACAAGTATGTCCCTCGCGCCGTCCTCGTCGATCTTGAGCCC |
| | | GGTACCATGGATGCTGTCCGTGCCGGACCCTTCGGTCAGCTCTTCCGTCCCGACAACTTCGTTTTCGGTCA |
| | | GTCCGGTGCCGGCAACAACTGGGCCAAGGGTCATTACACTGAGGGTGCCGAGCTCGTTGACCAGGTCCT |
| | | CGACGTTGTTCGTCGTGAGGCCGAAGGCTGCGACTGCCTCCAGGGTTTCCAGATCACCCACTCTCTTGGT |
| | | GGTGGTACTGGTGCTGGTATGGGTACTCTGCTCATCTCCAAGATCCGCGAAGAGTTTCCCGACCGCATGA |
| | | TGGCCACCTTTTCCGTGGTTCCCTCTCCCGGCAACTCCGACACCGTTGTCGAGCCCTACAACGCTACTCTC |
| | | TCCGTTCACCAGCTCGTTGAGAACTCCGACGAGACCTTCTGTATCGACAACCAGGCTCTGTACGATATCT |
| | | GCATGCGTACCCTGAAGCTATCCAATCCTTCGTACGGTGACCTGAACCACCTCGTTTCCGTCGTCATGTCC |
| | | GGCATCACCACCTGCCTGCGTTTCCCTGGTCAGCTTAACTCTGATCTTCGCAAGCTCGCCGTCAACATGGT |
| | | TCCTTTCCCTCGTCTTCACTTTTTCATGGTCGGCTTTGCTCCCCTGACGAGCCGTGGTGCCCACTCCTTCCG |
| | | CGCCGTCTCTGTTCCCGAGCTCACTCAGCAGATGTTCGACCCTAAGAACATGATGGCTGCTTCTGACTTCC |
| | | GCAACGGTCGCTACCTGACCTGCTCTGCCATTTTCCGTGGTAAGGTTGCCATGAAGGAGGTTGAGGACCA |
| | | GATGCGTAATGTGCAGACCAAGAACTCCAGCTACTTCGTCGAGTGGATCCCCAACAACATCCAGAACGC |
| | | TCTCTGCGCCGTTCCCCCCCGCGGACTTAAGATGTCGTCTACCTTCATTGGTAACTCGACCTCTATCCAGG |
| | | ATCTCTTCAAGCGTGTCGGTGAGCAGTTCTCCGCCATGTTCCGTCGCAAGGCTTTCCTTCATTGGTACACT |
| | | GGTGAAGGTATGGACGAGATGGAGTTCACTGAGGCTGAGTCCAACATGAACGATCTTATCTCCGAATAC |
| | | CAGCAGTACCAGGACGCTGGCATTGATGATGAGGAAGAGGAATACGAGGAGGAGCTCCCTGTCGAGGG |
| | | CGAGGAGCCTTTGTAA |
| 36 | US-52 | ATGCGTGAGATTGTTCACCTCCAGACCGGCCAGTGCGGTAACCAAATCGGTGCTGCTTTCTGGCAGACCA |
| | | TCTCTGGCGAGCACGGCCTCGACTCCAGCGGTGTTTACAATGGCACTTCTGAGCTTCAGCTCGAGCGCAT |
| | | GAATGTCTACTTCAACGAGGCCTCCGGCAACAAGTATGTCCCTCGCGCCGTCCTCGTCGATCTTGAGCCC |
| | | GGTACCATGGATGCTGTCCGTGCCGGACCCTTCGGTCAGCTCTTCCGTCCCGACAACTTCGTTTTCGGTCA |
| | | GTCCGGTGCCGGCAACAACTGGGCCAAGGGTCATTACACTGAGGGTGCCGAGCTCGTTGACCAGGTCCT |
| | | CGACGTTGTTCGTCGTGAGGCCGAAGGCTGCGACTGCCTCCAGGGTTTCCAGATCACCCACTCTCTTGGT |
| | | GGTGGTACTGGTGCTGGTATGGGTACTCTGCTCATCTCCAAGATCCGCGAAGAGTTTCCCGACCGCATGA |
| | | TGGCCACCTTTTCCGTGGTTCCCTCTCCCGGCAACTCCGACACCGTTGTCGAGCCCTACAACGCTACTCTC |
| | | TCCGTTCACCAGCTCGTTGAGAACTCCGACGAGACCTTCTGTATCGACAACCAGGCTCTGTACGATATCT |
| | | GCATGCGTACCCTGAAGCTATCCAATCCTTCGTACGGTGACCTGAACCACCTCGTTTCCGTCGTCATGTCC |
| | | GGCATCACCACCTGCCTGCGTTTCCCTGGTCAGCTTAACTCTGATCTTCGCAAGCTCGCCGTCAACATGGT |
| | | TCCTTTCCCTCGTCTTCACTTTTTCATGGTCGGCTTTGCTCCCCTGACGAGCCGTGGTGCCCACTCCTTCCG |
| | | CGCCGTCTCTGTTCCCGAGCTCACTCAGCAGATGTTCGACCCTAAGAACATGATGGCTGCTTCTGACTTCC |
| | | GCAACGGTCGCTACCTGACCTGCTCTGCCATTTTCCGTGGTAAGGTTGCCATGAAGGAGGTTGAGGACCA |
| | | GATGCGTAATGTGCAGACCAAGAACTCCAGCTACTTCGTCGAGTGGATCCCCAACAACATCCAGAACGC |
| | | TCTCTGCGCCGTTCCCCCCCGCGGACTTAAGATGTCGTCTACCTTCATTGGTAACTCGACCTCTATCCAGG |
| | | ATCTCTTCAAGCGTGTCGGTGAGCAGTTCTCCGCCATGTTCCGTCGCAAGGCTTTCCTTCATTGGTACACT |
| | | GGTGAAGGTATGGACGAGATGGAGTTCACTGAGGCTGAGTCCAACATGAACGATCTTATCTCCGAATAC |
| | | CAGCAGTACCAGGACGCTGGCATTGATGATGAGGAAGAGGAATACGAGGAGGAGCTCCCTGTCGAGGG |
| | | CGAGGAGCCTTTGTAA |
| 37 | US-675 | ATGCGTGAGATTGTTCACCTCCAGACCGGCCAGTGCGGTAACCAAATCGGTGCTGCTTTCTGGCAGACCA |
| | | TCTCTGGCGAGCACGGCCTCGACTCCAGCGGTGTTTACAATGGCACTTCTGAGCTTCAGCTCGAGCGCAT |
| | | GAATGTCTACTTCAACGAGGCCTCCGGCAACAAGTATGTCCCTCGCGCCGTCCTCGTCGATCTTGAGCCC |
| | | GGTACCATGGATGCTGTCCGTGCCGGACCCTTCGGTCAGCTCTTCCGTCCCGACAACTTCGTTTTCGGTCA |
| | | GTCCGGTGCCGGCAACAACTGGGCCAAGGGTCATTACACTGAGGGTGCCGAGCTCGTTGACCAGGTCCT |
| | | CGACGTTGTTCGTCGTGAGGCCGAAGGCTGCGACTGCCTCCAGGGTTTCCAGATCACCCACTCTCTTGGT |
| | | GGTGGTACTGGTGCTGGTATGGGTACTCTGCTCATCTCCAAGATCCGCGAAGAGTTTCCCGACCGCATGA |
| | | TGGCCACCTTTTCCGTGGTTCCCTCTCCCGGCAACTCCGACACCGTTGTCGAGCCCTACAACGCTACTCTC |
| | | TCCGTTCACCAGCTCGTTGAGAACTCCGACGAGACCTTCTGTATCGACAACCAGGCTCTGTACGATATCT |
| | | GCATGCGTACCCTGAAGCTATCCAATCCTTCGTACGGTGACCTGAACCACCTCGTTTCCGTCGTCATGTCC |
| | | GGCATCACCACCTGCCTGCGTTTCCCTGGTCAGCTTAACTCTGATCTTCGCAAGCTCGCCGTCAACATGGT |
| | | TCCTTTCCCTCGTCTTCACTTTTTCATGGTCGGCTTTGCTCCCCTGACGAGCCGTGGTGCCCACTCCTTCCG |
| | | CGCCGTCTCTGTTCCCGAGCTCACTCAGCAGATGTTCGACCCTAAGAACATGATGGCTGCTTCTGACTTCC |
| | | GCAACGGTCGCTACCTGACCTGCTCTGCCATTTTCCGTGGTAAGGTTGCCATGAAGGAGGTTGAGGACCA |
| | | GATGCGTAATGTGCAGACCAAGAACTCCAGCTACTTCGTCGAGTGGATCCCCAACAACATCCAGAACGC |
| | | TCTCTGCGCCGTTCCCCCCCGCGGACTTAAGATGTCGTCTACCTTCATTGGTAACTCGACCTCTATCCAGG |
| | | ATCTCTTCAAGCGTGTCGGTGAGCAGTTCTCCGCCATGTTCCGTCGCAAGGCTTTCCTTCATTGGTACACT |
| | | GGTGAAGGTATGGACGAGATGGAGTTCACTGAGGCTGAGTCCAACATGAACGATCTTATCTCCGAATAC |
| | | CAGCAGTACCAGGACGCTGGCATTGATGATGAGGAAGAGGAATACGAGGAGGAGCTCCCTGTCGAGGG |
| | | CGAGGAGCCTTTGTAA |
| 38 | US-699 | ATGCGTGAGATTGTTCACCTCCAGACCGGCCAGTGCGGTAACCAAATCGGTGCTGCTTTCTGGCAGACCA |
| | | TCTCTGGCGAGCACGGCCTCGACTCCAGCGGTGTTTACAATGGCACTTCTGAGCTTCAGCTCGAGCGCAT |
| | | GAATGTCTACTTCAACGAGGCCTCCGGCAACAAGTATGTCCCTCGCGCCGTCCTCGTCGATCTTGAGCCC |
| | | GGTACCATGGATGCTGTCCGTGCCGGACCCTTCGGTCAGCTCTTCCGTCCCGACAACTTCGTTTTCGGTCA |
| | | GTCCGGTGCCGGCAACAACTGGGCCAAGGGTCATTACACTGAGGGTGCCGAGCTCGTTGACCAGGTCCT |
| | | CGACGTTGTTCGTCGTGAGGCCGAAGGCTGCGACTGCCTCCAGGGTTTCCAGATCACCCACTCTCTTGGT |
| | | GGTGGTACTGGTGCTGGTATGGGTACTCTGCTCATCTCCAAGATCCGCGAAGAGTTTCCCGACCGCATGA |
| | | TGGCCACCTTTTCCGTGGTTCCCTCTCCCGGCAACTCCGACACCGTTGTCGAGCCCTACAACGCTACTCTC |
| | | TCCGTTCACCAGCTCGTTGAGAACTCCGACGAGACCTTCTGTATCGACAACCAGGCTCTGTACGATATCT |
| | | GCATGCGTACCCTGAAGCTATCCAATCCTTCGTACGGTGACCTGAACCACCTCGTTTCCGTCGTCATGTCC |
| | | GGCATCACCACCTGCCTGCGTTTCCCTGGTCAGCTTAACTCTGATCTTCGCAAGCTCGCCGTCAACATGGT |
| | | TCCTTTCCCTCGTCTTCACTTTTTCATGGTCGGCTTTGCTCCCCTGACGAGCCGTGGTGCCCACTCCTTCCG |
| | | CGCCGTCTCTGTTCCCGAGCTCACTCAGCAGATGTTCGACCCTAAGAACATGATGGCTGCTTCTGACTTCC |
| | | GCAACGGTCGCTACCTGACCTGCTCTGCCATTTTCCGTGGTAAGGTTGCCATGAAGGAGGTTGAGGACCA |
| | | GATGCGTAATGTGCAGACCAAGAACTCCAGCTACTTCGTCGAGTGGATCCCCAACAACATCCAGAACGC |
| | | TCTCTGCGCCGTTCCCCCCCGCGGACTTAAGATGTCGTCTACCTTCATTGGTAACTCGACCTCTATCCAGG |
| | | ATCTCTTCAAGCGTGTCGGTGAGCAGTTCTCCGCCATGTTCCGTCGCAAGGCTTTCCTTCATTGGTACACT |
| | | GGTGAAGGTATGGACGAGATGGAGTTCACTGAGGCTGAGTCCAACATGAACGATCTTATCTCCGAATAC |
| | | CAGCAGTACCAGGACGCTGGCATTGATGATGAGGAAGAGGAATACGAGGAGGAGCTCCCTGTCGAGGG |
| | | CGAGGAGCCTTTGTAA |
| 39 | US-707 | ATGCGTGAGATTGTTCACCTCCAGACCGGCCAGTGCGGTAACCAAATCGGTGCTGCTTTCTGGCAGACCA |
| | | TCTCTGGCGAGCACGGCCTCGACTCCAGCGGTGTTTACAATGGCACTTCTGAGCTTCAGCTCGAGCGCAT |
| | | GAATGTCTACTTCAACGAGGCCTCCGGCAACAAGTATGTTCCTCGCGCCGTCCTCGTCGATCTTGAGCCC |
| | | GGTACCATGGATGCTGTCCGTGCCGGTCCCTTCGGTCAGCTCTTCCGTCCCGACAACTTCGTTTTCGGTCA |
| | | GTCCGGTGCCGGCAACAACTGGGCCAAGGGTCATTACACTGAGGGTGCCGAGCTCGTTGACCAGGTCCT |
| | | CGACGTTGTTCGTCGTGAGGCCGAAGGCTGCGACTGCCTCCAGGGTTTCCAGATCACCCACTCTCTTGGT |
| | | GGTGGTACTGGTGCTGGTATGGGTACTCTGCTCATCTCCAAGATCCGCGAAGAGTTTCCCGACCGCATGA |
| | | TGGCCACCTTTTCCGTGGTTCCCTCTCCCGGCAACTCCGACACCGTTGTCGAGCCCTACAACGCTACTCTC |
| | | TCCGTTCACCAGCTCGTTGAGAACTCCGACGAGACCTTCTGTATCGACAACCAGGCTCTGTACGATATCT |
| | | GCATGCGTACCCTGAAGCTATCTAATCCTTCGTACGGTGACCTGAACCACCTCGTTTCCGTCGTCATGTCC |
| | | GGCATCACCACCTGCCTGCGTTTCCCTGGTCAGCTTAACTCTGATCTTCGCAAGCTCGCCGTCAACATGGT |
| | | TCCTTTCCCTCGTCTTCACTTTTTCATGGTCGGCTTTGCTCCCCTGACGAGCCGTGGTGCACACTCCTTCCG |
| | | CGCTGTCTCTGTTCCTGAGCTCACTCAGCAGATGTTCGACCCTAAGAACATGATGGCTGCTTCTGACTTCC |
| | | GTAACGGTCGCTACCTGACCTGCTCTGCCATTTTCCGTGGCAAGGTTGCCATGAAGGAGGTTGAGGACCA |
| | | GATGCGTAATGTGCAGACCAAGAACTCCAGCTACTTCGTCGAGTGGATCCCCAACAACATCCAGAACGC |
| | | TCTCTGCGCCGTTCCCCCCCGCGGACTTAAGATGTCGTCTACCTTCATTGGTAACTCGACCTCTATCCAGG |
| | | ATCTCTTCAAGCGTGTCGGTGAACAGTTCTCCGCCATGTTCCGTCGCAAGGCTTTCCTTCATTGGTACACT |
| | | GGCGAAGGTATGGACGAGATGGAGTTCACTGAGGCTGAGTCCAACATGAACGATCTTATCTCCGAATAC |
| | | CAGCAGTACCAGGACGCTGGCATTGATGATGAGGAAGAGGAATACGAGGAGGAGCTCCCTGTCGAGGG |
| | | CGAGGAGCCTTTGTAA |
| 40 | US-803 | ATGCGTGAGATTGTTCACCTCCAGACCGGCCAGTGCGGTAACCAAATCGGTGCTGCTTTCTGGCAGACCA |
| | | TCTCTGGCGAGCACGGCCTCGACTCCAGCGGTGTTTACAATGGCACTTCTGAGCTTCAGCTCGAGCGCAT |
| | | GAATGTCTACTTCAACGAGGCCTCCGGCAACAAGTATGTCCCTCGCGCCGTCCTCGTCGATCTTGAGCCC |
| | | GGTACCATGGATGCTGTCCGTGCCGGACCCTTCGGTCAGCTCTTCCGTCCCGACAACTTCGTTTTCGGTCA |
| | | GTCCGGTGCCGGCAACAACTGGGCCAAGGGTCATTACACTGAGGGTGCCGAGCTCGTTGACCAGGTCCT |
| | | CGACGTTGTTCGTCGTGAGGCCGAAGGCTGCGACTGCCTCCAGGGTTTCCAGATCACCCACTCTCTTGGT |
| | | GGTGGTACTGGTGCTGGTATGGGTACTCTGCTCATCTCCAAGATCCGCGAAGAGTTTCCCGACCGCATGA |
| | | TGGCCACCTTTTCCGTGGTTCCCTCTCCCGGCAACTCCGACACCGTTGTCGAGCCCTACAACGCTACTCTC |
| | | TCCGTTCACCAGCTCGTTGAGAACTCCGACGAGACCTTCTGTATCGACAACCAGGCTCTGTACGATATCT |
| | | GCATGCGTACCCTGAAGCTATCCAATCCTTCGTACGGTGACCTGAACCACCTCGTTTCCGTCGTCATGTCC |
| | | GGCATCACCACCTGCCTGCGTTTCCCTGGTCAGCTTAACTCTGATCTTCGCAAGCTCGCCGTCAACATGGT |
| | | TCCTTTCCCTCGTCTTCACTTTTTCATGGTCGGCTTTGCTCCCCTGACGAGCCGTGGTGCCCACTCCTTCCG |
| | | CGCCGTCTCTGTTCCCGAGCTCACTCAGCAGATGTTCGACCCTAAGAACATGATGGCTGCTTCTGACTTCC |
| | | GCAACGGTCGCTACCTGACCTGCTCTGCCATTTTCCGTGGTAAGGTTGCCATGAAGGAGGTTGAGGACCA |
| | | GATGCGTAATGTGCAGACCAAGAACTCCAGCTACTTCGTCGAGTGGATCCCCAACAACATCCAGAACGC |
| | | TCTCTGCGCCGTTCCCCCCCGCGGACTTAAGATGTCGTCTACCTTCATTGGTAACTCGACCTCTATCCAGG |
| | | ATCTCTTCAAGCGTGTCGGTGAGCAGTTCTCCGCCATGTTCCGTCGCAAGGCTTTCCTTCATTGGTACACT |
| | | GGTGAAGGTATGGACGAGATGGAGTTCACTGAGGCTGAGTCCAACATGAACGATCTTATCTCCGAATAC |
| | | CAGCAGTACCAGGACGCTGGCATTGATGATGAGGAAGAGGAATACGAGGAGGAGCTCCCTGTCGAGGG |
| | | CGAGGAGCCTTTGTAA |
| 41 | US-670 | ATGCGTGAGATTGTTCACCTCCAGACCGGCCAGTGCGGTAACCAAATCGGTGCTGCTTTCTGGCAGACCA |
| | | TCTCTGGCGAGCACGGCCTCGACTCCAGCGGTGTTTACAATGGCACTTCTGAGCTTCAGCTCGAGCGCAT |
| | | GAATGTCTACTTCAACGAGGCCTCCGGCAACAAGTATGTCCCTCGCGCCGTCCTCGTCGATCTTGAGCCC |
| | | GGTACCATGGATGCTGTCCGTGCCGGACCCTTCGGTCAGCTCTTCCGTCCCGACAACTTCGTTTTCGGTCA |
| | | GTCCGGTGCCGGCAACAACTGGGCCAAGGGTCATTACACTGAGGGTGCCGAGCTCGTTGACCAGGTCCT |
| | | CGACGTTGTTCGTCGTGAGGCCGAAGGCTGCGACTGCCTCCAGGGTTTCCAGATCACCCACTCTCTTGGT |
| | | GGTGGTACTGGTGCTGGTATGGGTACTCTGCTCATCTCCAAGATCCGCGAAGAGTTTCCCGACCGCATGA |
| | | TGGCCACCTTTTCCGTGGTTCCCTCTCCCGGCAACTCCGACACCGTTGTCGAGCCCTACAACGCTACTCTC |
| | | TCCGTTCACCAGCTCGTTGAGAACTCCGACGAGACCTTCTGTATCGACAACCAGGCTCTGTACGATATCT |
| | | GCATGCGTACCCTGAAGCTATCCAATCCTTCGTACGGTGACCTGAACCACCTCGTTTCCGTCGTCATGTCC |
| | | GGCATCACCACCTGCCTGCGTTTCCCTGGTCAGCTTAACTCTGATCTTCGCAAGCTCGCCGTCAACATGGT |
| | | TCCTTTCCCTCGTCTTCACTTTTTCATGGTCGGCTTTGCTCCCCTGACGAGCCGTGGTGCCCACTCCTTCCG |
| | | CGCCGTCTCTGTTCCCGAGCTCACTCAGCAGATGTTCGACCCTAAGAACATGATGGCTGCTTCTGACTTCC |
| | | GCAACGGTCGCTACCTGACCTGCTCTGCCATTTTCCGTGGTAAGGTTGCCATGAAGGAGGTTGAGGACCA |
| | | GATGCGTAATGTGCAGACCAAGAACTCCAGCTACTTCGTCGAGTGGATCCCCAACAACATCCAGAACGC |
| | | TCTCTGCGCCGTTCCCCCCCGCGGACTTAAGATGTCGTCTACCTTCATTGGTAACTCGACCTCTATCCAGG |
| | | ATCTCTTCAAGCGTGTCGGTGAGCAGTTCTCCGCCATGTTCCGTCGCAAGGCTTTCCTTCATTGGTACACT |
| | | GGTGAAGGTATGGACGAGATGGAGTTCACTGAGGCTGAGTCCAACATGAACGATCTTATCTCCGAATAC |
| | | CAGCAGTACCAGGACGCTGGCATTGATGATGAGGAAGAGGAATACGAGGAGGAGCTCCCTGTCGAGGG |
| | | CGAGGAGCCTTTGTAA |
| 42 | US-935 | ATGCGTGAGATTGTTCACCTCCAGACCGGCCAGTGCGGTAACCAAATCGGTGCTGCTTTCTGGCAGACCA |
| | | TCTCTGGCGAGCACGGCCTCGACTCCAGCGGTGTTTACAATGGCACTTCTGAGCTTCAGCTCGAGCGCAT |
| | | GAATGTCTACTTCAACGAGGCCTCCGGCAACAAGTATGTCCCTCGCGCCGTCCTCGTCGATCTTGAGCCC |
| | | GGTACCATGGATGCTGTCCGTGCCGGACCCTTCGGTCAGCTCTTCCGTCCCGACAACTTCGTTTTCGGTCA |
| | | GTCCGGTGCCGGCAACAACTGGGCCAAGGGTCATTACACTGAGGGTGCCGAGCTCGTTGACCAGGTCCT |
| | | CGACGTTGTTCGTCGTGAGGCCGAAGGCTGCGACTGCCTCCAGGGTTTCCAGATCACCCACTCTCTTGGT |
| | | GGTGGTACTGGTGCTGGTATGGGTACTCTGCTCATCTCCAAGATCCGCGAAGAGTTTCCCGACCGCATGA |
| | | TGGCCACCTTTTCCGTGGTTCCCTCTCCCGGCAACTCCGACACCGTTGTCGAGCCCTACAACGCTACTCTC |
| | | TCCGTTCACCAGCTCGTTGAGAACTCCGACGAGACCTTCTGTATCGACAACCAGGCTCTGTACGATATCT |
| | | GCATGCGTACCCTGAAGCTATCCAATCCTTCGTACGGTGACCTGAACCACCTCGTTTCCGTCGTCATGTCC |
| | | GGCATCACCACCTGCCTGCGTTTCCCTGGTCAGCTTAACTCTGATCTTCGCAAGCTCGCCGTCAACATGGT |
| | | TCCTTTCCCTCGTCTTCACTTTTTCATGGTCGGCTTTGCTCCCCTGACGAGCCGTGGTGCCCACTCCTTCCG |
| | | CGCCGTCTCTGTTCCCGAGCTCACTCAGCAGATGTTCGACCCTAAGAACATGATGGCTGCTTCTGACTTCC |
| | | GCAACGGTCGCTACCTGACCTGCTCTGCCATTTTCCGTGGTAAGGTTGCCATGAAGGAGGTTGAGGACCA |
| | | GATGCGTAATGTGCAGACCAAGAACTCCAGCTACTTCGTCGAGTGGATCCCCAACAACATCCAGAACGC |
| | | TCTCTGCGCCGTTCCCCCCCGCGGACTTAAGATGTCGTCTACCTTCATTGGTAACTCGACCTCTATCCAGG |
| | | ATCTCTTCAAGCGTGTCGGTGAGCAGTTCTCCGCCATGTTCCGTCGCAAGGCTTTCCTTCATTGGTACACT |
| | | GGTGAAGGTATGGACGAGATGGAGTTCACTGAGGCTGAGTCCAACATGAACGATCTTATCTCCGAATAC |
| | | CAGCAGTACCAGGACGCTGGCATTGATGATGAGGAAGAGGAATACGAGGAGGAGCTCCCTGTCGAGGG |
| | | CGAGGAGCCTTTGTAA |

### Crop Plants

A “plant” means any plant of economic importance and includes cereals (such as wheat, barley, rye, triticale, millet, oats), maize (corn), canola, cotton, soya bean, rice, potatoes, sunflowers, beans, coffee, beets (e.g. sugar beets and fodder beets), peanuts, oilseed rape, poppies, olives, coconuts, cacao, sugar cane, tobacco, vegetables (such as tomatoes, cucumbers, onions and lettuce), lawn and ornamental plants. In a preferred embodiment, the plant is a crop plant. “Crop plant” generally means any cultivated plant grown to produce a harvested horticultural product for sale and/or profit, as well as subsistence crops that may be grown to support other agricultural products, such as livestock. The crop plant may be any crop of agronomic importance cultivated for food, animal feed, fiber, fuel, and/or industrial purposes. The crop plant may vary from region to region worldwide, wherein the variance may depend on factors such as dietary requirements and environmental conditions.

“Plant cultivars” are understood to mean plants that have new properties (“traits”) and have been obtained by conventional breeding, mutagenesis or recombinant DNA techniques. They can be cultivars, varieties, bio- or genotypes.

“Plant parts” are understood to mean all parts and organs of plants above and below the ground, such as shoots, leaves, needles, stalks, stems, flowers, fruit bodies, fruits, seeds, roots, tubers, and rhizomes. The plant parts also include harvested material and vegetative and generative propagation material, for example, cuttings, tubers, rhizomes, slips and seeds.

An “increase in yield” of a crop plant treated with one or more fungal species includes an increase in fruit, grain or vegetative tissue production of the treated plant relative to that of a crop plant that is the same, but which has not been treated with the one or more fungal species described herein when the treated and untreated plant is grown under the same growing conditions. An “untreated control plant” used herein is grown in a similar soil type under similar conditions (e.g., fertilizer application, watering, etc.) except that no fungal strain is applied to the plant. For example, an increase in yield of a treated wheat plant is an increase in the number and/or weight of wheat grains produced by the treated wheat plant relative to that of an untreated wheat plant grown under the same growth conditions. Typically, the increase in yield of a plant treated with one or more fungal species is an increase in fruit, grain or vegetative tissue production of the treated plant relative to that of a healthy plant of the same type that has not been treated with the one or more fungal species described herein when the treated and untreated plant is grown under the same growing conditions. A healthy plant is a plant that is not infected with, or affected by, a plant pathogen. Typically, a healthy plant is a plant that is not infected with, or affected by, a plant pathogen and which is grown under conditions for normal growth of that plant (e.g., is not under stress, such as nutrient or drought stress), such as, for example, the conditions under which the crop plant would be grown under during commercial crop production.

Increased yield in plants can result from improved plant physiology, growth and development, such as water use efficiency, water retention efficiency, improved nitrogen use, enhanced carbon assimilation, improved photosynthesis, increased germination efficiency and accelerated growth maturation. Yield can furthermore be affected by improved plant architecture (under stress and non-stress conditions), including but not limited to, early flowering, flowering control for hybrid seed production, seedling vigor, plant size, chlorophyll content, nodulation, internode number and distance, root growth (e.g., root number, root length, root mass, root volume), shoot growth (e.g., shoot mass, leaf area, leaf number, plant height) seed size, fruit size, pod size, pod or ear number, seed number per pod or ear, seed mass, enhanced seed filling, reduced seed dispersal, reduced pod dehiscence and lodging resistance. As used herein, “agronomic benefits” means improving one or more of these factors thereby increasing the yield of the plant.

The crop plant may, for example, be one or more compatible crops selected from the group consisting of species of the genus Triticum, Glycine, Brassica, Gossypium, Zea, Corchorus, Saccharum, Medicago, Lolium, Coffea, Camellia, Oryza, Hordeum, Boehmeria, Nicotiana, Cannabis, oilseeds, grain legumes, vegetables, fruits, and/or combinations or hybrids thereof. It is contemplated that the list of the crop plants disclosed herein are mere examples for the skilled persons to understand the present disclosure. The crop plants may further include new future species and breeds as well as hybrids produced by grafting or transgenic species.

In preferred embodiments of the invention, the crop plant may, for example, be one or more crops selected from the group consisting of the species Triticum aestivum, Brassica napus, Brassica rapa, Brassica juncea, Gossypium hirsutum, Gossypium barbadense, Gossypium arboretum, Gossypium Herbaceum, Zea mays, Medicago sativa, Lolium multiflorum, Corchorus capsularis, Saccharum officinarum, Cannabis sativa, Coffea Arabica, Coffea Robusta, Camellia sinensis, Oryza sativa, Hordeum vulgare, Boehmeria nivea and Nicotiana tabacum.

In one embodiment, the crop plant is a cereal plant. Cereal plants include, for example, wheat, rice, corn (maize), canola, rye, oats, barley, sorghum, and some of the millets. In various embodiments, the crop plant is a cereal plant selected from the group consisting of wheat, rice and corn. In one aspect, the plant is a millet selected from the group consisting of finger millet (Eleusine coracana), foxtail millet (Setaria italica), browntop millet (Urochloa ramose), pearl millet (Pennisetum glaucum), Japanese millet/barnyard millet (Echinochloa esculenta), little millet (Panicum sumatrense), broomcorn millet/proso millet (Panicum miliaceum), Kodo millet (Paspalum scrobiculatum), fonio millet (Digitaria exilis), guinea millet (Brachiaria deflexa), great millet (Sorghum bicolor), Sonoran millet (Panicum hirticaule), Polish millet (Digitaria sanguinalis), adlay millet (Coix lacryma-jobi), and Taiwan oil millet (Spodiopogon formosanus).

In another embodiment, the crop plant is a fibre plant. Fibre plants include, for example, flax, hemp, jute, and cotton. In various embodiments, the crop plant is a fibre plant that is cotton.

In one embodiment, the crop plant is a legume. Legume plants include, for example, soybeans, alfalfa, clover, peanuts, lentils, lupins, peas, Desmanthus, and chickpea. In various embodiments, the crop plant is a legume that is soybeans. In one aspect, the crop plant is Desmanthus. In another aspect, the Desmanthus is Desmanthus virgatus, Desmanthus leptophyllus, or Desmanthus pubescens.

In some aspects, the crop plant is a cover crop. Cover crops include but are not limited to ryegrass, clover, red clover, white clover, crimson clover, annual medics, annual ryegrass, Italian ryegrass, canola, fine fescue, Kentucky bluegrass, orchard grass, and other grasses.

In other aspects, the crop plant is a grass such as switchgrass, tall fescue, meadow fescue, perennial ryegrass, Italian ryegrass, orchard grass, guinea grass, foxtail millet, pearl millet, Bahia grass and Miscanthus.

In embodiments of the invention, the plant is a “non-native” plant host of the fungal strain. “Non-native” plant host means that the fungi are heterologous to said plant insofar as the fungal strain was collected from a host other than said crop plant.

Endophytic fungi are known to have preferred hosts and growth conditions. Consequently, they will not necessarily flourish, and, therefore, will not produce the desired stable SOC, in the absence of their typical growth environment or an association with their native hosts. Moreover, when considering the survival of the fungi in non-native plant hosts, it is difficult to anticipate whether the fungi will be pathogenic to the non-native host. Therefore, fungal species may not readily be compatible with a non-native crop plant host.

### Inoculation

As used herein, the terms “inoculate,” “apply,” “treat,” and “deploy” are used interchangeably, as are their associated nouns (i.e., “inoculation, “application,” “treatment,” and “deployment”). The inoculation of the plant with one or more fungal species may be achieved by any suitable means, such as direct addition to the soil and/or plant roots and/or to soil proximal to plant roots, or may be achieved by an initial fungal inoculation of any propagation material, seeds, seedlings and/or immature plants of the crop plant prior to placement of the seed, seedling or immature plant in the soil within which the plant will grow. The inoculation of the one or more fungal species may also be achieved by direct addition to a cultivated soil prior to sowing seeds or planting seedlings that are coated or partially coated with one or more fungal species such that the fungi will become associated with, or grow proximal to, or grow into the roots of a crop plant as the crop matures.

By means of the inoculation, the fungi are deliberately encouraged to become established in the soil and/or grow proximal to, or grow into the roots of a plant (i.e., become associated with) that is a crop plant, wherein it would be understood the fungi may exist and grow in the soil or exist within the plant, or in both simultaneously. In aspects of the invention wherein the treatments or methods rely on the inoculation of a plant with a fungus, it would be understood that the fungus need only be associated with the plant for parts of the fungus' lifecycle and that the fungus may survive in the soil in the absence of a plant host or host crop plant.

In other embodiments, the inoculation may be considered a semi-permanent inoculation to a plot of cultivated soil, such that the fungus is deployed to said plot of soil and is retained by the soil as the crops are rotated, even in the absence of crops for periods of time.

In some embodiments, the soil is inoculated with the one or more fungal species. The soil may be inoculated with one or more fungal species prior to planting the plant, for example, before, during, or after tilling the soil in preparation for planting. In other embodiments, the soil may be inoculated with the one or more fungal species after the plant has been planted. In some embodiment, the soil is inoculated with the one or more fungal species by planting in the soil plants that have been inoculated with the one or more fungal species.

In some embodiments, the step of inoculating a crop plant comprises applying the one or more fungal species to the seeds of the plant prior to planting.

In some embodiments, the step of inoculating a crop plant comprises applying the one or more fungal species to seedlings of the plant.

In some embodiments, the step of inoculating soil comprises deploying the one or more fungal species to a plot of cultivated soil, such that the fungus is retained by the soil as the crops are rotated, even in the absence of crops for periods of time.

In one embodiment, the plants are inoculated with one or more fungal species as a seed coating before, during or after one or more of the seed germination stages or as a root inoculant of a seedling. For example, the treatment may be applied as a seed coating to seeds en masse prior to sowing a crop.

The one or more fungal species for inoculation may be in any suitable form, including, for example, as hyphae, mycelia, conidia and/or combinations thereof. In general, the one or more fungal species for inoculating the plant will be in a form that is substantially free of contaminating microorganisms, with the exception that additional desirable microbes may be added for additional benefits.

In some embodiments, the inoculant may be in the form of a dried powder, a spray, a slurry, a sachet, a liquid, a jelly, a seed coating, an enhancer, and/or combinations thereof.

In some embodiments, the inoculant is in the form of a seed coating, a foliar spray, granule, powder, soil drench or a root dip.

In one embodiment, the inoculant is in the form of a seed coating.

In one embodiment, the inoculant is in the form of a foliar spray.

In one embodiment, the inoculant is in the form of a root dip.

In one embodiment, the inoculant is a granule.

In one embodiment, the inoculant is a powder.

In one embodiment, the composition is a soil drench.

In some aspects, the fungal inoculant is applied to seed at a rate of about 1×10⁶ colony forming units (CFU) per kg seed to about 1×10¹² CFU per kg seed, about 1×10⁷ (CFU) per kg seed to about 1×10¹² CFU per kg seed, about 1×10⁸ (CFU) per kg seed to about 1×10¹² CFU per kg seed, about 1×10⁹ (CFU) per kg seed to about 1×10¹² CFU per kg seed, or about 1×10¹⁰ (CFU) per kg seed to about 1×10¹² CFU per kg seed.

In other aspects, the fungal inoculant is applied to seed at a rate of about 1×10² CFU per seed to about 1×10⁵ CFU per seed, about 1×10³ CFU per seed to about 1×10⁵ CFU per seed, about 1×10⁴ CFU per seed to about 1×10⁵ CFU per seed, about 1×10² CFU per seed to about 1×10⁴ CFU per seed, or about 1×10³ CFU per seed to about 1×10⁴ CFU per seed.

In other aspects, the fungal inoculant is applied as a soil amendment at a rate of about 1×10⁴ CFU per hectare to about 1×10¹² CFU per hectare, about 1×10⁵ CFU per hectare to about 1×10¹² CFU per hectare, about 1×10⁶ CFU per hectare to about 1×10¹² CFU per hectare, about 1×10⁷ CFU per hectare to about 1×10¹² CFU per hectare, about 1×10⁸ CFU per hectare to about 1×10¹² CFU per hectare, about 1×10⁹ CFU per hectare to about 1×10¹² CFU per hectare, about 1×10¹⁰ CFU per hectare to about 1×10¹² CFU per hectare, about 1×10⁴ CFU per hectare to about 1×10¹⁰ CFU per hectare, about 1×10⁵ CFU per hectare to about 1×10¹⁰ CFU per hectare, about 1×10⁶ CFU per hectare to about 1×10¹⁰ CFU per hectare, about 1×10⁷ CFU per hectare to about 1×10¹⁰ CFU per hectare, about 1×10⁸ CFU per hectare to about 1×10¹⁰ CFU per hectare, about 1×10⁴ CFU per hectare to about 1×10⁸ CFU per hectare, about 1×10⁵ CFU per hectare to about 1×10⁸ CFU per hectare, or about 1×10⁶ CFU per hectare to about 1×10⁸ CFU per hectare.

In some embodiments, the one or more fungal species are compatible with commonly used agricultural fungicides. “Compatible” means the one or more fungal species in the treatment is not killed or substantially inhibited (growth or germination or otherwise) by the fungicide, thereby allowing the fungi in the treatment to flourish while restricting the growth of undesirable fungal strains that may have a deleterious effect on the soil, the proximal crops or plants, and/or the level of carbon sequestration and stable carbon production. The fungicide may be any synthetic or natural compound that has a fungistatic or fungicidal function and are commonly used in agriculture. Based on their mode of action, they may kill the fungi or inhibit the germination of fungal spores.

The composition and/or inoculant may comprise suitable solid or liquid carriers and/or adhesive agents.

Suitable solid carriers include mineral earths (e.g., calcium phosphate, calk, clay, diatomaceous earth, dolomite, kaolin, silicates, silica gels, talc, etc), cellulose, and starch. Suitable liquid carriers include water, or any other liquid solvents which are not toxic to the fungus or the plant.

The composition may be prepared in a known manner, by mixing it with customary adjuvants, such as, for example, customary extenders and also solvents or diluents, colorants, wetters, dispersants, emulsifiers, antifoams, preservatives, secondary thickeners, stickers, and also water.

Colorants which may be present in the composition which can be used in accordance with the invention include all colorants which are customary for such purposes. In this context it is possible to use not only pigments, which are of low solubility in water, but also water-soluble dyes. Examples include the colorants known under the designations Rhodamine B, C.I. Pigment Red 112 and C.I. Solvent Red 1.

Wetters that may be present in the composition include all of the substances which promote wetting and which are customary in the formulation of active agrochemical ingredients. Use may be made preferably of alkylnaphthalenesulphonates, such as diisopropyl- or diisobutyl-naphthalenesulphonates.

Dispersants and/or emulsifiers which may be present in the composition include all of the nonionic, anionic and cationic dispersants that are customary in the formulation of active agrochemical ingredients. Use may be made preferably of nonionic or anionic dispersants or of mixtures of nonionic or anionic dispersants. Suitable nonionic dispersants are, in particular, ethylene oxide-propylene oxide block polymers, alkylphenol polyglycol ethers and also tristryrylphenol polyglycol ethers, and the phosphated or sulphated derivatives of these. Suitable anionic dispersants are, in particular, lignosulphonates, salts of polyacrylic acid, and arylsulphonate-formaldehyde condensates.

Antifoams which may be present in the composition include all of the foam inhibitors that are customary in the formulation of active agrochemical ingredients. Use may be made preferably of silicone antifoams and magnesium stearate.

Preservatives which may be present in the composition include all of the substances which can be employed for such purposes in agrochemical compositions. Examples include dichlorophen and benzyl alcohol hemiformal.

Secondary thickeners which may be present in the composition include all substances which can be used for such purposes in agrochemical compositions. Those contemplated with preference include cellulose derivatives, acrylic acid derivatives, xanthan, modified clays and highly disperse silica.

Stickers which may be present in the composition include all customary binders which can be used in seed-dressing products. Preferred mention may be made of polyvinylpyrrolidone, polyvinyl acetate, polyvinyl alcohol and tylose.

The purified fungal populations described herein can be formulated using an agriculturally compatible carrier. The formulation useful for these embodiments generally typically include at least one member selected from the group consisting of a tackifier, a microbial stabilizer, a fungicide, an antibacterial agent, an herbicide, a nematicide, an insecticide, a plant growth regulator, a rodenticide, a desiccant, and a nutrient.

In some cases, the purified fungal population is mixed with an agriculturally compatible carrier. The carrier can be a solid carrier or liquid carrier, and in various forms including microspheres, powders, emulsions and the like. The carrier may be any one or more of a number of carriers that confer a variety of properties, such as increased stability, wettability, or dispersibility. Wetting agents such as natural or synthetic surfactants, which can be nonionic or ionic surfactants, or a combination thereof can be included in a composition of the invention. Water-in-oil emulsions can also be used to formulate a composition that includes the purified fungal population (see, for example, U.S. Pat. No. 7,485,451, which is incorporated herein by reference in its entirety). Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc. The formulation may include grain or legume products, for example, ground grain or beans, broth or flour derived from grain or beans, starch, sugar, or oil.

In some embodiments, the agricultural carrier may be soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the cultured organisms, such as barley, rice, or other biological materials such as seed, plant parts, sugar cane bagasse, hulls or stalks from grain processing, ground plant material or wood from building site refuse, sawdust or small fibers from recycling of paper, fabric, or wood. Other suitable formulations will be known to those skilled in the art.

In one embodiment, the formulation can include a tackifier or adherent. Such agents are useful for combining the fungal population of the invention with carriers that can contain other compounds (e.g., control agents that are not biologic), to yield a coating composition. Such compositions help create coatings around the plant or seed to maintain contact between the microbe and other agents with the plant or plant part. In one embodiment, adherents are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers. Other examples of adherent compositions that can be used in the synthetic preparation include those described in EP 0818135, CA 1229497, WO 2013090628, EP 0192342, WO 2008103422 and CA 1041788, each of which is incorporated herein by reference in its entirety.

The formulation can also contain a surfactant. Non-limiting examples of surfactants include nitrogen-surfactant blends such as Prefer 28 (Cenex), Surf-N (US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

In certain cases, the formulation includes a microbial stabilizer. Such an agent can include a desiccant. As used herein, a “desiccant” can include any compound or mixture of compounds that can be classified as a desiccant regardless of whether the compound or compounds are used in such concentrations that they in fact have a desiccating effect on the liquid inoculant. Such desiccants are ideally compatible with the fungal population used, and should promote the ability of the microbial population to survive application on the seeds and to survive desiccation. Examples of suitable desiccants include one or more of trehalose, sucrose, glycerol, and Methylene glycol. Other suitable desiccants include, but are not limited to, non reducing sugars and sugar alcohols (e.g., mannitol or sorbitol). The amount of desiccant introduced into the formulation can range from about 5% to about 50% by weight/volume, for example, between about 10% to about 40%, between about 15% and about 35%, or between about 20% and about 30%.

In some cases, it is advantageous for the formulation to contain agents such as a fungicide, an antibacterial agent, an herbicide, a nematicide, an insecticide, a plant growth regulator, a rodenticide, or a nutrient. Such agents are ideally compatible with the agricultural seed or seedling onto which the formulation is applied (e.g., it should not be deleterious to the growth or health of the plant). Furthermore, the agent is ideally one which does not cause safety concerns for human, animal or industrial use (e.g., no safety issues, or the compound is sufficiently labile that the commodity plant product derived from the plant contains negligible amounts of the compound).

In the liquid form, for example, solutions or suspensions, the fungal endophytic populations of the present invention can be mixed or suspended in water or in aqueous solutions. Suitable liquid diluents or carriers include water, aqueous solutions, petroleum distillates, or other liquid carriers.

Solid compositions can be prepared by dispersing the fungal endophytic populations of the invention in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

The solid carriers used upon formulation include, for example, mineral carriers such as kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran may be used. The liquid carriers include vegetable oils such as soybean oil and cottonseed oil, glycerol, ethylene glycol, polyethylene glycol, propylene glycol, polypropylene glycol, etc.

In one particular embodiment, the formulation is ideally suited for coating of the endophytic microbial population onto seeds. The fungal endophytic populations described in the present invention are capable of conferring many fitness benefits to the host plants. The ability to confer such benefits by coating the fungal populations on the surface of seeds has many potential advantages, particularly when used in a commercial (agricultural) scale.

The fungal endophytic populations herein can be combined with one or more of the agents described above to yield a formulation suitable for combining with an agricultural seed or seedling. The fungal population can be obtained from growth in culture, for example, using a synthetic growth medium. In addition, the microbe can be cultured on solid media, for example on petri dishes, scraped off and suspended into the preparation. Microbes at different growth phases can be used. For example, microbes at lag phase, early-log phase, mid-log phase, late-log phase, stationary phase, early death phase, or death phase can be used.

The formulations comprising the fungal endophytic population of the present invention typically contains between about 0.1 to 95% by weight, for example, between about 1% and 90%, between about 3% and 75%, between about 5% and 60%, between about 10% and 50% in wet weight of the fungal population of the present invention. It is preferred that the formulation contains at least about 103 CFU per ml of formulation, for example, at least about 104, at least about 105, at least about 106, at least 107 CFU, at least 108 CFU per ml of formulation.

### Soil Organic Carbon

The fungi used in the methods described herein will generally be capable of stabilizing and fixing carbon sequestered by plants from atmospheric carbon dioxide and converting this carbon to complex polysaccharides for storage as stable carbon in the soil. The sequestered and fixed carbon may also be converted and stored as a stable carbon source by the fungi in the fungi itself as, for example, melanin, chitin, lignin, suberin and carotenoid compounds, or the fungi may exude these compounds to increase the stable carbon in the soil. The deployed fungal endophyte may also convert simple polysaccharide exudate from a host plant into complex polysaccharides for storage as stable carbon in the soil, or within the fungi itself. Lastly, the stability of organic carbon may be enhanced in soil with more stable soil aggregates and minerals.

The methods and treatments of the present invention may increase the overall levels of carbon in the soil, but even in cases where overall carbon remains the same or is only slightly increased, it would be understood that the levels of stable carbon in the soil may be increased due to the production and exudation in the soil of complex polysaccharides by the disclosed fungal species.

The fungi may be particularly useful to increase overall levels of carbon in the soil and/or levels of stable carbon in the soil where the soil has a soil organic carbon (SOC) level below a particular threshold. In some aspects, the threshold is a SOC level below 5%, 4%, 3%, 2%, 1%, 0.9%, 0.8%, 0.7%, 0.6%, 0.5%, 0.4%, 0.3%, 0.2%, or 0.1%. SOC levels (% wt/wt) are calculated with the following formula:

$$S O C \% wt / wt \text{?} = Weight of Organic Carbon \text{?} Weight of Soil Sample \text{?} \text{?} \times 100$$

The increase in overall soil carbon and stable soil carbon of a soil that is subjected to the treatments and/or methods of the present disclosure compared to an untreated control (i.e., a “non-inoculated control soil”) may be quantified by any methods known to those skilled in the art. The control would be a similar soil sample that had not been exposed to an endophytic fungus as claimed herein (i.e., a fungus had not been deployed in the soil or associated with a plant that had been cultivated in said soil). “Similar soil sample” means that the soil would be from a proximal area with a similar climate and, if the soil had been cultivated, the control sample would have been cultivated by the same plant as the test soil.

In one embodiment, an increase in soil organic carbon is an increase in stable carbon. An increase in the sequestration of atmospheric carbon for storage as stable carbon in the soil and increasing the levels of stable carbon in the soil, is an increase relative to the amount of sequestration of atmospheric carbon for storage as stable carbon in the soil, and levels of stable carbon in the soil, produced by a plant that has not been treated with the methods of the present disclosure.

Application of the fungi to the plant and/or soil may have one or more desirable effects on the soil and/or associated crops cultivated in the treated soil, including for example, sequestering atmospheric carbon for storage as stable carbon in the soil; and/or increasing the levels of stabilised carbon in the soil.

The inoculation of the soil and/or plants with the fungi may have simultaneous beneficial effects on the soil. For example, sequestration of atmospheric carbon by endophytic fungi as described herein can lead to an increase in the complex polysaccharides in the soil resulting in long-term storage of sequestered atmospheric carbon in a stable form.

Soil organic carbon is the overall soil carbon content of a soil and may be also generally referred to as total organic carbon (TOC) or total carbon (TC) (the terms may be used interchangeably), and this refers only to the carbon component of the organic matter in the soil. However, fluctuations in soil organic carbon may not necessarily correlate to the same fluctuations in stable soil carbon. Indeed, soils subjected to the treatments and methods may demonstrate minimal increases in TOC, but the percentage of said TOC that is captured in a stable form in the soil or in the fungi proliferating in the soil (i.e., complex polysaccharides, melanin, chitin, lignin, suberin and carotenoid compounds) may increase. The skilled addressee would also understand that changes in TOC and stable carbon in soil as a result of the treatments and methods of the present may take weeks, months or years, and therefore appropriate measurement timeframes must be applied. In one embodiment, the increase in soil soil organic carbon (SOC) comprises an increase in stable carbon in the soil.

The soil carbon may be measured by methods including, but not limited to, dry combustion or elemental tests that may be analysed using, for example, the LECO method, and loss on ignition (LOI) tests that may be analysed using the Walkley-Black method (see, for example, Walkley A, and Black IA (1934) An examination of the Degtjareff method for determining soil organic matter, and a proposed modification of the chromic acid titration method. Soil Science 37, 29-38.). To assess the prevalence of different types of carbon on the TOC (i.e., to measure the stable, or “recalcitrant” organic carbon), methods may be employed to fractionate to TOC by, for example, measuring soil respiration or the bulk density of the soil.

In embodiments of the invention, the fungal inoculation of soil and/or the plant results in an increase in soil aggregate stability. The increase in soil aggregate stability, or soil aggregation per se, of a soil that is subjected to the treatments and/or methods described herein compared to a control may be quantified by any methods known to those skilled in the art. The control would be a similar soil sample that had not been exposed to the relevant fungus (i.e., a fungus had not been deployed in the soil or associated with a plant that had been cultivated in said soil). “Similar soil sample” means that the soil would be from a proximal area with a similar climate and, if the soil had been cultivated, the control sample would have been cultivated by the same plant as the test soil. The soil aggregate stability may be quantified by measurements compared to controls such as, but not limited to, soil mean weight diameter (MWD), geometric mean diameter (GMD), fractal dimension (D), percentage of aggregates destruction (PAD) and water-stable aggregates stability rate (WSAR). An increase in the MWD, GMD, WSAR and D values are indicative of an increase in soil aggregate stability, while a decrease in PAD value is indicative of an increase in soil aggregate stability.

In various embodiments of the invention, the fungal inoculation may have one or more desirable effects on the soil and/or associated crop plants cultivated in the treated soil, including, but not limited to, sequestering atmospheric carbon for storage as stable carbon in the soil; providing agronomic benefits to the crop plants; increasing the levels of stabilised carbon in the soil used to cultivate the crop plants; and/or increasing the soil aggregate stability of the soil used to cultivate crop plants. In other embodiments of the invention, the fungal inoculation may have two or more of the aforementioned desirable effects on the soil and/or associated crop plants cultivated in the treated soil, or three or more of the aforementioned desirable effects on the soil and/or associated crop plants cultivated in the treated soil.

That the fungal inoculation of the methods of the invention may have numerous, simultaneous effects on the soil and/or associated crop plants cultivated in the treated soil is, in part, possible because some of the desirable effects contribute to other desirable effects. For example, increasing soil aggregate stability is related to the enhanced (and/or longer-term) storage of sequestered atmospheric carbon as well as providing agronomic benefits to said crop plants by virtue of stably aggregated soil being more productive through, for example, improved water retention. In another example, sequestration of atmospheric carbon by the melanised fungi as described herein can lead to an increase in the complex polysaccharides in the soil resulting in long-term storage of sequestered atmospheric carbon in a stable form.

### Fractionation of Soil Organic Carbon

In certain aspects, the disclosed mixtures increase organic carbon in the soil. Soil organic C (SOC) concentration in mineral soils (0-10 cm depth) varies from <0.2% to ≥11.6% C; above this concentration, a soil is classified as a ‘peat’ soil, for example, in peatlands or tundra lands.

Soil organic C in mineral soils contains a range of organic substances at various stages of decomposition such as plant materials-both produced aboveground (straw, litter) and belowground (roots, root exudates), fungal hyphae, soil fauna, and microbial biomass and their products. Organic compounds include lipids, proteins, carbohydrates, quinones, and their derivatives. Major functional groups include alkyl C (10-45 ppm), N-alkyl and methoxy C (45-60 ppm), O-alkyl C (60-110 ppm), aromatic C (110-145 ppm), phenolic C (145-165 ppm), and amide and carboxyl C (165-215 ppm), as identified in 13C NMR spectra of SOC (Almeida et al. 2021). These are also grouped as aromatic, aliphatic and polysaccharide groups.

Since SOC consists of different C substances, which turnover (decompose) at different rates, persist in soil for different periods, stabilize with minerals with different mechanisms, and contribute to bio-physico-chemical functions in separate ways (Chenu et al. 2015). Most components of SOC are separated by chemical oxidation (acids, alkali, oxidants), biological (decomposition rates, microbial respiration), and physical methods. Of these, physical methods are preferred because these methods cause minimum disturbance, disruption, and alteration of SOC substances in soil. The physical methods are based on density, size, and sedimentation of soil, and justification of these methods are given by Poeplau et al. (2018).

Density, size, and sedimentation procedures broadly fractionate SOC or SOM in three groups, fPOC or fPOM, aggregate occluded particulate organic carbon (POC or POM) and silt+clay size associated or fine mineral-associated organic C (MAOC or MAOM). Dissolved organic C, DOC or DOM are also measured and plays a significant role in MAOM formation although it accounts for <2% of total Plant C or SOC.

The stabilized MAOM are separated from labile organic matter using density and size fractionation procedure (Poeplau et al. 2018; Mayer et al. 2022; Rodrigues et al. 2022). The organic matter that floats in the heavy density liquid, either sodium iodide (NAI) solution or sodium polytungstate (SPT, Nₐ₆[H₂W₁₂O₄₀] or 3Na₂WO₄·9WO₃·H₂O) at 1.8 Mg m⁻³ (1.8 g cm⁻³) or <1.8 Mg m⁻³ density soil organic matter are considered as fPOM. After separation of fPOM, occluded particulate organic matter within aggregates, oPOM and MAOM are usually dispersed either using ultrasonic energy or sodium hexametaphosphate (Na₆[(PO₃)₆] solution to disperse the soil particles. In the former, ultrasonic energy, ~400-500 J mL⁻¹ is applied to the soil in the SPT solution (1.8 Mg m-3) contained in a temperature-controlled container. Excess oPOM is separated, and the remaining soil is sieved through 53 μm sieve to collect<53 μm as MAOM. The >53 μm fraction remaining on the sieve is considered the organic matter in the sand-size fraction, usually containing a small amount of organic C and could be added to the fPOM fraction. This is circumvented when after separation of fPOM, excess SPT is washed from the remaining soil and the soil is dispersed in sodium hexametaphosphate and sieved through a 53 μm sieve to collect<53 μm as MAOM. The >53 μm fraction that remained on the sieve is considered the organic matter occluded in the sand-size aggregates or aggregate C since sand fraction contains only a small amount of organic C. Thus, SOC is separated into three fractions: fPOM, oPOM, and MAOM. Obviously, dissolved organic matter (DOM) is either lost or added to the MAOM fraction, although, in most mineral soils, DOM constitutes <2% of SOC (Poeplau et al. 2018). For practical purposes and routine SOC fractionation, SOM can be separated into >53 μm POM (fPOM+oPOM) and <53 μm MAOM (Lavallee et al. 2019).

The MAOM fraction provides the long-term storage of SOC (Kleber et al. 2015; Hemingway et al. 2019). However, MAOM is subject to the C saturation of fine silt+clay (<53 μm) or fine mineral fraction, which is dependent on silt+clay contents (Feng et al. 2013) and their mineralogy, Fe and Al (hydro-) oxides, specific surface area, soil architecture, nature of organic C inputs, especially their C and N contents, and soil pH. Once the C saturation of mineral fraction is achieved, further SOC sequestration for the long-term storage as MAOM is not likely to occur. However, the potential turnover through C mineralization and fresh C addition may still be required (Mayer et al. 2022; Rodrigues et al. 2022). From the boundary line approach, Feng et al. (2013) estimated that the silt±clay size fraction (<2 μm) may store 84±1 g C kg⁻¹ silt±clay size fraction in 2:1 clay dominant (smectite, illite, vermiculite) soil, and 43±1 g C kg⁻¹ silt±clay in 1:1 clay dominant (kaolinite) soil. This provides a ‘rule of thumb’ estimate to identify soil, in which SOC may be sequestered long-term in the MAOM fraction. It is worth noting here that the 20-53 μm MAOM fraction may contain silt-size micro-aggregates, which may have a faster turnover rate than the <20 μm MAOM.

Further, organic C. may not uniformly cover the surface of the fine mineral fraction. For example, Schweizer et al. (2021) found that clay surfaces of the soil containing low clay contents (5-18%) had twice as much organic C in the MAOM than the high clay soils.

It is sobering to note that long-term field experiments have shown that it is less likely that SOC will be sequestered in the stabilized MAOM fraction if this fraction is already saturated (Mayer et al., 2022; Rodrigues et al., 2022), and further C inputs will be stabilized in the oPOM fraction (occluded in aggregates) or remain fPOM. However, oPOM is readily lost when the soil is disturbed. Furthermore, it is not known whether there is a saturation limit for the oPOM fraction. fPOM can be increased in the soil indefinitely although this fraction is affected by global warming and quantity and quality of continuous C inputs more than the other SOM fractions (Lugato et al. 2021; Rocci et al. 2021).

SOC fractionation protocols vary widely. Therefore, Poeplau et al (2018) compared two protocols in use at the time of the study. Details of these protocols and a preferred protocol are given by Poeplau et al. (2018). In conclusion, they found that no SOC fraction identified the rapid turnover rate component, that particle-size separation was better for separating the fPOM than that the oPOM incorporated into aggregates, and the separation of silt+clay-size fraction from the sand-size fraction was the most effective protocol in identifying fractions of different turnover rates. Admittedly, since microbial inoculants in the rhizosphere may be involved in aggregation (Mugerwa and McGee 2017; Buss et al. 2021) and, therefore, soil structure, it is recommended that in such situations oPOM separation may be desirable to detect the effect of inoculants in C accumulation in the rhizosphere of the microbial inoculated plants. However, as stated above, since the turnover rate of oPOM is uncertain, and subject to disturbance, it should not be considered for long-term C sequestration in soil.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

All headings are for the reader's convenience and should not be used to limit the meaning of the text that follows the heading, unless so specified.

The present invention is further illustrated by the following examples that should not be construed as limiting. The contents of all references, patents, and published patent applications cited throughout this application and the Figures are incorporated herein by reference in their entirety for all purposes. In order to exemplify the nature of the present invention such that it may be more clearly understood, the following non-limiting examples are provided.

### EXAMPLES

### Example 1. Beauveria bassiana AU-16727 (NMI Accession No. V23/003855) Applied to Cotton Increases Yield and Soil Carbon

### Background

To evaluate the effects of Beauveria bassiana AU-16727 (NMI Accession No. V23/003855) on soil carbon and crop yield, a cotton field trial was conducted in Toowoomba, Queensland, Australia.

### Materials and Methods

Beauveria bassiana AU-16727 (NMI Accession No. V23/003855) was applied directly to cotton seed as a slurry at a rate of 20 mL per kg of seed immediately prior to sowing with a carrier. Untreated cotton seeds were sown as a control. Six replicates were evaluated for each group. The average total carbon (TC) and average yield in each group were determined at harvest. TC measurements were performed with either a HONE CARBON® sensor using near infrared spectroscopy or a LECO® instrument using combustion of carbon.

### Results

Results for TC and crop yield are shown in Tables 8 and 9, respectively.

TABLE 8

| Treatment | TC (%) | Change Compared to Control |
|---|---|---|
| Untreated Control | 1.55 | — |
| AU-16727 | 1.58 | +1.9% |

TABLE 9

| Treatment | Yield (T/Ha) | Change Compared to Control |
|---|---|---|
| Untreated Control | 3.19 | — |
| AU-16727 | 3.47 | +8.8% |

Beauveria bassiana AU-16727 (NMI Accession No. V23/003855) improved both TC and yield in the cotton field trial.

### Example 2. Beauveria bassiana AU-16727 (NMI Accession No. V23/003855) Applied to Barley Increases Yield and Soil Carbon

### Background

To evaluate the effects of Beauveria bassiana AU-16727 (NMI Accession No. V23/003855) on soil carbon and crop yield, a barley field trial was conducted in Corowa, New South, Australia.

### Materials and Methods

Beauveria bassiana AU-16727 (NMI Accession No. V23/003855) was applied directly to barley seed at a rate of 0.1 g fungal inoculum per kg seed immediately prior to sowing with a carrier. The fungal inoculum contained about 1.4×10⁹ colony forming units (CFU) per gram, so the application rate was about 1.4×10⁸ CFU per kg seed. Untreated barley seeds were sown as a control. Between 18 and 20 replicates were evaluated for each group for carbon measurements, and six replicates were evaluated for each group for yield measurements. The average total carbon (TC) and average yield in each group were determined at harvest. TC measurements were performed with either a HONE CARBON® sensor using near infrared spectroscopy or a LECO® instrument using combustion of carbon.

### Results

Results for TC and crop yield are shown in Tables 10 and 11, respectively.

TABLE 10

| Treatment | TC (%) | Change Compared to Control |
|---|---|---|
| Untreated Control | 1.40 | — |
| AU-16727 | 1.57 | +12.1% |

TABLE 11

| Treatment | Yield (T/Ha) | Change Compared to Control |
|---|---|---|
| Untreated Control | 4.06 | — |
| AU-16727 | 4.11 | +1.23% |

Beauveria bassiana AU-16727 (NMI Accession No. V23/003855) improved both TC and yield in the barley field trial.

### Example 3. Beauveria bassiana AU-16727 (NMI Accession No. V23/003855) Applied to Soybeans Increases Soil Carbon

### Background

To evaluate the effects of Beauveria bassiana AU-16727 (NMI Accession No. V23/003855) on soil carbon, a soybean field trial was conducted in Bundaberg, Queensland, Australia.

### Materials and Methods

Beauveria bassiana AU-16727 (NMI Accession No. V23/003855) was applied directly to soybean seed at a rate of 0.5 g per 1.5 kg seed immediately prior to sowing with a carrier. The fungal inoculum contained about 1.4×10⁹ colony forming units (CFU) per gram, so the application rate was about 4.7×10⁸ CFU per kg seed. Untreated soybean seeds were sown as a control. All soybeans were grown under standard conditions which included the application of rhizobium. Six replicates were evaluated for each group. The average total carbon (TC) in each group was determined at harvest. TC measurements were performed with either a HONE CARBON® sensor using near infrared spectroscopy or a LECO® instrument using combustion of carbon.

### Results

Results for TC are shown in Table 12.

TABLE 12

| Treatment | TC (%) | Change Compared to Control |
|---|---|---|
| Untreated Control | 1.168 | — |
| AU-16727 | 1.181 | +1.1% |

Beauveria bassiana AU-16727 (NMI Accession No. V23/003855) improved TC in the soybean field trial.

### Example 4. Beauveria bassiana AU-16727 (NMI Accession No. V23/003855) Applied to Ryegrass Increases Soil Carbon

### Background

To evaluate the effects of Beauveria bassiana AU-16727 (NMI Accession No. V23/003855) on soil carbon, three ryegrass field trials were conducted in Canowindra, New South Wales; Naracoorte, South Australia; and Young, New South Wales in Australia.

### Materials and Methods

Beauveria bassiana AU-16727 (NMI Accession No. V23/003855) was applied directly to ryegrass seeds at a rate of 0.6 g per 150 g seed at a concentration of about 1.4×10⁹ colony forming units (CFU) per gram immediately prior to sowing with a carrier. Thus, the application rate was about 5.6×10⁹ CFU per kg seed. Untreated ryegrass seeds were sown as a control. 19 or 20 replicates were evaluated for each group. The average total carbon (TC) in each group was determined at harvest. TC measurements were performed with either a HONE CARBON® sensor using near infrared spectroscopy or a LECO® instrument using combustion of carbon.

### Results

Results for TC are shown in Table 13.

TABLE 13

| Trial | | | Change Compared |
|---|---|---|---|
| Location | Treatment | TC (%) | to Control |
| Canowindra | Untreated Control | 0.744 | — |
| | AU-16727 | 0.920 | +23.7% |
| Naracoorte | Untreated Control | 0.707 | — |
| | AU-16727 | 0.780 | +10.3% |
| Young | Untreated Control | 1.47 | — |
| | AU-16727 | 1.54 | +4.8% |

Beauveria bassiana AU-16727 (NMI Accession No. V23/003855) improved TC across all three ryegrass field trials.

### Example 5. Beauveria bassiana AU-16727 (NMI Accession No. V23/003855) Applied to Wheat Increases Soil Carbon

### Background

To evaluate the effects of Beauveria bassiana AU-16727 (NMI Accession No. V23/003855) on soil carbon, a wheat field trial was conducted in Beverley, Western Australia.

### Materials and Methods

Beauveria bassiana AU-16727 (NMI Accession No. V23/003855) was applied directly to wheat seeds at a rate of 10 g per hectare at a concentration of about 1.4×10⁹ colony forming units (CFU) per gram immediately prior to sowing with a carrier. Thus, the application rate was about 1.4×10¹⁰ CFU per hectare. Untreated wheat seeds were sown as a control. Six replicates were evaluated for each group. The average total carbon (TC) in each group was determined at harvest. TC measurements were performed with either a HONE CARBON® sensor using near infrared spectroscopy or a LECO® instrument using combustion of carbon.

### Results

Results for TC are shown in Table 14.

TABLE 14

| Treatment | TC (%) | Change Compared to Control |
|---|---|---|
| Untreated Control | 1.61 | — |
| AU-16727 | 1.76 | +9.3% |

Beauveria bassiana AU-16727 (NMI Accession No. V23/003855) improved TC in the wheat field trial.

### Example 6. Beauveria bassiana US-52 (ATCC Accession No. PTA-127541) Applied to Corn Increases Soil Carbon

### Background

To evaluate the effects of Beauveria bassiana US-52 (ATCC Accession No. PTA-127541) on soil carbon, two corn field trials were conducted in Greenville, Mississippi and in Troy, Ohio in the United States.

### Materials and Methods

Beauveria bassiana US-52 (ATCC Accession No. PTA-127541) was applied directly to corn seeds at a rate of about 5,000 CFU per seed immediately prior to sowing with a carrier. Untreated corn seeds were sown as a control. Five replicates were evaluated for each group. The average total carbon (TC) in each group was determined at harvest. TC measurements were performed with either a HONE CARBON® sensor using near infrared spectroscopy or a LECO® instrument using combustion of carbon.

### Results

Results for TC are shown in Table 15.

TABLE 15

| Trial | | | Change Compared |
|---|---|---|---|
| Location | Treatment | TC (%) | to Control |
| Greenville | Untreated Control | 0.7392 | — |
| | US-52 | 0.7860 | +6.3% |
| Troy | Untreated Control | 1.6900 | — |
| | US-52 | 1.7198 | +1.8% |

Beauveria bassiana US-52 (ATCC Accession No. PTA-127541) improved TC in the two corn field trials.

### Example 7. Beauveria bassiana US-52 (ATCC Accession No. PTA-127541) Applied to Barley Increases Yield

### Background

To evaluate the effects of Beauveria bassiana US-52 (ATCC Accession No. PTA-127541) on crop yield, a barley field trial were conducted in Olivia, Minnesota in the United States.

### Materials and Methods

Beauveria bassiana US-52 (ATCC Accession No. PTA-127541) was applied directly to barley seeds at a rate of about 5,000 CFU per seed immediately prior to sowing with a carrier. Untreated barley seeds were sown as a control. Three replicates were evaluated for each group. The average yield in each group was determined at harvest.

### Results

Results for yield measurements are shown in Table 16.

TABLE 16

| | | Change Compared |
|---|---|---|
| Treatment | Yield (Bushels/Acre) | to Control |
| Untreated Control | 60.81 | — |
| US-52 | 69.87 | +14.9% |

Beauveria bassiana US-52 (ATCC Accession No. PTA-127541) improved yield in the barley field trial.

### Example 8. Beauveria bassiana US-52 (ATCC Accession No. PTA-127541) Applied to Soybeans Increases Yield

### Background

To evaluate the effects of Beauveria bassiana US-52 (ATCC Accession No. PTA-127541) on crop yield, two soybean field trials were conducted in Sanborn, Iowa and in Olivia, Minnesota in the United States.

### Materials and Methods

Beauveria bassiana US-52 (ATCC Accession No. PTA-127541) was applied directly to soybean seeds at a rate of about 5,000 CFU per seed immediately prior to sowing with a carrier. Untreated soybean seeds were sown as a control. Five replicates were evaluated for each group. The average yield in each group was determined at harvest.

### Results

Results for yield measurements are shown in Table 17.

TABLE 17

| Trial | | Yield | Change Compared |
|---|---|---|---|
| Location | Treatment | (Bushels/Acre) | to Control |
| Sanborn | Untreated Control | 66.34 | — |
| | US-52 | 71.34 | +7.5% |
| Olivia | Untreated Control | 51.996 | — |
| | US-52 | 53.160 | +2.2% |

Beauveria bassiana US-52 (ATCC Accession No. PTA-127541) improved yield in the two soybean field trials.

### Example 9. Beauveria bassiana US-803 (ATCC Accession No. PTA-127539) Applied to Barley Increases Soil Carbon and Yield

### Background

To evaluate the effects of Beauveria bassiana US-803 (ATCC Accession No. PTA-127539) on soil carbon and crop yield, a barley field trial was conducted in Olivia, Minnesota in the United States.

### Materials and Methods

Beauveria bassiana US-803 (ATCC Accession No. PTA-127539) was applied directly to barley seeds at a rate of about 5,000 CFU per seed immediately prior to sowing with a carrier. Untreated barley seeds were sown as a control. Three TC replicates for each group were evaluated at baseline (i.e., prior to sowing) and at harvest, and the changes in TC measurements over time for each group were determined. Three yield replicates were also evaluated for each group.

### Results

Results for TC and yield measurements are shown in Tables 18 and 19, respectively.

TABLE 18

| | | | Harvest TC − | % Change |
|---|---|---|---|---|
| | Baseline TC | Harvest TC | Baseline TC | Relative to |
| Treatment | (%) | (%) | (%) | Baseline TC |
| Untreated | 4.163 | 4.047 | −0.116 | −2.8% |
| Control | | | | |
| US-803 | 2.908 | 3.319 | +0.411 | +14.1% |

TABLE 19

| | | Change Compared |
|---|---|---|
| Treatment | Yield (Bushels/Acre) | to Control |
| Untreated Control | 60.81 | — |
| US-803 | 71.70 | +17.9% |

Beauveria bassiana US-803 (ATCC Accession No. PTA-127539) improved TC over time whereas the untreated control decreased in TC. Beauveria bassiana US-803 (ATCC Accession No. PTA-127539) also improved yield in the barley field trial.

### Example 10. Beauveria bassiana US-52 (ATCC Accession No. PTA-127541) Spores Demonstrate Stability at 24° C. and 30° C.

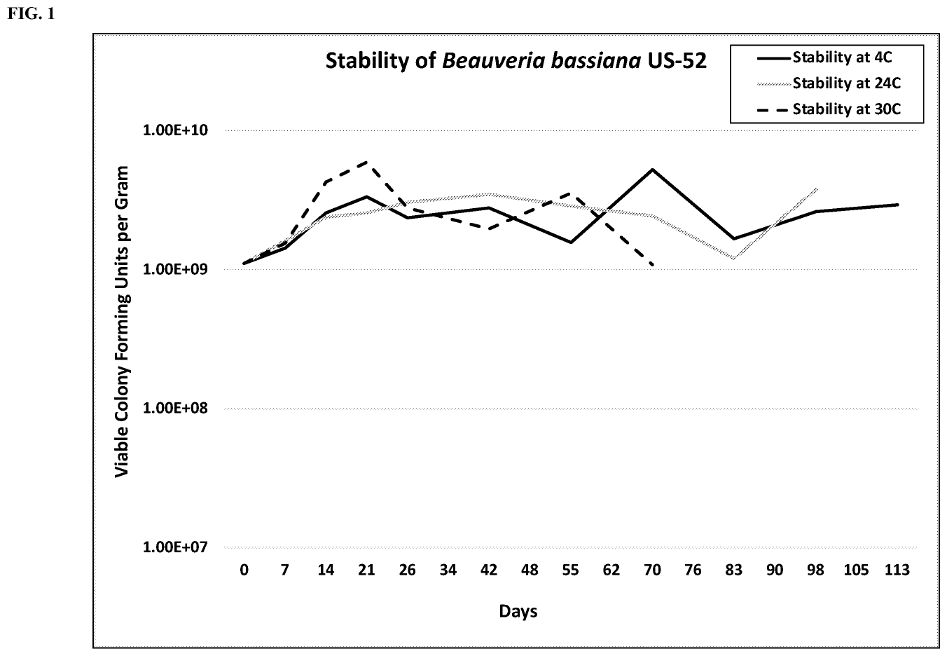

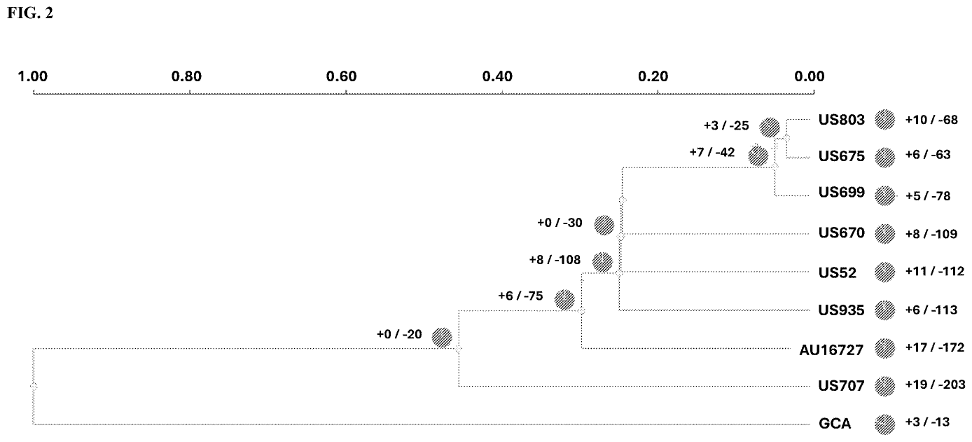

A powder spore preparation of Beauveria bassiana US-52 (ATCC Accession No. PTA-127541) spores was separated into three groups to be stored at 4° C., 24° C., or 30° C. No stabilizing agents were added to the spore preparation. At intervals of about every 7 days, each group was evaluated for the number of viable colony forming units per gram of spore preparation with two replicates of each group assessed. This study continued for between 70 days and 113 days with average values of viable CFU/g presented in FIG. 1. Beauveria bassiana US-52 (ATCC Accession No. PTA-127541) spores were stable for at least 70 days at 24° C. and 30° C.

### Example 11. Bioinformatic Assessment of Beauveria bassiana Strains

### Background

Certain strains of Beauveria bassiana are used commercially as biological control agents due to their broad host range that covers many insect orders. B. bassiana is known for having a broad genetic host and virulence diversity. Thus, high sequence DNA variation between different isolates is found and has been reported to be associated with different levels of virulence (Valero-Jiménez et al. 2016).

B. bassiana strains are also able to colonize plants and they are proposed to have an impact on root growth (Ortiz-Urquiza et al. 2021, Mantzoukas et al. 2021). Here we investigated genetic factors that contribute to carbon sequestering capabilities in B. bassiana. We aimed to identify B. bassiana strains with increased carbon sequestration properties and decreased potential for insect virulence.

The genomes of the following B. bassiana strains were used for bioinformatic analysis and comparison: B. bassiana US-52 (ATCC Accession No. PTA-127541), B. bassiana US-675 (ATCC Accession No. PTA-127540), B. bassiana US-699 (ATCC Accession No. PTA-127538), B. bassiana US-707 (ATCC Accession No. PTA-127542), B. bassiana US-803 (ATCC Accession No. PTA-127539), B. bassiana AU-16727 (NMI Accession No. V23/003855), B. bassiana US-935 (ATCC Accession No. PTA-127743), and B. bassiana US-670 (ATCC Accession No. PTA-127741).

Comparative Analysis of B. bassiana Genomes

Genome Assembly: Quality trimming was performed using Trimmomatic version 0.39, using a sliding window of 4:15, trimming off basses from the start and end of the reads below a quality threshold of 3, and dropping any reads below a length of 35 bp (Bolger et al. 2014). De novo assembly of paired end reads were performed using SPAdes (version 3.15.2) using the “--isolate” option with kmer lengths of 21, 33, 55, 77, 99, and 127 (Bankevich et al. 2012).

Gene Prediction: The assembled genomes were annotated using the Funannotate pipeline (https://funannotate.readthedocs.io/en/latest/predict.html#predict). Repeats were first masked using RepeatMasker version 4.1.2 (Smit, A F A, Hubley, R & Green, P. RepeatMasker Open-4.0. 2013-2015<http://www.repeatmasker.org>.). Genes were predicted using multiple ab initio gene prediction models, including: Augustus, snap, glimmerHMM, and GeneMark-ES/ET, which were all fed into Evidence Modeler to create a consensus gene model (Stanke et al. 2006, Korf, Majoros et al. 2004, Ter-Hovhannisyan et al. 2008, Haas et al. 2008).

Table 20 shows the genome size of B. bassiana strains. The B. bassiana US-707 (ATCC Accession No. PTA-127542) genome is larger by about 2 Mb compared to the other B. bassiana genomes.

TABLE 20

| Strain | Total Genome Length | GC Content (%) |
|---|---|---|
| B. bassiana US-803 | 34570320 | 49.84 |
| B. bassiana US-707 | 36616240 | 48.38 |
| B. bassiana US-699 | 34576819 | 49.84 |
| B. bassiana US-675 | 34584885 | 49.84 |
| B. bassiana US-52 | 34552029 | 49.85 |
| B. bassiana US-670 | 34165975 | 50.08 |
| B. bassiana US-935 | 34293267 | 49.93 |
| B. bassiana AU-16727 | 34314819 | 49.82 |

The proteomes were clustered using Orthovenn3 to identify and annotate orthologous clusters and infer phylogenetic relationships across a range of species (Sun et al., 2023). The genome of a virulent B. bassiana strain (i.e., GCA_001682635) know to infect mosquitos was included in the analysis as a point of comparison (Valero-Jiménez et al., 2016)). Together with B. bassiana GCA_001682635, B. bassiana US-707 (ATCC Accession No. PTA-127542) and B. bassiana AU-16727 (NMI Accession No. V23/003855) had the most singletons indicating the widest divergence from the other strains (see Table 21).

TABLE 21

| Strain | Proteins | Clusters | Singletons |
|---|---|---|---|
| B. bassiana | 10210 | 9220 | 807 |
| GCA_001682635 | | | |
| B. bassiana US-707 | 9834 | 9353 | 331 |
| B. bassiana AU-16727 | 9651 | 9323 | 209 |
| B. bassiana US-699 | 9698 | 9567 | 21 |
| B. bassiana US-803 | 9681 | 9540 | 29 |
| B. bassiana US-675 | 9684 | 9539 | 33 |
| B. bassiana US-52 | 9700 | 9558 | 36 |
| B. bassiana US-670 | 9662 | 9531 | 26 |
| B. bassiana US-935 | 9698 | 9547 | 52 |

Orthovenn3 uses CAFE5 to calculate the contraction and expansion of a given gene family. Expansions are increases (indicated with “+”) in gene families while contractions are decreases (indicated with “−”) (Mendez et al., 2020). B. bassiana US-707 (ATCC Accession No. PTA-127542) was found to have the most expansions (+19) and contractions (−203) of the strains.

Potential of Beauveria bassiana Strains for Carbon Sequestration

Carbon sequestration driven by fungal species involves multiple mechanisms that are the result of the plant-microbial community-soil chemistry biosystem. While there is a need for understanding the implications of each mechanism on carbon sequestration, there is evidence of several molecular pathways that lead to increased organic carbon in soil. To analyze B. bassiana carbon sequestration capabilities, we analyzed the following know mechanims:
Determination of copy number of proteins involved in the melanin biosynthetic pathway
Analysis of carbohydrate active enzymes
Analysis of enzymes involved in lignin degradation

### Analysis of Domains Involved in Melanin Biosynthetic Pathway

One way in which fungi stabilize soil organic carbon (SOC) is by transforming carbon compounds to stable carbon compounds such as chitin and melanin (Fernandez et al. 2019, Gayathri et al. 2021, Lenaers et al. 2018, McGee et al. 2013). The genomic analysis of proteins involved in the melanin biosynthetic pathway allows correlation of fungal soil organic carbon sequestration capabilities with melanin associated proteins.

The copy number of various proteins involved in the melanin biosynthetic pathway was determined with the genomes of B. bassiana US-52 (ATCC Accession No. PTA-127541), B. bassiana US-675 (ATCC Accession No. PTA-127540), B. bassiana US-699 (ATCC Accession No. PTA-127538), B. bassiana US-707 (ATCC Accession No. PTA-127542), B. bassiana US-803 (ATCC Accession No. PTA-127539), B. bassiana AU-16727 (NMI Accession No. V23/003855), B. bassiana US-935 (ATCC Accession No. PTA-127743), and B. bassiana US-670 (ATCC Accession No. PTA-127741). Protein domains were identified using interproscan (Quevillon E, et al. 2015). Polyketide synthase dehydratase domains were identified searching for the PFAM domain, PF14765 (Mistry et al. 2021). Of these strains, all strains contains 20 PKS domains, with the exception of B. bassiana AU-16727 (NMI Accession No. V23/003855) and B. bassiana US-707 (ATCC Accession No. PTA-127542), with 15 and 14, respectively (see Table 22).

TABLE 22

| | Polyketide Synthase Dehydratase |
|---|---|
| Strain | (PKS) Domains |
| B. bassiana US-935 | 20 |
| B. bassiana US-675 | 20 |
| B. bassiana US-52 | 20 |
| B. bassiana US-699 | 20 |
| B. bassiana US-803 | 20 |
| B. bassiana US-670 | 20 |
| B. bassiana AU-16727 | 15 |
| B. bassiana US-707 | 14 |

### Carbohydrate Active Enzymes

Microbial communities catabolize energy-rich carbohydrates such as cellulose, starch, glycogen. Carbohydrate active enzymes are key enzymes in the degradation of soil organic matter. Carbohydrate active enzymes are classified into different groups that are related to specific activities, some of which are important in the dynamics of soil carbon (Salam 2018; Gong et al., 2022; Merino et al., 2016; Yan et al., 2022). The CAZy database was used to compare B. bassiana genes with the corresponding enzymes. Table 23 represents the copy number for every enzyme group present in each strain.

TABLE 23

| ID | US-935 | US-52 | US-670 | US-675 | AU-16727 | US-803 | US-707 | US-699 |
|---|---|---|---|---|---|---|---|---|
| AA1 | 1 | 1 | 1 | 1 | 1 | 1 | 1 | 1 |
| AA11 | 4 | 4 | 4 | 4 | 3 | 4 | 5 | 5 |
| AA12 | 2 | 2 | 2 | 2 | 2 | 2 | 2 | 2 |
| AA16 | 0 | 0 | 0 | 0 | 0 | 0 | 0 | 0 |
| AA1_2 | 2 | 2 | 2 | 2 | 2 | 2 | 2 | 2 |
| AA1_3 | 3 | 3 | 2 | 3 | 1 | 3 | 3 | 3 |
| AA2 | 2 | 2 | 2 | 2 | 2 | 2 | 2 | 2 |
| AA3_1 | 1 | 1 | 1 | 1 | 1 | 1 | 1 | 1 |
| AA3_2 | 3 | 3 | 3 | 3 | 3 | 3 | 3 | 3 |
| AA5_2 | 1 | 1 | 1 | 1 | 0 | 1 | 1 | 1 |
| AA7 | 8 | 10 | 10 | 9 | 10 | 9 | 9 | 9 |
| AA8 | 2 | 2 | 2 | 2 | 1 | 2 | 2 | 2 |
| AA9 | 1 | 0 | 0 | 1 | 0 | 0 | 0 | 0 |
| CBM20 | 1 | 1 | 1 | 1 | 1 | 1 | 1 | 1 |
| CBM38 | 0 | 0 | 0 | 0 | 0 | 0 | 0 | 0 |
| CBM42 | 1 | 1 | 1 | 1 | 1 | 1 | 1 | 1 |
| CBM43 | 1 | 1 | 1 | 1 | 1 | 1 | 1 | 1 |
| CBM52 | 1 | 1 | 1 | 1 | 1 | 1 | 1 | 1 |
| CBM63 | 0 | 0 | 0 | 0 | 0 | 0 | 0 | 0 |
| CBM66 | 3 | 3 | 3 | 3 | 3 | 3 | 3 | 3 |
| CE1 | 1 | 1 | 1 | 1 | 2 | 1 | 1 | 1 |
| CE12 | 0 | 0 | 0 | 0 | 0 | 0 | 0 | 0 |
| CE16 | 0 | 0 | 0 | 0 | 0 | 0 | 1 | 0 |
| CE2 | 0 | 0 | 0 | 0 | 0 | 0 | 0 | 0 |
| CE3 | 1 | 1 | 1 | 1 | 1 | 1 | 1 | 1 |
| CE4 | 0 | 0 | 0 | 0 | 0 | 0 | 0 | 0 |
| CE5 | 4 | 5 | 4 | 5 | 5 | 4 | 5 | 5 |
| CE8 | 0 | 0 | 0 | 0 | 0 | 0 | 0 | 0 |
| GH10 | 1 | 1 | 1 | 1 | 1 | 1 | 1 | 1 |
| GH106 | 0 | 0 | 0 | 0 | 0 | 0 | 0 | 0 |
| GH11 | 0 | 0 | 0 | 0 | 0 | 0 | 0 | 0 |
| GH12 | 1 | 1 | 1 | 1 | 1 | 1 | 1 | 1 |
| GH125 | 3 | 3 | 3 | 3 | 3 | 3 | 3 | 3 |
| GH128 | 3 | 3 | 3 | 3 | 3 | 3 | 3 | 3 |
| GH132 | 2 | 2 | 2 | 2 | 2 | 2 | 2 | 2 |
| GH135 | 0 | 0 | 0 | 0 | 0 | 0 | 0 | 0 |
| GH136 | 1 | 1 | 1 | 1 | 1 | 1 | 1 | 1 |
| GH13_1 | 1 | 1 | 1 | 1 | 1 | 1 | 1 | 1 |
| GH13_22 | 0 | 0 | 0 | 0 | 0 | 0 | 0 | 0 |
| GH15 | 1 | 1 | 1 | 1 | 1 | 1 | 1 | 1 |
| GH162 | 0 | 0 | 0 | 0 | 0 | 0 | 0 | 0 |
| GH16_1 | 5 | 5 | 5 | 5 | 5 | 5 | 5 | 5 |
| GH16_18 | 5 | 5 | 6 | 6 | 6 | 6 | 6 | 6 |
| GH16_19 | 1 | 1 | 1 | 1 | 1 | 1 | 0 | 1 |
| GH16_22 | 1 | 1 | 1 | 1 | 1 | 1 | 1 | 1 |
| GH16_23 | 2 | 2 | 2 | 2 | 1 | 2 | 2 | 2 |
| GH16_3 | 2 | 2 | 2 | 2 | 2 | 2 | 2 | 2 |
| GH17 | 1 | 1 | 1 | 1 | 1 | 1 | 1 | 1 |
| GH171 | 1 | 1 | 1 | 1 | 1 | 1 | 1 | 1 |
| GH18 | 10 | 12 | 11 | 12 | 11 | 12 | 10 | 12 |
| GH2 | 2 | 2 | 2 | 3 | 2 | 2 | 2 | 3 |
| GH20 | 2 | 2 | 2 | 2 | 2 | 2 | 3 | 2 |
| GH25 | 1 | 1 | 1 | 1 | 1 | 1 | 1 | 1 |
| GH27 | 2 | 2 | 2 | 2 | 2 | 2 | 2 | 2 |
| GH28 | 1 | 1 | 1 | 1 | 1 | 1 | 0 | 1 |
| GH29 | 2 | 2 | 2 | 2 | 2 | 2 | 2 | 2 |
| GH3 | 4 | 5 | 4 | 4 | 3 | 5 | 3 | 5 |
| GH30_3 | 0 | 0 | 0 | 0 | 0 | 0 | 0 | 0 |
| GH31 | 4 | 4 | 4 | 4 | 4 | 4 | 4 | 4 |
| GH32 | 1 | 1 | 1 | 1 | 1 | 1 | 1 | 1 |
| GH35 | 3 | 3 | 3 | 3 | 3 | 3 | 3 | 3 |
| GH36 | 1 | 1 | 1 | 1 | 1 | 1 | 1 | 1 |
| GH37 | 1 | 1 | 1 | 1 | 1 | 1 | 1 | 1 |
| GH43_30 | 0 | 0 | 0 | 0 | 0 | 0 | 0 | 0 |
| GH43_6 | 0 | 0 | 0 | 0 | 0 | 0 | 0 | 0 |
| GH47 | 2 | 3 | 3 | 3 | 2 | 3 | 2 | 3 |
| GH49 | 0 | 0 | 0 | 0 | 0 | 0 | 0 | 0 |
| GH53 | 0 | 0 | 0 | 0 | 0 | 0 | 0 | 0 |
| GH54 | 1 | 1 | 1 | 1 | 1 | 1 | 1 | 1 |
| GH55 | 3 | 2 | 2 | 3 | 2 | 3 | 3 | 3 |
| GH5_11 | 1 | 1 | 1 | 1 | 1 | 1 | 1 | 1 |
| GH5_15 | 1 | 1 | 1 | 1 | 1 | 1 | 1 | 1 |
| GH5_24 | 0 | 0 | 0 | 0 | 0 | 0 | 0 | 0 |
| GH5_27 | 1 | 1 | 1 | 1 | 1 | 1 | 1 | 1 |
| GH5_5 | 1 | 1 | 1 | 1 | 1 | 1 | 1 | 1 |
| GH5_7 | 0 | 0 | 0 | 0 | 0 | 0 | 0 | 0 |
| GH5_9 | 0 | 0 | 0 | 0 | 0 | 0 | 0 | 0 |
| GH62 | 0 | 0 | 0 | 0 | 0 | 0 | 0 | 0 |
| GH63 | 1 | 1 | 1 | 1 | 1 | 1 | 1 | 1 |
| GH65 | 0 | 0 | 0 | 0 | 0 | 0 | 0 | 0 |
| GH67 | 0 | 0 | 0 | 0 | 0 | 0 | 0 | 0 |
| GH7 | 0 | 0 | 0 | 0 | 0 | 0 | 0 | 0 |
| GH72 | 6 | 6 | 6 | 6 | 6 | 6 | 6 | 6 |
| GH75 | 2 | 2 | 2 | 2 | 1 | 2 | 2 | 2 |
| GH76 | 7 | 6 | 7 | 7 | 6 | 7 | 7 | 7 |
| GH78 | 0 | 0 | 0 | 0 | 0 | 0 | 0 | 0 |
| GH79 | 2 | 2 | 2 | 2 | 2 | 2 | 2 | 2 |
| GH84 | 1 | 1 | 1 | 1 | 1 | 1 | 1 | 1 |
| GH89 | 2 | 2 | 2 | 2 | 1 | 2 | 2 | 2 |
| GH92 | 4 | 4 | 3 | 4 | 2 | 3 | 3 | 3 |
| GH93 | 0 | 0 | 0 | 0 | 0 | 0 | 0 | 0 |
| GH95 | 0 | 0 | 0 | 0 | 0 | 0 | 1 | 0 |
| GT109 | 1 | 1 | 1 | 1 | 1 | 1 | 1 | 1 |
| GT17 | 1 | 1 | 1 | 1 | 1 | 1 | 1 | 1 |
| GT22 | 1 | 1 | 2 | 1 | 1 | 1 | 2 | 1 |
| GT24 | 1 | 1 | 0 | 1 | 0 | 1 | 0 | 0 |
| GT2_Glyco_trans_2_3 | 0 | 0 | 0 | 0 | 0 | 0 | 0 | 0 |
| GT32 | 1 | 1 | 0 | 1 | 0 | 1 | 1 | 1 |
| GT48 | 0 | 1 | 0 | 0 | 0 | 0 | 0 | 0 |
| GT59 | 0 | 0 | 0 | 0 | 0 | 0 | 0 | 0 |
| GT8 | 0 | 0 | 0 | 0 | 0 | 0 | 0 | 0 |
| GT90 | 1 | 1 | 1 | 1 | 1 | 1 | 1 | 1 |
| PL1_4 | 0 | 0 | 0 | 0 | 0 | 0 | 0 | 0 |
| PL20 | 0 | 0 | 0 | 0 | 0 | 0 | 0 | 0 |
| PL38 | 0 | 0 | 0 | 0 | 0 | 0 | 0 | 0 |
| PL7_4 | 0 | 1 | 1 | 1 | 1 | 1 | 1 | 1 |
| PL8_4 | 1 | 1 | 1 | 1 | 1 | 1 | 1 | 1 |
| Total | 150 | 156 | 151 | 158 | 141 | 155 | 153 | 157 |

B. bassiana US-675 (ATCC Accession No. PTA-127540) was found to have the most predicted CAZymes with 158 while B. bassiana AU-16727 (NMI Accession No. V23/003855) had the least with 141. There are differences in copy number for several enzymes. B. bassiana US-707 (ATCC Accession No. PTA-127542) contains one enzyme count for groups CE16 and GH95. Carbohydrate esterases (CEs) involve enzymes that catalyze the de-O and de-N-acylation to remove esters from carbohydrates. The majority of CE enzymes remove acylated moieties of polysaccharides, which enhance their degradation (Salam et al., 2018). Furthermore, it has been proposed that CE16 collaborates with xylanase to complete degradation of xylooligosaccharides (Biely et al., 2014). In addition, GH95 present in B. bassiana US-707 (ATCC Accession No. PTA-127542) but not in the other strains is a glycoside hydrolase (GH). GHs are enzymes that cleave glycosidic bonds in glycosides, glucan, and glycoconjugates. GHs are known to play a role in global carbon cycling (Gougoulias et al., 2014).

### Analysis of Enzymes Involved in Lignin Degradation

In a previous study where enzymes involved in lignin depolymerization were analyzed, it was proposed that lignin degrading enzymes are important in soil ecosystems to facilitate carbon sequestration by fungi via the modification and utilization of aromatic lignin-derivatives (del Cerro et al., 2021). This analysis was extended to the B. bassiana strains with in silico genome analysis to predict catabolic pathways for the conversion of 4-hydroxybenzoic acid (4HBA).

To investigate the catabolic pathways related to the conversion of lignin-derived aromatic compounds, the genomes of B. bassiana US-52 (ATCC Accession No. PTA-127541), B. bassiana US-675 (ATCC Accession No. PTA-127540), B. bassiana US-699 (ATCC Accession No. PTA-127538), B. bassiana US-707 (ATCC Accession No. PTA-127542), B. bassiana US-803 (ATCC Accession No. PTA-127539), B. bassiana AU-16727 (NMI Accession No. V23/003855), B. bassiana US-935 (ATCC Accession No. PTA-127743), and B. bassiana US-670 (ATCC Accession No. PTA-127741) were compared. This comparison evaluates the known aromatic catabolic enzymes present in microorganisms including fungi (del Cerro et al., 2021; Holesova et al., 2011; Lah et al., 2011).

A homology comparison shows that the B. bassiana strains contain five enzymes involved in catabolism of the lignin derived compound 4HBA. Table 24 shows the copy number of enzymes involved in the conversion of the lignin derived compound 4HBA using an E-value cut-off of ≤1E-30.

TABLE 24

| | | | | Intradiol | |
|---|---|---|---|---|---|
| | | | | Dioxygenase | Fad-Binding |
| | 4-Hydroxymuconic | 2-Hydroxymuconic | | Domain- | 3 Domain- |
| | Semialdehyde | Semialdehyde | Benzenetriol | Containing | Containing |
| Strain | Dehydrogenase¹ | Dehydrogenase² | Dioxygenase³ | Protein⁴ | Protein⁵ |
| US-670 | 16 | 15 | 3 | 3 | 2 |
| AU-16727 | 16 | 15 | 3 | 3 | 2 |
| US-52 | 16 | 15 | 3 | 3 | 2 |
| US-803 | 16 | 15 | 3 | 3 | 2 |
| US-675 | 16 | 15 | 3 | 3 | 2 |
| US-699 | 16 | 15 | 3 | 3 | 2 |
| US-935 | 16 | 15 | 3 | 3 | 2 |
| US-707 | 16 | 15 | 3 | 3 | 2 |

¹tr|F8TW85|F8TW85_9 SPHN OS = Sphingomonas sp. TTNP3 OX = 436446 GN = hqdC PE = 4 SV = 1
²sp|P23105|XYLG_PSEPU OS = Pseudomonas putida OX = 303 GN = xylG PE = 1 SV = 1
³tr|Q6PW31|Q6PW31_9RALS OS = Ralstonia sp. SJ98 OX = 270371 GN = pnpC PE = 3 SV = 1
⁴tr|G8BJM5|G8BJM5_CANPC OS = Candida parapsilosis (strain CDC 317/ATCC MYA-4646) OX = 578454 GN = HDX1 PE = 3 SV = 1
⁵tr|G8B709|G8B709_CANPC OS = Candida parapsilosis (strain CDC 317/ATCC MYA-4646) OX = 578454 GN = MNX1 PE = 4 SV = 1

The previous analysis indicated that B. bassiana US-707 (ATCC Accession No. PTA-127542) contains two extra copies of the enzyme benzoate para-hydroxylase. Another comparison focusing specifically on the benzoate para-hydroxylase gene CYP53A15 demonstrated that B. bassiana US-707 (ATCC Accession No. PTA-127542) has two more copies of this enzyme than any of the other strains (see Table 25). CYP53A15 is a cytochrome P450 enzyme identified in the fungus Cochliobolus lunatus. This enzyme is a key player in lignin degradation and is important for promoting stable soil carbon. Copy numbers were found by using BlastP to compare genes to B8QM33.

TABLE 25

| Strain | CYP53A15 Gene Copy Number |
|---|---|
| B. bassiana US-707 | 22 |
| B. bassiana US-52 | 20 |
| B. bassiana US-935 | 19 |
| B. bassiana US-670 | 19 |
| B. bassiana US-675 | 19 |
| B. bassiana US-803 | 19 |
| B. bassiana US-699 | 19 |
| B. bassiana AU-16727 | 18 |

Additional Genetic Factors Analysed in the B. bassiana Strains

### Analysis of Secondary Metabolite Clusters and Known Clusters

Secondary metabolite clusters and known clusters in each of the B. bassiana strains were identified using antiSMASH (Medema et al., 2011). The secondary metabolic clusters included non-alpha poly-amino acids like e-polylysin (NAPAA) gene clusters, non-ribosomal peptide synthetase (NRPS) gene clusters, NRPS-like gene clusters, type 1 polyketide synthase (T1PKS) gene clusters, terpene gene clusters, fungal ribosomally synthesized and post-translationally modified peptide (fungal RiPP) clusters, and beta-lactone clusters. The results are presented in Table 26.

TABLE 26

| Cluster | US-52 | US-675 | US-699 | US-707 | US-803 | AU-16727 | US-670 | US-935 |
|---|---|---|---|---|---|---|---|---|
| Beta-Lactone | 0 | 0 | 0 | 0 | 0 | 0 | 0 | 0 |
| Fungal RiPP | 0 | 0 | 0 | 0 | 0 | 0 | 0 | 0 |
| NAPAA | 1 | 1 | 1 | 1 | 1 | 1 | 1 | 1 |
| NRPS | 14 | 15 | 15 | 14 | 15 | 13 | 14 | 15 |
| NRPS-like | 4 | 4 | 4 | 4 | 4 | 4 | 4 | 4 |
| NRPS, T1PKS | 3 | 4 | 4 | 3 | 4 | 2 | 3 | 2 |
| NRPS, Terpene | 1 | 1 | 1 | 1 | 1 | 1 | 1 | 1 |
| T1PKS | 12 | 12 | 11 | 7 | 13 | 7 | 14 | 11 |
| T1PKS, NRPS | 2 | 2 | 1 | 1 | 1 | 2 | 2 | 3 |
| T1PKS, NRPS-like | 0 | 0 | 0 | 0 | 0 | 1 | 0 | 0 |
| Terpene | 5 | 5 | 6 | 5 | 5 | 6 | 6 | 6 |

In particular, B. bassiana US-707 (ATCC Accession No. PTA-127542) was found to be the only strain to be predicted to have a secondary metabolite cluster producing tenelin. Tenelin is a secondary metabolite that has been found to decrease iron-induced oxidative stress (Jirakkakul et al., 2015). Tenelin is also produced to help certain fungi outcompete other fungal species through iron sequestration (Chen et al., 2021).

The known clusters identified in each of the strains are listed below. All strains contain beauvericin. B. bassiana AU-16727 (NMI Accession No. V23/003855) is the only strain that does not have a cluster that produces aphidicolin/aphidicolan-16β-ol/3-deoxyaphidicolin/17-deoxyaphidicolin. B. bassiana AU-16727 (NMI Accession No. V23/003855) is also the only strain found to produce phomasetin.

B. bassiana US-52 (ATCC Accession No. PTA-127541): aphidicolin/aphidicolan-16B-ol/3-deoxyaphidicolin/17-deoxyaphidicolin; bassianolide; beauvericin; clavaric acid; dimethylcoprogen; fumosorinone; nivalenol/deoxynivalenol/3-acetyldeoxynivalenol/15-acetyldeoxynivalenol/neosolaniol/calonectrin/apotrichodiol/isotrichotriol/15-decalonectrin/T-2 toxin/3-acetyl T-2 toxin/trichodiene; oosporein; squalestatin S1.

B. bassiana US-675 (ATCC Accession No. PTA-127540): aphidicolin/aphidicolan-16ß-ol/3-deoxyaphidicolin/17-deoxyaphidicolin; bassianolide; beauvericin; clavaric acid; dimethylcoprogen; fumosorinone; nivalenol/deoxynivalenol/3-acetyldeoxynivalenol/15-acetyldeoxynivalenol/neosolaniol/calonectrin/apotrichodiol/isotrichotriol/15-decalonectrin/T-2 toxin/3-acetyl T-2 toxin/trichodiene; oosporein; secalonic acids; squalestatin S1.

B. bassiana US-699 (ATCC Accession No. PTA-127538): aphidicolin/aphidicolan-16B-ol/3-deoxyaphidicolin/17-deoxyaphidicolin; bassianolide; beauvericin; clavaric acid; dimethylcoprogen; fumosorinone; nivalenol/deoxynivalenol/3-acetyldeoxynivalenol/15-acetyldeoxynivalenol/neosolaniol/calonectrin/apotrichodiol/isotrichotriol/15-decalonectrin/T-2 toxin/3-acetyl T-2 toxin/trichodiene; oosporein; squalestatin S1.

B. bassiana US-707 (ATCC Accession No. PTA-127542): bassianolide; beauvericin; clavaric acid; dimethylcoprogen; nivalenol/deoxynivalenol/3-acetyldeoxynivalenol/15-acetyldeoxynivalenol/neosolaniol/calonectrin/apotrichodiol/isotrichotriol/15-decalonectrin/T-2 toxin/3-acetyl T-2 toxin/trichodiene; oosporein; squalestatin S1; tenellin.

B. bassiana US-803 (ATCC Accession No. PTA-127539): aphidicolin/aphidicolan-16ß-ol/3-deoxyaphidicolin/17-deoxyaphidicolin; bassianolide; beauvericin; clavaric acid; dimethylcoprogen; fumosorinone; nivalenol/deoxynivalenol/3-acetyldeoxynivalenol/15-acetyldeoxynivalenol/neosolaniol/calonectrin/apotrichodiol/isotrichotriol/15-decalonectrin/T-2 toxin/3-acetyl T-2 toxin/trichodiene; oosporein; squalestatin S1.

B. bassiana AU-16727 (NMI Accession No. V23/003855): bassianolide; beauvericin; clavaric acid; dimethylcoprogen; fumosorinone; nivalenol/deoxynivalenol/3-acetyldeoxynivalenol/15-acetyldeoxynivalenol/neosolaniol/calonectrin/apotrichodiol/isotrichotriol/15-decalonectrin/T-2 toxin/3-acetyl T-2 toxin/trichodiene; oosporein; phomasetin; squalestatin S1.

B. bassiana US-670 (ATCC Accession No. PTA-127741): aphidicolin/aphidicolan-16ß-ol/3-deoxyaphidicolin/17-deoxyaphidicolin; bassianolide; beauvericin; clavaric acid; dimethylcoprogen; fumosorinone.

B. bassiana US-935 (ATCC Accession No. PTA-127743): aphidicolin/aphidicolan-16B-ol/3-deoxyaphidicolin/17-deoxyaphidicolin; bassianolide; beauvericin; clavaric acid; dimethylcoprogen; fumosorinone.

### Effector Proteins

Effector proteins were identified first by using SignalP 5 to identify secreted enzymes (Almagro Armenteros). Then, Phobius was used to eliminate any potential transmembrane proteins (Käll et al., 2004). EffectorP 3.0 was used to predict secreted effector proteins (Sperschneider et al., 2022). All effector proteins were then blasted to the Pathogen-Host-Interactions database (PHI-base) and filtered with an E-value cut-off of ≤1E-30 (Urban et al., 2019). PHI-base contains pathogenicity, virulence and effector genes from fungal species. The genome of a virulent B. bassiana strain know to infect mosquitos was included in our analysis for comparison (i.e., GCA_001682635) taken from (Valero-Jiménez et al., 2016)).

Each of the B. bassiana strains had fewer PHI gene counts than the known mosquito pathogen, B. bassiana GCA_001682635, with B. bassiana AU-16727 (NMI Accession No. V23/003855) having the fewest PHI gene counts (see Table 27).

TABLE 27

| Strain | PHI Gene Count |
|---|---|
| B. bassiana GCA_001682635 | 51 |
| B. bassiana US-675 | 50 |
| B. bassiana US-670 | 50 |
| B. bassiana US-699 | 49 |
| B. bassiana US-803 | 48 |
| B. bassiana US-52 | 48 |
| B. bassiana US-935 | 48 |
| B. bassiana US-707 | 47 |
| B. bassiana AU-16727 | 44 |

### Example 12. Phylogenetic Identification of the Strains

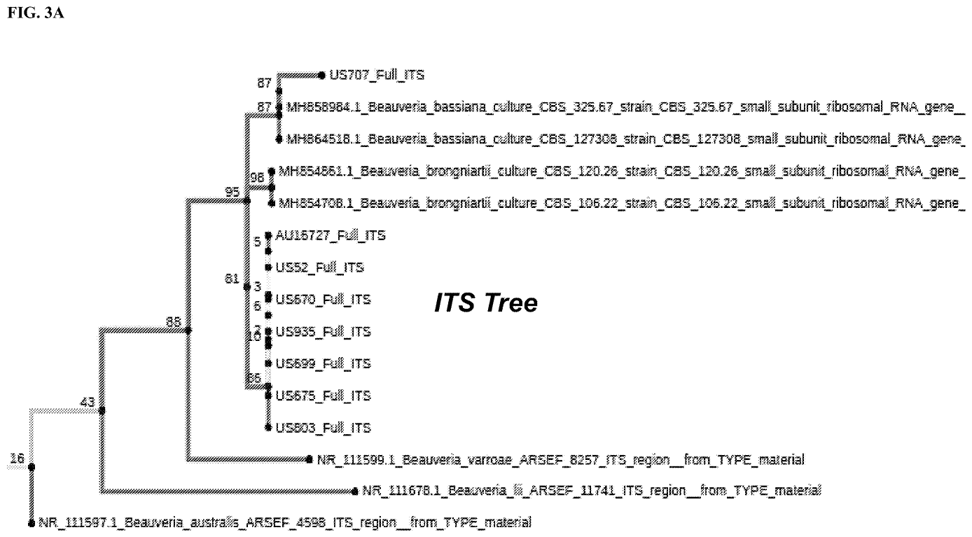

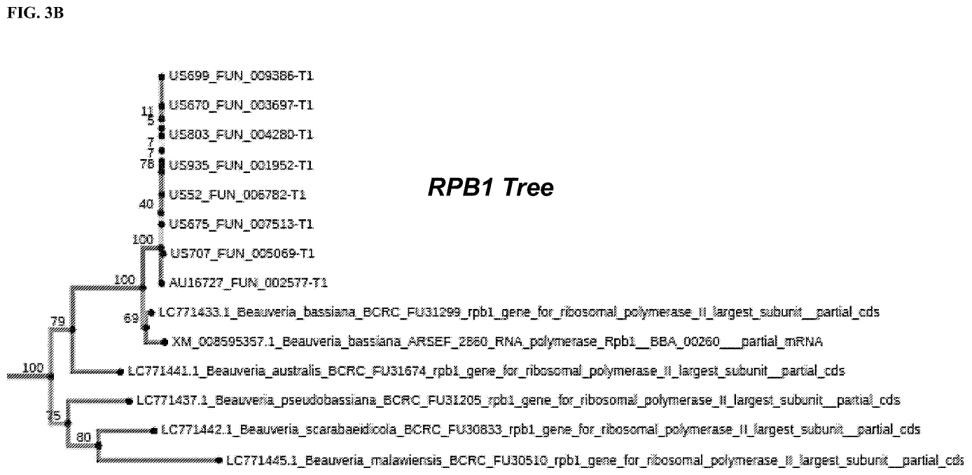

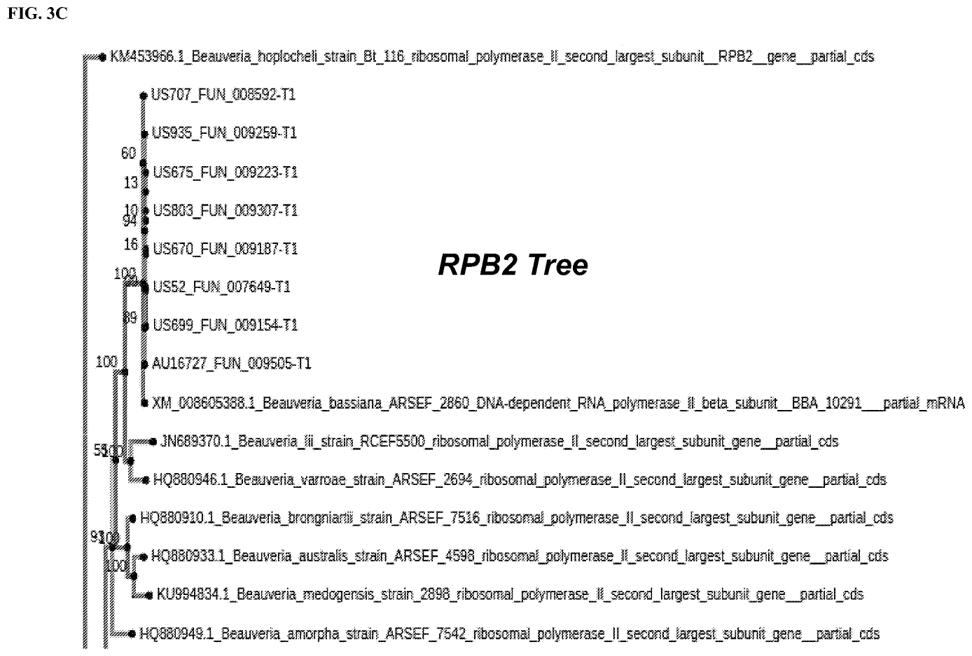

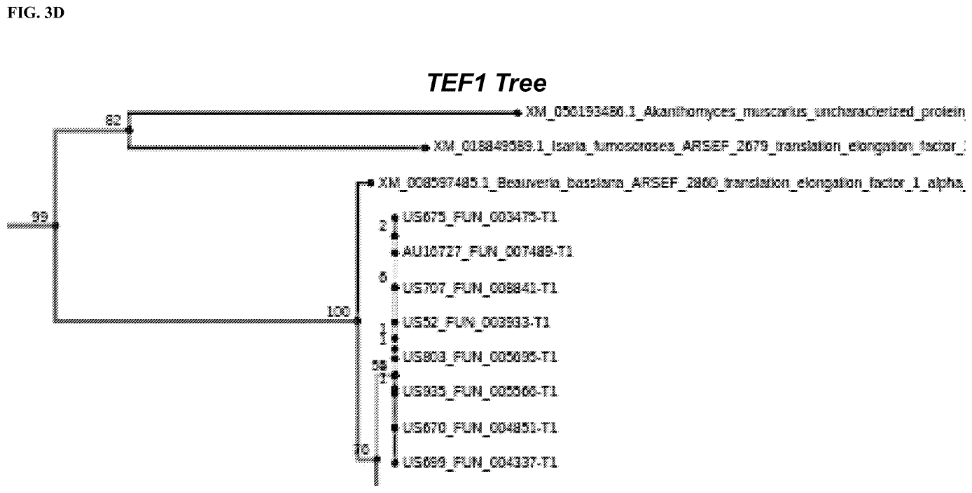

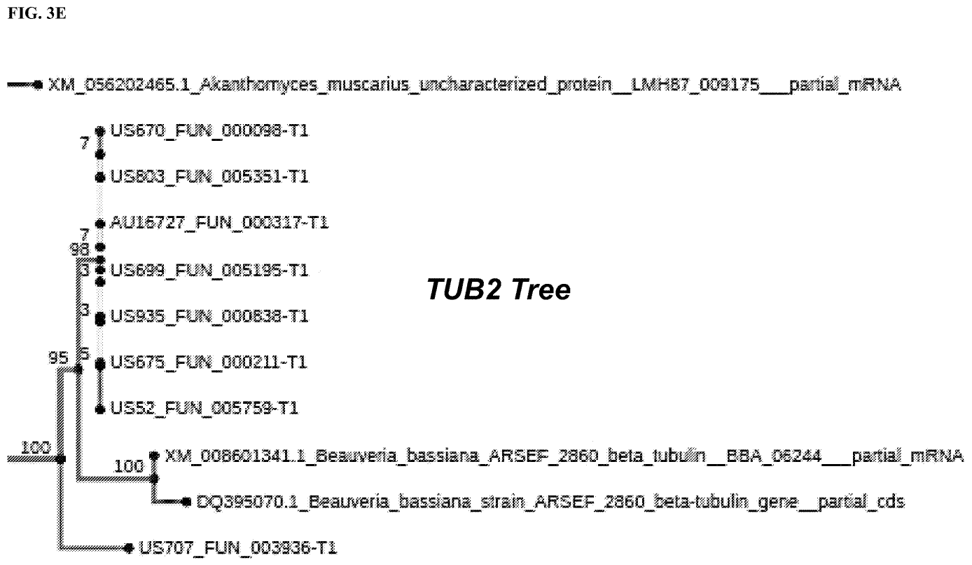

Phylogenetic identification was conducted using five genomic loci: ITS, RPB1, RPB2, TEF1, and TUB2, which were identified as DNA barcode markers by (Lücking et al. 2020). Sequences for each locus were obtained and subjected to BLAST searches against the NCBI nucleotide database, and the top 20 hits were extracted for further analyses. ITS sequences were blasted against both the NCBI database as well as the CBS fungal biobank (Vu et al. 2019). The resulting sequences were aligned using MAFFT with the ‘--auto’ setting (Kato et al. 2002). Phylogenetic trees were inferred from the alignments using RAxML-NG with the “GTR+G” model and 1000 bootstraps (Kozlov et al., 2019). The resulting trees were visualized with Phylo.io (Robinson et al. 2016). The phylogenetic analysis indicated that all strains (i.e., US-52, US-675, US-699, US-707, US-803, AU-16727, US-935, and US-670) are most closely related to Beauveria bassiana (see FIGS. 3A-3E).

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

### REFERENCES

Alexander, D. B., & Zuberer, D. A. (1991). Use of chrome azurol S reagents to evaluate siderophore production by rhizosphere bacteria. Biology and Fertility of soils, 12 (1), 39-45.
Almagro Armenteros J J, Tsirigos K D, Sønderby C K, Petersen T N, Winther O, Brunak S, et al. SignalP 5.0 improves signal peptide predictions using deep neural networks. Nat Biotechnol. 2019 April; 37 (4): 420-3.
Almeida, L. F. J., Souza, I. F., Hurtarte, L. C. C., Teixeira, P. P. C., Inagaki, T. M., Silva, I. R., Mueller, C. W. 2021. Forest litter constraints on the pathways controlling soil organic matter formation. Soil Biology and Biochemistry, 163, 106447.
Bankevich A, Nurk S, Antipov D, Gurevich A A, Dvorkin M, Kulikov A S, et al. SPAdes: A New Genome Assembly Algorithm and Its Applications to Single-Cell Sequencing. J Comput Biol. 2012 May; 19(5):455-77.
Biely P, Cziszárová M, Agger J W, Li X L, Puchart V, Vršanská M, et al. Trichoderma reesei CE16 acetyl esterase and its role in enzymatic degradation of acetylated hemicellulose. Biochim Biophys Acta BBA-Gen Subj. 2014 January; 1840 (1): 516-25.
Bolger A M, Lohse M, Usadel B. Trimmomatic: a flexible trimmer for Illumina sequence data. Bioinformatics. 2014 Aug. 1; 30(15):2114-20.
Buss, W., Sharma, R., Ferguson, C., Borevitz, J. (2021) Soil organic carbon fractionation and metagenomics pipeline to link carbon content and stability with microbial composition-first results investigating fungal endophytes. Genomics.
Chen B, Sun Y, Li S, Yin Y, Wang C. Inductive Production of the Iron-Chelating 2-Pyridones Benefits the Producing Fungus To Compete for Diverse Niches. Turgeon BG, editor. mBio. 2021 Dec. 21; 12(6):e03279-21.
Chenu, C., Rumpel, C., Lehmann, J. (2015) Methods for studying soil organic matter: nature, dynamics, spatial accessibility, and interactions with minerals. Soil Microbiology, Ecology and Biochemistry.
del Cerro C, Erickson E, Dong T, Wong A R, Eder E K, Purvine S O, et al. Intracellular pathways for lignin catabolismin white-rot fungi. Proc Natl Acad Sci. 2021 Mar. 2; 118(9):e2017381118.
Doilom, M., Guo, J. W., Phookamsak, R., Mortimer, P. E., Karunarathna, S. C., Dong, W., . . . & Xu, J. C. (2020). Screening of phosphate-solubilizing fungi from air and soil in Yunnan, China: four novel species in Aspergillus, Gongronella, Penicillium, and Talaromyces. Frontiers in microbiology, 11, 585215.
Feng, W., Plante, A. F., Six, J. (2013) Improving estimates of maximal organic carbon stabilization by fine soil particles. Biogeochemistry, 112, 81-93.
Fernandez C W, Heckman K, Kolka R, Kennedy P G. Melanin mitigates the accelerated decay of mycorrhizal necromass with peatland warming. Klironomos J, editor. Ecol Lett. 2019 March; 22(3):498-505.
Friedlingstein, P, Jones, M. W., O'Sullivan, M., Andrew, R. M., Dorothee, C., Bakker, C. E., Hauck, J, Le Quere, C., Peters, G. P., Peters, W., Pongratz, J., Sitch, S., Canadell, J. G., Ciais, P., Jackson, R. B., Alin, S. R., Anthoni, P., Bates, N. R., Becker, M., Bellouin, N., Bopp, L., Chau, T. T. T., Chevallier, F., Chini, L. P., Cronin, M., Currie, K. I., Decharme, B., Djeutchouang, L. M., Dou, X., Evans W, Feely, R. A., Feng, L., Gasser, T., Gilfillan, D., Gkritzalis, T., Grassi, G., Gregor, L., Gruber, N., Gurses, O., Harris, I., Houghton, R. A., Hurtt, G. C., Ilyina, T., Luijkx, I. C., Jain, A. K., Jones, S. D., Kato, E., Kennedy, D., Goldewijk, K. K., Knauer, J., Korsbakken, J. I., Kortzinger, A., Landschützer, P., Lauvset, S. K., Lefèvre, N., Lienert, S., Liu, J., Marland, G., McGuire, P. C., Melton, J. R., Munro, D. R., Nabel, J. E. M. S., Nakaoka, S. I., Niwa, Y., Ono, T., Pierrot, D., Poulter, B., Rehder, G., Resplandy, L., Robertson, E., Rödenbeck, C., Rosan, T. M., Schwinger, J., Schwingshacki, C., Séférian, R., Sutton, A. J., Sweeney, C., Tanhua, T., Tans, P. P., Tian, H., Tilbrook, B., Tubiello, F., van der Werf, G., Vuichard, N., Wada, C., Wanninkhof, R., Watson, A. J., Willis, D., Wiltshire, A. J., Yuan, W., Yue, C., Yue, X., Zachle, S. Zheng, J. (2021) Global Carbon Budget 2021. Earth Syst Sci Data 14:1917-2005.
Gayathri R, Mahboob S, Govindarajan M, Al-Ghanim K A, Ahmed Z, Al-Mulhm N, et al. A review on biological carbon sequestration: A sustainable solution for a cleaner air environment, less pollution and lower health risks. J King Saud Univ-Sci. 2021 March; 33(2):101282.
Gong Y, Lebreton A, Zhang F, Martin F. Role of carbohydrate-active enzymes in mycorrhizal symbioses. Essays Biochem. 2022 Dec. 23; EBC20220127.
Gougoulias C, Clark J M, Shaw L J. The role of soil microbes in the global carbon cycle: tracking the below-ground microbial processing of plant-derived carbon for manipulating carbon dynamics in agricultural systems: Role of soil microbes in global carbon cycle: carbon tracking & agro-cosystem management. J Sci Food Agric. 2014 September; 94 (12): 2362-71.
Haas B J, Salzberg S L, Zhu W, Pertea M, Allen J E, Orvis J, et al. Automated eukaryotic gene structure annotation using EVidenceModeler and the Program to Assemble Spliced Alignments. Genome Biol. 2008; 9(1):R7.
Hemingway, J. D., Rothman, D. H., Grant, K. E., Rosengard, S. Z., Elington, T. I., Derry, L. A., Galy V. V. (2019). Mineral protection regulates long-term global preservation of natural organic carbon. Nature, 570, 228-231).
Hider, R. C., & Kong, X. (2010). Chemistry and biology of siderophores. Natural product reports, 27(5), 637-657.
Holesova Z, Jakubkova M, Zavadiakova I, Zeman I, Tomaska L, Nosek J. Gentisate and 3-oxoadipate pathways in the yeast Candida parapsilosis: identification and functional analysis of the genes coding for 3-hydroxybenzoate 6-hydroxylase and 4-hydroxybenzoate 1-hydroxylase. Microbiology. 2011 Jul. 1; 157(7):2152-63.
IPCC (2021) Summary for policymakers. In: V Masson-Delmotte, P Zhai, A Pirani, S L Connors, C Péan, S Berger, N Caud, Y Chen, L Goldfarb, M I Gomis, M Huang, K Leitzell, E Lonnoy, J B R Matthews, T K Maycock, T Waterfield, O Yelekçi, R Yu, B Zhou (eds) Climate Change 2021: The Physical Science Basis Contribution of Working Group I to the Sixth Assessment Report of the Intergovernmental Panel on Climate Change. Cambridge University Press.
Jirakkakul J, Cheevadhanarak S, Punya J, Chutrakul C, Senachak J, Buajarern T, et al. Tenellin acts as an iron chelator to prevent iron-generated reactive oxygen species toxicity in the entomopathogenic fungus Beauveria bassiana. FEMS Microbiol Lett. 2015 Jan. 1; 362(2):1-8.
Käll L, Krogh A, Sonnhammer ELL. A Combined Transmembrane Topology and Signal Peptide Prediction Method. J Mol Biol. 2004 May; 338(5):1027-36.
Kleber, M., Eusterhues, K., Keiluweit, M., Mikutta, C., Mikutta, R., Nico, P. S. (2015). Mineral-organic associations: Formation, properties, and relevance in soil environments. Advances in Agronomy, 130, 3-140.
Katoh K, Misawa K, Kuma K, Miyata T. MAFFT: a novel method for rapid multiple sequence alignment based on fast Fourier transform. Nucleic Acids Res. 2002 Jul. 15; 30(14):3059-66.
Korf I. [No title found]. BMC Bioinformatics. 2004; 5(1):59.
Kozlov, A M, et al., RAXML-NG: a fast, scalable and user-friendly tool for maximum likelihood phylogenetic inference, Bioinformatics, Volume 35, Issue 21, November 2019, Pages 4453-4455
Lah L, Podobnik B, Novak M, Korošec B, Berne S, Vogelsang M, et al. The versatility of the fungal cytochrome P450 monooxygenase system is instrumental in xenobiotic detoxification: Fungal P450 systems in xenobiotic detoxification. Mol Microbiol. 2011 September; 81(5):1374-89.
Lal, R. (2018) Digging deeper. A holistic perspective of factors affecting soil carbon sequestration in agroecosystems. Global Change Biology, 24, 3285-3301.
Lavallee, J. M., Soong, J. L, Cotrufo, M. F. (2019) Conceptualizing soil organic matter into particulate and mineral-associated forms to address global change in the 21st century. Global Change Biology, 26, 261-273.
Lenaers M, Reyns W, Czech J, Carleer R, Basak I, Deferme W, et al. Links Between Heathland Fungal Biomass Mineralization, Melanization, and Hydrophobicity. Microb Ecol. 2018 October; 76(3):762-70.
Lücking, R., Aime, M. C., Robbertse, B. et al. Unambiguous identification of fungi: where do we stand and how accurate and precise is fungal DNA barcoding?. IMA Fungus 11, 14 (2020).
Lugato, E., Lavallee, J. M., Haddix, M. L., Panagos, P., Cotrufo M. F. (2021) Different climate sensitivity of particulate and mineral-associated soil organic matter. Nature Geoscience, 14, 295-300.
Majoros W H, Pertea M, Salzberg S L. TigrScan and GlimmerHMM: two open source ab initio eukaryotic gene-finders. Bioinformatics. 2004 Nov. 1; 20(16):2878-9.
Mantzoukas S, Lagogiannis I, Mpousia D, Ntoukas A, Karmakolia K, Eliopoulos P A, et al. Beauveria bassiana Endophytic Strain as Plant Growth Promoter: The Case of the Grape Vine Vitis vinifera. J Fungi. 2021 Feb. 16; 7(2):142.
Mayer M., Krause, H-M., Fliessbach, A., Mader, P., Steffens, M. (2022) Fertilizer quality and labile soil organic matter fractions are vital for organic carbon sequestration in temperate arable soils within a long-term trial in Switzerland. Geoderma, 426, 116080.
McGee P, Mukasa Mugerwa T. Melanised endophytic fungi may increase stores of organic carbon in soil. 2013 Apr. 1; EGU2013-2136.
Medema M H, Blin K, Cimermancic P, de Jager V, Zakrzewski P, Fischbach M A, et al. antiSMASH: rapid identification, annotation and analysis of secondary metabolite biosynthesis gene clusters in bacterial and fungal genome sequences. Nucleic Acids Res. 2011 Jul. 1; 39(suppl_2):W339-46.
Mendes, F K, et al., CAFE 5 models variation in evolutionary rates among gene families, Bioinformatics, Volume 36, Issue 22-23, December 2020, Pages 5516-5518.
Merino C, Godoy R, Matus F. Soil enzymes and biological activity at different levels of organic matter stability. Journal of Soil Science and Plant Nutrition. 2016; (16):14-30.
Mistry, J. et al., Pfam: The protein families database in 2021, Nucleic Acids Research, Volume 49, Issue D1, 8 Jan. 2021, Pages D412-D419.
Mugerwa, M. T. T., McGee, P. A. (2017) Potential effect of melanised endophytic fungi on levels of organic carbon within an Alfisol. Soil Research 55, 245-252.
Ortiz-Urquiza A. The Split Personality of Beauveria bassiana: Understanding the Molecular Basis of Fungal Parasitism and Mutualism. mSystems. 2021 Aug. 31; 6(4):e00766-21.
Poeplau, C., Don, A., Six, J., Kaiser, M., Benbi, D., Chenu, C., Cotrufo, M. F., Derrien, D., Gioacchini, P., Grand, S., Gregorich, E., Griepentrog, M., Gunina, A., Haddix, M., Kuzyakov, Y., Kuhnel, A., Macdonald, L. M., Soong, J., Trigalet, S., Vermeire, M-L., Rovira, P., van Wesemael, B., Wiesmeier, M., Yeasmin, S., Yevdokimov, I., Nieder, R. (2018) Isolating organic carbon fractions with varying turnover rates in temperate agricultural soils-A comprehensive method comparison. Soil Biology and Biochemistry, 125, 10-26.
Quevillon E, Silventoinen V, Pillai S, Harte N, Mulder N, Apweiler R, Lopez R. InterProScan: protein domains identifier. Nucleic Acids Res. 2005 Jul. 1; 33 (Web Server issue):W116-20.
Rehner, S. A., Posada, F., Buckley, E. P., Infante, F., Castillo, A., & Vega, F. E. (2006). Phylogenetic origins of African and Neotropical Beauveria bassiana sl pathogens of the coffee berry borer, Hypothenemus hampei. Journal of invertebrate pathology, 93(1), 11-21.
Robinson, O. et al., Phylo.io: Interactive Viewing and Comparison of Large Phylogenetic Trees on the Web, Molecular Biology and Evolution, Volume 33, Issue 8, August 2016, Pages 2163-2166.
Rocci, K. S., Lavallee, J. M., Stewart C. E., Cotrufo, M. F. (2021) Soil organic carbon response to global environmental change depends on its distribution between mineral-associated and particulate organic matter. Science of the Total Environment, 793, 148569.
Rodrigues, L. A. T., Giacomini, S. J., Diecknow, J., Cherubin, R., Ottonelli, A. S., Bayer, C. (2022) Carbon saturation deficit and litter quality drive the stabilization of litter-derived C in mineral-associated organic matter in long-term no-till soil. Catena, 219, 106590.
Salam L B. Detection of carbohydrate-active enzymes and genes in a spent engine oil-perturbed agricultural soil. Bull Natl Res Cent. 2018 December; 42(1):10.
Sanderman, J., Hengl, T., Fiske, G. J. (2017) Soil carbon debt of 12,000 years of human land use. Proceedings of the National Academy of Sciences, 114, 9575-9580.
Schweizer, S. A., Mueller, C. W., Hoschen, C., Ivanov, P., Kogel-Knabner, I. (2021) The role of clay content and mineral surface area for soil organic carbon storage in an arable toposequence. Biogeochemistry, 156, 401-420.
Sharma, S. B., Sayyed, R. Z., Trivedi, M. H., & Gobi, T. A. (2013). Phosphate solubilizing microbes: sustainable approach for managing phosphorus deficiency in agricultural soils. SpringerPlus, 2(1), 1-14.
Six, J. (1999). Recycling of sodium polytungstate used in soil organic matter studies. Soil Biology and Biochemistry, 31 (8), 1193-1196.
Sperschneider J, Dodds P N. EffectorP 3.0: Prediction of Apoplastic and Cytoplasmic Effectors in Fungi and Oomycetes. Mol Plant-Microbe Interactions®. 2022 February; 35(2):146-56.
Stanke M, Keller O, Gunduz I, Hayes A, Waack S, Morgenstern B. AUGUSTUS: ab initio prediction of alternative transcripts. Nucleic Acids Res. 2006 Jul. 1; 34(Web Server):W435-9.
Sun, J. et al., OrthoVenn3: an integrated platform for exploring and visualizing orthologous data across genomes, Nucleic Acids Research, Volume 51, Issue W1, 5 Jul. 2023, Pages W397-W403.
Ter-Hovhannisyan V, Lomsadze A, Chernoff Y O, Borodovsky M. Gene prediction in novel fungal genomes using an ab initio algorithm with unsupervised training. Genome Res. 2008 December; 18(12):1979-90.
Urban M, Cuzick A, Seager J, Wood V, Rutherford K, Venkatesh S Y, et al. PHI-base: the pathogen-host interactions database. Nucleic Acids Res. 2019 Nov. 16; gkz904.
Valero-Jiménez C A, Faino L, Spring in't Veld D, Smit S, Zwaan B J, van Kan J A L. Comparative genomics of Beauveria bassiana: uncovering signatures of virulence against mosquitoes. BMC Genomics. 2016 December; 17(1):986.
Vu D, Groenewald M, de Vries M, Gehrmann T, Stielow B, Eberhardt U, Al-Hatmi A, Groenewald J Z, Cardinali G, Houbraken J, Boekhout T, Crous P W, Robert V, Verkley G J M. Large-scale generation and analysis of filamentous fungal DNA barcodes boosts coverage for kingdom fungi and reveals thresholds for fungal species and higher taxon delimitation. Stud Mycol. 2019 March; 92:135-154.
Xu L, Dong Z, Fang L, Luo Y, Wei Z, Guo H, et al. Ortho Venn2: a web server for whole-genome comparison and annotation of orthologous clusters across multiple species. Nucleic Acids Res. 2019 Jul. 2; 47(W1):W52-8.
Yan Z, Kang E, Zhang K, Hao Y, Wang X, Li Y, et al. Asynchronous responses of microbial CAZymes genes and the net CO2 exchange in alpine peatland following 5 years of continuous extreme drought events. ISME Commun. 2022 Nov. 16; 2(1):115.

1. A method of increasing soil organic carbon (SOC), comprising:
heterologously disposing one or more fungal strains to treat a plant element,
wherein the one or more fungal strains comprises a nuclear ribosomal internal transcribed spacer (ITS) sequence that is at least 97% identical to the nucleotide sequence of any one of SEQ ID NOs: 1-8; and
the one or more fungal strains are heterologously disposed in an effective amount to increase organic carbon in soil supporting a plant derived from the treated plant element relative to soil supporting a reference plant derived from a reference plant element.

2. (canceled)

3. The method of claim 1, wherein the one or more fungal strains further comprises:
(i) a B Locus Nuclear Intergenic Region (Bloc) sequence that is at least 97% identical to the nucleotide sequence of SEQ ID NO: 9 or 10;
(ii) an RPB1 sequence that is at least 97% identical to the nucleotide sequence of any one of SEQ ID NOs: 11-18;
(iii) an RPB2 sequence that is at least 97% identical to the nucleotide sequence of any one of SEQ ID NOs: 19-26;
(iv) a TEF1 sequence that is at least 97% identical to the nucleotide sequence of any one of SEQ ID NOs: 27-34;
(v) a TUB2 sequence that is at least 97% identical to the nucleotide sequence of any one of SEQ ID NOs: 35-42; or
(vi) any combination (i) to (v).

4. The method of claim 3, wherein the one or more fungal strains are selected from the group consisting of: Beauveria bassiana AU-16727 (NMI Accession No. V23/003855), B. bassiana US-52 (ATCC Accession No. PTA-127541), B. bassiana US-675 (ATCC Accession No. PTA-127540), B. bassiana US-699 (ATCC Accession No. PTA-127538), B. bassiana US-707 (ATCC Accession No. PTA-127542), B. bassiana US-803 (ATCC Accession No. PTA-127539), B. bassiana US-670 (ATCC Accession No. PTA-127741), B. bassiana US-935 (ATCC Accession No. PTA-127743), and a mutant thereof having all identifying characteristics of the respective strain.

5. The method of claim 1, further comprising an initial step of identifying the soil as having a soil organic carbon (SOC) (% wt/wt) below a threshold level of 5%.

6. (canceled)

7. The method of claim 1, wherein the plant element is non-native to the one or more fungal strains and the non-native plant element is from a plant selected from the group consisting of wheat, rice, corn (maize), canola, rye, oats, barley, sorghum, millet, flax, hemp, jute, cotton, sugar cane, soybeans, alfalfa, clover, Desmanthus, peanuts, lentils, lupins, peas, and chickpea.

8-10. (canceled)

11. A method for sequestering atmospheric carbon for storage as soil organic carbon (SOC), comprising:
heterologously disposing one or more fungal strains to treat a plant element,
wherein the one or more fungal strains comprises a nuclear ribosomal internal transcribed spacer (ITS) sequence that is at least 97% identical to the nucleotide sequence of any one of SEQ ID NOs: 1-8; and
the one or more fungal strains are in an effective amount to increase sequestered atmospheric carbon in soil supporting a plant derived from the treated plant element relative to soil supporting a reference plant derived from a reference plant element.

12. (canceled)

13. The method of claim 11, wherein the one or more fungal strains further comprises:
(i) a B Locus Nuclear Intergenic Region (Bloc) sequence that is at least 97% identical to the nucleotide sequence of SEQ ID NO: 9 or 10;
(ii) an RPB1 sequence that is at least 97% identical to the nucleotide sequence of any one of SEQ ID NOs: 11-18;
(iii) an RPB2 sequence that is at least 97% identical to the nucleotide sequence of any one of SEQ ID NOs: 19-26;
(iv) a TEF1 sequence that is at least 97% identical to the nucleotide sequence of any one of SEQ ID NOs: 27-34;
(v) a TUB2 sequence that is at least 97% identical to the nucleotide sequence of any one of SEQ ID NOs: 35-42; or
(vi) any combination (i) to (v).

14. The method of claim 13, wherein the one or more fungal strains are selected from the group consisting of: Beauveria bassiana AU-16727 (NMI Accession No. V23/003855), B. bassiana US-52 (ATCC Accession No. PTA-127541), B. bassiana US-675 (ATCC Accession No. PTA-127540), B. bassiana US-699 (ATCC Accession No. PTA-127538), B. bassiana US-707 (ATCC Accession No. PTA-127542), and B. bassiana US-803 (ATCC Accession No. PTA-127539), B. bassiana US-670 (ATCC Accession No. PTA-127741), B. bassiana US-935 (ATCC Accession No. PTA-127743), and a mutant thereof having all identifying characteristics of the respective strain.

15. The method of claim 11, wherein the plant element is non-native to the one or more fungal strains and the non-native plant element is from a plant selected from the group consisting of wheat, rice, corn (maize), canola, rye, oats, barley, sorghum, millet, flax, hemp, jute, cotton, sugar cane, soybeans, alfalfa, clover, Desmanthus, peanuts, lentils, lupins, peas, and chickpea.

16-18. (canceled)

19. The method of claim 11, wherein the one or more fungal strains increase organic carbon in the soil by expressing enzymes involved in the melanin biosynthetic pathway, carbohydrate active enzymes, lignin degrading enzymes, or a combination thereof.

20-27. (canceled)

28. A synthetic combination comprising a purified population of one or more fungal strains heterologously disposed to a plant element,
wherein the one or more fungal strains are heterologous to the plant element and comprise a nuclear ribosomal internal transcribed spacer (ITS) sequence that is at least 97% identical to the nucleotide sequence of any one of SEQ ID NOs: 1-8; and an agriculturally acceptable carrier;
wherein the one or more fungal strains are present in an effective amount to increase soil organic carbon (SOC) in soil supporting a plant derived from the plant element in the synthetic combination relative to soil supporting a reference plant derived from a reference plant element.

29. The synthetic combination of claim 28, wherein the one or more fungal strains further comprises:
(i) a B Locus Nuclear Intergenic Region (Bloc) sequence that is at least 97% identical to the nucleotide sequence of SEQ ID NO: 9 or 10;
(ii) an RPB1 sequence that is at least 97% identical to the nucleotide sequence of any one of SEQ ID NOs: 11-18;
(iii) an RPB2 sequence that is at least 97% identical to the nucleotide sequence of any one of SEQ ID NOs: 19-26;
(iv) a TEF1 sequence that is at least 97% identical to the nucleotide sequence of any one of SEQ ID NOs: 27-34;
(v) a TUB2 sequence that is at least 97% identical to the nucleotide sequence of any one of SEQ ID NOs: 35-42; or
(vi) any combination (i) to (v).

30. The synthetic combination of claim 28, wherein the one or more fungal strains are selected from the group consisting of: Beauveria bassiana AU-16727 (NMI Accession No. V23/003855), B. bassiana US-52 (ATCC Accession No. PTA-127541), B. bassiana US-675 (ATCC Accession No. PTA-127540), B. bassiana US-699 (ATCC Accession No. PTA-127538), B. bassiana US-707 (ATCC Accession No. PTA-127542), and B. bassiana US-803 (ATCC Accession No. PTA-127539), B. bassiana US-670 (ATCC Accession No. PTA-127741), B. bassiana US-935 (ATCC Accession No. PTA-127743), and a mutant thereof having all identifying characteristics of the respective strain.

31. The synthetic combination of claim 28, wherein the plant element is non-native to the one or more fungal strains.

32. The synthetic combination of claim 31, wherein the non-native plant element is from a plant selected from the group consisting of wheat, rice, corn (maize), canola, rye, oats, barley, sorghum, millet, flax, hemp, jute, cotton, sugar cane, soybeans, alfalfa, clover, Desmanthus, peanuts, lentils, lupins, peas, and chickpea.

33. The synthetic combination of claim 31, wherein the non-native plant element is from a pasture crop or cover crop selected from the group consisting of lucerne, arrow leaf clover, balansa clover, chicory, plantain, phalaris, cocksfoot, fescue, prairie grass, Warrego summer grass, Italian rye grass, perennial rye grass, biserrula, serradella, gland clover, bladder clover, switchgrass, radish, medic, buckwheat, cow pea, lablab, sunn hemp, sunflower, tillage radish, and subterranean clover.

34. The synthetic combination of claim 28, wherein (a) the combination is formulated as a solid, liquid or gel; (b) the combination is formulated as a powder, pellet or granules; or (c) the combination is formulated as an emulsion, colloid, suspension or solution.

35. The synthetic combination of claim 28, wherein the one or more fungal strains are present in the combination at a concentration of at least 10³ colony forming units (CFU) per milliliter or gram.

36. The synthetic combination of claim 28, wherein the plant element is a whole plant, seedling, meristematic tissue, ground tissue, vascular tissue, dermal tissue, seed, leaf, root, shoot, stem, flower, fruit, stolon, bulb, tuber, corm, keikis, shoot, or bud.

37. (canceled)

38. A bag or container comprising the synthetic combination of claim 28.

39-44. (canceled)
